SEQUENCE LISTING (635 sequences)

SEQ ID NO: 1 - DNA, 8811 bp, synthetic construct
  misc_feature 1..8811: note = Synthetic Construct
  source 1..8811: mol_type = other DNA
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 2 - DNA, 8232 bp, synthetic construct
  misc_feature 1..8232: note = Synthetic Construct
  source 1..8232: mol_type = other DNA
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggctagcatgaaacggacagccgacggaagcgagttcgagtcaccaaagaagaagcggaaagtcggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacaggccatgccgaaattatggccctgaggcagggcggcctggtcatgcagaactacagactgattgacgccaccctgtacgggacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 3 - DNA, 9254 bp, synthetic construct
  misc_feature 1..9254: note = Synthetic Construct
  source 1..9254: mol_type = other DNA
aaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattccatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagcgcgtgatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgcccactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgccaccatggctagcatgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgtctcagttatgacaccgaaatcctgacagtcgagtatggatttctgccgatcggcaagattgtggaggagagaattgaatgtacggtctatacggtcgacaagaatggtttcgtctacacccaaccaattgctcaatggcataatcgaggggagcaggaggtgtttgagtattgcctggaggacgggtcaatcattagagctacaaaggaccataagtttatgacaaccgatggtcaaatgctgccgatagatgaaatattcgaaaggggactggatcttaagcaagtcgatggccttccaaactagtagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttatctcctgttctgcagcgtttcagagctatgctggaaacagcatagcaagttgaaataaggctagtccgttatcaacttgaaaaagtggcaccgagtcggtgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaa

SEQ ID NO: 4 - DNA, 8538 bp, synthetic construct
  misc_feature 1..8538: note = Synthetic Construct
  source 1..8538: mol_type = other DNA
aaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattccatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagcgcgtgatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgcccactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgccaccatggtcaagattatcagccgcaaatccttggggacacagaatgtatatgacatcggcgtggaaaaggatcacaattttctgctgaagaatggtcttgttgcttccaattgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaactagtagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttatctcctgttctgcagcgtttcagagctatgctggaaacagcatagcaagttgaaataaggctagtccgttatcaacttgaaaaagtggcaccgagtcggtgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaa

SEQ ID NO: 5 - DNA, 7098 bp, synthetic construct
  misc_feature 1..7098: note = Synthetic Construct
  source 1..7098: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagcgcgtgatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgcccactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgccaccatggctagcatgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgtttggatctgaaaacgcaagttcaaacgccacagggtatgaaagaaatatccaatatacaggtcggcgatctcgtcttgtctaacactggctataacgaggtgctgaatgtatttccaaaaagcaagaaaaaaagttacaagataactctggaagatggaaaagaaattatctgttctgaggagcatctgtttccgacccaaacaggggagatgaatatcagtggcggtctcaaagagggtatgtgtttgtatgtcaaggaataactagtagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttatctcctgttctgcagcgtttcagagctatgctggaaacagcatagcaagttgaaataaggctagtccgttatcaacttgaaaaagtggcaccgagtcggtgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 6441 bp, synthetic construct
  misc_feature 1..6441: note = Synthetic Construct
  source 1..6441: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagcgcgtgatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgcccactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagccaccatgatgctcaagaagatcctcaagattgaagagttggacgagcgcgagcttatagacatagaagtcagtggtaatcaccttttctacgcaaatgacattttgactcacaactcctcttcagacgtttgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaactagtagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttatctcctgttctgcagcgtttcagagctatgctggaaacagcatagcaagttgaaataaggctagtccgttatcaacttgaaaaagtggcaccgagtcggtgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - DNA, 6644 bp, synthetic construct
  misc_feature 1..6644: note = Synthetic Construct
  source 1..6644: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgactgtagatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgccctccctggggacagcccctcctggctagtcacaccctgtaggctcctctatataacccaggggcacaggggctgccctcattctaccaccacctccacagcacagacagacactcaggagccagcccaccatggctagcatgaaacggacagccgacggaagcgagttcgagtcaccaaagaagaagcggaaagtcggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacaggccatgccgaaattatggccctgaggcagggcggcctggtcatgcagaactacagactgattgacgccaccctgtacgggacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgtttggatctgaaaacgcaagttcaaacgccacagggtatgaaagaaatatccaatatacaggtcggcgatctcgtcttgtctaacactggctataacgaggtgctgaatgtatttccaaaaagcaagaaaaaaagttacaagataactctggaagatggaaaagaaattatctgttctgaggagcatctgtttccgacccaaacaggggagatgaatatcagtggcggtctcaaagagggtatgtgtttgtatgtcaaggaataactagtagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttatctcctgttctgcagcgtttcagagctatgctggaaacagcatagcaagttgaaataaggctagtccgttatcaacttgaaaaagtggcaccgagtcggtgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 6575 bp, synthetic construct
  misc_feature 1..6575: note = Synthetic Construct
  source 1..6575: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgactgtagatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgccctccctggggacagcccctcctggctagtcacaccctgtaggctcctctatataacccaggggcacaggggctgccctcattctaccaccacctccacagcacagacagacactcaggagccagctagccaccatgatgctcaagaagatcctcaagattgaagagttggacgagcgcgagcttatagacatagaagtcagtggtaatcaccttttctacgcaaatgacattttgactcacaactcctcttcagacgtttgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaactagtagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttatctcctgttctgcagcgtttcagagctatgctggaaacagcatagcaagttgaaataaggctagtccgttatcaacttgaaaaagtggcaccgagtcggtgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 9962 bp, synthetic construct
  misc_feature 1..9962: note = Synthetic Construct
  source 1..9962: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagctagcaggtacctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttatctcctgttctgcagcgtttcagagctatgctggaaacagcatagcaagttgaaataaggctagtccgttatcaacttgaaaaagtggcaccgagtcggtgcttttttgcggccgcggatcctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagaattcgatatcaagcttatcggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 10 - DNA, 7580 bp, synthetic construct
  misc_feature 1..7580: note = Synthetic Construct
  source 1..7580: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccctgtacaagcaatgtactaactacgctttgttgaaactcgctggcgatgttgaaagtaaccccggtcctgaattccaagaacagctgcagaacaggagataacagttgggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 11 - protein, 1368 aa, synthetic construct
  REGION 1..1368: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 1309 aa, synthetic construct
  REGION 1..1309: note = Synthetic Construct
EATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 13 - protein, 1368 aa, synthetic construct
  REGION 1..1368: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 1368 aa, synthetic construct
  REGION 1..1368: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 15 - protein, 1368 aa, synthetic construct
  REGION 1..1368: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKQYRSTKEVLDATLIHQSITGLYETRIDLSQLGGD

SEQ ID NO: 16 - protein, 1368 aa, synthetic construct
  REGION 1..1368: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKEYRSTKEVLDATLIHQSITGLYETRIDLSQLGGD

SEQ ID NO: 17 - protein, 1368 aa, synthetic construct
  REGION 1..1368: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKQYRSTKEVLDATLIHQSITGLYETRIDLSQLGGD

SEQ ID NO: 18 - protein, 1368 aa, synthetic construct
  REGION 1..1368: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKEYRSTKEVLDATLIHQSITGLYETRIDLSQLGGD

SEQ ID NO: 19 - protein, 1378 aa, synthetic construct
  REGION 1..1378: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYVGADKKLRKRSSKLATEEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 20 - protein, 1378 aa, synthetic construct
  REGION 1..1378: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYVGADKKLRKRSSKLATEEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 21 - protein, 1375 aa, synthetic construct
  REGION 1..1375: note = Synthetic Construct
MEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 22 - protein, 364 aa, synthetic construct
  REGION 1..364: note = Synthetic Construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 23 - protein, 166 aa, synthetic construct
  REGION 1..166: note = Synthetic Construct
SEVEFSHEYWMRHALTLAKRARDEREVPVGAVLVLNNRVIGEGWNRAIGLHDPTGHAEIMALRQGGLVMQNYRLIDATLYGTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 24 - protein, 88 aa, synthetic construct
  REGION 1..88: note = Synthetic Construct
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 25 - protein, 38 aa, synthetic construct
  REGION 1..38: note = Synthetic Construct
MMLKKILKIEELDERELIDIEVSGNHLFYANDILTHNS

SEQ ID NO: 26 - protein, 102 aa, synthetic construct
  REGION 1..102: note = Synthetic Construct
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPN

SEQ ID NO: 27 - protein, 36 aa, synthetic construct
  REGION 1..36: note = Synthetic Construct
MVKIISRKSLGTQNVYDIGVEKDHNFLLKNGLVASN

SEQ ID NO: 28 - DNA, 353 bp, synthetic construct
  misc_feature 1..353: note = Synthetic Construct
  source 1..353: mol_type = other DNA
tcgagcgcgtgatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgcccactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 29 - DNA, 483 bp, synthetic construct
  misc_feature 1..483: note = Synthetic Construct
  source 1..483: mol_type = other DNA
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgactgtagatgagagcagccactacgggtctaggctgcccatgtaaggaggcaaggcctggggacacccgagatgcctggttataattaacccagacatgtggctgcccccccccccccaacacctgctgcctgctaaaaataaccctgtccctggtggccctgcatgccctccctggggacagcccctcctggctagtcacaccctgtaggctcctctatataacccaggggcacaggggctgccctcattctaccaccacctccacagcacagacagacactcaggagccagc

SEQ ID NO: 30 - DNA, 4104 bp, synthetic construct
  misc_feature 1..4104: note = Synthetic Construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 31 - DNA, 4104 bp, synthetic construct
  misc_feature 1..4104: note = Synthetic Construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgac

SEQ ID NO: 32 - DNA, 4048 bp, synthetic construct
  misc_feature 1..4048: note = Synthetic Construct
  source 1..4048: mol_type = other DNA
cgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggataccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagctgtacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcatcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgagaaggtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgaccagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatccagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgtgctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 33 - DNA, 4104 bp, synthetic construct
  misc_feature 1..4104: note = Synthetic Construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggataccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagctgtacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcatcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgagaaggtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgaccagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatccagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgac

SEQ ID NO: 34 - DNA, 4104 bp, synthetic construct
  misc_feature 1..4104: note = Synthetic Construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaagcagtaccggagcaccaaagaggtgctggacgccaccctgatccaccagagcatcaccggcctgtacgagacacggatcgacctgtctcagctgggaggtgac

SEQ ID NO: 35 - DNA, 4104 bp, synthetic construct
  misc_feature 1..4104: note = Synthetic Construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggagtaccggagcaccaaagaggtgctggacgccaccctgatccaccagagcatcaccggcctgtacgagacacggatcgacctgtctcagctgggaggtgac

SEQ ID NO: 36 - DNA, 4104 bp, synthetic construct
  misc_feature 1..4104: note = Synthetic Construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggataccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagctgtacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcatcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgagaaggtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgaccagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatccagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaagcagtaccggagcaccaaagaggtgctggacgccaccctgatccaccagagcatcaccggcctgtacgagacacggatcgacctgtctcagctgggaggtgac

SEQ ID NO: 37 - DNA, 4104 bp, synthetic construct
  misc_feature 1..4104: note = Synthetic Construct
  source 1..4104: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggataccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagctgtacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcatcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgagaaggtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgaccagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatccagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggagtaccggagcaccaaagaggtgctggacgccaccctgatccaccagagcatcaccggcctgtacgagacacggatcgacctgtctcagctgggaggtgac

SEQ ID NO: 38 - DNA, 4134 bp, synthetic construct
  misc_feature 1..4134: note = Synthetic Construct
  source 1..4134: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacgtgggcgccgacaagaagctgcgcaagcgcagctctaaactggccacagaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgac

SEQ ID NO: 39 - DNA, 4134 bp, synthetic construct
  misc_feature 1..4134: note = Synthetic Construct
  source 1..4134: mol_type = other DNA
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggataccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagctgtacgacgagcaccaccaggacctgaccctgctgaaagctctcgtgcggcagcagctgcctgagaagtacaaagagattttcttcgaccagagcaagaacggctacgccggctacgtgggcgccgacaagaagctgcgcaagcgcagctctaaactggccacagaggaagagttctacaagttcatcaagcccatcctggaaaagatggacggcaccgaggaactgctcgtgaagctgaacagagaggacctgctgcggaagcagcggaccttcgacaacggcatcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgagaaggtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgaccagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatccagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatccggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgctttctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctcgggccttcaagtactttgacaccaccatcgaccggaaggtgtaccggagcaccaaagaggtgctggacgccaccctgatccaccagagcatcaccggcctgtacgagacacggatcgacctgtctcagctgggaggtgac

SEQ ID NO: 40 - DNA, 4125 bp, synthetic construct
  misc_feature 1..4125: note = Synthetic Construct
  source 1..4125: mol_type = other DNA
atggagaagaagtacagcatcggcctggccatcggcaccaacagcgtgggctgggccgtgatcaccgacgactacaaggtgcccagcaagaagttcaaggtgctgggcaacaccaaccgcaagagcatcaagaagaacctgatgggcgccctgctgttcgacagcggcgagaccgccgaggccacccgcctgaagcgcaccgcccgccgccgctacacccgccgcaagaaccgcatccgctacctgcaggagatattcgccaacgagatggccaagctggacgacagcttcttccagcgcctggaggagagcttcctggtggaggaggacaagaagaacgagcgccaccccatcttcggcaacctggccgacgaggtggcctaccaccgcaactaccccaccatctaccacctgcgcaagaagctggccgacagccccgagaaggccgacctgcgcctgatctacctggccctggcccacatcatcaagttccgcggccacttcctgatcgagggcaagctgaacgccgagaacagcgacgtggccaagctgttctaccagctgatccagacctacaaccagctgttcgaggagagccccctggacgagatcgaggtggacgccaagggcatcctgagcgcccgcctgagcaagagcaagcgcctggagaagctgatcgccgtgttccccaacgagaagaagaacggcctgttcggcaacatcatcgccctggccctgggcctgacccccaacttcaagagcaacttcgacctgaccgaggacgccaagctgcagctgagcaaggacacctacgacgacgacctggacgagctgctgggccagatcggcgaccagtacgccgacctgttcagcgccgccaagaacctgagcgacgccatcctgctgagcgacatcctgcgcagcaacagcgaggtgaccaaggcccccctgagcgccagcatggtgaagcgctacgacgagcaccaccaggacctggccctgctgaagaccctggtgcgccagcagttccccgagaagtacgccgagatattcaaggacgacaccaagaacggctacgccggctacgtgggcatcggcatcaagcaccgcaagcgcaccaccaagctggccacccaggaggagttctacaagttcatcaagcccatcctggagaagatggacggcgccgaggagctgctggccaagctgaaccgcgacgacctgctgcgcaagcagcgcaccttcgacaacggcagcatcccccaccagatccacctgaaggagctgcacgccatcctgcgccgccaggaggagttctaccccttcctgaaggagaaccgcgagaagatcgagaagatcctgaccttccgcatcccctactacgtgggccccctggcccgcggcaacagccgcttcgcctggctgacccgcaagagcgaggaggccatcaccccctggaacttcgaggaggtggtggacaagggcgccagcgcccagagcttcatcgagcgcatgaccaacttcgacgagcagctgcccaacaagaaggtgctgcccaagcacagcctgctgtacgagtacttcaccgtgtacaacgagctgaccaaggtgaagtacgtgaccgagcgcatgcgcaagcccgagttcctgagcggcgagcagaagaaggccatcgtggacctgctgttcaagaccaaccgcaaggtgaccgtgaagcagctgaaggaggactacttcaagaagatcgagtgcttcgacagcgtggagatcatcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccactacaccggctggggccgcctgagccgcaagatgatcaacggcatccgcgacaagcagagcggcaagaccatcctggacttcctgaagagcgacggcttcagcaaccgcaacttcatgcagctgatccacgacgacagcctgaccttcaaggaggagatcgagaaggcccaggtgagcggccagggcgacagcctgcacgagcagatcgccgacctggccggcagccccgccatcaagaagggcatcctgcagaccgtgaagatcgtggacgagctggtgaaggtgatgggccacaagcccgagaacatcgtgatcgagatggcccgcgagaaccagaccaccaccaagggcctgcagcagagccgcgagcgcaagaagcgcatcgaggagggcatcaaggagctggagagccagatcctgaaggagaaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcatcaaggacgacagcatcgacaacaaggtgctgacccgcagcgtggagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgaggccgacaaggccggcttcatcaagcgccagctggtggagacccgccagatcaccaagcacgtggcccgcatcctggacagccgcatgaacaccaagcgcgacaagaacgacaagcccatccgcgaggtgaaggtgatcaccctgaagagcaagctggtgagcgacttccgcaaggacttccagctgtacaaggtgcgcgacatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagcgcttcttctacagcaacatcatgaacttcttcaagaccgaggtgaagctggccaacggcgagatccgcaagcgccccctgatcgagaccaacggcgagaccggcgaggtggtgtggaacaaggagaaggacttcgccaccgtgcgcaaggtgctggccatgccccaggtgaacatcgtgaagaagaccgaggtgcagaccggcggcttcagcaaggagagcatcctgagcaagcgcgagagcgccaagctgatcccccgcaagaagggctgggacacccgcaagtacggcggcttcggcagccccaccgtggcctacagcatcctggtggtggccaaggtggagaagggcaaggccaagaagctgaagagcgtgaaggtgctggtgggcatcaccatcatggagaagggcagctacgagaaggaccccatcggcttcctggaggccaagggctacaaggacatcaagaaggagctgatcttcaagctgcccaagtacagcctgttcgagctggagaacggccgccgccgcatgctggccagcgccaccgagctgcagaaggccaacgagctggtgctgccccagcacctggtgcgcctgctgtactacacccagaacatcagcgccaccaccggcagcaacaacctgggctacatcgagcagcaccgcgaggagttcaaggagatattcgagaagatcatcgacttcagcgagaagtacatcctgaagaacaaggtgaacagcaacctgaagagcagcttcgacgagcagttcgccgtgagcgacagcatcctgctgagcaacagcttcgtgagcctgctgaagtacaccagcttcggcgccagcggcggcttcaccttcctggacctggacgtgaagcagggccgcctgcgctaccagaccgtgaccgaggtgctggacgccaccctgatctaccagagcatcaccggcctgtacgagacccgcaccgacctgagccagctgggcggcgac

SEQ ID NO: 41 - DNA, 1092 bp, synthetic construct
  misc_feature 1..1092: note = Synthetic Construct
  source 1..1092: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgac

SEQ ID NO: 42 - DNA, 498 bp, synthetic construct
  misc_feature 1..498: note = Synthetic Construct
  source 1..498: mol_type = other DNA
tctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacaggccatgccgaaattatggccctgaggcagggcggcctggtcatgcagaactacagactgattgacgccaccctgtacgggacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgac

SEQ ID NO: 43 - DNA, 267 bp, synthetic construct
  misc_feature 1..267: note = Synthetic Construct
  source 1..267: mol_type = other DNA
tgtttggatctgaaaacgcaagttcaaacgccacagggtatgaaagaaatatccaatatacaggtcggcgatctcgtcttgtctaacactggctataacgaggtgctgaatgtatttccaaaaagcaagaaaaaaagttacaagataactctggaagatggaaaagaaattatctgttctgaggagcatctgtttccgacccaaacaggggagatgaatatcagtggcggtctcaaagagggtatgtgtttgtatgtcaaggaataa

SEQ ID NO: 44 - DNA, 114 bp, synthetic construct
  misc_feature 1..114: note = Synthetic Construct
  source 1..114: mol_type = other DNA
atgatgctcaagaagatcctcaagattgaagagttggacgagcgcgagcttatagacatagaagtcagtggtaatcaccttttctacgcaaatgacattttgactcacaactcc

SEQ ID NO: 45 - DNA, 306 bp, synthetic construct
  misc_feature 1..306: note = Synthetic Construct
  source 1..306: mol_type = other DNA
tgtctcagttatgacaccgaaatcctgacagtcgagtatggatttctgccgatcggcaagattgtggaggagagaattgaatgtacggtctatacggtcgacaagaatggtttcgtctacacccaaccaattgctcaatggcataatcgaggggagcaggaggtgtttgagtattgcctggaggacgggtcaatcattagagctacaaaggaccataagtttatgacaaccgatggtcaaatgctgccgatagatgaaatattcgaaaggggactggatcttaagcaagtcgatggccttccaaac

SEQ ID NO: 46 - DNA, 108 bp, synthetic construct
  misc_feature 1..108: note = Synthetic Construct
  source 1..108: mol_type = other DNA
atggtcaagattatcagccgcaaatccttggggacacagaatgtatatgacatcggcgtggaaaaggatcacaattttctgctgaagaatggtcttgttgcttccaat

SEQ ID NO: 47 - protein, 166 aa, synthetic construct
  REGION 1..166: note = Synthetic Construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 48 - DNA, 45 bp, synthetic construct
  misc_feature 1..45: note = Synthetic Construct
  source 1..45: mol_type = other DNA
tgagtatcatcgtgtgaaagctgaggggacgaggcaggcctataa

SEQ ID NO: 49 - DNA, 45 bp, synthetic construct
  misc_feature 1..45: note = Synthetic Construct
  source 1..45: mol_type = other DNA
aaaaacatcaacttcagccatccatttcttcagggtttgtatgtg

SEQ ID NO: 50 - DNA, 45 bp, synthetic construct
  misc_feature 1..45: note = Synthetic Construct
  source 1..45: mol_type = other DNA
tatatcataatgaaaacgccgccatttctcaacagatctgtcaaa

SEQ ID NO: 51 - DNA, 45 bp, synthetic construct
  misc_feature 1..45: note = Synthetic Construct
  source 1..45: mol_type = other DNA
ctgtgtgaaatggctgcaaatcgatggttgagctctgagatttgg

SEQ ID NO: 52 - DNA, 45 bp, synthetic construct
  misc_feature 1..45: note = Synthetic Construct
  source 1..45: mol_type = other DNA
gttctgcttttgctactactcacgtttccatgttgtccccctcta

SEQ ID NO: 53 - DNA, 45 bp, synthetic construct
  misc_feature 1..45: note = Synthetic Construct
  source 1..45: mol_type = other DNA
attttatggccttttgcaactcgaccagaaaaaaagcagctttgg

SEQ ID NO: 54 - DNA, 45 bp, synthetic construct
  misc_feature 1..45: note = Synthetic Construct
  source 1..45: mol_type = other DNA
tgaggagatcgcccacgggctgccaggatcccttgatcacctcag

SEQ ID NO: 55 - DNA, 45 bp, synthetic construct
  misc_feature 1..45: note = Synthetic Construct
  source 1..45: mol_type = other DNA
ggctgctctgtcagaaatattcgtacagtctcaagagtactcatg

SEQ ID NO: 56 - DNA, 45 bp, synthetic construct
  misc_feature 1..45: note = Synthetic Construct
  source 1..45: mol_type = other DNA
gtgtaggcatagctcttgaatcgaggcttaggggaagaagttctc

SEQ ID NO: 57 - DNA, 45 bp, synthetic construct
  misc_feature 1..45: note = Synthetic Construct
  source 1..45: mol_type = other DNA
cctgttcttcagtaagacgttgccatttgagaaggatgtcttgta

SEQ ID NO: 58 - DNA, 45 bp, synthetic construct
  misc_feature 1..45: note = Synthetic Construct
  source 1..45: mol_type = other DNA
gccattttaggctttttacttacttgtctgtagctctttctctct

SEQ ID NO: 59 - DNA, 45 bp, synthetic construct
  misc_feature 1..45: note = Synthetic Construct
  source 1..45: mol_type = other DNA
ctagtttctcacacatgacacacctgttcttcagtaagacgttgc

SEQ ID NO: 60 - DNA, 45 bp, synthetic construct
  misc_feature 1..45: note = Synthetic Construct
  source 1..45: mol_type = other DNA
gtgaagttgattacattaacctgtggataattacgagttgattgt

SEQ ID NO: 61 - DNA, 45 bp, synthetic construct
  misc_feature 1..45: note = Synthetic Construct
  source 1..45: mol_type = other DNA
ttttgtatatctgagttaaactgctccaattccttcaaaggaatg

SEQ ID NO: 62 - DNA, 45 bp, synthetic construct
  misc_feature 1..45: note = Synthetic Construct
  source 1..45: mol_type = other DNA
tctgcaatataagctgccaactgcttgtcaatgaatgtgagggac

SEQ ID NO: 63 - DNA, 45 bp, synthetic construct
  misc_feature 1..45: note = Synthetic Construct
  source 1..45: mol_type = other DNA
ggactggggttccagtctcatccagtctaggaagagggccgcttc

SEQ ID NO: 64 - DNA, 45 bp, synthetic construct
  misc_feature 1..45: note = Synthetic Construct
  source 1..45: mol_type = other DNA
agtcgttgtgtggctgactgctggcaaaccacactattccagtca

SEQ ID NO: 65 - DNA, 45 bp, synthetic construct
  misc_feature 1..45: note = Synthetic Construct
  source 1..45: mol_type = other DNA
atttgtgtctttctgagaaactgttcagcttctgttagccactga

SEQ ID NO: 66 - DNA, 45 bp, synthetic construct
  misc_feature 1..45: note = Synthetic Construct
  source 1..45: mol_type = other DNA
aagaacccagcggtcttctgtccatctacagatgtttgcccatcg

SEQ ID NO: 67 - DNA, 45 bp, synthetic construct
  misc_feature 1..45: note = Synthetic Construct
  source 1..45: mol_type = other DNA
agactttttccgaagttcactccacttgaagttcatgttatccaa

SEQ ID NO: 68 - DNA, 45 bp, synthetic construct
  misc_feature 1..45: note = Synthetic Construct
  source 1..45: mol_type = other DNA
tacctgttggcacatgtgatcccactgagtgttaagttctttgag

SEQ ID NO: 69 - DNA, 45 bp, synthetic construct
  misc_feature 1..45: note = Synthetic Construct
  source 1..45: mol_type = other DNA
caaagggccttctgcagtcttcggagtttcatggcagtcctataa

SEQ ID NO: 70 - DNA, 45 bp, synthetic construct
  misc_feature 1..45: note = Synthetic Construct
  source 1..45: mol_type = other DNA
atggttaatgtctaacctttatccactggagatttgtctgcttga

SEQ ID NO: 71 - DNA, 45 bp, synthetic construct
  misc_feature 1..45: note = Synthetic Construct
  source 1..45: mol_type = other DNA
caaaataatctgaccttaagttgttcttccaaagcagcagttgcg

SEQ ID NO: 72 - DNA, 45 bp, synthetic construct
  misc_feature 1..45: note = Synthetic Construct
  source 1..45: mol_type = other DNA
catctacagatgtttgcccatcgatctcccaatacctggagaaga

SEQ ID NO: 73 - DNA, 45 bp, synthetic construct
  misc_feature 1..45: note = Synthetic Construct
  source 1..45: mol_type = other DNA
aaggtgttcttgtacttcatcccactgattctgaattctttcaac

SEQ ID NO: 74 - DNA, 45 bp, synthetic construct
  misc_feature 1..45: note = Synthetic Construct
  source 1..45: mol_type = other DNA
gaaggtgttcttgtacttcatcccactgattctgaattctttcaa

SEQ ID NO: 75 - DNA, 45 bp, synthetic construct
  misc_feature 1..45: note = Synthetic Construct
  source 1..45: mol_type = other DNA
gctatgctttgaatttttaatcgttcaatttgaggttgaagatct

SEQ ID NO: 76 - DNA, 45 bp, synthetic construct
  misc_feature 1..45: note = Synthetic Construct
  source 1..45: mol_type = other DNA
tctaggaggcgcctcccatcctgtaggtcactgaagaggttctca

SEQ ID NO: 77 - DNA, 45 bp, synthetic construct
  misc_feature 1..45: note = Synthetic Construct
  source 1..45: mol_type = other DNA
gtgtaattaccattcaccatctgttccaccagggcctgagctgat

SEQ ID NO: 78 - DNA, 45 bp, synthetic construct
  misc_feature 1..45: note = Synthetic Construct
  source 1..45: mol_type = other DNA
tgagcatgctttaccaggatctgttcccttgtggtcaccgtagtt

SEQ ID NO: 79 - DNA, 45 bp, synthetic construct
  misc_feature 1..45: note = Synthetic Construct
  source 1..45: mol_type = other DNA
ctactgtatagggaccctccttccatgactcaagcttggctctgg

SEQ ID NO: 80 - DNA, 45 bp, synthetic construct
  misc_feature 1..45: note = Synthetic Construct
  source 1..45: mol_type = other DNA
cagcttcttccttagcttccagccattgtgttgaatcctttaaca

SEQ ID NO: 81 - DNA, 45 bp, synthetic construct
  misc_feature 1..45: note = Synthetic Construct
  source 1..45: mol_type = other DNA
agctgcccaaggtcttttatttgagcttcaatttctccttgtttc

SEQ ID NO: 82 - DNA, 45 bp, synthetic construct
  misc_feature 1..45: note = Synthetic Construct
  source 1..45: mol_type = other DNA
tgccagtaacaactcacaatttgtgcaaagttgagtcttcgaaac

SEQ ID NO: 83 - DNA, 45 bp, synthetic construct
  misc_feature 1..45: note = Synthetic Construct
  source 1..45: mol_type = other DNA
agagtaacagtctgagtaggagctaaaatattttgggtttttgca

SEQ ID NO: 84 - DNA, 45 bp, synthetic construct
  misc_feature 1..45: note = Synthetic Construct
  source 1..45: mol_type = other DNA
cttcagcaaaaaaagtactcacgcagaatctactggccagaagtt

SEQ ID NO: 85 - DNA, 45 bp, synthetic construct
  misc_feature 1..45: note = Synthetic Construct
  source 1..45: mol_type = other DNA
acatctacatttgtctgccactggcggaggtctttggccaactgc

SEQ ID NO: 86 - DNA, 45 bp, synthetic construct
  misc_feature 1..45: note = Synthetic Construct
  source 1..45: mol_type = other DNA
ctgagatagtataggccactttgttgctcttgcagagaactttgt

SEQ ID NO: 87 - DNA, 45 bp, synthetic construct
  misc_feature 1..45: note = Synthetic Construct
  source 1..45: mol_type = other DNA
tagctgtcctttacacactttacctgttgagaatagtgcatttga

SEQ ID NO: 88 - DNA, 45 bp, synthetic construct
  misc_feature 1..45: note = Synthetic Construct
  source 1..45: mol_type = other DNA
gcctgggcttcctgaggcatttgagctgcgtccaccttgtctgca

SEQ ID NO: 89 - DNA, 45 bp, synthetic construct
  misc_feature 1..45: note = Synthetic Construct
  source 1..45: mol_type = other DNA
tgcactggcaggtagcccattcggggatgcttcgcaaaatacctt

SEQ ID NO: 90 - DNA, 45 bp, synthetic construct
  misc_feature 1..45: note = Synthetic Construct
  source 1..45: mol_type = other DNA
ttatagttccacattcaattacctctgggctcctggtagagtttc

SEQ ID NO: 91 - DNA, 45 bp, synthetic construct
  misc_feature 1..45: note = Synthetic Construct
  source 1..45: mol_type = other DNA
cgtcaggctggcgtcaaacttaccggagtgcaatattccaccatg

SEQ ID NO: 92 - DNA, 45 bp, synthetic construct
  misc_feature 1..45: note = Synthetic Construct
  source 1..45: mol_type = other DNA
tcatccaaaagtgtgtcagcctgaatgatccactttgtgatgtgg

SEQ ID NO: 93 - DNA, 45 bp, synthetic construct
  misc_feature 1..45: note = Synthetic Construct
  source 1..45: mol_type = other DNA
tgtagccacaccagaagttcctgcagagaaaggtgcagacgcttc

SEQ ID NO: 94 - DNA, 45 bp, synthetic construct
  misc_feature 1..45: note = Synthetic Construct
  source 1..45: mol_type = other DNA
tgtaaggatttttcagtctcctgggcagactggatgctctgttca

SEQ ID NO: 95 - DNA, 45 bp, synthetic construct
  misc_feature 1..45: note = Synthetic Construct
  source 1..45: mol_type = other DNA
agaatgggatccagtatacttacaggctccaatagtggtcagtcc

SEQ ID NO: 96 - DNA, 45 bp, synthetic construct
  misc_feature 1..45: note = Synthetic Construct
  source 1..45: mol_type = other DNA
ttcagaggcgcaatttctcctcgaagtgcctgtgtgcaatagtca

SEQ ID NO: 97 - DNA, 45 bp, synthetic construct
  misc_feature 1..45: note = Synthetic Construct
  source 1..45: mol_type = other DNA
gtttcttccaaagcagcctctcgctcactcaccctgcaaaggacc

SEQ ID NO: 98 - DNA, 45 bp, synthetic construct
  misc_feature 1..45: note = Synthetic Construct
  source 1..45: mol_type = other DNA
ctgaacttctcagctttttctcgctctatggcctgcagcatgaga

SEQ ID NO: 99 - DNA, 45 bp, synthetic construct
  misc_feature 1..45: note = Synthetic Construct
  source 1..45: mol_type = other DNA
agatttaaccactcttctgctcgggaggtgacagctatccagtta

SEQ ID NO: 100 - DNA, 45 bp, synthetic construct
  misc_feature 1..45: note = Synthetic Construct
  source 1..45: mol_type = other DNA
tgttttatctttatttcctctcgctttctctcatctgtgattctt

SEQ ID NO: 101 - DNA, 45 bp, synthetic construct
  misc_feature 1..45: note = Synthetic Construct
  source 1..45: mol_type = other DNA
ccagctgggaggagagcttcttccagcgtccctcaatttcttcaa

SEQ ID NO: 102 - DNA, 45 bp, synthetic construct
  misc_feature 1..45: note = Synthetic Construct
  source 1..45: mol_type = other DNA
acacagcttctgagcgagtaatccagctgtgaagttcagttatat

SEQ ID NO: 103 - DNA, 45 bp, synthetic construct
  misc_feature 1..45: note = Synthetic Construct
  source 1..45: mol_type = other DNA
aagtaaacggtttaccgccttccactcagagctcagatcttctaa

SEQ ID NO: 104 - DNA, 45 bp, synthetic construct
  misc_feature 1..45: note = Synthetic Construct
  source 1..45: mol_type = other DNA
aaatagaaaaattagatgacttgccaaaggtcacaaaggtaactg

SEQ ID NO: 105 - DNA, 45 bp, synthetic construct
  misc_feature 1..45: note = Synthetic Construct
  source 1..45: mol_type = other DNA
ttgactttctcgaggtgatcttggagagagtcaatgaggagatcg

SEQ ID NO: 106 - DNA, 45 bp, synthetic construct
  misc_feature 1..45: note = Synthetic Construct
  source 1..45: mol_type = other DNA
tctaaaatcatcttactttcttgtagacgctgctcaaaattggct

SEQ ID NO: 107 - DNA, 45 bp, synthetic construct
  misc_feature 1..45: note = Synthetic Construct
  source 1..45: mol_type = other DNA
gaattgaccctgacttgttcttgttctagatcttcttgaagcacc

SEQ ID NO: 108 - DNA, 45 bp, synthetic construct
  misc_feature 1..45: note = Synthetic Construct
  source 1..45: mol_type = other DNA
tggatggcttcaatgctcacttgttgaggcaaaacttggaagagt

SEQ ID NO: 109 - DNA, 45 bp, synthetic construct
  misc_feature 1..45: note = Synthetic Construct
  source 1..45: mol_type = other DNA
aacagtcctctacttcttcccaccaaagcattttgaaaagtgtat

SEQ ID NO: 110 - DNA, 45 bp, synthetic construct
  misc_feature 1..45: note = Synthetic Construct
  source 1..45: mol_type = other DNA
aggcctcctttctggcatagaccttccacaaaacaaacaaacaaa

SEQ ID NO: 111 - DNA, 45 bp, synthetic construct
  misc_feature 1..45: note = Synthetic Construct
  source 1..45: mol_type = other DNA
tttggtttctgactgctggacccatgtcctgatggcactcatggt

SEQ ID NO: 112 - DNA, 45 bp, synthetic construct
  misc_feature 1..45: note = Synthetic Construct
  source 1..45: mol_type = other DNA
atcttactttcttgtagacgctgctcaaaattggctggtttctgg

SEQ ID NO: 113 - DNA, 45 bp, synthetic construct
  misc_feature 1..45: note = Synthetic Construct
  source 1..45: mol_type = other DNA
agatttttcacttatcttcatacctcttcatgtagttccctccaa

SEQ ID NO: 114 - DNA, 45 bp, synthetic construct
  misc_feature 1..45: note = Synthetic Construct
  source 1..45: mol_type = other DNA
ctgttcagttgttctgaggcttgtttgatgctatctgcattaaca

SEQ ID NO: 115 - DNA, 45 bp, synthetic construct
  misc_feature 1..45: note = Synthetic Construct
  source 1..45: mol_type = other DNA
cagcattaatatacacgacttacatctgtacttgtcttccaaatg

SEQ ID NO: 116 - DNA, 45 bp, synthetic construct
  misc_feature 1..45: note = Synthetic Construct
  source 1..45: mol_type = other DNA
tcatgacttgtcaaatcagattggattttctgttgggaggatagc

SEQ ID NO: 117 - DNA, 45 bp, synthetic construct
  misc_feature 1..45: note = Synthetic Construct
  source 1..45: mol_type = other DNA
atctgctccaattgttgtagctgattatagaaagcgatgatgttg

SEQ ID NO: 118 - DNA, 45 bp, synthetic construct
  misc_feature 1..45: note = Synthetic Construct
  source 1..45: mol_type = other DNA
caaatttgctctcaatttcccgccagcgcttgctgagctggatct

SEQ ID NO: 119 - DNA, 45 bp, synthetic construct
  misc_feature 1..45: note = Synthetic Construct
  source 1..45: mol_type = other DNA
cattcaaagccaggccatcagaccagctggtggtgaagttgatta

SEQ ID NO: 120 - DNA, 45 bp, synthetic construct
  misc_feature 1..45: note = Synthetic Construct
  source 1..45: mol_type = other DNA
ttcatctcttcaactgctttctgtaattcatctggagttttatat

SEQ ID NO: 121 - DNA, 45 bp, synthetic construct
  misc_feature 1..45: note = Synthetic Construct
  source 1..45: mol_type = other DNA
attgaaagctagaaagtacatacggccagtttttgaagacttgat

SEQ ID NO: 122 - DNA, 45 bp, synthetic construct
  misc_feature 1..45: note = Synthetic Construct
  source 1..45: mol_type = other DNA
ttcaaatactggccaatacttacagcaaagggccttctgcagtct

SEQ ID NO: 123 - DNA, 45 bp, synthetic construct
  misc_feature 1..45: note = Synthetic Construct
  source 1..45: mol_type = other DNA
gttgtctgtgttagtgatggctgagtggtggtgacagcctgtgaa

SEQ ID NO: 124 - DNA, 45 bp, synthetic construct
  misc_feature 1..45: note = Synthetic Construct
  source 1..45: mol_type = other DNA
tcatcagcctgcctcttgtactgataccactgatgagaaatttct

SEQ ID NO: 125 - DNA, 45 bp, synthetic construct
  misc_feature 1..45: note = Synthetic Construct
  source 1..45: mol_type = other DNA
tactgtatagggaccctccttccatgactcaagcttggctctggc

SEQ ID NO: 126 - DNA, 45 bp, synthetic construct
  misc_feature 1..45: note = Synthetic Construct
  source 1..45: mol_type = other DNA
atgttgaatgcatgttccagtcgttgtgtggctgactgctggcaa

SEQ ID NO: 127 - DNA, 45 bp, synthetic construct
  misc_feature 1..45: note = Synthetic Construct
  source 1..45: mol_type = other DNA
tgccatttgagaaggatgtcttgtaaaagaacccagcggtcttct

SEQ ID NO: 128 - DNA, 45 bp, synthetic construct
  misc_feature 1..45: note = Synthetic Construct
  source 1..45: mol_type = other DNA
gagactttttccgaagttcactccacttgaagttcatgttatcca

SEQ ID NO: 129 - DNA, 45 bp, synthetic construct
  misc_feature 1..45: note = Synthetic Construct
  source 1..45: mol_type = other DNA
ttagcaactggcagaattcgatccaccggctgttcagttgttctg

SEQ ID NO: 130 - DNA, 45 bp, synthetic construct
  misc_feature 1..45: note = Synthetic Construct
  source 1..45: mol_type = other DNA
ttgccacatctacatttgtctgccactggcggaggtctttggcca

SEQ ID NO: 131 - DNA, 45 bp, synthetic construct
  misc_feature 1..45: note = Synthetic Construct
  source 1..45: mol_type = other DNA
tcccattcagcctagtgcagagccactggtagttggtggttagag

SEQ ID NO: 132 - DNA, 45 bp, synthetic construct
  misc_feature 1..45: note = Synthetic Construct
  source 1..45: mol_type = other DNA
gacttactggaaagaaagtgctgagatgctggaccaaagtccctg

SEQ ID NO: 133 - DNA, 45 bp, synthetic construct
  misc_feature 1..45: note = Synthetic Construct
  source 1..45: mol_type = other DNA
tttaatcgttcaatttgaggttgaagatctgatagccggttgact

SEQ ID NO: 134 - DNA, 45 bp, synthetic construct
  misc_feature 1..45: note = Synthetic Construct
  source 1..45: mol_type = other DNA
aaagagattgtctatacctgttggcacatgtgatcccactgagtg

SEQ ID NO: 135 - DNA, 45 bp, synthetic construct
  misc_feature 1..45: note = Synthetic Construct
  source 1..45: mol_type = other DNA
aagtttttggactaaattatcccaacaccgggcaaagttatccag

SEQ ID NO: 136 - DNA, 45 bp, synthetic construct
  misc_feature 1..45: note = Synthetic Construct
  source 1..45: mol_type = other DNA
agctcagcatcccggggactctggggagaggtgggcatcatttca

SEQ ID NO: 137 - DNA, 45 bp, synthetic construct
  misc_feature 1..45: note = Synthetic Construct
  source 1..45: mol_type = other DNA
ttgtccccctctaagacagtctgcactggcaggtagcccattcgg

SEQ ID NO: 138 - DNA, 45 bp, synthetic construct
  misc_feature 1..45: note = Synthetic Construct
  source 1..45: mol_type = other DNA
ttcgcaaaataccttttggttcgaaatttgttttttagtaccttg

SEQ ID NO: 139 - DNA, 45 bp, synthetic construct
  misc_feature 1..45: note = Synthetic Construct
  source 1..45: mol_type = other DNA
tgcaactcgaccagaaaaaaagcagctttggcagatgtcataatt

SEQ ID NO: 140 - DNA, 45 bp, synthetic construct
  misc_feature 1..45: note = Synthetic Construct
  source 1..45: mol_type = other DNA
ttgcagatgttacatttggcctgatgcttggcagtttctgcagca

SEQ ID NO: 141 - DNA, 45 bp, synthetic construct
  misc_feature 1..45: note = Synthetic Construct
  source 1..45: mol_type = other DNA
aaataaaaacatgccatacgtacgtatcataaacattcagcagcc

SEQ ID NO: 142 - DNA, 45 bp, synthetic construct
  misc_feature 1..45: note = Synthetic Construct
  source 1..45: mol_type = other DNA
tacttacagcaaagggccttctgcagtcttcggagtttcatggca

SEQ ID NO: 143 - DNA, 45 bp, synthetic construct
  misc_feature 1..45: note = Synthetic Construct
  source 1..45: mol_type = other DNA
ttgacctcctcagcctgctttcgtagaagccgagtgacattctgg

SEQ ID NO: 144 - DNA, 45 bp, synthetic construct
  misc_feature 1..45: note = Synthetic Construct
  source 1..45: mol_type = other DNA
attcaattacctctgggctcctggtagagtttctctagtccttcc

SEQ ID NO: 145 - DNA, 45 bp, synthetic construct
  misc_feature 1..45: note = Synthetic Construct
  source 1..45: mol_type = other DNA
aatgcctgacttacttgccattgtttcatcagctcttttactccc

SEQ ID NO: 146 - DNA, 45 bp, synthetic construct
  misc_feature 1..45: note = Synthetic Construct
  source 1..45: mol_type = other DNA
tgtacttcatcccactgattctgaattctttcaactagaataaaa

SEQ ID NO: 147 - DNA, 45 bp, synthetic construct
  misc_feature 1..45: note = Synthetic Construct
  source 1..45: mol_type = other DNA
tgcttcattaccttcactggctgagtggctggtttttccttgtac

SEQ ID NO: 148 - DNA, 45 bp, synthetic construct
  misc_feature 1..45: note = Synthetic Construct
  source 1..45: mol_type = other DNA
tttaattgtttgagaattccctggcgcaggggcaactcttccacc

SEQ ID NO: 149 - DNA, 45 bp, synthetic construct
  misc_feature 1..45: note = Synthetic Construct
  source 1..45: mol_type = other DNA
atatgtgttacctacccttgtcggtccttgtacattttgttaact

SEQ ID NO: 150 - DNA, 45 bp, synthetic construct
  misc_feature 1..45: note = Synthetic Construct
  source 1..45: mol_type = other DNA
tcaagctgggagagagcttcctgtagcttcaccctttccacaggc

SEQ ID NO: 151 - DNA, 45 bp, synthetic construct
  misc_feature 1..45: note = Synthetic Construct
  source 1..45: mol_type = other DNA
atgtcaatccgacctgagctttgttgtagactatcttttatattc

SEQ ID NO: 152 - DNA, 45 bp, synthetic construct
  misc_feature 1..45: note = Synthetic Construct
  source 1..45: mol_type = other DNA
gttgtagactatcttttatattctgtaatataaaaattttaaaac

SEQ ID NO: 153 - DNA, 45 bp, synthetic construct
  misc_feature 1..45: note = Synthetic Construct
  source 1..45: mol_type = other DNA
tcccgccagcgcttgctgagctggatctgagttggctccactgcc

SEQ ID NO: 154 - DNA, 45 bp, synthetic construct
  misc_feature 1..45: note = Synthetic Construct
  source 1..45: mol_type = other DNA
ttatgttttgtctgtaacagctgctgttttatctttatttcctct

SEQ ID NO: 155 - DNA, 45 bp, synthetic construct
  misc_feature 1..45: note = Synthetic Construct
  source 1..45: mol_type = other DNA
tgttttgtctgtaacagctgctgttttatctttatttcctctcgc

SEQ ID NO: 156 - DNA, 45 bp, synthetic construct
  misc_feature 1..45: note = Synthetic Construct
  source 1..45: mol_type = other DNA
tttctctcatctgtgattctttgttgtaagttgtctcctctttgc

SEQ ID NO: 157 - DNA, 45 bp, synthetic construct
  misc_feature 1..45: note = Synthetic Construct
  source 1..45: mol_type = other DNA
accttaagcacgtcttctttttgctggggtttctttttctctgat

SEQ ID NO: 158 - DNA, 45 bp, synthetic construct
  misc_feature 1..45: note = Synthetic Construct
  source 1..45: mol_type = other DNA
ttaagcacgtcttctttttgctggggtttctttttctctgattca

SEQ ID NO: 159 - DNA, 45 bp, synthetic construct
  misc_feature 1..45: note = Synthetic Construct
  source 1..45: mol_type = other DNA
atactcttcaggtgcaccttctgtttctcaatctctttttgagta

SEQ ID NO: 160 - DNA, 45 bp, synthetic construct
  misc_feature 1..45: note = Synthetic Construct
  source 1..45: mol_type = other DNA
agctgtgactgtactacttcctgttccacactctttgtttccaat

SEQ ID NO: 161 - DNA, 45 bp, synthetic construct
  misc_feature 1..45: note = Synthetic Construct
  source 1..45: mol_type = other DNA
ttggctggtttctggaataatcgaaacttcatggagacatcttgt

SEQ ID NO: 162 - DNA, 45 bp, synthetic construct
  misc_feature 1..45: note = Synthetic Construct
  source 1..45: mol_type = other DNA
tgcatctctgatagatctttctggaggcttacagttttctccaaa

SEQ ID NO: 163 - DNA, 45 bp, synthetic construct
  misc_feature 1..45: note = Synthetic Construct
  source 1..45: mol_type = other DNA
acagtgaaagagattgtctatacctgttggcacatgtgatcccac

SEQ ID NO: 164 - DNA, 45 bp, synthetic construct
  misc_feature 1..45: note = Synthetic Construct
  source 1..45: mol_type = other DNA
aaggcatcatataaaaatcttactctgcactgtttcagctgcttt

SEQ ID NO: 165 - DNA, 45 bp, synthetic construct
  misc_feature 1..45: note = Synthetic Construct
  source 1..45: mol_type = other DNA
cttactctgcactgtttcagctgcttttttagaatttctgaatcc

SEQ ID NO: 166 - DNA, 45 bp, synthetic construct
  misc_feature 1..45: note = Synthetic Construct
  source 1..45: mol_type = other DNA
tcttgaattacctgaatttttcggagtttattcatttgctcctct

SEQ ID NO: 167 - DNA, 45 bp, synthetic construct
  misc_feature 1..45: note = Synthetic Construct
  source 1..45: mol_type = other DNA
tgttgctcttgcagagaactttgtaaagcctaaaaaacaattttt

SEQ ID NO: 168 - DNA, 45 bp, synthetic construct
  misc_feature 1..45: note = Synthetic Construct
  source 1..45: mol_type = other DNA
attggtggcaaagtgtcaaaaactttatcaaaagggaaaaaagaa

SEQ ID NO: 169 - DNA, 45 bp, synthetic construct
  misc_feature 1..45: note = Synthetic Construct
  source 1..45: mol_type = other DNA
taggctttttacttacttgtctgtagctctttctctctggcctgc

SEQ ID NO: 170 - DNA, 45 bp, synthetic construct
  misc_feature 1..45: note = Synthetic Construct
  source 1..45: mol_type = other DNA
gcctgcacatcagaaaagacttgcttaaaatgatttgtaaaggcc

SEQ ID NO: 171 - DNA, 45 bp, synthetic construct
  misc_feature 1..45: note = Synthetic Construct
  source 1..45: mol_type = other DNA
atggaaggagaagagattcttaccttacaaatttttaactgactt

SEQ ID NO: 172 - DNA, 45 bp, synthetic construct
  misc_feature 1..45: note = Synthetic Construct
  source 1..45: mol_type = other DNA
ggtggtgggttggattttcaaccagttttcagcagtagttgtcat

SEQ ID NO: 173 - DNA, 45 bp, synthetic construct
  misc_feature 1..45: note = Synthetic Construct
  source 1..45: mol_type = other DNA
tcgatccaccggctgttcagttgttctgaggcttgtttgatgcta

SEQ ID NO: 174 - DNA, 45 bp, synthetic construct
  misc_feature 1..45: note = Synthetic Construct
  source 1..45: mol_type = other DNA
tgagctgatctgctggcatcttgcagttttctgaacttctcagct

SEQ ID NO: 175 - DNA, 45 bp, synthetic construct
  misc_feature 1..45: note = Synthetic Construct
  source 1..45: mol_type = other DNA
ataaaagcttaagatgctctcaccttttcctaatttcagaatcca

SEQ ID NO: 176 - DNA, 45 bp, synthetic construct
  misc_feature 1..45: note = Synthetic Construct
  source 1..45: mol_type = other DNA
atttcagaatccacagtaatctgcctcttcttttggggaggtggt

SEQ ID NO: 177 - DNA, 45 bp, synthetic construct
  misc_feature 1..45: note = Synthetic Construct
  source 1..45: mol_type = other DNA
tgataattggtatcactaacctgtgctgtactcttttcaagtttt

SEQ ID NO: 178 - DNA, 45 bp, synthetic construct
  misc_feature 1..45: note = Synthetic Construct
  source 1..45: mol_type = other DNA
tccagccatgcttccgtcttctgggtcactgacttattcttcagt

SEQ ID NO: 179 - DNA, 45 bp, synthetic construct
  misc_feature 1..45: note = Synthetic Construct
  source 1..45: mol_type = other DNA
gaaggatgtcttgtaaaagaacccagcggtcttctgtccatctac

SEQ ID NO: 180 - DNA, 45 bp, synthetic construct
  misc_feature 1..45: note = Synthetic Construct
  source 1..45: mol_type = other DNA
tgttcttgttctagatcttcttgaagcacctgaaagataaaatgt

SEQ ID NO: 181 - DNA, 45 bp, synthetic construct
  misc_feature 1..45: note = Synthetic Construct
  source 1..45: mol_type = other DNA
cctaccttatgttgttgtacttggcgttttaggtcttcaagatca

SEQ ID NO: 182 - DNA, 45 bp, synthetic construct
  misc_feature 1..45: note = Synthetic Construct
  source 1..45: mol_type = other DNA
tctttcttctgtttttgttagccagtcattcaactctttcagttt

SEQ ID NO: 183 - DNA, 45 bp, synthetic construct
  misc_feature 1..45: note = Synthetic Construct
  source 1..45: mol_type = other DNA
attaaaaacaaataaggacttacttgctttgtttttccatgctag

SEQ ID NO: 184 - DNA, 45 bp, synthetic construct
  misc_feature 1..45: note = Synthetic Construct
  source 1..45: mol_type = other DNA
caaataaggacttacttgctttgtttttccatgctagctaccctg

SEQ ID NO: 185 - DNA, 45 bp, synthetic construct
  misc_feature 1..45: note = Synthetic Construct
  source 1..45: mol_type = other DNA
tttaggagattcatctgctcttgtacttcagtttcttcatcttct

SEQ ID NO: 186 - DNA, 45 bp, synthetic construct
  misc_feature 1..45: note = Synthetic Construct
  source 1..45: mol_type = other DNA
acatcattagaaatctctccttgtgcttgcaatgtgtcctcagca

SEQ ID NO: 187 - DNA, 45 bp, synthetic construct
  misc_feature 1..45: note = Synthetic Construct
  source 1..45: mol_type = other DNA
tggtagtccagaaatttaccaaccttcaggatcgagtagtttctc

SEQ ID NO: 188 - DNA, 45 bp, synthetic construct
  misc_feature 1..45: note = Synthetic Construct
  source 1..45: mol_type = other DNA
tatttttcattacatttttgacctacatgtggaaataaattttca

SEQ ID NO: 189 - DNA, 45 bp, synthetic construct
  misc_feature 1..45: note = Synthetic Construct
  source 1..45: mol_type = other DNA
ccattcatcaggattcttacctgccagtggaggattatattccaa

SEQ ID NO: 190 - DNA, 45 bp, synthetic construct
  misc_feature 1..45: note = Synthetic Construct
  source 1..45: mol_type = other DNA
tttcttaaaaataagtcacataccagtttttgccctgtcaggcct

SEQ ID NO: 191 - DNA, 45 bp, synthetic construct
  misc_feature 1..45: note = Synthetic Construct
  source 1..45: mol_type = other DNA
aataagtcacataccagtttttgccctgtcaggccttcgaggagg

SEQ ID NO: 192 - DNA, 45 bp, synthetic construct
  misc_feature 1..45: note = Synthetic Construct
  source 1..45: mol_type = other DNA
gtaaagtaacaaaccattcttaccttagaaaattgtgcatttacc

SEQ ID NO: 193 - DNA, 45 bp, synthetic construct
  misc_feature 1..45: note = Synthetic Construct
  source 1..45: mol_type = other DNA
tttactaagcaaaataatctgaccttaagttgttcttccaaagca

SEQ ID NO: 194 - DNA, 45 bp, synthetic construct
  misc_feature 1..45: note = Synthetic Construct
  source 1..45: mol_type = other DNA
acggatcctccctgttcgtcccctattatgaagaatcaaagcaga

SEQ ID NO: 195 - DNA, 45 bp, synthetic construct
  misc_feature 1..45: note = Synthetic Construct
  source 1..45: mol_type = other DNA
ttctcaacagatctgtcaaatcgcctgcaggtaaaagcatatgga

SEQ ID NO: 196 - DNA, 45 bp, synthetic construct
  misc_feature 1..45: note = Synthetic Construct
  source 1..45: mol_type = other DNA
ctgtcaaatccatcatgtacccctgacaaagaaggaagttaacaa

SEQ ID NO: 197 - DNA, 45 bp, synthetic construct
  misc_feature 1..45: note = Synthetic Construct
  source 1..45: mol_type = other DNA
tctcaatatgctgcttcccaaactgaaattaaaaaaaatacactc

SEQ ID NO: 198 - DNA, 45 bp, synthetic construct
  misc_feature 1..45: note = Synthetic Construct
  source 1..45: mol_type = other DNA
cttaattcatcatctttcagctgtagccacaccagaagttcctgc

SEQ ID NO: 199 - DNA, 45 bp, synthetic construct
  misc_feature 1..45: note = Synthetic Construct
  source 1..45: mol_type = other DNA
gtcaagacattcatttcctttcgcatcttacgggacaatttcaag

SEQ ID NO: 200 - DNA, 45 bp, synthetic construct
  misc_feature 1..45: note = Synthetic Construct
  source 1..45: mol_type = other DNA
tgtgtcctcagcagaaagaagccacgataatacttcttctaaagc

SEQ ID NO: 201 - DNA, 45 bp, synthetic construct
  misc_feature 1..45: note = Synthetic Construct
  source 1..45: mol_type = other DNA
tagaaagcgatgatgttgttctgatactccagccagttaagtctc

SEQ ID NO: 202 - DNA, 45 bp, synthetic construct
  misc_feature 1..45: note = Synthetic Construct
  source 1..45: mol_type = other DNA
ctctctaaggaaatcaagatctgggcaggactacgaggctggctc

SEQ ID NO: 203 - DNA, 45 bp, synthetic construct
  misc_feature 1..45: note = Synthetic Construct
  source 1..45: mol_type = other DNA
tcaaaagtttccatgtgtttctggtattccttaattgtacagaga

SEQ ID NO: 204 - DNA, 45 bp, synthetic construct
  misc_feature 1..45: note = Synthetic Construct
  source 1..45: mol_type = other DNA
actgtttccattacagttgtctgtgttagtgatggctgagtggtg

SEQ ID NO: 205 - DNA, 45 bp, synthetic construct
  misc_feature 1..45: note = Synthetic Construct
  source 1..45: mol_type = other DNA
tttagtaccttggcaaagtctcgaacatcttctcctgatgtagtc

SEQ ID NO: 206 - DNA, 45 bp, synthetic construct
  misc_feature 1..45: note = Synthetic Construct
  source 1..45: mol_type = other DNA
atttgtgcaaagttgagtcttcgaaactgagcaaatttgctctca

SEQ ID NO: 207 - DNA, 45 bp, synthetic construct
  misc_feature 1..45: note = Synthetic Construct
  source 1..45: mol_type = other DNA
tggggacgcctctgttccaaatcctgcattgttgcctgtaagaac

SEQ ID NO: 208 - DNA, 45 bp, synthetic construct
  misc_feature 1..45: note = Synthetic Construct
  source 1..45: mol_type = other DNA
gtctcctatgaactcgagaagccgcaaaaccaaggaagagaaaga

SEQ ID NO: 209 - DNA, 45 bp, synthetic construct
  misc_feature 1..45: note = Synthetic Construct
  source 1..45: mol_type = other DNA
gagagtttggtttctgactgctggacccatgtcctgatggcactc

SEQ ID NO: 210 - DNA, 45 bp, synthetic construct
  misc_feature 1..45: note = Synthetic Construct
  source 1..45: mol_type = other DNA
tgcgtatttgccaccagaaatacataccacacaatgatttagctg

SEQ ID NO: 211 - DNA, 45 bp, synthetic construct
  misc_feature 1..45: note = Synthetic Construct
  source 1..45: mol_type = other DNA
tttgggttatcctctgaatgtcgcatcaaattttcaagtgactga

SEQ ID NO: 212 - DNA, 45 bp, synthetic construct
  misc_feature 1..45: note = Synthetic Construct
  source 1..45: mol_type = other DNA
aggacacggatcctccctgttcgtcccctattatgaagaatcaaa

SEQ ID NO: 213 - DNA, 45 bp, synthetic construct
  misc_feature 1..45: note = Synthetic Construct
  source 1..45: mol_type = other DNA
tgcttgttaaaaaacttacttcgatccgtaatgattgttctagcc

SEQ ID NO: 214 - DNA, 45 bp, synthetic construct
  misc_feature 1..45: note = Synthetic Construct
  source 1..45: mol_type = other DNA
ttttgctccacatcttttcctacctaatgttgagagactttttcc

SEQ ID NO: 215 - DNA, 45 bp, synthetic construct
  misc_feature 1..45: note = Synthetic Construct
  source 1..45: mol_type = other DNA
cctgccagtggaggattatattccaaatcaaaccaagagtcagtt

SEQ ID NO: 216 - DNA, 45 bp, synthetic construct
  misc_feature 1..45: note = Synthetic Construct
  source 1..45: mol_type = other DNA
ggataattacgagttgattgtcggacccagctcaggagaatcttt

SEQ ID NO: 217 - DNA, 45 bp, synthetic construct
  misc_feature 1..45: note = Synthetic Construct
  source 1..45: mol_type = other DNA
tttagactgggctgaattgtctgaatatcactgactaaaagctaa

SEQ ID NO: 218 - DNA, 45 bp, synthetic construct
  misc_feature 1..45: note = Synthetic Construct
  source 1..45: mol_type = other DNA
gtactacttacattattgttctgcaaaacccgcagtgccttgttg

SEQ ID NO: 219 - DNA, 45 bp, synthetic construct
  misc_feature 1..45: note = Synthetic Construct
  source 1..45: mol_type = other DNA
ttcatttgctcctctagcttttgacaatgctcaaccagctgggag

SEQ ID NO: 220 - DNA, 45 bp, synthetic construct
  misc_feature 1..45: note = Synthetic Construct
  source 1..45: mol_type = other DNA
tcaatctgagacaggactctttgggcagcctccttcccctgatta

SEQ ID NO: 221 - DNA, 45 bp, synthetic construct
  misc_feature 1..45: note = Synthetic Construct
  source 1..45: mol_type = other DNA
ttcagctcctctttcttcttctgcaattcccgatcaatttcctat

SEQ ID NO: 222 - DNA, 23 bp, synthetic construct
  misc_feature 1..23: note = Synthetic Construct
  source 1..23: mol_type = other DNA
aaagctaaggggacgaggcaggc

SEQ ID NO: 223 - DNA, 23 bp, synthetic construct
  misc_feature 1..23: note = Synthetic Construct
  source 1..23: mol_type = other DNA
gaaagctaaggggacgaggcagg

SEQ ID NO: 224 - DNA, 23 bp, synthetic construct
  misc_feature 1..23: note = Synthetic Construct
  source 1..23: mol_type = other DNA
aaatagatggctgaagttgatgt

SEQ ID NO: 225 - DNA, 23 bp, synthetic construct
  misc_feature 1..23: note = Synthetic Construct
  source 1..23: mol_type = other DNA
gaaatagatggctgaagttgatg

SEQ ID NO: 226 - DNA, 23 bp, synthetic construct
  misc_feature 1..23: note = Synthetic Construct
  source 1..23: mol_type = other DNA
aagaaatagatggctgaagttga

SEQ ID NO: 227 - DNA, 27 bp, synthetic construct
  misc_feature 1..27: note = Synthetic Construct
  source 1..27: mol_type = other DNA
ctgaagaaatagatggctgaagttgat

SEQ ID NO: 228 - DNA, 23 bp, synthetic construct
  misc_feature 1..23: note = Synthetic Construct
  source 1..23: mol_type = other DNA
aaatagcggcgttttcattatga

SEQ ID NO: 229 - DNA, 27 bp, synthetic construct
  misc_feature 1..27: note = Synthetic Construct
  source 1..27: mol_type = other DNA
gagaaatagcggcgttttcattatgat

SEQ ID NO: 230 - DNA, 23 bp, synthetic construct
  misc_feature 1..23: note = Synthetic Construct
  source 1..23: mol_type = other DNA
aaatcaatggttgagctctgaga

SEQ ID NO: 231 - DNA, 23 bp, synthetic construct
  misc_feature 1..23: note = Synthetic Construct
  source 1..23: mol_type = other DNA
gcaaatcaatggttgagctctga

SEQ ID NO: 232 - DNA, 27 bp, synthetic construct
  misc_feature 1..27: note = Synthetic Construct
  source 1..27: mol_type = other DNA
tgcaaatcaatggttgagctctgagat

SEQ ID NO: 233 - DNA, 23 bp, synthetic construct
  misc_feature 1..23: note = Synthetic Construct
  source 1..23: mol_type = other DNA
aacatgagtagtagcaaaagcag

SEQ ID NO: 234 - DNA, 23 bp, synthetic construct
  misc_feature 1..23: note = Synthetic Construct
  source 1..23: mol_type = other DNA
gaaacatgagtagtagcaaaagc

SEQ ID NO: 235 - DNA, 23 bp, synthetic construct
  misc_feature 1..23: note = Synthetic Construct
  source 1..23: mol_type = other DNA
ggaaacatgagtagtagcaaaag

SEQ ID NO: 236 - DNA, 23 bp, synthetic construct
  misc_feature 1..23: note = Synthetic Construct
  source 1..23: mol_type = other DNA
tggaaacatgagtagtagcaaaa

SEQ ID NO: 237 - DNA, 23 bp, synthetic construct
  misc_feature 1..23: note = Synthetic Construct
  source 1..23: mol_type = other DNA
aactcaaccagaaaaaaagcagc

SEQ ID NO: 238 - DNA, 23 bp, synthetic construct
  misc_feature 1..23: note = Synthetic Construct
  source 1..23: mol_type = other DNA
caactcaaccagaaaaaaagcag

SEQ ID NO: 239 - DNA, 27 bp, synthetic construct
  misc_feature 1..27: note = Synthetic Construct
  source 1..27: mol_type = other DNA
aagggatcctgacagcccgtgggcgat

SEQ ID NO: 240 - DNA, 23 bp, synthetic construct
  misc_feature 1..23: note = Synthetic Construct
  source 1..23: mol_type = other DNA
aatattcatacagtctcaagagt

SEQ ID NO: 241 - DNA, 23 bp, synthetic construct
  misc_feature 1..23: note = Synthetic Construct
  source 1..23: mol_type = other DNA
aatcaaggcttaggggaagaagt

SEQ ID NO: 242 - DNA, 23 bp, synthetic construct
  misc_feature 1..23: note = Synthetic Construct
  source 1..23: mol_type = other DNA
gaatcaaggcttaggggaagaag

SEQ ID NO: 243 - DNA, 23 bp, synthetic construct
  misc_feature 1..23: note = Synthetic Construct
  source 1..23: mol_type = other DNA
tgaatcaaggcttaggggaagaa

SEQ ID NO: 244 - DNA, 23 bp, synthetic construct
  misc_feature 1..23: note = Synthetic Construct
  source 1..23: mol_type = other DNA
ttgaatcaaggcttaggggaaga

SEQ ID NO: 245 - DNA, 27 bp, synthetic construct
  misc_feature 1..27: note = Synthetic Construct
  source 1..27: mol_type = other DNA
cttgaatcaaggcttaggggaagaagt

SEQ ID NO: 246 - DNA, 23 bp, synthetic construct
  misc_feature 1..23: note = Synthetic Construct
  source 1..23: mol_type = other DNA
aatgacaacgtcttactgaagaa

SEQ ID NO: 247 - DNA, 23 bp, synthetic construct
  misc_feature 1..23: note = Synthetic Construct
  source 1..23: mol_type = other DNA
aaatgacaacgtcttactgaaga

SEQ ID NO: 248 - DNA, 23 bp, synthetic construct
  misc_feature 1..23: note = Synthetic Construct
  source 1..23: mol_type = other DNA
caaatgacaacgtcttactgaag

SEQ ID NO: 249 - DNA, 23 bp, synthetic construct
  misc_feature 1..23: note = Synthetic Construct
  source 1..23: mol_type = other DNA
tcaaatgacaacgtcttactgaa

SEQ ID NO: 250 - DNA, 23 bp, synthetic construct
  misc_feature 1..23: note = Synthetic Construct
  source 1..23: mol_type = other DNA
acaaataagtaaaaagcctaaaa

SEQ ID NO: 251 - DNA, 23 bp, synthetic construct
  misc_feature 1..23: note = Synthetic Construct
  source 1..23: mol_type = other DNA
acagatgtgtcatgtgtgagaaa

SEQ ID NO: 252 - DNA, 23 bp, synthetic construct
  misc_feature 1..23: note = Synthetic Construct
  source 1..23: mol_type = other DNA
aacagatgtgtcatgtgtgagaa

SEQ ID NO: 253 - DNA, 23 bp, synthetic construct
  misc_feature 1..23: note = Synthetic Construct
  source 1..23: mol_type = other DNA
gaacagatgtgtcatgtgtgaga

SEQ ID NO: 254 - DNA, 23 bp, synthetic construct
  misc_feature 1..23: note = Synthetic Construct
  source 1..23: mol_type = other DNA
agaacagatgtgtcatgtgtgag

SEQ ID NO: 255 - DNA, 23 bp, synthetic construct
  misc_feature 1..23: note = Synthetic Construct
  source 1..23: mol_type = other DNA
acctatggataattacgagttga

SEQ ID NO: 256 - DNA, 23 bp, synthetic construct
  misc_feature 1..23: note = Synthetic Construct
  source 1..23: mol_type = other DNA
aacctatggataattacgagttg

SEQ ID NO: 257 - DNA, 23 bp, synthetic construct
  misc_feature 1..23: note = Synthetic Construct
  source 1..23: mol_type = other DNA
ttaacctatggataattacgagt

SEQ ID NO: 258 - DNA, 27 bp, synthetic construct
  misc_feature 1..27: note = Synthetic Construct
  source 1..27: mol_type = other DNA
ttaacctatggataattacgagttgat

SEQ ID NO: 259 - DNA, 23 bp, synthetic construct
  misc_feature 1..23: note = Synthetic Construct
  source 1..23: mol_type = other DNA
actactccaattccttcaaagga

SEQ ID NO: 260 - DNA, 23 bp, synthetic construct
  misc_feature 1..23: note = Synthetic Construct
  source 1..23: mol_type = other DNA
aactactccaattccttcaaagg

SEQ ID NO: 261 - DNA, 27 bp, synthetic construct
  misc_feature 1..27: note = Synthetic Construct
  source 1..27: mol_type = other DNA
aaactactccaattccttcaaaggaat

SEQ ID NO: 262 - DNA, 23 bp, synthetic construct
  misc_feature 1..23: note = Synthetic Construct
  source 1..23: mol_type = other DNA
actacttgtcaatgaatgtgagg

SEQ ID NO: 263 - DNA, 23 bp, synthetic construct
  misc_feature 1..23: note = Synthetic Construct
  source 1..23: mol_type = other DNA
aactacttgtcaatgaatgtgag

SEQ ID NO: 264 - DNA, 23 bp, synthetic construct
  misc_feature 1..23: note = Synthetic Construct
  source 1..23: mol_type = other DNA
caactacttgtcaatgaatgtga

SEQ ID NO: 265 - DNA, 23 bp, synthetic construct
  misc_feature 1..23: note = Synthetic Construct
  source 1..23: mol_type = other DNA
ccaactacttgtcaatgaatgtg

SEQ ID NO: 266 - DNA, 23 bp, synthetic construct
  misc_feature 1..23: note = Synthetic Construct
  source 1..23: mol_type = other DNA
gccaactacttgtcaatgaatgt

SEQ ID NO: 267 - DNA, 23 bp, synthetic construct
  misc_feature 1..23: note = Synthetic Construct
  source 1..23: mol_type = other DNA
actagatgagactggaaccccag

SEQ ID NO: 268 - DNA, 27 bp, synthetic construct
  misc_feature 1..27: note = Synthetic Construct
  source 1..27: mol_type = other DNA
tagactagatgagactggaaccccagt

SEQ ID NO: 269 - DNA, 27 bp, synthetic construct
  misc_feature 1..27: note = Synthetic Construct
  source 1..27: mol_type = other DNA
actgctagcaaaccacactattccagt

SEQ ID NO: 270 - DNA, 23 bp, synthetic construct
  misc_feature 1..23: note = Synthetic Construct
  source 1..23: mol_type = other DNA
agaaactattcagcttctgttag

SEQ ID NO: 271 - DNA, 23 bp, synthetic construct
  misc_feature 1..23: note = Synthetic Construct
  source 1..23: mol_type = other DNA
agatagacagaagaccgctgggt

SEQ ID NO: 272 - DNA, 23 bp, synthetic construct
  misc_feature 1..23: note = Synthetic Construct
  source 1..23: mol_type = other DNA
tagatagacagaagaccgctggg

SEQ ID NO: 273 - DNA, 23 bp, synthetic construct
  misc_feature 1..23: note = Synthetic Construct
  source 1..23: mol_type = other DNA
gtagatagacagaagaccgctgg

SEQ ID NO: 274 - DNA, 23 bp, synthetic construct
  misc_feature 1..23: note = Synthetic Construct
  source 1..23: mol_type = other DNA
tgtagatagacagaagaccgctg

SEQ ID NO: 275 - DNA, 27 bp, synthetic construct
  misc_feature 1..27: note = Synthetic Construct
  source 1..27: mol_type = other DNA
ctgtagatagacagaagaccgctgggt

SEQ ID NO: 276 - DNA, 23 bp, synthetic construct
  misc_feature 1..23: note = Synthetic Construct
  source 1..23: mol_type = other DNA
agtagagtgaacttcggaaaaag

SEQ ID NO: 277 - DNA, 23 bp, synthetic construct
  misc_feature 1..23: note = Synthetic Construct
  source 1..23: mol_type = other DNA
aagtagagtgaacttcggaaaaa

SEQ ID NO: 278 - DNA, 23 bp, synthetic construct
  misc_feature 1..23: note = Synthetic Construct
  source 1..23: mol_type = other DNA
caagtagagtgaacttcggaaaa

SEQ ID NO: 279 - DNA, 23 bp, synthetic construct
  misc_feature 1..23: note = Synthetic Construct
  source 1..23: mol_type = other DNA
tcaagtagagtgaacttcggaaa

SEQ ID NO: 280 - DNA, 27 bp, synthetic construct
  misc_feature 1..27: note = Synthetic Construct
  source 1..27: mol_type = other DNA
tcaagtagagtgaacttcggaaaaagt

SEQ ID NO: 281 - DNA, 23 bp, synthetic construct
  misc_feature 1..23: note = Synthetic Construct
  source 1..23: mol_type = other DNA
agtaggatcacatgtgccaacag

SEQ ID NO: 282 - DNA, 23 bp, synthetic construct
  misc_feature 1..23: note = Synthetic Construct
  source 1..23: mol_type = other DNA
ctcagtaggatcacatgtgccaa

SEQ ID NO: 283 - DNA, 27 bp, synthetic construct
  misc_feature 1..27: note = Synthetic Construct
  source 1..27: mol_type = other DNA
tcagtaggatcacatgtgccaacaggt

SEQ ID NO: 284 - DNA, 23 bp, synthetic construct
  misc_feature 1..23: note = Synthetic Construct
  source 1..23: mol_type = other DNA
agtcttcagagtttcatggcagt

SEQ ID NO: 285 - DNA, 27 bp, synthetic construct
  misc_feature 1..27: note = Synthetic Construct
  source 1..27: mol_type = other DNA
ctgcagtcttcagagtttcatggcagt

SEQ ID NO: 286 - DNA, 23 bp, synthetic construct
  misc_feature 1..23: note = Synthetic Construct
  source 1..23: mol_type = other DNA
agtgaataaaggttagacattaa

SEQ ID NO: 287 - DNA, 23 bp, synthetic construct
  misc_feature 1..23: note = Synthetic Construct
  source 1..23: mol_type = other DNA
agttattcttccaaagcagcagt

SEQ ID NO: 288 - DNA, 23 bp, synthetic construct
  misc_feature 1..23: note = Synthetic Construct
  source 1..23: mol_type = other DNA
aagttattcttccaaagcagcag

SEQ ID NO: 289 - DNA, 23 bp, synthetic construct
  misc_feature 1..23: note = Synthetic Construct
  source 1..23: mol_type = other DNA
ttaagttattcttccaaagcagc

SEQ ID NO: 290 - DNA, 27 bp, synthetic construct
  misc_feature 1..27: note = Synthetic Construct
  source 1..27: mol_type = other DNA
cttaagttattcttccaaagcagcagt

SEQ ID NO: 291 - DNA, 23 bp, synthetic construct
  misc_feature 1..23: note = Synthetic Construct
  source 1..23: mol_type = other DNA
atcaatctcccaatacctggaga

SEQ ID NO: 292 - DNA, 23 bp, synthetic construct
  misc_feature 1..23: note = Synthetic Construct
  source 1..23: mol_type = other DNA
catcaatctcccaatacctggag

SEQ ID NO: 293 - DNA, 23 bp, synthetic construct
  misc_feature 1..23: note = Synthetic Construct
  source 1..23: mol_type = other DNA
ccatcaatctcccaatacctgga

SEQ ID NO: 294 - DNA, 23 bp, synthetic construct
  misc_feature 1..23: note = Synthetic Construct
  source 1..23: mol_type = other DNA
cccatcaatctcccaatacctgg

SEQ ID NO: 295 - DNA, 23 bp, synthetic construct
  misc_feature 1..23: note = Synthetic Construct
  source 1..23: mol_type = other DNA
gcccatcaatctcccaatacctg

SEQ ID NO: 296 - DNA, 23 bp, synthetic construct
  misc_feature 1..23: note = Synthetic Construct
  source 1..23: mol_type = other DNA
atcagtaggatgaagtacaagaa

SEQ ID NO: 297 - DNA, 23 bp, synthetic construct
  misc_feature 1..23: note = Synthetic Construct
  source 1..23: mol_type = other DNA
aatcagtaggatgaagtacaaga

SEQ ID NO: 298 - DNA, 23 bp, synthetic construct
  misc_feature 1..23: note = Synthetic Construct
  source 1..23: mol_type = other DNA
atcagtgagatgaagtacaagaa

SEQ ID NO: 299 - DNA, 23 bp, synthetic construct
  misc_feature 1..23: note = Synthetic Construct
  source 1..23: mol_type = other DNA
atcattcaatttgaggttgaaga

SEQ ID NO: 300 - DNA, 23 bp, synthetic construct
  misc_feature 1..23: note = Synthetic Construct
  source 1..23: mol_type = other DNA
aatcattcaatttgaggttgaag

SEQ ID NO: 301 - DNA, 23 bp, synthetic construct
  misc_feature 1..23: note = Synthetic Construct
  source 1..23: mol_type = other DNA
taatcattcaatttgaggttgaa

SEQ ID NO: 302 - DNA, 23 bp, synthetic construct
  misc_feature 1..23: note = Synthetic Construct
  source 1..23: mol_type = other DNA
ttaatcattcaatttgaggttga

SEQ ID NO: 303 - DNA, 23 bp, synthetic construct
  misc_feature 1..23: note = Synthetic Construct
  source 1..23: mol_type = other DNA
tttaatcattcaatttgaggttg

SEQ ID NO: 304 - DNA, 23 bp, synthetic construct
  misc_feature 1..23: note = Synthetic Construct
  source 1..23: mol_type = other DNA
atcctataggtcactgaagaggt

SEQ ID NO: 305 - DNA, 23 bp, synthetic construct
  misc_feature 1..23: note = Synthetic Construct
  source 1..23: mol_type = other DNA
catcctataggtcactgaagagg

SEQ ID NO: 306 - DNA, 23 bp, synthetic construct
  misc_feature 1..23: note = Synthetic Construct
  source 1..23: mol_type = other DNA
ccatcctataggtcactgaagag

SEQ ID NO: 307 - DNA, 27 bp, synthetic construct
  misc_feature 1..27: note = Synthetic Construct
  source 1..27: mol_type = other DNA
tcccatcctataggtcactgaagaggt

SEQ ID NO: 308 - DNA, 23 bp, synthetic construct
  misc_feature 1..23: note = Synthetic Construct
  source 1..23: mol_type = other DNA
atctattccaccagggcctgagc

SEQ ID NO: 309 - DNA, 23 bp, synthetic construct
  misc_feature 1..23: note = Synthetic Construct
  source 1..23: mol_type = other DNA
accatctattccaccagggcctg

SEQ ID NO: 310 - DNA, 27 bp, synthetic construct
  misc_feature 1..27: note = Synthetic Construct
  source 1..27: mol_type = other DNA
atctattccaccagggcctgagctgat

SEQ ID NO: 311 - DNA, 23 bp, synthetic construct
  misc_feature 1..23: note = Synthetic Construct
  source 1..23: mol_type = other DNA
atctattcccttgtggtcaccgt

SEQ ID NO: 312 - DNA, 23 bp, synthetic construct
  misc_feature 1..23: note = Synthetic Construct
  source 1..23: mol_type = other DNA
gatctattcccttgtggtcaccg

SEQ ID NO: 313 - DNA, 27 bp, synthetic construct
  misc_feature 1..27: note = Synthetic Construct
  source 1..27: mol_type = other DNA
gatctattcccttgtggtcaccgtagt

SEQ ID NO: 314 - DNA, 23 bp, synthetic construct
  misc_feature 1..23: note = Synthetic Construct
  source 1..23: mol_type = other DNA
atgaaaggagggtccctatacag

SEQ ID NO: 315 - DNA, 27 bp, synthetic construct
  misc_feature 1..27: note = Synthetic Construct
  source 1..27: mol_type = other DNA
gtcatgaaaggagggtccctatacagt

SEQ ID NO: 316 - DNA, 23 bp, synthetic construct
  misc_feature 1..23: note = Synthetic Construct
  source 1..23: mol_type = other DNA
atgactggaagctaaggaagaag

SEQ ID NO: 317 - DNA, 23 bp, synthetic construct
  misc_feature 1..23: note = Synthetic Construct
  source 1..23: mol_type = other DNA
aatgactggaagctaaggaagaa

SEQ ID NO: 318 - DNA, 23 bp, synthetic construct
  misc_feature 1..23: note = Synthetic Construct
  source 1..23: mol_type = other DNA
caatgactggaagctaaggaaga

SEQ ID NO: 319 - DNA, 23 bp, synthetic construct
  misc_feature 1..23: note = Synthetic Construct
  source 1..23: mol_type = other DNA
acaatgactggaagctaaggaag

SEQ ID NO: 320 - DNA, 23 bp, synthetic construct
  misc_feature 1..23: note = Synthetic Construct
  source 1..23: mol_type = other DNA
cacaatgactggaagctaaggaa

SEQ ID NO: 321 - DNA, 23 bp, synthetic construct
  misc_feature 1..23: note = Synthetic Construct
  source 1..23: mol_type = other DNA
atttaagcttcaatttctccttg

SEQ ID NO: 322 - DNA, 23 bp, synthetic construct
  misc_feature 1..23: note = Synthetic Construct
  source 1..23: mol_type = other DNA
atttatgcaaagttgagtcttcg

SEQ ID NO: 323 - DNA, 23 bp, synthetic construct
  misc_feature 1..23: note = Synthetic Construct
  source 1..23: mol_type = other DNA
attttaactcctactcagactgt

SEQ ID NO: 324 - DNA, 23 bp, synthetic construct
  misc_feature 1..23: note = Synthetic Construct
  source 1..23: mol_type = other DNA
tattttaactcctactcagactg

SEQ ID NO: 325 - DNA, 23 bp, synthetic construct
  misc_feature 1..23: note = Synthetic Construct
  source 1..23: mol_type = other DNA
cacacagaatctactggccagaa

SEQ ID NO: 326 - DNA, 23 bp, synthetic construct
  misc_feature 1..23: note = Synthetic Construct
  source 1..23: mol_type = other DNA
ctcacacagaatctactggccag

SEQ ID NO: 327 - DNA, 23 bp, synthetic construct
  misc_feature 1..23: note = Synthetic Construct
  source 1..23: mol_type = other DNA
cactagcggaggtctttggccaa

SEQ ID NO: 328 - DNA, 23 bp, synthetic construct
  misc_feature 1..23: note = Synthetic Construct
  source 1..23: mol_type = other DNA
cactttattgctcttgcagagaa

SEQ ID NO: 329 - DNA, 23 bp, synthetic construct
  misc_feature 1..23: note = Synthetic Construct
  source 1..23: mol_type = other DNA
ccactttattgctcttgcagaga

SEQ ID NO: 330 - DNA, 23 bp, synthetic construct
  misc_feature 1..23: note = Synthetic Construct
  source 1..23: mol_type = other DNA
cagataaagtgtgtaaaggacag

SEQ ID NO: 331 - DNA, 23 bp, synthetic construct
  misc_feature 1..23: note = Synthetic Construct
  source 1..23: mol_type = other DNA
caacagataaagtgtgtaaagga

SEQ ID NO: 332 - DNA, 23 bp, synthetic construct
  misc_feature 1..23: note = Synthetic Construct
  source 1..23: mol_type = other DNA
tcaacagataaagtgtgtaaagg

SEQ ID NO: 333 - DNA, 23 bp, synthetic construct
  misc_feature 1..23: note = Synthetic Construct
  source 1..23: mol_type = other DNA
catttaagctgcgtccaccttgt

SEQ ID NO: 334 - DNA, 23 bp, synthetic construct
  misc_feature 1..23: note = Synthetic Construct
  source 1..23: mol_type = other DNA
gcatttaagctgcgtccaccttg

SEQ ID NO: 335 - DNA, 23 bp, synthetic construct
  misc_feature 1..23: note = Synthetic Construct
  source 1..23: mol_type = other DNA
ccattcagggatgcttcgcaaaa

SEQ ID NO: 336 - DNA, 23 bp, synthetic construct
  misc_feature 1..23: note = Synthetic Construct
  source 1..23: mol_type = other DNA
cccattcagggatgcttcgcaaa

SEQ ID NO: 337 - DNA, 23 bp, synthetic construct
  misc_feature 1..23: note = Synthetic Construct
  source 1..23: mol_type = other DNA
cccagaagtaattgaatgtggaa

SEQ ID NO: 338 - DNA, 23 bp, synthetic construct
  misc_feature 1..23: note = Synthetic Construct
  source 1..23: mol_type = other DNA
gcccagaagtaattgaatgtgga

SEQ ID NO: 339 - DNA, 23 bp, synthetic construct
  misc_feature 1..23: note = Synthetic Construct
  source 1..23: mol_type = other DNA
ccgataagtttgacgccagcctg

SEQ ID NO: 340 - DNA, 23 bp, synthetic construct
  misc_feature 1..23: note = Synthetic Construct
  source 1..23: mol_type = other DNA
actccgataagtttgacgccagc

SEQ ID NO: 341 - DNA, 23 bp, synthetic construct
  misc_feature 1..23: note = Synthetic Construct
  source 1..23: mol_type = other DNA
cactccgataagtttgacgccag

SEQ ID NO: 342 - DNA, 23 bp, synthetic construct
  misc_feature 1..23: note = Synthetic Construct
  source 1..23: mol_type = other DNA
cctaaatgatccactttgtgatg

SEQ ID NO: 343 - DNA, 23 bp, synthetic construct
  misc_feature 1..23: note = Synthetic Construct
  source 1..23: mol_type = other DNA
cctacagagaaaggtgcagacgc

SEQ ID NO: 344 - DNA, 23 bp, synthetic construct
  misc_feature 1..23: note = Synthetic Construct
  source 1..23: mol_type = other DNA
tcctacagagaaaggtgcagacg

SEQ ID NO: 345 - DNA, 23 bp, synthetic construct
  misc_feature 1..23: note = Synthetic Construct
  source 1..23: mol_type = other DNA
gttcctacagagaaaggtgcaga

SEQ ID NO: 346 - DNA, 23 bp, synthetic construct
  misc_feature 1..23: note = Synthetic Construct
  source 1..23: mol_type = other DNA
agttcctacagagaaaggtgcag

SEQ ID NO: 347 - DNA, 23 bp, synthetic construct
  misc_feature 1..23: note = Synthetic Construct
  source 1..23: mol_type = other DNA
cctaggcagactggatgctctgt

SEQ ID NO: 348 - DNA, 23 bp, synthetic construct
  misc_feature 1..23: note = Synthetic Construct
  source 1..23: mol_type = other DNA
tcctaggcagactggatgctctg

SEQ ID NO: 349 - DNA, 27 bp, synthetic construct
  misc_feature 1..27: note = Synthetic Construct
  source 1..27: mol_type = other DNA
ctattggagcctataagtatactggat

SEQ ID NO: 350 - DNA, 23 bp, synthetic construct
  misc_feature 1..23: note = Synthetic Construct
  source 1..23: mol_type = other DNA
ctcaaagtgcctgtgtgcaatag

SEQ ID NO: 351 - DNA, 23 bp, synthetic construct
  misc_feature 1..23: note = Synthetic Construct
  source 1..23: mol_type = other DNA
ctcctcaaagtgcctgtgtgcaa

SEQ ID NO: 352 - DNA, 27 bp, synthetic construct
  misc_feature 1..27: note = Synthetic Construct
  source 1..27: mol_type = other DNA
ctcctcaaagtgcctgtgtgcaatagt

SEQ ID NO: 353 - DNA, 27 bp, synthetic construct
  misc_feature 1..27: note = Synthetic Construct
  source 1..27: mol_type = other DNA
tttctcctcaaagtgcctgtgtgcaat

SEQ ID NO: 354 - DNA, 23 bp, synthetic construct
  misc_feature 1..23: note = Synthetic Construct
  source 1..23: mol_type = other DNA
ctcactcactcaccctgcaaagg

SEQ ID NO: 355 - DNA, 23 bp, synthetic construct
  misc_feature 1..23: note = Synthetic Construct
  source 1..23: mol_type = other DNA
tctcactcactcaccctgcaaag

SEQ ID NO: 356 - DNA, 23 bp, synthetic construct
  misc_feature 1..23: note = Synthetic Construct
  source 1..23: mol_type = other DNA
ctctcactcactcaccctgcaaa

SEQ ID NO: 357 - DNA, 23 bp, synthetic construct
  misc_feature 1..23: note = Synthetic Construct
  source 1..23: mol_type = other DNA
cctctcactcactcaccctgcaa

SEQ ID NO: 358 - DNA, 23 bp, synthetic construct
  misc_feature 1..23: note = Synthetic Construct
  source 1..23: mol_type = other DNA
ctcactctatggcctgcagcatg

SEQ ID NO: 359 - DNA, 23 bp, synthetic construct
  misc_feature 1..23: note = Synthetic Construct
  source 1..23: mol_type = other DNA
tttctcactctatggcctgcagc

SEQ ID NO: 360 - DNA, 23 bp, synthetic construct
  misc_feature 1..23: note = Synthetic Construct
  source 1..23: mol_type = other DNA
ttttctcactctatggcctgcag

SEQ ID NO: 361 - DNA, 23 bp, synthetic construct
  misc_feature 1..23: note = Synthetic Construct
  source 1..23: mol_type = other DNA
ctcaggaggtgacagctatccag

SEQ ID NO: 362 - DNA, 27 bp, synthetic construct
  misc_feature 1..27: note = Synthetic Construct
  source 1..27: mol_type = other DNA
ctgctcaggaggtgacagctatccagt

SEQ ID NO: 363 - DNA, 23 bp, synthetic construct
  misc_feature 1..23: note = Synthetic Construct
  source 1..23: mol_type = other DNA
ctctcactttctctcatctgtga

SEQ ID NO: 364 - DNA, 23 bp, synthetic construct
  misc_feature 1..23: note = Synthetic Construct
  source 1..23: mol_type = other DNA
cctctcactttctctcatctgtg

SEQ ID NO: 365 - DNA, 23 bp, synthetic construct
  misc_feature 1..23: note = Synthetic Construct
  source 1..23: mol_type = other DNA
tcctctcactttctctcatctgt

SEQ ID NO: 366 - DNA, 27 bp, synthetic construct
  misc_feature 1..27: note = Synthetic Construct
  source 1..27: mol_type = other DNA
ttcctctcactttctctcatctgtgat

SEQ ID NO: 367 - DNA, 23 bp, synthetic construct
  misc_feature 1..23: note = Synthetic Construct
  source 1..23: mol_type = other DNA
ctgaaagaagctctcctcccagc

SEQ ID NO: 368 - DNA, 23 bp, synthetic construct
  misc_feature 1..23: note = Synthetic Construct
  source 1..23: mol_type = other DNA
gctgaaagaagctctcctcccag

SEQ ID NO: 369 - DNA, 23 bp, synthetic construct
  misc_feature 1..23: note = Synthetic Construct
  source 1..23: mol_type = other DNA
ctgaattactcgctcagaagctg

SEQ ID NO: 370 - DNA, 23 bp, synthetic construct
  misc_feature 1..23: note = Synthetic Construct
  source 1..23: mol_type = other DNA
agctgaattactcgctcagaagc

SEQ ID NO: 371 - DNA, 23 bp, synthetic construct
  misc_feature 1..23: note = Synthetic Construct
  source 1..23: mol_type = other DNA
cagctgaattactcgctcagaag

SEQ ID NO: 372 - DNA, 23 bp, synthetic construct
  misc_feature 1..23: note = Synthetic Construct
  source 1..23: mol_type = other DNA
acagctgaattactcgctcagaa

SEQ ID NO: 373 - DNA, 23 bp, synthetic construct
  misc_feature 1..23: note = Synthetic Construct
  source 1..23: mol_type = other DNA
ctgagtagaaggcggtaaaccgt

SEQ ID NO: 374 - DNA, 23 bp, synthetic construct
  misc_feature 1..23: note = Synthetic Construct
  source 1..23: mol_type = other DNA
tctgagtagaaggcggtaaaccg

SEQ ID NO: 375 - DNA, 23 bp, synthetic construct
  misc_feature 1..23: note = Synthetic Construct
  source 1..23: mol_type = other DNA
cttaccaaaggtcacaaaggtaa

SEQ ID NO: 376 - DNA, 23 bp, synthetic construct
  misc_feature 1..23: note = Synthetic Construct
  source 1..23: mol_type = other DNA
gacttaccaaaggtcacaaaggt

SEQ ID NO: 377 - DNA, 23 bp, synthetic construct
  misc_feature 1..23: note = Synthetic Construct
  source 1..23: mol_type = other DNA
tgacttaccaaaggtcacaaagg

SEQ ID NO: 378 - DNA, 23 bp, synthetic construct
  misc_feature 1..23: note = Synthetic Construct
  source 1..23: mol_type = other DNA
atgacttaccaaaggtcacaaag

SEQ ID NO: 379 - DNA, 27 bp, synthetic construct
  misc_feature 1..27: note = Synthetic Construct
  source 1..27: mol_type = other DNA
agatgacttaccaaaggtcacaaaggt

SEQ ID NO: 380 - DNA, 23 bp, synthetic construct
  misc_feature 1..23: note = Synthetic Construct
  source 1..23: mol_type = other DNA
cttagagagagtcaatgaggaga

SEQ ID NO: 381 - DNA, 23 bp, synthetic construct
  misc_feature 1..23: note = Synthetic Construct
  source 1..23: mol_type = other DNA
tcttagagagagtcaatgaggag

SEQ ID NO: 382 - DNA, 23 bp, synthetic construct
  misc_feature 1..23: note = Synthetic Construct
  source 1..23: mol_type = other DNA
atcttagagagagtcaatgagga

SEQ ID NO: 383 - DNA, 23 bp, synthetic construct
  misc_feature 1..23: note = Synthetic Construct
  source 1..23: mol_type = other DNA
gatcttagagagagtcaatgagg

SEQ ID NO: 384 - DNA, 23 bp, synthetic construct
  misc_feature 1..23: note = Synthetic Construct
  source 1..23: mol_type = other DNA
tgatcttagagagagtcaatgag

SEQ ID NO: 385 - DNA, 27 bp, synthetic construct
  misc_feature 1..27: note = Synthetic Construct
  source 1..27: mol_type = other DNA
gatcttagagagagtcaatgaggagat

SEQ ID NO: 386 - DNA, 23 bp, synthetic construct
  misc_feature 1..23: note = Synthetic Construct
  source 1..23: mol_type = other DNA
cttatagacgctgctcaaaattg

SEQ ID NO: 387 - DNA, 23 bp, synthetic construct
  misc_feature 1..23: note = Synthetic Construct
  source 1..23: mol_type = other DNA
tttcttatagacgctgctcaaaa

SEQ ID NO: 388 - DNA, 23 bp, synthetic construct
  misc_feature 1..23: note = Synthetic Construct
  source 1..23: mol_type = other DNA
ctttcttatagacgctgctcaaa

SEQ ID NO: 389 - DNA, 27 bp, synthetic construct
  misc_feature 1..27: note = Synthetic Construct
  source 1..27: mol_type = other DNA
tactttcttatagacgctgctcaaaat

SEQ ID NO: 390 - DNA, 23 bp, synthetic construct
  misc_feature 1..23: note = Synthetic Construct
  source 1..23: mol_type = other DNA
cttattctagatcttcttgaagc

SEQ ID NO: 391 - DNA, 23 bp, synthetic construct
  misc_feature 1..23: note = Synthetic Construct
  source 1..23: mol_type = other DNA
cttattgaggcaaaacttggaag

SEQ ID NO: 392 - DNA, 23 bp, synthetic construct
  misc_feature 1..23: note = Synthetic Construct
  source 1..23: mol_type = other DNA
tcacttattgaggcaaaacttgg

SEQ ID NO: 393 - DNA, 23 bp, synthetic construct
  misc_feature 1..23: note = Synthetic Construct
  source 1..23: mol_type = other DNA
ctcacttattgaggcaaaacttg

SEQ ID NO: 394 - DNA, 27 bp, synthetic construct
  misc_feature 1..27: note = Synthetic Construct
  source 1..27: mol_type = other DNA
acttattgaggcaaaacttggaagagt

SEQ ID NO: 395 - DNA, 23 bp, synthetic construct
  misc_feature 1..23: note = Synthetic Construct
  source 1..23: mol_type = other DNA
ctttgatgggaagaagtagagga

SEQ ID NO: 396 - DNA, 23 bp, synthetic construct
  misc_feature 1..23: note = Synthetic Construct
  source 1..23: mol_type = other DNA
gctttgatgggaagaagtagagg

SEQ ID NO: 397 - DNA, 23 bp, synthetic construct
  misc_feature 1..23: note = Synthetic Construct
  source 1..23: mol_type = other DNA
tgctttgatgggaagaagtagag

SEQ ID NO: 398 - DNA, 23 bp, synthetic construct
  misc_feature 1..23: note = Synthetic Construct
  source 1..23: mol_type = other DNA
gaaagtctatgccagaaaggagg

SEQ ID NO: 399 - DNA, 23 bp, synthetic construct
  misc_feature 1..23: note = Synthetic Construct
  source 1..23: mol_type = other DNA
ggaaagtctatgccagaaaggag

SEQ ID NO: 400 - DNA, 23 bp, synthetic construct
  misc_feature 1..23: note = Synthetic Construct
  source 1..23: mol_type = other DNA
tggaaagtctatgccagaaagga

SEQ ID NO: 401 - DNA, 23 bp, synthetic construct
  misc_feature 1..23: note = Synthetic Construct
  source 1..23: mol_type = other DNA
gtggaaagtctatgccagaaagg

SEQ ID NO: 402 - DNA, 23 bp, synthetic construct
  misc_feature 1..23: note = Synthetic Construct
  source 1..23: mol_type = other DNA
tgtggaaagtctatgccagaaag

SEQ ID NO: 403 - DNA, 23 bp, synthetic construct
  misc_feature 1..23: note = Synthetic Construct
  source 1..23: mol_type = other DNA
gacataggtccagcagtcagaaa

SEQ ID NO: 404 - DNA, 23 bp, synthetic construct
  misc_feature 1..23: note = Synthetic Construct
  source 1..23: mol_type = other DNA
ggacataggtccagcagtcagaa

SEQ ID NO: 405 - DNA, 23 bp, synthetic construct
  misc_feature 1..23: note = Synthetic Construct
  source 1..23: mol_type = other DNA
aggacataggtccagcagtcaga

SEQ ID NO: 406 - DNA, 23 bp, synthetic construct
  misc_feature 1..23: note = Synthetic Construct
  source 1..23: mol_type = other DNA
gacgctactcaaaattggctggt

SEQ ID NO: 407 - DNA, 23 bp, synthetic construct
  misc_feature 1..23: note = Synthetic Construct
  source 1..23: mol_type = other DNA
agacgctactcaaaattggctgg

SEQ ID NO: 408 - DNA, 27 bp, synthetic construct
  misc_feature 1..27: note = Synthetic Construct
  source 1..27: mol_type = other DNA
tgtagacgctactcaaaattggctggt

SEQ ID NO: 409 - DNA, 23 bp, synthetic construct
  misc_feature 1..23: note = Synthetic Construct
  source 1..23: mol_type = other DNA
gagatatgaagataagtgaaaaa

SEQ ID NO: 410 - DNA, 23 bp, synthetic construct
  misc_feature 1..23: note = Synthetic Construct
  source 1..23: mol_type = other DNA
agagatatgaagataagtgaaaa

SEQ ID NO: 411 - DNA, 23 bp, synthetic construct
  misc_feature 1..23: note = Synthetic Construct
  source 1..23: mol_type = other DNA
aagagatatgaagataagtgaaa

SEQ ID NO: 412 - DNA, 23 bp, synthetic construct
  misc_feature 1..23: note = Synthetic Construct
  source 1..23: mol_type = other DNA
gaagagatatgaagataagtgaa

SEQ ID NO: 413 - DNA, 23 bp, synthetic construct
  misc_feature 1..23: note = Synthetic Construct
  source 1..23: mol_type = other DNA
tgaagagatatgaagataagtga

SEQ ID NO: 414 - DNA, 27 bp, synthetic construct
  misc_feature 1..27: note = Synthetic Construct
  source 1..27: mol_type = other DNA
gaagagatatgaagataagtgaaaaat

SEQ ID NO: 415 - DNA, 23 bp, synthetic construct
  misc_feature 1..23: note = Synthetic Construct
  source 1..23: mol_type = other DNA
gaggcttatttgatgctatctgc

SEQ ID NO: 416 - DNA, 23 bp, synthetic construct
  misc_feature 1..23: note = Synthetic Construct
  source 1..23: mol_type = other DNA
gatataagtcgtgtatattaatg

SEQ ID NO: 417 - DNA, 23 bp, synthetic construct
  misc_feature 1..23: note = Synthetic Construct
  source 1..23: mol_type = other DNA
cagatataagtcgtgtatattaa

SEQ ID NO: 418 - DNA, 27 bp, synthetic construct
  misc_feature 1..27: note = Synthetic Construct
  source 1..27: mol_type = other DNA
gtacagatataagtcgtgtatattaat

SEQ ID NO: 419 - DNA, 23 bp, synthetic construct
  misc_feature 1..23: note = Synthetic Construct
  source 1..23: mol_type = other DNA
gattagattttctgttgggagga

SEQ ID NO: 420 - DNA, 23 bp, synthetic construct
  misc_feature 1..23: note = Synthetic Construct
  source 1..23: mol_type = other DNA
agattagattttctgttgggagg

SEQ ID NO: 421 - DNA, 23 bp, synthetic construct
  misc_feature 1..23: note = Synthetic Construct
  source 1..23: mol_type = other DNA
cagattagattttctgttgggag

SEQ ID NO: 422 - DNA, 23 bp, synthetic construct
  misc_feature 1..23: note = Synthetic Construct
  source 1..23: mol_type = other DNA
tcagattagattttctgttggga

SEQ ID NO: 423 - DNA, 27 bp, synthetic construct
  misc_feature 1..27: note = Synthetic Construct
  source 1..27: mol_type = other DNA
tcagattagattttctgttgggaggat

SEQ ID NO: 424 - DNA, 23 bp, synthetic construct
  misc_feature 1..23: note = Synthetic Construct
  source 1..23: mol_type = other DNA
gctaattatagaaagcgatgatg

SEQ ID NO: 425 - DNA, 23 bp, synthetic construct
  misc_feature 1..23: note = Synthetic Construct
  source 1..23: mol_type = other DNA
tagctaattatagaaagcgatga

SEQ ID NO: 426 - DNA, 23 bp, synthetic construct
  misc_feature 1..23: note = Synthetic Construct
  source 1..23: mol_type = other DNA
gtagctaattatagaaagcgatg

SEQ ID NO: 427 - DNA, 27 bp, synthetic construct
  misc_feature 1..27: note = Synthetic Construct
  source 1..27: mol_type = other DNA
ttgtagctaattatagaaagcgatgat

SEQ ID NO: 428 - DNA, 23 bp, synthetic construct
  misc_feature 1..23: note = Synthetic Construct
  source 1..23: mol_type = other DNA
gctgacgggaaattgagagcaaa

SEQ ID NO: 429 - DNA, 23 bp, synthetic construct
  misc_feature 1..23: note = Synthetic Construct
  source 1..23: mol_type = other DNA
cgctgacgggaaattgagagcaa

SEQ ID NO: 430 - DNA, 23 bp, synthetic construct
  misc_feature 1..23: note = Synthetic Construct
  source 1..23: mol_type = other DNA
agcgctgacgggaaattgagagc

SEQ ID NO: 431 - DNA, 27 bp, synthetic construct
  misc_feature 1..27: note = Synthetic Construct
  source 1..27: mol_type = other DNA
agcgctgacgggaaattgagagcaaat

SEQ ID NO: 432 - DNA, 23 bp, synthetic construct
  misc_feature 1..23: note = Synthetic Construct
  source 1..23: mol_type = other DNA
gctgatctgatggcctggctttg

SEQ ID NO: 433 - DNA, 27 bp, synthetic construct
  misc_feature 1..27: note = Synthetic Construct
  source 1..27: mol_type = other DNA
agctgatctgatggcctggctttgaat

SEQ ID NO: 434 - DNA, 23 bp, synthetic construct
  misc_feature 1..23: note = Synthetic Construct
  source 1..23: mol_type = other DNA
gctttctataattcatctggagt

SEQ ID NO: 435 - DNA, 27 bp, synthetic construct
  misc_feature 1..27: note = Synthetic Construct
  source 1..27: mol_type = other DNA
aactgctttctataattcatctggagt

SEQ ID NO: 436 - DNA, 27 bp, synthetic construct
  misc_feature 1..27: note = Synthetic Construct
  source 1..27: mol_type = other DNA
ggccatatgtactttctagctttcaat

SEQ ID NO: 437 - DNA, 27 bp, synthetic construct
  misc_feature 1..27: note = Synthetic Construct
  source 1..27: mol_type = other DNA
ggccctttgctataagtattggccagt

SEQ ID NO: 438 - DNA, 23 bp, synthetic construct
  misc_feature 1..23: note = Synthetic Construct
  source 1..23: mol_type = other DNA
ggctaagtggtggtgacagcctg

SEQ ID NO: 439 - DNA, 23 bp, synthetic construct
  misc_feature 1..23: note = Synthetic Construct
  source 1..23: mol_type = other DNA
gatggctaagtggtggtgacagc

SEQ ID NO: 440 - DNA, 23 bp, synthetic construct
  misc_feature 1..23: note = Synthetic Construct
  source 1..23: mol_type = other DNA
gtactaataccactgatgagaaa

SEQ ID NO: 441 - DNA, 23 bp, synthetic construct
  misc_feature 1..23: note = Synthetic Construct
  source 1..23: mol_type = other DNA
tgtactaataccactgatgagaa

SEQ ID NO: 442 - DNA, 23 bp, synthetic construct
  misc_feature 1..23: note = Synthetic Construct
  source 1..23: mol_type = other DNA
ttgtactaataccactgatgaga

SEQ ID NO: 443 - DNA, 27 bp, synthetic construct
  misc_feature 1..27: note = Synthetic Construct
  source 1..27: mol_type = other DNA
cttgtactaataccactgatgagaaat

SEQ ID NO: 444 - DNA, 27 bp, synthetic construct
  misc_feature 1..27: note = Synthetic Construct
  source 1..27: mol_type = other DNA
gtcatagaaggagggtccctatacagt

SEQ ID NO: 445 - DNA, 23 bp, synthetic construct
  misc_feature 1..23: note = Synthetic Construct
  source 1..23: mol_type = other DNA
gtcattgtgtggctgactgctgg

SEQ ID NO: 446 - DNA, 23 bp, synthetic construct
  misc_feature 1..23: note = Synthetic Construct
  source 1..23: mol_type = other DNA
agtcattgtgtggctgactgctg

SEQ ID NO: 447 - DNA, 23 bp, synthetic construct
  misc_feature 1..23: note = Synthetic Construct
  source 1..23: mol_type = other DNA
ccagtcattgtgtggctgactgc

SEQ ID NO: 448 - DNA, 23 bp, synthetic construct
  misc_feature 1..23: note = Synthetic Construct
  source 1..23: mol_type = other DNA
tccagtcattgtgtggctgactg

SEQ ID NO: 449 - DNA, 23 bp, synthetic construct
  misc_feature 1..23: note = Synthetic Construct
  source 1..23: mol_type = other DNA
gtcttataaaagaacccagcggt

SEQ ID NO: 450 - DNA, 23 bp, synthetic construct
  misc_feature 1..23: note = Synthetic Construct
  source 1..23: mol_type = other DNA
tgtcttataaaagaacccagcgg

SEQ ID NO: 451 - DNA, 23 bp, synthetic construct
  misc_feature 1..23: note = Synthetic Construct
  source 1..23: mol_type = other DNA
atgtcttataaaagaacccagcg

SEQ ID NO: 452 - DNA, 27 bp, synthetic construct
  misc_feature 1..27: note = Synthetic Construct
  source 1..27: mol_type = other DNA
ggatgtcttataaaagaacccagcggt

SEQ ID NO: 453 - DNA, 23 bp, synthetic construct
  misc_feature 1..23: note = Synthetic Construct
  source 1..23: mol_type = other DNA
gtgaagtgaacttcggaaaaagt

SEQ ID NO: 454 - DNA, 23 bp, synthetic construct
  misc_feature 1..23: note = Synthetic Construct
  source 1..23: mol_type = other DNA
agtgaagtgaacttcggaaaaag

SEQ ID NO: 455 - DNA, 23 bp, synthetic construct
  misc_feature 1..23: note = Synthetic Construct
  source 1..23: mol_type = other DNA
aagtgaagtgaacttcggaaaaa

SEQ ID NO: 456 - DNA, 23 bp, synthetic construct
  misc_feature 1..23: note = Synthetic Construct
  source 1..23: mol_type = other DNA
caagtgaagtgaacttcggaaaa

SEQ ID NO: 457 - DNA, 23 bp, synthetic construct
  misc_feature 1..23: note = Synthetic Construct
  source 1..23: mol_type = other DNA
tcaagtgaagtgaacttcggaaa

SEQ ID NO: 458 - DNA, 27 bp, synthetic construct
  misc_feature 1..27: note = Synthetic Construct
  source 1..27: mol_type = other DNA
tcaagtgaagtgaacttcggaaaaagt

SEQ ID NO: 459 - DNA, 23 bp, synthetic construct
  misc_feature 1..23: note = Synthetic Construct
  source 1..23: mol_type = other DNA
gtgaatcgaattctgccagttgc

SEQ ID NO: 460 - DNA, 23 bp, synthetic construct
  misc_feature 1..23: note = Synthetic Construct
  source 1..23: mol_type = other DNA
ggtgaatcgaattctgccagttg

SEQ ID NO: 461 - DNA, 23 bp, synthetic construct
  misc_feature 1..23: note = Synthetic Construct
  source 1..23: mol_type = other DNA
gccggtgaatcgaattctgccag

SEQ ID NO: 462 - DNA, 23 bp, synthetic construct
  misc_feature 1..23: note = Synthetic Construct
  source 1..23: mol_type = other DNA
gtgacagacaaatgtagatgtgg

SEQ ID NO: 463 - DNA, 23 bp, synthetic construct
  misc_feature 1..23: note = Synthetic Construct
  source 1..23: mol_type = other DNA
agtgacagacaaatgtagatgtg

SEQ ID NO: 464 - DNA, 23 bp, synthetic construct
  misc_feature 1..23: note = Synthetic Construct
  source 1..23: mol_type = other DNA
cagtgacagacaaatgtagatgt

SEQ ID NO: 465 - DNA, 23 bp, synthetic construct
  misc_feature 1..23: note = Synthetic Construct
  source 1..23: mol_type = other DNA
ccagtgacagacaaatgtagatg

SEQ ID NO: 466 - DNA, 27 bp, synthetic construct
  misc_feature 1..27: note = Synthetic Construct
  source 1..27: mol_type = other DNA
ctccgccagtgacagacaaatgtagat

SEQ ID NO: 467 - DNA, 23 bp, synthetic construct
  misc_feature 1..23: note = Synthetic Construct
  source 1..23: mol_type = other DNA
gtgactctgcactaggctgaatg

SEQ ID NO: 468 - DNA, 23 bp, synthetic construct
  misc_feature 1..23: note = Synthetic Construct
  source 1..23: mol_type = other DNA
cagtgactctgcactaggctgaa

SEQ ID NO: 469 - DNA, 23 bp, synthetic construct
  misc_feature 1..23: note = Synthetic Construct
  source 1..23: mol_type = other DNA
ccagtgactctgcactaggctga

SEQ ID NO: 470 - DNA, 23 bp, synthetic construct
  misc_feature 1..23: note = Synthetic Construct
  source 1..23: mol_type = other DNA
accagtgactctgcactaggctg

SEQ ID NO: 471 - DNA, 27 bp, synthetic construct
  misc_feature 1..27: note = Synthetic Construct
  source 1..27: mol_type = other DNA
taccagtgactctgcactaggctgaat

SEQ ID NO: 472 - DNA, 23 bp, synthetic construct
  misc_feature 1..23: note = Synthetic Construct
  source 1..23: mol_type = other DNA
gtgctaagatgctggaccaaagt

SEQ ID NO: 473 - DNA, 23 bp, synthetic construct
  misc_feature 1..23: note = Synthetic Construct
  source 1..23: mol_type = other DNA
agtgctaagatgctggaccaaag

SEQ ID NO: 474 - DNA, 23 bp, synthetic construct
  misc_feature 1..23: note = Synthetic Construct
  source 1..23: mol_type = other DNA
aagtgctaagatgctggaccaaa

SEQ ID NO: 475 - DNA, 27 bp, synthetic construct
  misc_feature 1..27: note = Synthetic Construct
  source 1..27: mol_type = other DNA
gaaagtgctaagatgctggaccaaagt

SEQ ID NO: 476 - DNA, 23 bp, synthetic construct
  misc_feature 1..23: note = Synthetic Construct
  source 1..23: mol_type = other DNA
gttaaagatctgatagccggttg

SEQ ID NO: 477 - DNA, 23 bp, synthetic construct
  misc_feature 1..23: note = Synthetic Construct
  source 1..23: mol_type = other DNA
gaggttaaagatctgatagccgg

SEQ ID NO: 478 - DNA, 23 bp, synthetic construct
  misc_feature 1..23: note = Synthetic Construct
  source 1..23: mol_type = other DNA
tgaggttaaagatctgatagccg

SEQ ID NO: 479 - DNA, 27 bp, synthetic construct
  misc_feature 1..27: note = Synthetic Construct
  source 1..27: mol_type = other DNA
tttgaggttaaagatctgatagccggt

SEQ ID NO: 480 - DNA, 23 bp, synthetic construct
  misc_feature 1..23: note = Synthetic Construct
  source 1..23: mol_type = other DNA
gttagcacatgtgatcccactga

SEQ ID NO: 481 - DNA, 23 bp, synthetic construct
  misc_feature 1..23: note = Synthetic Construct
  source 1..23: mol_type = other DNA
tgttagcacatgtgatcccactg

SEQ ID NO: 482 - DNA, 27 bp, synthetic construct
  misc_feature 1..27: note = Synthetic Construct
  source 1..27: mol_type = other DNA
ctgttagcacatgtgatcccactgagt

SEQ ID NO: 483 - DNA, 23 bp, synthetic construct
  misc_feature 1..23: note = Synthetic Construct
  source 1..23: mol_type = other DNA
gttaggataatttagtccaaaaa

SEQ ID NO: 484 - DNA, 23 bp, synthetic construct
  misc_feature 1..23: note = Synthetic Construct
  source 1..23: mol_type = other DNA
tgttaggataatttagtccaaaa

SEQ ID NO: 485 - DNA, 23 bp, synthetic construct
  misc_feature 1..23: note = Synthetic Construct
  source 1..23: mol_type = other DNA
gtgttaggataatttagtccaaa

SEQ ID NO: 486 - DNA, 23 bp, synthetic construct
  misc_feature 1..23: note = Synthetic Construct
  source 1..23: mol_type = other DNA
ggtgttaggataatttagtccaa

SEQ ID NO: 487 - DNA, 23 bp, synthetic construct
  misc_feature 1..23: note = Synthetic Construct
  source 1..23: mol_type = other DNA
taagatcagattattttgcttag

SEQ ID NO: 488 - DNA, 23 bp, synthetic construct
  misc_feature 1..23: note = Synthetic Construct
  source 1..23: mol_type = other DNA
taataagggacgaacagggagga

SEQ ID NO: 489 - DNA, 23 bp, synthetic construct
  misc_feature 1..23: note = Synthetic Construct
  source 1..23: mol_type = other DNA
ataataagggacgaacagggagg

SEQ ID NO: 490 - DNA, 23 bp, synthetic construct
  misc_feature 1..23: note = Synthetic Construct
  source 1..23: mol_type = other DNA
cataataagggacgaacagggag

SEQ ID NO: 491 - DNA, 27 bp, synthetic construct
  misc_feature 1..27: note = Synthetic Construct
  source 1..27: mol_type = other DNA
tcataataagggacgaacagggaggat

SEQ ID NO: 492 - DNA, 23 bp, synthetic construct
  misc_feature 1..23: note = Synthetic Construct
  source 1..23: mol_type = other DNA
tcaaatcacctgcaggtaaaagc

SEQ ID NO: 493 - DNA, 23 bp, synthetic construct
  misc_feature 1..23: note = Synthetic Construct
  source 1..23: mol_type = other DNA
tcaagggtacatgatggatttga

SEQ ID NO: 494 - DNA, 23 bp, synthetic construct
  misc_feature 1..23: note = Synthetic Construct
  source 1..23: mol_type = other DNA
gtcaagggtacatgatggatttg

SEQ ID NO: 495 - DNA, 27 bp, synthetic construct
  misc_feature 1..27: note = Synthetic Construct
  source 1..27: mol_type = other DNA
cttctttgtcaagggtacatgatggat

SEQ ID NO: 496 - DNA, 23 bp, synthetic construct
  misc_feature 1..23: note = Synthetic Construct
  source 1..23: mol_type = other DNA
tcaatttgggaagcagcatattg

SEQ ID NO: 497 - DNA, 23 bp, synthetic construct
  misc_feature 1..23: note = Synthetic Construct
  source 1..23: mol_type = other DNA
tcagctatagccacaccagaagt

SEQ ID NO: 498 - DNA, 23 bp, synthetic construct
  misc_feature 1..23: note = Synthetic Construct
  source 1..23: mol_type = other DNA
ttcagctatagccacaccagaag

SEQ ID NO: 499 - DNA, 27 bp, synthetic construct
  misc_feature 1..27: note = Synthetic Construct
  source 1..27: mol_type = other DNA
tctttcagctatagccacaccagaagt

SEQ ID NO: 500 - DNA, 23 bp, synthetic construct
  misc_feature 1..23: note = Synthetic Construct
  source 1..23: mol_type = other DNA
tcctttcacatcttacgggacaa

SEQ ID NO: 501 - DNA, 27 bp, synthetic construct
  misc_feature 1..27: note = Synthetic Construct
  source 1..27: mol_type = other DNA
atttcctttcacatcttacgggacaat

SEQ ID NO: 502 - DNA, 23 bp, synthetic construct
  misc_feature 1..23: note = Synthetic Construct
  source 1..23: mol_type = other DNA
tcgtagcttctttctgctgagga

SEQ ID NO: 503 - DNA, 23 bp, synthetic construct
  misc_feature 1..23: note = Synthetic Construct
  source 1..23: mol_type = other DNA
atcgtagcttctttctgctgagg

SEQ ID NO: 504 - DNA, 23 bp, synthetic construct
  misc_feature 1..23: note = Synthetic Construct
  source 1..23: mol_type = other DNA
tatcgtagcttctttctgctgag

SEQ ID NO: 505 - DNA, 23 bp, synthetic construct
  misc_feature 1..23: note = Synthetic Construct
  source 1..23: mol_type = other DNA
ttatcgtagcttctttctgctga

SEQ ID NO: 506 - DNA, 23 bp, synthetic construct
  misc_feature 1..23: note = Synthetic Construct
  source 1..23: mol_type = other DNA
tctaatactccagccagttaagt

SEQ ID NO: 507 - DNA, 23 bp, synthetic construct
  misc_feature 1..23: note = Synthetic Construct
  source 1..23: mol_type = other DNA
ttctaatactccagccagttaag

SEQ ID NO: 508 - DNA, 23 bp, synthetic construct
  misc_feature 1..23: note = Synthetic Construct
  source 1..23: mol_type = other DNA
gttctaatactccagccagttaa

SEQ ID NO: 509 - DNA, 27 bp, synthetic construct
  misc_feature 1..27: note = Synthetic Construct
  source 1..27: mol_type = other DNA
ttgttctaatactccagccagttaagt

SEQ ID NO: 510 - DNA, 27 bp, synthetic construct
  misc_feature 1..27: note = Synthetic Construct
  source 1..27: mol_type = other DNA
tgatgttgttctaatactccagccagt

SEQ ID NO: 511 - DNA, 23 bp, synthetic construct
  misc_feature 1..23: note = Synthetic Construct
  source 1..23: mol_type = other DNA
tctaggcaggactacgaggctgg

SEQ ID NO: 512 - DNA, 23 bp, synthetic construct
  misc_feature 1..23: note = Synthetic Construct
  source 1..23: mol_type = other DNA
atctaggcaggactacgaggctg

SEQ ID NO: 513 - DNA, 23 bp, synthetic construct
  misc_feature 1..23: note = Synthetic Construct
  source 1..23: mol_type = other DNA
agatctaggcaggactacgaggc

SEQ ID NO: 514 - DNA, 23 bp, synthetic construct
  misc_feature 1..23: note = Synthetic Construct
  source 1..23: mol_type = other DNA
aagatctaggcaggactacgagg

SEQ ID NO: 515 - DNA, 23 bp, synthetic construct
  misc_feature 1..23: note = Synthetic Construct
  source 1..23: mol_type = other DNA
tctagtattccttaattgtacag

SEQ ID NO: 516 - DNA, 23 bp, synthetic construct
  misc_feature 1..23: note = Synthetic Construct
  source 1..23: mol_type = other DNA
tgtttctagtattccttaattgt

SEQ ID NO: 517 - DNA, 23 bp, synthetic construct
  misc_feature 1..23: note = Synthetic Construct
  source 1..23: mol_type = other DNA
tctatgttagtgatggctgagtg

SEQ ID NO: 518 - DNA, 23 bp, synthetic construct
  misc_feature 1..23: note = Synthetic Construct
  source 1..23: mol_type = other DNA
gtctatgttagtgatggctgagt

SEQ ID NO: 519 - DNA, 23 bp, synthetic construct
  misc_feature 1..23: note = Synthetic Construct
  source 1..23: mol_type = other DNA
tgtctatgttagtgatggctgag

SEQ ID NO: 520 - DNA, 23 bp, synthetic construct
  misc_feature 1..23: note = Synthetic Construct
  source 1..23: mol_type = other DNA
ttgtctatgttagtgatggctga

SEQ ID NO: 521 - DNA, 23 bp, synthetic construct
  misc_feature 1..23: note = Synthetic Construct
  source 1..23: mol_type = other DNA
gttgtctatgttagtgatggctg

SEQ ID NO: 522 - DNA, 27 bp, synthetic construct
  misc_feature 1..27: note = Synthetic Construct
  source 1..27: mol_type = other DNA
tgtctatgttagtgatggctgagtggt

SEQ ID NO: 523 - DNA, 27 bp, synthetic construct
  misc_feature 1..27: note = Synthetic Construct
  source 1..27: mol_type = other DNA
agttgtctatgttagtgatggctgagt

SEQ ID NO: 524 - DNA, 23 bp, synthetic construct
  misc_feature 1..23: note = Synthetic Construct
  source 1..23: mol_type = other DNA
tctcaaacatcttctcctgatgt

SEQ ID NO: 525 - DNA, 23 bp, synthetic construct
  misc_feature 1..23: note = Synthetic Construct
  source 1..23: mol_type = other DNA
gtctcaaacatcttctcctgatg

SEQ ID NO: 526 - DNA, 23 bp, synthetic construct
  misc_feature 1..23: note = Synthetic Construct
  source 1..23: mol_type = other DNA
aagtctcaaacatcttctcctga

SEQ ID NO: 527 - DNA, 27 bp, synthetic construct
  misc_feature 1..27: note = Synthetic Construct
  source 1..27: mol_type = other DNA
gtctcaaacatcttctcctgatgtagt

SEQ ID NO: 528 - DNA, 23 bp, synthetic construct
  misc_feature 1..23: note = Synthetic Construct
  source 1..23: mol_type = other DNA
tcttcaaaactgagcaaatttgc

SEQ ID NO: 529 - DNA, 23 bp, synthetic construct
  misc_feature 1..23: note = Synthetic Construct
  source 1..23: mol_type = other DNA
gtcttcaaaactgagcaaatttg

SEQ ID NO: 530 - DNA, 23 bp, synthetic construct
  misc_feature 1..23: note = Synthetic Construct
  source 1..23: mol_type = other DNA
tgcagaatttggaacagaggcgt

SEQ ID NO: 531 - DNA, 23 bp, synthetic construct
  misc_feature 1..23: note = Synthetic Construct
  source 1..23: mol_type = other DNA
atgcagaatttggaacagaggcg

SEQ ID NO: 532 - DNA, 23 bp, synthetic construct
  misc_feature 1..23: note = Synthetic Construct
  source 1..23: mol_type = other DNA
aatgcagaatttggaacagaggc

SEQ ID NO: 533 - DNA, 23 bp, synthetic construct
  misc_feature 1..23: note = Synthetic Construct
  source 1..23: mol_type = other DNA
tgcagcttctcgagttcatagga

SEQ ID NO: 534 - DNA, 23 bp, synthetic construct
  misc_feature 1..23: note = Synthetic Construct
  source 1..23: mol_type = other DNA
ttgcagcttctcgagttcatagg

SEQ ID NO: 535 - DNA, 23 bp, synthetic construct
  misc_feature 1..23: note = Synthetic Construct
  source 1..23: mol_type = other DNA
tgctagacccatgtcctgatggc

SEQ ID NO: 536 - DNA, 23 bp, synthetic construct
  misc_feature 1..23: note = Synthetic Construct
  source 1..23: mol_type = other DNA
ctgctagacccatgtcctgatgg

SEQ ID NO: 537 - DNA, 23 bp, synthetic construct
  misc_feature 1..23: note = Synthetic Construct
  source 1..23: mol_type = other DNA
actgctagacccatgtcctgatg

SEQ ID NO: 538 - DNA, 27 bp, synthetic construct
  misc_feature 1..27: note = Synthetic Construct
  source 1..27: mol_type = other DNA
ttctgactgctagacccatgtcctgat

SEQ ID NO: 539 - DNA, 23 bp, synthetic construct
  misc_feature 1..23: note = Synthetic Construct
  source 1..23: mol_type = other DNA
tggtatatatttctggtggcaaa

SEQ ID NO: 540 - DNA, 23 bp, synthetic construct
  misc_feature 1..23: note = Synthetic Construct
  source 1..23: mol_type = other DNA
gtggtatatatttctggtggcaa

SEQ ID NO: 541 - DNA, 27 bp, synthetic construct
  misc_feature 1..27: note = Synthetic Construct
  source 1..27: mol_type = other DNA
gtgtggtatatatttctggtggcaaat

SEQ ID NO: 542 - DNA, 23 bp, synthetic construct
  misc_feature 1..23: note = Synthetic Construct
  source 1..23: mol_type = other DNA
tgtcacatcaaattttcaagtga

SEQ ID NO: 543 - DNA, 23 bp, synthetic construct
  misc_feature 1..23: note = Synthetic Construct
  source 1..23: mol_type = other DNA
atgtcacatcaaattttcaagtg

SEQ ID NO: 544 - DNA, 23 bp, synthetic construct
  misc_feature 1..23: note = Synthetic Construct
  source 1..23: mol_type = other DNA
tgttcatcccctattatgaagaa

SEQ ID NO: 545 - DNA, 23 bp, synthetic construct
  misc_feature 1..23: note = Synthetic Construct
  source 1..23: mol_type = other DNA
ctgttcatcccctattatgaaga

SEQ ID NO: 546 - DNA, 23 bp, synthetic construct
  misc_feature 1..23: note = Synthetic Construct
  source 1..23: mol_type = other DNA
cctgttcatcccctattatgaag

SEQ ID NO: 547 - DNA, 27 bp, synthetic construct
  misc_feature 1..27: note = Synthetic Construct
  source 1..27: mol_type = other DNA
ccctgttcatcccctattatgaagaat

SEQ ID NO: 548 - DNA, 23 bp, synthetic construct
  misc_feature 1..23: note = Synthetic Construct
  source 1..23: mol_type = other DNA
ttacttcaatccgtaatgattgt

SEQ ID NO: 549 - DNA, 23 bp, synthetic construct
  misc_feature 1..23: note = Synthetic Construct
  source 1..23: mol_type = other DNA
ttagataggaaaagatgtggagc

SEQ ID NO: 550 - DNA, 23 bp, synthetic construct
  misc_feature 1..23: note = Synthetic Construct
  source 1..23: mol_type = other DNA
attagataggaaaagatgtggag

SEQ ID NO: 551 - DNA, 23 bp, synthetic construct
  misc_feature 1..23: note = Synthetic Construct
  source 1..23: mol_type = other DNA
cattagataggaaaagatgtgga

SEQ ID NO: 552 - DNA, 23 bp, synthetic construct
  misc_feature 1..23: note = Synthetic Construct
  source 1..23: mol_type = other DNA
acattagataggaaaagatgtgg

SEQ ID NO: 553 - DNA, 23 bp, synthetic construct
  misc_feature 1..23: note = Synthetic Construct
  source 1..23: mol_type = other DNA
ttgaaatataatcctccactggc

SEQ ID NO: 554 - DNA, 23 bp, synthetic construct
  misc_feature 1..23: note = Synthetic Construct
  source 1..23: mol_type = other DNA
tttgaaatataatcctccactgg

SEQ ID NO: 555 - DNA, 23 bp, synthetic construct
  misc_feature 1..23: note = Synthetic Construct
  source 1..23: mol_type = other DNA
atttgaaatataatcctccactg

SEQ ID NO: 556 - DNA, 23 bp, synthetic construct
  misc_feature 1..23: note = Synthetic Construct
  source 1..23: mol_type = other DNA
ttgtcagacccagctcaggagaa

SEQ ID NO: 557 - DNA, 23 bp, synthetic construct
  misc_feature 1..23: note = Synthetic Construct
  source 1..23: mol_type = other DNA
attgtcagacccagctcaggaga

SEQ ID NO: 558 - DNA, 23 bp, synthetic construct
  misc_feature 1..23: note = Synthetic Construct
  source 1..23: mol_type = other DNA
gattgtcagacccagctcaggag

SEQ ID NO: 559 - DNA, 27 bp, synthetic construct
  misc_feature 1..27: note = Synthetic Construct
  source 1..27: mol_type = other DNA
tgattgtcagacccagctcaggagaat

SEQ ID NO: 560 - DNA, 23 bp, synthetic construct
  misc_feature 1..23: note = Synthetic Construct
  source 1..23: mol_type = other DNA
ttgtctaaatatcactgactaaa

SEQ ID NO: 561 - DNA, 23 bp, synthetic construct
  misc_feature 1..23: note = Synthetic Construct
  source 1..23: mol_type = other DNA
attgtctaaatatcactgactaa

SEQ ID NO: 562 - DNA, 23 bp, synthetic construct
  misc_feature 1..23: note = Synthetic Construct
  source 1..23: mol_type = other DNA
ttgttctacaaaacccgcagtgc

SEQ ID NO: 563 - DNA, 23 bp, synthetic construct
  misc_feature 1..23: note = Synthetic Construct
  source 1..23: mol_type = other DNA
tttaacaatgctcaaccagctgg

SEQ ID NO: 564 - DNA, 23 bp, synthetic construct
  misc_feature 1..23: note = Synthetic Construct
  source 1..23: mol_type = other DNA
ttttaacaatgctcaaccagctg

SEQ ID NO: 565 - DNA, 23 bp, synthetic construct
  misc_feature 1..23: note = Synthetic Construct
  source 1..23: mol_type = other DNA
gcttttaacaatgctcaaccagc

SEQ ID NO: 566 - DNA, 23 bp, synthetic construct
  misc_feature 1..23: note = Synthetic Construct
  source 1..23: mol_type = other DNA
agcttttaacaatgctcaaccag

SEQ ID NO: 567 - DNA, 23 bp, synthetic construct
  misc_feature 1..23: note = Synthetic Construct
  source 1..23: mol_type = other DNA
tttaggcagcctccttcccctga

SEQ ID NO: 568 - DNA, 23 bp, synthetic construct
  misc_feature 1..23: note = Synthetic Construct
  source 1..23: mol_type = other DNA
ctttaggcagcctccttcccctg

SEQ ID NO: 569 - DNA, 27 bp, synthetic construct
  misc_feature 1..27: note = Synthetic Construct
  source 1..27: mol_type = other DNA
tttcttcttctacaattcccgatcaat

SEQ ID NO: 570 - DNA, 20 bp, synthetic construct
  misc_feature 1..20: note = Synthetic Construct
  source 1..20: mol_type = other DNA
gttatctcctgttctgcagc

SEQ ID NO: 571 - DNA, 20 bp, synthetic construct
  misc_feature 1..20: note = Synthetic Construct
  source 1..20: mol_type = other DNA
gtttatgtcaccagagtaac

SEQ ID NO: 572 - DNA, 21 bp, synthetic construct
  misc_feature 1..21: note = Synthetic Construct
  source 1..21: mol_type = other DNA
gaggtaatagagccaagccct

SEQ ID NO: 573 - DNA, 25 bp, synthetic construct
  misc_feature 1..25: note = Synthetic Construct
  source 1..25: mol_type = other DNA
gcaagaattccacttttcacttcct

SEQ ID NO: 574 - DNA, 24 bp, synthetic construct
  misc_feature 1..24: note = Synthetic Construct
  source 1..24: mol_type = other DNA
ctgtcatctccaaactagaaatgc

SEQ ID NO: 575 - DNA, 22 bp, synthetic construct
  misc_feature 1..22: note = Synthetic Construct
  source 1..22: mol_type = other DNA
gcagcctcttgctcacttactc

SEQ ID NO: 576 - DNA, 20 bp, synthetic construct
  misc_feature 1..20: note = Synthetic Construct
  source 1..20: mol_type = other DNA
gatgacaggcaggggcaccg

SEQ ID NO: 577 - DNA, 20 bp, synthetic construct
  misc_feature 1..20: note = Synthetic Construct
  source 1..20: mol_type = other DNA
ttccagtggttcaatggtca

SEQ ID NO: 578 - DNA, 20 bp, synthetic construct
  misc_feature 1..20: note = Synthetic Construct
  source 1..20: mol_type = other DNA
ctttcaacccgaacggagac

SEQ ID NO: 579 - DNA, 20 bp, synthetic construct
  misc_feature 1..20: note = Synthetic Construct
  source 1..20: mol_type = other DNA
gagcgagcagcgtcttcgag

SEQ ID NO: 580 - DNA, 20 bp, synthetic construct
  misc_feature 1..20: note = Synthetic Construct
  source 1..20: mol_type = other DNA
gcagacggcagtcactaggg

SEQ ID NO: 581 - DNA, 20 bp, synthetic construct
  misc_feature 1..20: note = Synthetic Construct
  source 1..20: mol_type = other DNA
gggaagctgggtgaatggag

SEQ ID NO: 582 - DNA, 20 bp, synthetic construct
  misc_feature 1..20: note = Synthetic Construct
  source 1..20: mol_type = other DNA
agctgtttgggaggtcagaa

SEQ ID NO: 583 - DNA, 20 bp, synthetic construct
  misc_feature 1..20: note = Synthetic Construct
  source 1..20: mol_type = other DNA
agggagcaggaaagtgaggt

SEQ ID NO: 584 - DNA, 20 bp, synthetic construct
  misc_feature 1..20: note = Synthetic Construct
  source 1..20: mol_type = other DNA
gtcgcaggacagcttttcct

SEQ ID NO: 585 - DNA, 20 bp, synthetic construct
  misc_feature 1..20: note = Synthetic Construct
  source 1..20: mol_type = other DNA
tgtagctacgcctgtgatgg

SEQ ID NO: 586 - DNA, 20 bp, synthetic construct
  misc_feature 1..20: note = Synthetic Construct
  source 1..20: mol_type = other DNA
tgccctgagatcttttcctc

SEQ ID NO: 587 - DNA, 20 bp, synthetic construct
  misc_feature 1..20: note = Synthetic Construct
  source 1..20: mol_type = other DNA
gatccaggtgctgcagaagg

SEQ ID NO: 588 - DNA, 20 bp, synthetic construct
  misc_feature 1..20: note = Synthetic Construct
  source 1..20: mol_type = other DNA
ctcttgcctccactggttgt

SEQ ID NO: 589 - DNA, 20 bp, synthetic construct
  misc_feature 1..20: note = Synthetic Construct
  source 1..20: mol_type = other DNA
tcggtaggatgccctacatc

SEQ ID NO: 590 - DNA, 20 bp, synthetic construct
  misc_feature 1..20: note = Synthetic Construct
  source 1..20: mol_type = other DNA
atcctacagcatggtggctg

SEQ ID NO: 591 - DNA, 32 bp, synthetic construct
  misc_feature 1..32: note = Synthetic Construct
  source 1..32: mol_type = other DNA
agtggtctccggaaacctccgcgccccgcaac

SEQ ID NO: 592 - DNA, 20 bp, synthetic construct
  misc_feature 1..20: note = Synthetic Construct
  source 1..20: mol_type = other DNA
tccttgaagaagatggtgcg

SEQ ID NO: 593 - DNA, 55 bp, synthetic construct
  misc_feature 1..55: note = Synthetic Construct
  source 1..55: mol_type = other DNA
acactctttccctacacgacgctcttccgatctgaactcattactgctgcccaga

SEQ ID NO: 594 - DNA, 55 bp, synthetic construct
  misc_feature 1..55: note = Synthetic Construct
  source 1..55: mol_type = other DNA
gtgactggagttcagacgtgtgctcttccgatcgacctgttcggcttcttcctta

SEQ ID NO: 595 - DNA, 58 bp, synthetic construct
  misc_feature 1..58: note = Synthetic Construct
  source 1..58: mol_type = other DNA
acactctttccctacacgacgctcttccgatctaaatttccactgtcttctcttgagt

SEQ ID NO: 596 - DNA, 55 bp, synthetic construct
  misc_feature 1..55: note = Synthetic Construct
  source 1..55: mol_type = other DNA
gtgactggagttcagacgtgtgctcttccgatcgcttgcctctgacctgtcctat

SEQ ID NO: 597 - DNA, 55 bp, synthetic construct
  misc_feature 1..55: note = Synthetic Construct
  source 1..55: mol_type = other DNA
acactctttccctacacgacgctcttccgatctgtgactaggggcaaagcaagat

SEQ ID NO: 598 - DNA, 55 bp, synthetic construct
  misc_feature 1..55: note = Synthetic Construct
  source 1..55: mol_type = other DNA
gtgactggagttcagacgtgtgctcttccgatccttccaaactttctgcccattc

SEQ ID NO: 599 - DNA, 56 bp, synthetic construct
  misc_feature 1..56: note = Synthetic Construct
  source 1..56: mol_type = other DNA
acactctttccctacacgacgctcttccgatctaacacagcgtgctctttccttac

SEQ ID NO: 600 - DNA, 56 bp, synthetic construct
  misc_feature 1..56: note = Synthetic Construct
  source 1..56: mol_type = other DNA
gtgactggagttcagacgtgtgctcttccgatcgttcagaagaacatcccgttgac

SEQ ID NO: 601 - DNA, 45 bp, synthetic construct
  misc_feature 1..45: note = Synthetic Construct
  source 1..45: mol_type = other DNA
aatgatacggcgaccaccgagatctacactctttccctacacgac

SEQ ID NO: 602 - DNA, 49 bp, synthetic construct
  misc_feature 1..49: note = Synthetic Construct
  source 1..49: mol_type = other DNA
caagcagaagacggcatacgagatcttgtagtgactggagttcagacgt

SEQ ID NO: 603 - DNA, 49 bp, synthetic construct
  misc_feature 1..49: note = Synthetic Construct
  source 1..49: mol_type = other DNA
caagcagaagacggcatacgagatcagatcgtgactggagttcagacgt

SEQ ID NO: 604 - DNA, 49 bp, synthetic construct
  misc_feature 1..49: note = Synthetic Construct
  source 1..49: mol_type = other DNA
caagcagaagacggcatacgagatccgtccgtgactggagttcagacgt

SEQ ID NO: 605 - DNA, 49 bp, synthetic construct
  misc_feature 1..49: note = Synthetic Construct
  source 1..49: mol_type = other DNA
caagcagaagacggcatacgagatatgtcagtgactggagttcagacgt

SEQ ID NO: 606 - DNA, 49 bp, synthetic construct
  misc_feature 1..49: note = Synthetic Construct
  source 1..49: mol_type = other DNA
caagcagaagacggcatacgagatgtccgcgtgactggagttcagacgt

SEQ ID NO: 607 - DNA, 49 bp, synthetic construct
  misc_feature 1..49: note = Synthetic Construct
  source 1..49: mol_type = other DNA
caagcagaagacggcatacgagatttaggcgtgactggagttcagacgt

SEQ ID NO: 608 - DNA, 49 bp, synthetic construct
  misc_feature 1..49: note = Synthetic Construct
  source 1..49: mol_type = other DNA
caagcagaagacggcatacgagatcgatgtgtgactggagttcagacgt

SEQ ID NO: 609 - DNA, 49 bp, synthetic construct
  misc_feature 1..49: note = Synthetic Construct
  source 1..49: mol_type = other DNA
caagcagaagacggcatacgagattgaccagtgactggagttcagacgt

SEQ ID NO: 610 - DNA, 49 bp, synthetic construct
  misc_feature 1..49: note = Synthetic Construct
  source 1..49: mol_type = other DNA
caagcagaagacggcatacgagatagtcaagtgactggagttcagacgt

SEQ ID NO: 611 - DNA, 49 bp, synthetic construct
  misc_feature 1..49: note = Synthetic Construct
  source 1..49: mol_type = other DNA
caagcagaagacggcatacgagatagttccgtgactggagttcagacgt

SEQ ID NO: 612 - DNA, 49 bp, synthetic construct
  misc_feature 1..49: note = Synthetic Construct
  source 1..49: mol_type = other DNA
caagcagaagacggcatacgagatgatcaggtgactggagttcagacgt

SEQ ID NO: 613 - DNA, 49 bp, synthetic construct
  misc_feature 1..49: note = Synthetic Construct
  source 1..49: mol_type = other DNA
caagcagaagacggcatacgagatacagtggtgactggagttcagacgt

SEQ ID NO: 614 - DNA, 49 bp, synthetic construct
  misc_feature 1..49: note = Synthetic Construct
  source 1..49: mol_type = other DNA
caagcagaagacggcatacgagattatactgtgactggagttcagacgt

SEQ ID NO: 615 - DNA, 49 bp, synthetic construct
  misc_feature 1..49: note = Synthetic Construct
  source 1..49: mol_type = other DNA
caagcagaagacggcatacgagatcaacaagtgactggagttcagacgt

SEQ ID NO: 616 - DNA, 49 bp, synthetic construct
  misc_feature 1..49: note = Synthetic Construct
  source 1..49: mol_type = other DNA
caagcagaagacggcatacgagatgttgttgtgactggagttcagacgt

SEQ ID NO: 617 - DNA, 49 bp, synthetic construct
  misc_feature 1..49: note = Synthetic Construct
  source 1..49: mol_type = other DNA
caagcagaagacggcatacgagattcggttgtgactggagttcagacgt

SEQ ID NO: 618 - DNA, 49 bp, synthetic construct
  misc_feature 1..49: note = Synthetic Construct
  source 1..49: mol_type = other DNA
caagcagaagacggcatacgagatagtattgtgactggagttcagacgt

SEQ ID NO: 619 - DNA, 49 bp, synthetic construct
  misc_feature 1..49: note = Synthetic Construct
  source 1..49: mol_type = other DNA
caagcagaagacggcatacgagattcttgtgtgactggagttcagacgt

SEQ ID NO: 620 - DNA, 27 bp, synthetic construct
  misc_feature 1..27: note = Synthetic Construct
  source 1..27: mol_type = other DNA
gaacagctgcagaacaggagataacag

SEQ ID NO: 621 - DNA, 23 bp, synthetic construct
  misc_feature 1..23: note = Synthetic Construct
  source 1..23: mol_type = other DNA
gttatctcctgttctgcagctgt

SEQ ID NO: 622 - DNA, 22 bp, synthetic construct
  misc_feature 1..22: note = Synthetic Construct
  source 1..22: mol_type = other DNA
atgacaggcaggggcaccgcgg

SEQ ID NO: 623 - DNA, 23 bp, synthetic construct
  misc_feature 1..23: note = Synthetic Construct
  source 1..23: mol_type = other DNA
gagcgagcagcgtcttcgagagt

SEQ ID NO: 624 - DNA, 23 bp, synthetic construct
  misc_feature 1..23: note = Synthetic Construct
  source 1..23: mol_type = other DNA
gcagacggcagtcactagggggc

SEQ ID NO: 625 - DNA, 23 bp, synthetic construct
  misc_feature 1..23: note = Synthetic Construct
  source 1..23: mol_type = other DNA
gtcgcaggacagcttttcctaga

SEQ ID NO: 626 - DNA, 23 bp, synthetic construct
  misc_feature 1..23: note = Synthetic Construct
  source 1..23: mol_type = other DNA
gggaagctgggtgaatggagcga

SEQ ID NO: 627 - DNA, 23 bp, synthetic construct
  misc_feature 1..23: note = Synthetic Construct
  source 1..23: mol_type = other DNA
gatccaggtgctgcagaagggat

SEQ ID NO: 628 - DNA, 23 bp, synthetic construct
  misc_feature 1..23: note = Synthetic Construct
  source 1..23: mol_type = other DNA
gttatctcctgctctgcagcaga

SEQ ID NO: 629 - DNA, 23 bp, synthetic construct
  misc_feature 1..23: note = Synthetic Construct
  source 1..23: mol_type = other DNA
gatatctcctgttctgcaggaga

SEQ ID NO: 630 - DNA, 20 bp, synthetic construct
  misc_feature 1..20: note = Synthetic Construct
  source 1..20: mol_type = other DNA
ggatttccaagtctccaccc

SEQ ID NO: 631 - DNA, 19 bp, synthetic construct
  misc_feature 1..19: note = Synthetic Construct
  source 1..19: mol_type = other DNA
tcccaccgtacacgcctac

SEQ ID NO: 632 - DNA, 54 bp, synthetic construct
  misc_feature 1..54: note = Synthetic Construct
  source 1..54: mol_type = other DNA
ggtttcagacaaaatcaaaaagaaggaaggtgctcacattccttaaattaagga

SEQ ID NO: 633 - DNA, 54 bp, synthetic construct
  misc_feature 1..54: note = Synthetic Construct
  source 1..54: mol_type = other DNA
ggttttagacaaaatcaaaaagaaggaaggtgctcacattccttaaattaagga

SEQ ID NO: 634 - DNA, 24 bp, synthetic construct
  misc_feature 1..24: note = Synthetic Construct
  source 1..24: mol_type = other DNA
gtgctcacattccttaaattaagg

SEQ ID NO: 635 - DNA, 24 bp, synthetic construct
  misc_feature 1..24: note = Synthetic Construct
  source 1..24: mol_type = other DNA
ggctcacattccttaaattaagga

(19) United States
(12) Patent Application Publication
(10) Pub. No.: US 2026/0071202 A1
(43) Pub. Date: Mar. 12, 2026

(54) NUCLEOBASE EDITORS AND METHODS OF USE THEREOF

(71) Applicant: Ohio State Innovation Foundation, Columbus, OH (US)

(72) Inventors: Renzhi Han, Columbus, OH (US); Li Xu, Bedford, MA (US); Yandi Gao, Powell, OH (US)

(21) Appl. No.: 19/306,353

(22) Filed: Aug. 21, 2025

Related U.S. Application Data
(63) Continuation of application No. 17/799,440, now Pat. No. 12,416,001.
(60) Provisional application No. 62/976,590, filed on Feb. 14, 2020.

(51) Int. Cl. C12N 15/10; C12N 9/22; C12N 9/78; C12N 15/11; C12N 15/86

(52) U.S. Cl. CPC C12N 15/102; C12N 9/22; C12N 9/78; C12N 15/11; C12N 15/86; C12N 2310/20; C12N 2750/14143; C12N 2800/40; C12Y 305/04002; C12Y 305/04005

(57) ABSTRACT

The present disclosure relates to nucleobase editors and methods of use thereof.

### CROSS-REFERENCE TO RELATED APPLICATIONS

This application is a continuation of U.S. application Ser. No. 17/799,440, filed on Aug. 12, 2022, which claims priority from PCT/US2021/017868, filed Feb. 12, 2021, which claims the priority from U.S. Provisional Application No. 62/976,590, filed Feb. 14, 2020, which are expressly incorporated herein by reference.

### STATEMENT REGARDING FEDERALLY SPONSORED RESEARCH

This invention was made with government support under Grant No. R01 HL116546 awarded by the National Institutes of Health. The government has certain rights in the invention.

### REFERENCE TO SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on Jul. 7, 2025, is entitled “10336-646US2_ST26.xml”, and is 954,321 bytes in size.

### FIELD

The present disclosure relates to nucleobase editors and methods of use thereof.

### BACKGROUND

Many genetic diseases arise from single nucleotide alterations. Duchenne muscular dystrophy (DMD) is a fatal genetic muscle disease affecting approximately 1 in ˜5000 male births worldwide, which is caused by mutations in the DMD gene. Most of the DMD mutations are due to deletions or duplications with over 500 point mutations accounting for ˜10% of the cases. DMD codes for the dystrophin protein, a cytoskeletal protein that functions in the muscle force transmission and sarcolemmal stability of muscle fibers. Loss of dystrophin leads to progressive muscle weakness and wasting, loss of ambulation, respiratory impairment, cardiomyopathy, and eventual death. Previous studies showed that exon deletion through CRISPR genome editing can restore dystrophin expression and function. Although promising, this strategy has safety concerns as it relies on repair of the double strand DNA break (DSB) created by CRISPR/Cas9, which can cause unwanted large deletion and even chromosomal rearrangement.

Therefore, what is needed are compositions for precise correction of disease-associated mutations.

### SUMMARY

Disclosed herein are fusion proteins, systems, and compositions for editing disease-associated mutations and methods of use thereof.

In some aspects, disclosed herein is a fusion protein comprising a Cas9 nickase and a nucleotide deaminase, wherein the Cas9 nickase comprises a first amino acid substitution at a position selected from the group consisting of 262, 324, 409, 480, 543, 694, and 1219 when compared to SEQ ID NO: 11, and wherein the Cas9 nickase comprises a second amino acid substitution at a position selected from the group consisting of 1111, 1135, 1218, 1219, 1322, 1335, and 1337 when compared to SEQ ID NO: 11.

In some embodiments, the first amino acid substitution is selected from the group consisting of A262T, R324L, S409I, E480K, E543D, M694I, and E1219V when compared to SEQ ID NO: 11. In some embodiments, the second amino acid substitution is selected from the group consisting of L1111R, D1135V, G1218R, E1219F, A1322R, R1335V, R1335Q, R1335E, and T1337R when compared to SEQ ID NO: 11.

In some embodiments, the Cas9 nickase comprises the amino acid sequence selected from the group consisting of SEQ ID NOs: 12-20.

In some embodiments, the Cas9 nickase when in conjunction with a bound guide RNA (gRNA) specifically binds to a target nucleic acid sequence.

In some embodiments, the Cas9 nickase recognizes a NG protospacer adjacent motif (PAM) sequence.

In some embodiments, the nucleotide deaminase is a cytidine deaminase or an adenine deaminase. In some embodiments, the adenine deaminase is a dimeric adenine deaminase or a monomeric adenine deaminase domain. In some embodiments, the monomeric adenine deaminase comprises amino acid substitutions A56G and V82G when compared to SEQ ID NO: 47. In some embodiments, the adenine deaminase comprises the amino acid sequence set forth in SEQ ID NO: 22 or 23.

In some aspects, disclosed herein is a system for base editing comprising:
a first nucleotide sequence encoding a N-terminal portion of a Cas9 nickase fused at its C-terminus to an intein-N; and
a second nucleotide sequence encoding an intein-C fused to the N-terminus of a C-terminal portion of the Cas9 nickase;
wherein the first nucleotide sequence further comprises a nucleotide sequence encoding a nucleotide deaminase fused to the N-terminus of the N-terminal portion of the Cas9 nickase,
wherein the Cas9 nickase comprises a first amino acid substitution at a position selected from the group consisting of 262, 324, 409, 480, 543, 694, and 1219 when compared to SEQ ID NO: 11, and
wherein the Cas9 nickase comprises a second amino acid substitution at a position selected from the group consisting of 1111, 1135, 1218, 1219, 1322, 1335, and 1337 when compared to SEQ ID NO: 11.

In some embodiments, the N-terminal portion of the Cas9 nickase comprises the amino acid positions 2-573 of the amino acid sequence selected from the group consisting of SEQ ID NOs: 12-20 and the C-terminal portion of the Cas9 nickase comprises the amino acid positions 574-1368 of the amino acid sequence selected from the group consisting of SEQ ID NOs: 12-20.

In some embodiments, the first nucleotide sequence or the second nucleotide sequence further comprises a nucleotide encoding a guide RNA (gRNA).

In some aspects, disclosed herein is a composition comprising:
a first recombinant adeno-associated virus (AAV) particle comprising a first nucleotide sequence encoding a N-terminal portion of a Cas9 nickase fused at its C-terminus to an intein-N;
and a second recombinant AAV particle comprising a second nucleotide sequence encoding an intein-C fused to the N-terminus of a C-terminal portion of the Cas9 nickase;
wherein the first nucleotide sequence further comprises a nucleotide sequence encoding a nucleotide deaminase fused to the N-terminus of the N-terminal portion of the Cas9 nickase,
wherein the Cas9 nickase comprises a first amino acid substitution at a position selected from the group consisting of 262, 324, 409, 480, 543, 694, and 1219 when compared to SEQ ID NO: 11, and
wherein the Cas9 nickase comprises a second amino acid substitution at a position selected from the group consisting of 1111, 1135, 1218, 1219, 1322, 1335, and 1337 when compared to SEQ ID NO: 11.

In some embodiments, the first nucleotide sequence comprises the sequence as set forth in SEQ ID NO: 3 or SEQ ID NO: 5. In some embodiments, the second nucleotide sequence comprises the sequence as set forth in SEQ ID NO: 4 or SEQ ID NO: 6.

In some aspects, disclosed herein is a method of treating a genetic disease or disorder in a subject, comprising administering a therapeutically effective amount of the composition of any preceding aspect. In some embodiments, the genetic disease or disorder is Duchenne muscular dystrophy, dysferlinopathy, or spinal muscular atrophy. In some embodiments, the genetic disease or disorder is due to a point mutation in a causative gene from a G: C pair to an A:T pair.

### BRIEF DESCRIPTION OF THE DRAWINGS

The accompanying figures, which are incorporated in and constitute a part of this specification, illustrate several aspects described below.

FIGS. 1a-1e show in vitro studies of mdx^{4cv} mutation correction using ABE-NG. FIG. 1a shows genomic DNA, encoded amino acids and guide RNA with PAM (highlighted in blue) sequences at the stop codon mutation site (red). FIG. 1b shows the reporter construct containing a puromycin resistance cassette fused with E2A peptide, mdx^{4cv} target sequence and ATG-removed EGFP. Correction of the stop codon within the target sequence would allow EGFP expression. FIG. 1c shows fluorescence microscopy images of HEK293 cells transfected with reporter alone, or reporter, gRNA and one of the base editors (ABEmax, ABE-x and ABE-NG). Scale bar: 500 μm. FIGS. 1d-1e shows flow cytometry analysis of EGFP expression in HEK293 cells transfected as described in FIG. 1c. *p<0.05; ****p<0.0001 (one-way ANOVA). The sequences in FIG. 1 are GAACAGCTGCAGAACAGGAGATAACAG (SEQ ID NO: 620), GTTaTCTCCTGTTCTG CAGCTGT (SEQ ID NO: 621).

FIGS. 2a-2f show optimization of the PAM-interacting domain to improve the editing efficiency of ABE-NG at the NGN sites. FIGS. 2a-2f show measurements of the base editing efficiencies of different ABE variants on a NGG-PAM site and five different sites with NGH or GAT PAM. The gRNA sequences are listed on the top of each graph with the PAM sequences in red and the target adenines in blue. ns, not significant; **p<0.01; ***p<0.001; ****p<0.0001 (one-way ANOVA). The sequences in FIG. 2 are ATGACAGGCAGGGGCACCGCGG (SEQ ID NO: 622), GAGCGAGCAGCGTCTTCGAGAGT (SEQ ID NO: 623), GCAGACGGCA GTCACTAGGGGGC (SEQ ID NO: 624), GTCGCAGGACAGCTTTTCCTAGA (SEQ ID NO: 625), GGGAAGCTGGGTGAATGGAGCGA (SEQ ID NO: 626), GATCCAGGT GCTGCAGAAGGGAT (SEQ ID NO: 627).

FIGS. 3a-3d show re-engineering of the adenine deaminase domain to improve the efficiency and specificity of ABE-NG. FIG. 3a shows schematics of the adenine deaminase domain used in ABE-NG editors. FIG. 3b shows quantification of the editing efficiency of different ABE-NG variants with modified TadA* domain at the mdx^{4cv} target site. ****p<0.0001 (one-way ANOVA). FIG. 3c shows the number of off-target RNA editing events in Neuro-2a cells transfected with different ABE-NG variants. ***p<0.05; ***p<0.001 (one-way ANOVA). FIG. 3d shows quantification of the off-target RNA editing (A-to-I) activities on four RNA adenines previously identified as being efficiently modified by ABEmax in HEK293 cells. ****p<0.0001 (one-way ANOVA).

FIGS. 4a-4b show comparison of the editing efficiency of different ABEs at the mdx^{4cv} target site (FIG. 4a) and human dysferlin Q605X site (FIG. 4b). All base editors carry the SpCas9-NG and differ at the adenine deaminase domain. ns, not statistically significant; **p<0.01; ****p<0.0001 (one-way ANOVA for FIG. 4a; two-way ANOVA for FIG. 4b). The sequence in FIG. 4 is GTTATCTCCTGTTCTGCAGC (SEQ ID NO: 570) and ATCCTACAGCATGGTGGCTG (SEQ ID NO: 590).

FIGS. 5a-5b show the intein split of ABEmax had relatively low editing activity. FIG. 5a shows schematics showing the two halves of intein-split ABEmax. The TadA-TadA* was fused with Npu intein N-terminal fragment and SpCas9 nickase (nSpCas9) was fused with Npu intein C-terminal fragment. FIG. 5b shows genomic DNA PCR analysis of HEK293 cells at 5 days after transfection with S2-gRNA and different versions of ABEs.

FIGS. 6a-6h show intein-mediated assembly of full-length ABE-NG. FIG. 6a shows schematics of the intein split ABE-NG. The N-terminal and C-terminal intein sequences reconstitute the full-length protein when co-expressed within cells. FIG. 6b shows Western blot analysis of HEK293 cell lysates transfected with different versions of ABEs. FIG. 6c shows fluorescence microscopy images of HEK293 cells transfected with reporter alone, or reporter, gRNA and one of the base editors (ABE-NG, split_v1 N+C or Split_v2 N+C). Scale bar: 500 μm. FIG. 6d shows flow cytometry analysis of EGFP expression in HEK293 cells transfected as described in FIG. 6c. FIG. 6e shows Western blot analysis of HEK293 cell lysates transfected with full-length iABE-NGA, Gp41-1 or Npu split of iABE-NGA. FL, the full-length iABE-NGA band; N, the N-terminal fragment of the iABE-NGA. FIG. 6f shows densitometry quantification of the Western blot data shown in FIG. 6e. FIG. 6g shows the assembly efficiency of the Gp41-1 and Npu split of iABE-NGA (defined as the percentage of the full-length iABE-NGA bands). FIG. 6h shows quantification of the editing efficiency of full-length iABE-NGA, Gp41-1 split and Npu split of iABE-NGA at the mdx^{4cv} target site. Npu Split_1g is same as Npu Split except that only the C-terminal construct carries the gRNA. ns, not significant; *p<0.05; ***p<0.001; ****p<0.0001 (one-way ANOVA).

FIG. 7. Immunofluorescence staining of heart and gastrocnemius muscle sections with anti-dystrophin antibody and DAPI. The mdx^{4cv} mice at 5 weeks of age received either 5×10¹³, 1×10¹⁴ AAV9-iNG/mdx^{4cv}-gRNA or 1×10¹⁴ vg/kg AAV9-iNG/non-targeting-gRNA (NT-gRNA) through tail vein injection, and sacrificed at 10 weeks of age for immunofluorescence staining analysis. Scale bar: 100 μm.

FIGS. 8a-8k show widespread restoration of dystrophin expression in mdx^{4cv} mice following systemic administration of AAV9-iNG. FIG. 8a shows dystrophin and laminin-α2 co-immunostaining of heart sections from WT and mdx^{4cv} mice (10 weeks or 10 months of age) with or without tail vein injection of AAV9-iNG (a total of 1×10¹⁴ vg/kg, 1:1 of the N and C-terminal half). Scale bar: 100 μm. FIGS. 8b-8d show quantification of dystrophin-positive fibers in the heart (FIG. 8b), gastrocnemius (GA, FIG. 8c) and diaphragm (Diaph, FIG. 8d) muscles. 10w, 10 weeks old; 10 m, 10 months old. **p<0.01; ****p<0.0001 (Student's t-test). FIG. 8e shows Western blot analysis of heart homogenates from 10-week-old mice with anti-dystrophin, Cas9 and Gapdh antibodies. The WT muscle lysates were loaded at 5 μg/lane while the mdx^{4cv} muscle lysates were loaded at 25 μg/lane. FIG. 8f shows densitometry quantification of Western blot data shown in FIG. 8e. *p<0.05 (one-way ANOVA). FIG. 8g shows Western blot analysis of heart homogenates from 10-month-old mice with anti-dystrophin and Gapdh antibodies. FIG. 8h shows densitometry quantification of Western blot data in FIG. 8g. **p<0.01; ****p<0.0001 (one-way ANOVA). FIG. 8i shows representative sequencing trace of dystrophin transcripts of WT and mdx^{4cv} mouse hearts (10 weeks or 10 months old) with or without AAV9-iNG treatment. FIGS. 8j and 8k show quantification of the targeted T-to-C editing efficiency in the mdx^{4cv} mouse hearts (FIG. 8j, 10 weeks; FIG. 8k, 10 months) as assayed by sequencing of dystrophin transcripts. ****p<0.0001 (Student's t-test).

FIG. 9 shows stitched large images showing dystrophin and laminin-α2 immunostaining of the entire heart sections of a WT mouse at 10 weeks of age. Scale bars: 0.5 mm.

FIG. 10 shows stitched large images showing dystrophin and laminin-α2 immunostaining of the entire heart sections of a control mdx^{4cv} mouse at 10 weeks of age. Scale bars: 0.5 mm.

FIG. 11 shows stitched large images showing dystrophin and laminin-α2 immunostaining of the entire heart sections mdx^{4cv} mouse #142 five weeks after intravenous injection of AAV9-iNG at 5 weeks of age. Mouse number is shown in yellow. Scale bars: 0.5 mm.

FIG. 12 shows stitched large images showing dystrophin and laminin-2 immunostaining of the entire heart sections mdx^{4cv} mouse #147 five weeks after intravenous injection of AAV9-iNG at 5 weeks of age. Mouse number is shown in yellow. Scale bars: 0.5 mm.

FIG. 13 shows stitched large images showing dystrophin and laminin-α2 immunostaining of the entire heart sections mdx^{4cv} mouse #148 five weeks after intravenous injection of AAV9-iNG at 5 weeks of age. Mouse number is shown in yellow. Scale bars: 0.5 mm.

FIG. 14 shows stitched large images showing dystrophin and laminin-α2 immunostaining of the entire heart sections mdx^{4cv} mouse #149 five weeks after intravenous injection of AAV9-iNG at 5 weeks of age. Mouse number is shown in yellow. Scale bars: 0.5 mm.

FIG. 15 shows stitched large images showing dystrophin and laminin-α2 immunostaining of the entire heart sections mdx^{4cv} mouse #150 five weeks after intravenous injection of AAV9-iNG at 5 weeks of age. Mouse number is shown in yellow. Scale bars: 0.5 mm.

FIGS. 16a-16b show immunofluorescence staining of dystrophin and laminin α2 in the gastrocnemius (FIG. 16a) and diaphragm (FIG. 16b) muscles from WT and mdx^{4cv} (10 weeks of age) treated with or without tail vein injection of AAV9-iNG. Scale bar: 100 μm.

FIG. 17 shows Western blot analysis of gastrocnemius muscles from WT and mdx^{4cv} (10 weeks of age) treated with or without tail vein injection of AAV9-iNG.

FIG. 18 shows stitched large images showing dystrophin and laminin-α2 immunostaining of the entire heart sections of a WT mouse at 10 months of age. Scale bars: 0.5 mm.

FIG. 19 shows stitched large images showing dystrophin and laminin-α2 immunostaining of the entire heart sections of a control mdx^{4cv} mouse at 10 months of age. Scale bars: 0.5 mm.

FIG. 20 shows stitched large images showing dystrophin and laminin-α2 immunostaining of the entire heart sections mdx^{4cv} mouse #1976 9-10 months after intravenous injection of AAV9-iNG at 5 weeks of age. Mouse number is shown in yellow. Scale bars: 0.5 mm.

FIG. 21 shows stitched large images showing dystrophin and laminin-α2 immunostaining of the entire heart sections mdx^{4cv} mouse #1982 9-10 months after intravenous injection of AAV9-iNG at 5 weeks of age. Mouse number is shown in yellow. Scale bars: 0.5 mm.

FIG. 22 shows stitched large images showing dystrophin and laminin-α2 immunostaining of the entire heart sections mdx^{4cv} mouse #1985 9-10 months after intravenous injection of AAV9-iNG at 5 weeks of age. Mouse number is shown in yellow. Scale bars: 0.5 mm.

FIG. 23 shows stitched large images showing dystrophin and laminin-α2 immunostaining of the entire heart sections mdx^{4cv} mouse #1984 9-10 months after intravenous injection of AAV9-iNG at 5 weeks of age. Mouse number is shown in yellow. Scale bars: 0.5 mm.

FIGS. 24a-24c show restoration of dystrophin expression in the skeletal muscles of 10-month-old mdx^{4cv} mice after tail vein injection of AAV9-iNG at 5 weeks of age. FIG. 24a shows immunofluorescence staining of dystrophin in diaphragm and gastrocnemius muscles of WT and mdx^{4cv} mice with or without systemic AAV9-iNG delivery. FIG. 24b shows Western blot of dystrophin expression in gastrocnemius muscles. FIG. 24c shows quantification of Western blot data.

FIGS. 25a-25m show systemic delivery of AAV9-iNG improved histopathology and contractility in mdx^{4cv} mice. FIG. 25a shows trichrome staining of muscle and heart sections showing the extensive fibrosis in diaphragm and gastrocnemius (GA) muscles of mdx^{4cv} mice (10 months of age), which were substantially reduced following systemic AAV9-iNG delivery. The mdx^{4cv} mouse heart had little fibrosis at 10 months of age. Scale bar: 200 μm. FIGS. 25b-25d show quantification of fibrotic area of the diaphragm, gastrocnemius and heart muscles. ns, not significant; *p<0.05; **p<0.01; ***p<0.001; ****p<0.0001 (one-way ANOVA). FIGS. 25e-25h show measurement of CNF in the diaphragm (FIGS. 25e and 25f) and gastrocnemius (FIGS. 25g and 25h) muscles of mdx^{4cv} mice with or without AAV9-iNG treatment at 10 weeks (FIGS. 25e and 25g) or 10 months (FIGS. 25f and 25h) of age (two-tailed, unpaired Student's t-test). FIGS. 25i-25l show muscle fiber size measurement and distribution in diaphragm and gastrocnemius muscles of the mice (WT and mdx^{4cv} with or without AAV9-iNG) at 10 weeks of age. ns, not significant; **p<0.01 (one-way ANOVA). FIG. 25m shows tetanic torque measurements of the posterior compartment muscles. *p<0.05; ****p<0.0001 (one-way ANOVA).

FIGS. 26a-26d show measurement of muscle fiber size in diaphragm and gastrocnemius muscles at 10 months of age. FIGS. 26a-26b show average CSA of diaphragm (FIG. 26a) and gastrocnemius (FIG. 26b) muscle fibers. c, d, Distribution of diaphragm (FIG. 26c) and gastrocnemius (FIG. 26d) muscle fiber areas. ns, not significant; *p<0.05 (one-way ANOVA).

FIGS. 27a-27j show host immune response, toxicity and off-target studies of AAV9-iNG therapy in mdx^{4cv} mice. FIGS. 27a and 27b show host immune response to AAV9 capsid and the base editor transgene (anti-Cas9). IM, intramuscular injection; IV, intravenous injection. ****p<0.0001 (one-way ANOVA) compared to WT or mdx^{4cv}. FIGS. 27c-27e show measurements of serum AST (FIG. 27c), ALT (FIG. 27d) and BUN (FIG. 27e) of mice treated with or without AAV9-iNG. ns, not statistically significant (one-way ANOVA). FIG. 27f shows predicted off-target sites with different number of mismatches from the mdx^{4cv} target gRNA, and the sequences of two most similar off-target sites located on chromosome 16 and 1, respectively. FIGS. 27g and 27h show quantification of deep sequencing reads of the genomic DNA PCR amplicons of the chromosome 16 off-target site (FIG. 27g) or the chromosome 1 off-target site (FIG. 27h) from Neuro-2a cells transfected with ABE-NG, iABE-NGA or control plus the gRNA. ns, not significant (one-way ANOVA). FIG. 27i shows quantification of the A⁴-to-G editing in mdx^{4cv} mice treated with or without AAV9-iNG. ****p<0.0001 (student's t-test). FIG. 27j shows quantification of the bystander C6-to-T editing in mdx^{4cv} mice treated with or without AAV9-iNG. ***p<0.001 (student's t-test). The sequences in FIG. 27 are GTTATCTCCTGTTCTGCAGCTGT (SEQ ID NO: 621), GTTATCTCCTGCTCTGCAGCAGA (SEQ ID NO: 628), GATATCTCCTGT TCTGCAGGAGA (SEQ ID NO: 629).

FIG. 28 shows off-target activities of AAV9-iNG. The nucleotide frequency at the on-target site of the four mdx^{4cv} mice at 10 months after treatment with AAV9-iNG. The desired edit at A⁴ is highlighted in green and the bystander C6 edit in red. The sequence in FIG. 28 is GTTATCTCCTGTTCTGCAGCTGT (SEQ ID NO: 621).

FIGS. 29a-29c show off-target RNA editing activities in the mdx^{4cv} mouse hearts (10 weeks and 10 months of age) induced by systemic delivery of AAV9-iNG. FIG. 29a shows the number of RNA SNVs in three AAV9-iNG treated mouse hearts after filtering the RNA SNVs in three control mouse hearts. FIG. 29b shows the A-to-I editing rate of all RNA SNVs. FIG. 29c shows the A-to-I editing rate of the common RNA SNVs in the three AAV9-iNG treated mouse hearts.

FIGS. 30a-30b show iABE-NGA editing-mediated exon skipping of human DMD exon 55 in DMD-hiPSC-derived cardiomyocytes. FIG. 30a shows Sanger sequencing showing that iABE-NGA mediated efficient conversion of A to G at the splice acceptor site of human DMD exon 55. FIG. 30b shows RT-PCR analysis showing that iABE-NGA editing led to skipping of exon 55 in ΔE48-54 DMD-hiPSC-derived cardiomyocytes. The WT, ΔE48-54 (green arrow) and E55 skipped transcript (red arrow) were clearly discernable.

FIGS. 31a-31c show iABE-NGA editing-mediated exon 7 inclusion of human SMN2. FIG. 31a shows sequences of SMN1 and SMN2 exon 7 with the different nucleotides at position 6 highlighted in red. Two gRNAs were designed to edit A36 to G with the PAM highlighted in purple. FIG. 31b shows RT-PCR analysis of Neuro-2a cells transfected with a SMN2 reporter gene plus or minus the base editors and gRNAs. The exon 7 inclusion (FL) or exclusion (47) transcripts were measured. FIG. 31c shows quantification of the RT-PCR products. **p<0.01; ****p<0.0001; ns, not significant when compared with the control group, using one-way ANOVA with Turkey post test. The sequences in FIG. 31a are GGTTTCAGACAAAATCA AAAAGAAGGAAGGTGCTCACATTCCTTAAATTAAGGA (SEQ ID NO: 632), GGTTTT AGACAAAATCAAAAAGAAGGAAGGTGCTCACATTCCTTAAATTAAGGA (SEQ ID NO: 633), GTGCTCACATTCCTTAAATTAAGG (SEQ ID NO: 634), gGCTCACATTCCTTA AATTAAGGA (SEQ ID NO: 635).

FIGS. 32a-32c show the iABE-NGA editing of mouse Angpt13 (FIG. 32a), Apoc3 (FIG. 32b) and Asgr1 (FIG. 32c) in Neuro-2a cells. **p<0.01; ***p<0.001; ****p<0.0001

### DETAILED DESCRIPTION

Fusing the CRISPR-Cas9 nickase with nucleobase deaminases (e.g. cytidine or adenine deaminase), a new paradigm-shifting class of genome editing technology, termed “base editors”, have recently been developed. DNA base editors, via catalyzing the conversion of one base to another, directly and precisely install point mutations into chromosomal DNA without making DSBs. Therefore, base editing can be developed as promising therapeutics to correct the genetic diseases without DNA cleavage. In particular, the adenine base editors (ABEs) show remarkable fidelity in mouse embryos and rice as compared to cytosine base editors (CBEs), making them highly attractive in therapeutic development. Moreover, nearly half of the point mutations causing human diseases are G-to-A or C-to-T, highlighting the potential of ABEs in correcting a large number of human diseases. In particular, 174 out of 508 pathogenic point mutations for DMD are 5 due to G:C to A:T conversion (Table 5), which can be targeted by ABE editing.

In vivo base editing can correct a custom-made mouse model of Duchenne muscular dystrophy (DMD), which carries a nonsense mutation in exon 20 with a classical 5′-TGG protospacer adjacent motif (PAM) sequence in the noncoding strand for recognition by the Cas9 from Streptococcus pyogenes (SpCas9). In silico analysis of the ClinVar database showed that about 42.8% of the 53469 human disease-causing mutations can be potential targets for base editing correction; however, the majority (˜72.4%) of these potential targets cannot be suitable for SpCas9 base editing due to the lack of the 5′-NGG PAM sequence within the suitable distance from the mutations. Several variants of SpCas9 have recently been engineered with relaxed PAM (such as xCas9-3.7, SpCas9-NG and ScCas9) and non-G PAM. These enzymes greatly increase the target scope for correcting human mutations. However, their performance to correct genetic mutations in preclinical animal models remains to be determined. Here, the efficacy of correcting a commonly used mouse model of DMD, mdx^{4cv} mice were explored, using NG-targeting base editors.

The present disclosure provides fusion proteins, systems, and compositions for editing disease-associated mutations and uses thereof for treating a disease (for example, Duchenne muscular dystrophy).

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used herein, the terms “may.” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “about” and “approximately” are defined as being “‘close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

A “composition” is intended to include a combination of active agent and another compound or composition, inert (for example, a fusion protein, nucleic acid, or virus) or active, such as an adjuvant.

Dystrophinopathies are a group of muscular dystrophies resulting from mutations in the dystrophin gene, located on the short arm of the X chromosome in the Xp21 region (Kunkel et al. 1985; Monaco et al. 1985; Ray et al. 1985). Of these, “Duchenne muscular dystrophy” or “DMD” is the most common dystrophinopathy resulting from complete absence of the dystrophin gene product, the subsarcolemmal protein dystrophin (Hoffman et al. 1987a; Koenig et al. 1987; Hoffman et al. 1988). While dystrophin deficiency can be a primary cause of DMD, multiple secondary pathways are responsible for the progression of muscle necrosis, abnormal fibrosis and failure of regeneration that results in a progressively worsening clinical status. There is evidence supporting oxidative radical damage to myofibers (Rando 2002), inflammation (Spencer and Tidball 2001; Porter et al. 2002), abnormal calcium homeostasis (Allen 2010; Millay 2009), myonuclear apoptosis (Rando 2001b; Sandri et al. 2001; Tews 2002), abnormal fibrosis and failure of regeneration (Rando 2001b; Bernasconi 1995); (Melone 2000; Morrison 2000; Luz 2002). This body of literature has been validated by cross sectional genome-wide approaches that allow an overall analysis of multiple defective mechanisms in DMD (Chen et al. 2000; Porter 2003). The main symptom of DMD is muscle weakness associated with muscle wasting first with the voluntary muscles, e.g., the hips, pelvic area, thighs, shoulders, and calf muscles.

As used herein, the term “effective amount” refers to an amount of a composition necessary or sufficient to realize a desired biologic effect. An effective amount of the composition would be the amount that achieves a selected result, and such an amount could be determined as a matter of routine experimentation by a person skilled in the art. For example, an effective amount of the composition could be that amount necessary for preventing, treating and/or ameliorating Duchenne muscular dystrophy in a subject. The term is also synonymous with “sufficient amount.”

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASE; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

A gRNA is a component of the CRISPR/Cas system. A “gRNA” (guide ribonucleic acid) herein refers to a fusion of a CRISPR-targeting RNA (crRNA) and a trans-activation crRNA (tracrRNA), providing both targeting specificity and scaffolding/binding ability for Cas9 nuclease. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences. The native gRNA comprises a Specificity Determining Sequence (SDS), which specifies the DNA sequence to be targeted. At least a portion of the target DNA sequence is complementary to the SDS of the gRNA. For Cas9 to successfully bind to the DNA target sequence, a region of the target sequence is complementary to the SDS of the gRNA sequence and is immediately followed by the correct protospacer adjacent motif (PAM) sequence (e.g., NGG or NG for Cas9 used herein). In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence.

The term “linker,” as used herein, refers to a chemical group or a molecule linking two molecules or moieties, e.g., two domains of a fusion protein, such as, for example, a Cas9 domain and a nucleic acid editing domain (e.g., a deaminase domain). In some embodiments, a linker joins a gRNA binding domain of an RNA-programmable nuclease, including a Cas9 nuclease domain, and the catalytic domain of a nucleic acid editing protein. In some embodiments, a linker joins a Cas9 and a nucleic acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

The term “nickase” as used herein, refers to a nuclease that cleaves only a single DNA strand, either due to its natural function or because it has been engineered to cleave only a single DNA strand. Jinek et al., “A programmable dual-RNA-guided DNA endonuclease in adaptive bacterial immunity” Science 337(6096):816-821 (2012) and Cong et al. Multiplex genome engineering using CRISPR/Cas systems” Science 339(6121):819-823 (2013).

The term “nucleic acid editing domain,” as used herein refers to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments the nucleic acid editing domain comprises a deaminase (e.g., a cytidine deaminase or an adenine deaminase).

An “adenine deaminase” is an enzyme involved in purine metabolism. It is needed for the breakdown of adenosine from food and for the turnover of nucleic acids in tissues. Its primary function in humans is the development and maintenance of the immune system. An adenine deaminase catalyzes hydrolytic deamination of adenosine (forming inosine, which base pairs as G) in the context of DNA.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage. For example, Duchenne muscular dystrophy, may result in e.g., a slowing of muscle degeneration, decreased fatigue, increased muscle strength, reduced blood levels of creatine kinase (CK), decreased difficulty with motor skills, decreased muscle fiber deformities, decreased inflammation or fibrotic tissue infiltration in the muscle, stabilization of the progression of the disease (e.g., by halting progressive muscle weakness) etc.

“Therapeutically effective amount” or “therapeutically effective dose” of a composition (e.g. a fusion protein, a nucleic acid, or virus) refers to an amount that is effective to achieve a desired therapeutic result. In some embodiments, a desired therapeutic result is the prevention of Duchenne muscular dystrophy. In some embodiments, a desired therapeutic result is the treatment of Duchenne muscular dystrophy. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as coughing relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, lentiviral vectors, adenoviral vectors, adeno-associated virus vectors, retroviral vectors, and the like.

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions

In some aspects, disclosed herein is a fusion protein comprising a Cas9 nickase and a nucleotide deaminase, wherein the Cas9 nickase comprises a first amino acid substitution at a position selected from the group consisting of 262, 324, 409, 480, 543, 694, and 1219 when compared to SEQ ID NO: 11, and wherein the Cas9 nickase comprises a second amino acid substitution at a position selected from the group consisting of 1111, 1135, 1218, 1219, 1322, 1335, and 1337 when compared to SEQ ID NO: 11. In some embodiments, the Cas9 nickase and the nucleotide deaminase are operably linked.

In some embodiments, the first amino acid substitution is selected from the group consisting of A262T, R324L, S409I, E480K, E543D, M694I, and E1219V when compared to SEQ ID NO: 11. In some embodiments, the second amino acid substitution is selected from the group consisting of L1111R, D1135V, G1218R, E1219F, A1322R, R1335V, R1335Q, R1335E, and T1337R when compared to SEQ ID NO: 11.

In some embodiments, the fusion protein comprises a Cas9 nickase fused to a deaminase and further fused to a Uracil DNA glycosylase inhibitor (UGI) domain.

In some embodiments, the Cas9 nickase comprises an amino acid sequence at least 80% identity (at least 80%, at least 85%, at least 90%, at least 95%, at least 96%, at least 97%, at least 98%, at least 99%, or at least 99.5%) to the amino acid sequence selected from the group consisting of SEQ ID NOs: 12-20. In some embodiments, the Cas9 nickase comprises the amino acid sequence selected from the group consisting of SEQ ID NOs: 12-20.

In some embodiments, the Cas9 nickase when in conjunction with a bound guide RNA (gRNA) specifically binds to a target nucleic acid sequence.

In some embodiments, the Cas9 nickase recognizes a NG protospacer adjacent motif (PAM) sequence.

In some embodiments, the nucleotide deaminase is a cytidine deaminase or an adenine deaminase. In some embodiments, the nucleotide deaminase is a cytidine deaminase. In some embodiments, the nucleotide deaminase is an adenine deaminase. In some embodiments, the adenine deaminase is a dimeric adenine deaminase or a monomeric adenine deaminase domain. In some embodiments, the dimeric adenine deaminase comprises an amino acid sequence at least 80% identity (at least 80%, at least 85%, at least 90%, at least 95%, at least 96%, at least 97%, at least 98%, at least 99%, or at least 99.5%) to SEQ ID NOs: 22. In some embodiments, the monomeric adenine deaminase comprises an amino acid sequence at least 80% identity (at least 80%, at least 85%, at least 90%, at least 95%, at least 96%, at least 97%, at least 98%, at least 99%, or at least 99.5%) to SEQ ID NOs: 23. In some embodiments, the monomeric adenine deaminase comprises amino acid substitutions A56G and V82G when compared to SEQ ID NO: 47. In some embodiments, the adenine deaminase comprises the amino acid sequence set forth in SEQ ID NO: 22 or 23.

In some embodiments, the adenine deaminase is encoded by a nucleotide sequence at least 80% identity (at least 80%, at least 85%, at least 90%, at least 95%, at least 96%, at least 97%, at least 98%, at least 99%, or at least 99.5%) to SEQ ID NO: 41 or 42. In some embodiments, the adenine deaminase is encoded by the nucleotide sequence as set forth in SEQ ID NO: 41 or 42.

In some embodiments, the Cas9 nickase comprising the monomeric adenine deaminase domain has a lower off-target RNA editing activity than a Cas9 nickase comprising a dimeric adenine deaminase domain (e.g., at least 10% lower, at least 20% lower, at least 30% lower, at least 40% lower, at least 50% lower, at least 60% lower, at least 70% lower, at least 80% lower, at least 90% lower, at least 95% lower, at least 99% lower, or at least 2 times lower, at least 3 times lower, at least 4 times lower, at least 5 times lower, at least 6 times lower, at least 7 times lower, at least 8 times lower, at least 9 times lower, at least 10 times lower, at least 20 times lower, at least 50 times lower, at least 100 times lower, at least 150 times lower, at time 200 times lower, at least 500 times lower, or at least 1000 times lower).

In some aspects, disclosed herein is a construct comprising a nucleotide sequence encoding the fusion protein of any preceding aspect. In some embodiments, the construct further comprises a promoter operably linked to the nucleotide sequence, wherein the promoter is a CMV promoter. Accordingly, in some embodiments, the nucleotide sequence of any preceding aspect is at least 80% identity (at least 80%, at least 85%, at least 90%, at least 95%, at least 96%, at least 97%, at least 98%, at least 99%, or at least 99.5%) to SEQ ID NO: 1 or SEQ ID NO: 2.

In some aspects, disclosed herein is a system for base editing comprising:
a first nucleotide sequence encoding a N-terminal portion of a Cas9 nickase fused at its C-terminus to an intein-N; and
a second nucleotide sequence encoding an intein-C fused to the N-terminus of a C-terminal portion of the Cas9 nickase;
wherein the first nucleotide sequence further comprises a nucleotide sequence encoding a nucleotide deaminase fused to the N-terminus of the N-terminal portion of the Cas9 nickase,
wherein the Cas9 nickase comprises a first amino acid substitution at a position selected from the group consisting of 262, 324, 409, 480, 543, 694, and 1219 when compared to SEQ ID NO: 11, and
wherein the Cas9 nickase comprises a second amino acid substitution at a position selected from the group consisting of 1111, 1135, 1218, 1219, 1322, 1335, and 1337 when compared to SEQ ID NO: 11.

In some embodiments, the Cas9 nickase comprises an amino acid sequence at least 80% identity (at least 80%, at least 85%, at least 90%, at least 95%, at least 96%, at least 97%, at least 98%, at least 99%, or at least 99.5%) to the amino acid sequence selected from the group consisting of SEQ ID NOs: 12-20. In some embodiments, the Cas9 nickase comprises the amino acid sequence selected from the group consisting of SEQ ID NOs: 12-20.

Accordingly, in some embodiments, the Cas9 nickase is encoded by a nucleotide sequence at least 80% identity (at least 80%, at least 85%, at least 90%, at least 95%, at least 96%, at least 97%, at least 98%, at least 99%, or at least 99.5%) to the nucleotide sequence selected from the group consisting of SEQ ID NOs: 31-39. In some embodiments, the Cas9 nickase is encoded by a nucleotide sequence selected from the group consisting of SEQ ID NOs: 31-39.

In some embodiments, the N-terminal portion of the Cas9 nickase comprises the amino acid positions 2-560, 2-561, 2-562, 2-563, 2-564, 2-565, 2-566, 2-567, 2-568, 2-569, 2-570, 2-571, 2-572, 2-573, 2-574, 2-575, 2-576, 2-577, 2-578, 2-579, or 2-580 of the amino acid sequence selected from the group consisting of SEQ ID NOs: 12-20. In some embodiments, the C-terminal portion of the Cas9 nickase comprises the amino acid positions 574-1368 of the amino acid sequence selected from the group consisting of SEQ ID NOs: 12-20.

An “intein” is a segment of a protein that is able to excise itself and join the remaining portions (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein. The inteins used herein can be, for example, Npu DnaE intein, Cfa DnaE intein or pg41-1 intein. The intein encoded by the DnaE-n gene is herein referred as “intein-N.” The intein encoded by the DnaE-c gene is herein referred as “intein-C.”

Other intein systems may also be used. For example, a synthetic intein based on the dnaE intein, the Cfa-N and Cfa-C intein pair, has been described (e.g., in Stevens et al., J Am Chem Soc. 2016 Feb. 24; 138(7):2162-5, incorporated herein by reference). Non-limiting examples of intein pairs that may be used in accordance with the present disclosure include: Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

In some embodiments, the intein-N is a Cfa intein-N or a gp41-1 intein-N. Accordingly, in some embodiments, the intein-N comprises the amino acid sequence of SEQ ID NO: 24 or 26. In some embodiments, the intein-N is encoded by the nucleotide sequence of SEQ ID NO: 43 or 45. Accordingly, the first nucleotide sequence of any preceding aspects comprises SEQ ID NO: 43 or 45.

In some embodiments, the intein-C is a Cfa intein-C or a gp41-1 intein-C. Accordingly, in some embodiments, the intein-C comprises the amino acid sequence of SEQ ID NO: 25 or 27. In some embodiments, the intein-C is encoded by the nucleotide sequence of SEQ ID NO: 44 or 46. Accordingly, the second nucleotide sequence of any preceding aspects comprises SEQ ID NO: 44 or 46.

In some embodiments, the first nucleotide sequence or the second nucleotide sequence further comprises a nucleotide encoding a guide RNA (gRNA).

In some embodiments, the first nucleotide sequence comprises an N terminal portion of SEQ ID NO: 1 or 2. In some embodiments, the first nucleotide comprises a portion of SEQ ID NO: 1 or 2.

In some embodiments, the second nucleotide sequence comprises a C terminal portion of SEQ ID NO: 1 or 2. In some embodiments, the second nucleotide comprises a portion of SEQ ID NO: 1 or 2

In some aspects, disclosed herein is a composition comprising:
a first recombinant viral particle comprising a first nucleotide sequence encoding a N-terminal portion of a Cas9 nickase fused at its C-terminus to an intein-N; and
a second recombinant viral particle comprising a second nucleotide sequence encoding an intein-C fused to the N-terminus of a C-terminal portion of the Cas9 nickase;
wherein the first nucleotide sequence further comprises a nucleotide sequence encoding a nucleotide deaminase fused to the N-terminus of the N-terminal portion of the Cas9 nickase,
wherein the Cas9 nickase comprises a first amino acid substitution at a position selected from the group consisting of 262, 324, 409, 480, 543, 694, and 1219 when compared to SEQ ID NO: 11, and
wherein the Cas9 nickase comprises a second amino acid substitution at a position selected from the group consisting of 1111, 1135, 1218, 1219, 1322, 1335, and 1337 when compared to SEQ ID NO: 11.

In some embodiments, the Cas9 nickase comprises an amino acid sequence at least 80% identity (at least 80%, at least 85%, at least 90%, at least 95%, at least 96%, at least 97%, at least 98%, at least 99%, or at least 99.5%) to the amino acid sequence selected from the group consisting of SEQ ID NOs: 12-20. In some embodiments, the Cas9 nickase comprises the amino acid sequence selected from the group consisting of SEQ ID NOs: 12-20.

Accordingly, in some embodiments, the Cas9 nickase is encoded by a nucleotide sequence at least 80% identity (at least 80%, at least 85%, at least 90%, at least 95%, at least 96%, at least 97%, at least 98%, at least 99%, or at least 99.5%) to the nucleotide sequence selected from the group consisting of SEQ ID NOs: 31-39. In some embodiments, the Cas9 nickase is encoded by a nucleotide sequence selected from the group consisting of SEQ ID NOs: 31-39.

In some embodiments, the N-terminal portion of the Cas9 nickase comprises the amino acid positions 2-560, 2-561, 2-562, 2-563, 2-564, 2-565, 2-566, 2-567, 2-568, 2-569, 2-570, 2-571, 2-572, 2-573, 2-574, 2-575, 2-576, 2-577, 2-578, 2-579, or 2-580 of the amino acid sequence selected from the group consisting of SEQ ID NOs: 12-20. In some embodiments, the C-terminal portion of the Cas9 nickase comprises the amino acid positions 574-1368 of the amino acid sequence selected from the group consisting of SEQ ID NOs: 12-20.

In some embodiments, the intein-N is a Cfa intein-N or a gp41-1 intein-N. Accordingly, in some embodiments, the intein-N comprises the amino acid sequence of SEQ ID NO: 24 or 26. In some embodiments, the intein-N is encoded by the nucleotide sequence of SEQ ID NO: 43 or 45. Accordingly, the first nucleotide sequence of any preceding aspects comprises SEQ ID NO: 43 or 45.

In some embodiments, the intein-C is a Cfa intein-C or a gp41-1 intein-C. Accordingly, in some embodiments, the intein-C comprises the amino acid sequence of SEQ ID NO: 25 or 27. In some embodiments, the intein-C is encoded by the nucleotide sequence of SEQ ID NO: 44 or 46. Accordingly, the second nucleotide sequence of any preceding aspects comprises SEQ ID NO: 44 or 46.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used

AAV is a preferred vector because it can infect many cell types and is nonpathogenic to humans. AAV type vectors can transport about 4 to 5 kb and wild type AAV is known to stably insert into chromosome 19. Vectors which contain this site-specific integration property are preferred. In some embodiments, the AAV vector of any preceding aspect further a herpes simplex virus thymidine kinase gene, HSV-tk, and/or a marker gene, such as the gene encoding the green fluorescent protein, GFP.

Accordingly, in some aspects, disclosed herein is a composition comprising:
a first recombinant adeno-associated virus (AAV) particle comprising a first nucleotide sequence encoding a N-terminal portion of a Cas9 nickase fused at its C-terminus to an intein-N; and
a second recombinant AAV particle comprising a second nucleotide sequence encoding an intein-C fused to the N-terminus of a C-terminal portion of the Cas9 nickase;
wherein the first nucleotide sequence further comprises a nucleotide sequence encoding a nucleotide deaminase fused to the N-terminus of the N-terminal portion of the Cas9 nickase,
wherein the Cas9 nickase comprises a first amino acid substitution at a position selected from the group consisting of 262, 324, 409, 480, 543, 694, and 1219 when compared to SEQ ID NO: 11, and
wherein the Cas9 nickase comprises a second amino acid substitution at a position selected from the group consisting of 1111, 1135, 1218, 1219, 1322, 1335, and 1337 when compared to SEQ ID NO: 11.

In some embodiments, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration. U.S. Pat. No. 6,261,834 is herein incorporated by reference for material related to the AAV vector.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements. The spacing between promoter elements frequently is flexible, so that promoter function is preserved when elements are inverted or moved relative to one another. Depending on the promoter, it appears that individual elements can function either cooperatively or independently to activate transcription.

The term “promoter” as used herein is defined as a DNA sequence recognized by the synthetic machinery of the cell, or introduced synthetic machinery, required to initiate the specific transcription of a polynucleotide sequence. One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1 alpha (EF-1α). However, other promoter sequences may also be used, including, but not limited to the simian virus 40 (SV40), early promoter, mouse mammary tumor virus (MMTV), human immunodeficiency virus (HIV) long terminal repeat (LTR) promoter, MoMul.V promoter, PGK-1 promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter as well as synthetic protein, such as a CAG promoter. Further, the invention should not be limited to the use of constitutive promoters, inducible promoters are also contemplated as part of the invention. The use of an inducible promoter provides a molecular switch capable of turning on expression of the polynucleotide sequence which it is operatively linked when such expression is desired, or turning off the expression when expression is not desired. In some embodiments, the AAV of any preceding aspect comprises a constitutive promoter or a muscle tissue specific promoter, e.g., a muscle-specific MHP1 promoter.

In some embodiments, the promoter is a CMV promoter. In some embodiments, the CMV promoter comprises the nucleotide sequence of SEQ ID NO: 28. Accordingly, in some embodiments, the first recombinant adeno-associated virus (AAV) particle of any preceding aspect comprises a first nucleotide sequence that is at least 80% identity (at least 80%, at least 85%, at least 90%, at least 95%, at least 96%, at least 97%, at least 98%, at least 99%, or at least 99.5%) to SEQ ID NO: 3 or SEQ ID NO: 5, wherein the second recombinant adeno-associated virus (AAV) particle of any preceding aspect comprises a first nucleotide sequence that is at least 80% identity (at least 80%, at least 85%, at least 90%, at least 95%, at least 96%, at least 97%, at least 98%, at least 99%, or at least 99.5%) to SEQ ID NO: 4 or SEQ ID NO: 6.

In some embodiments, the promoter is a MHP1 promoter. In some embodiments, the MHP1 promoter comprises the nucleotide sequence of SEQ ID NO: 29. Accordingly, in some embodiments, the first recombinant adeno-associated virus (AAV) particle of any preceding aspect comprises a first nucleotide sequence that is at least 80% identity (at least 80%, at least 85%, at least 90%, at least 95%, at least 96%, at least 97%, at least 98%, at least 99%, or at least 99.5%) to SEQ ID NO: 7, wherein the second recombinant adeno-associated virus (AAV) particle of any preceding aspect comprises a first nucleotide sequence that is at least 80% identity (at least 80%, at least 85%, at least 90%, at least 95%, at least 96%, at least 97%, at least 98%, at least 99%, or at least 99.5%) to SEQ ID NO: 8.

In some embodiments, viral vector is a lentivirus vector. Accordingly, disclosed herein is a composition comprising:
a first recombinant lentivirus particle comprising a first nucleotide sequence encoding a N-terminal portion of a Cas9 nickase fused at its C-terminus to an intein-N; and
a second recombinant lentivirus particle comprising a second nucleotide sequence encoding an intein-C fused to the N-terminus of a C-terminal portion of the Cas9 nickase;
wherein first nucleotide sequence further comprises a nucleotide sequence encoding a nucleotide deaminase fused to the N-terminus of the N-terminal portion of the Cas9 nickase,
wherein the Cas9 nickase comprises a first amino acid substitution at a position selected from the group consisting of 262, 324, 409, 480, 543, 694, and 1219 when compared to SEQ ID NO: 11, and
wherein the Cas9 nickase comprises a second amino acid substitution at a position selected from the group consisting of 1111, 1135, 1218, 1219, 1322, 1335, and 1337 when compared to SEQ ID NO: 11.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. See, e.g., WO2012079000A1, incorporated by reference herein in their entireties.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to mod late promoter-driven transcription. In some embodiments, the recombinant nucleic acid of any preceding aspect further comprises a reporter gene. In some embodiments, the reporter gene in invention is GFP.

In some embodiments, the fusion protein, nucleotide, system, or composition of any preceding aspect can be further formulated in a pharmaceutically acceptable carrier.

### Genetic Diseases and Methods of Treatment

It is estimated that over 10,000 human diseases are caused by genetic disorders, which are abnormalities in genes or chromosomes. See, e.g., McClellan, J. and M. C. King, Genetic heterogeneity in human disease. Cell. 141(2): p. 210-7; Leachman, S. A., et al., Therapeutic siRNAs for dominant genetic skin disorders including pachyonychia congenita. J Dermatol Sci, 2008. 51(3): p. 151-7. The compositions disclosed herein can be used to treat a number of these genetic disorders.

In some aspects, disclosed herein is a method of treating a genetic disease in a subject, comprising administering to the subject a therapeutically effective amount of the fusion protein, system, or composition of any preceding aspect.

In some embodiments, the genetic disease is a muscular dystrophy. Muscular dystrophies are a group of muscle diseases caused by mutations in a person's genes. Over time, muscle weakness decreases mobility, making everyday tasks difficult. The methods and compositions disclosed herein can be used for treating a muscular dystrophy, including, for example, Duchenne muscular dystrophy, Becker muscular dystrophy, myotonic dystrophy, limb-girdle muscular dystrophy, facioscapulohumeral dystrophy, congenital muscular dystrophy, distal muscular dystrophy, oculopharyngeal muscular dystrophy, or Emery-Dreifuss muscular dystrophy.

In some embodiments, the genetic disease or disorder is Duchenne muscular dystrophy, dysferlinopathy, or spinal muscular atrophy. In some embodiments, the methods and compositions disclosed herein can be used for treating Duchenne muscular dystrophy. In some embodiments, the genetic disease is spinal muscular atrophy.

In some embodiments, the genetic disease or disorder is hypercholesterolemia. For treating hypercholesterolemia, iABE-NGA is used to create a protective silent mutation in functional genes, for example ANGPTL3, APOC3, PCSK9, or ASGR1, to lower plasma cholesterol levels.

In some embodiments, the genetic disease or disorder is due to a point mutation in a causative gene from a G:C pair to an A:T pair.

In some aspects, disclosed herein is a method of treating a genetic disease in a subject, comprising administering to the subject a therapeutically effective amount of the fusion protein, system, and composition of any preceding aspect.

The disclosed methods can be performed any time prior to the onset of a genetic disease, even prior to the apparent of any symptom. In one aspect, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of the genetic disease or any symptom thereof; or 1, 2, 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 45, or 60 years after the onset of the genetic disease or any symptom thereof.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, once every 10 min, once every 5 min, or once per min. Administration can also be continuous and adjusted to maintaining a level of the compound within any desired and specified range.

The compositions of the present invention can be administered to the appropriate subject in any manner known in the art, e.g., orally, intramuscularly, intravenously, sublingual mucosal, intraarterially, intrathecally, intradermally, intraperitoneally, intranasally, intrapulmonarily, intraocularly, intravaginally, intrarectally, subcutaneously, or through by inhalation. Parenteral administration, if used, is generally characterized by injection. Injectables can be prepared in conventional forms, either as liquid solutions or suspensions, solid forms suitable for solution or suspension in liquid prior to injection, or as emulsions.

### EXAMPLES

The following examples are set forth below to illustrate the compounds, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. In Vitro Reporter Assay Demonstrates the Feasibility to Correct the Mdx^{4cv} Mutation Using ABE-NG

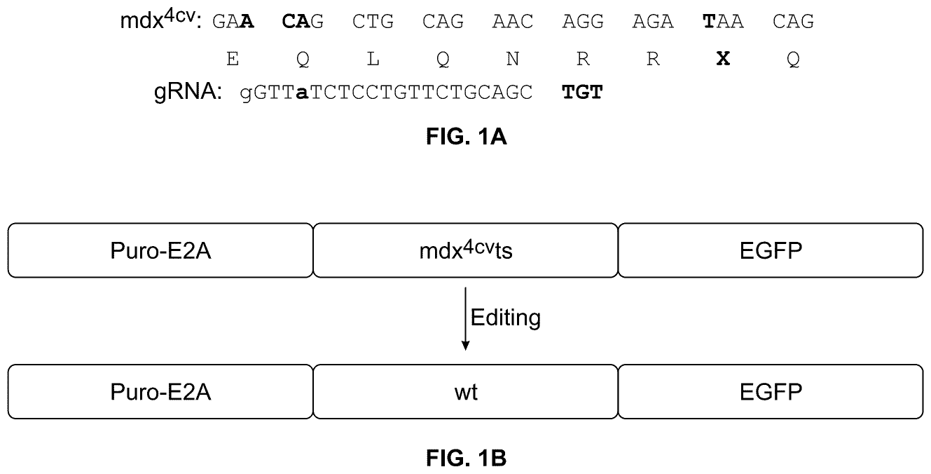

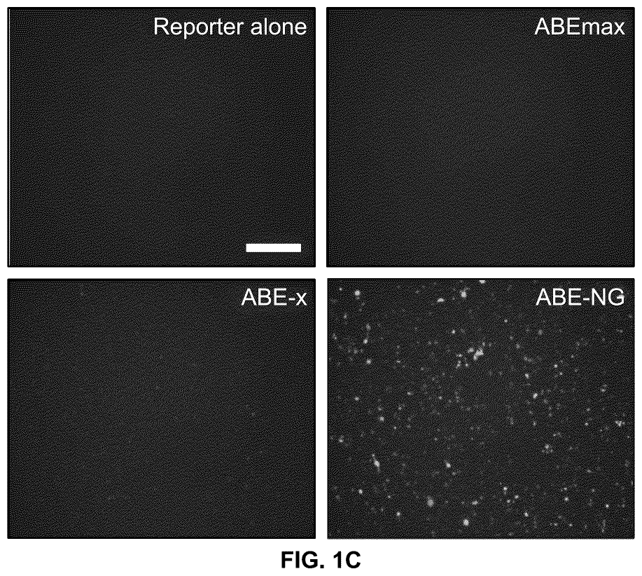

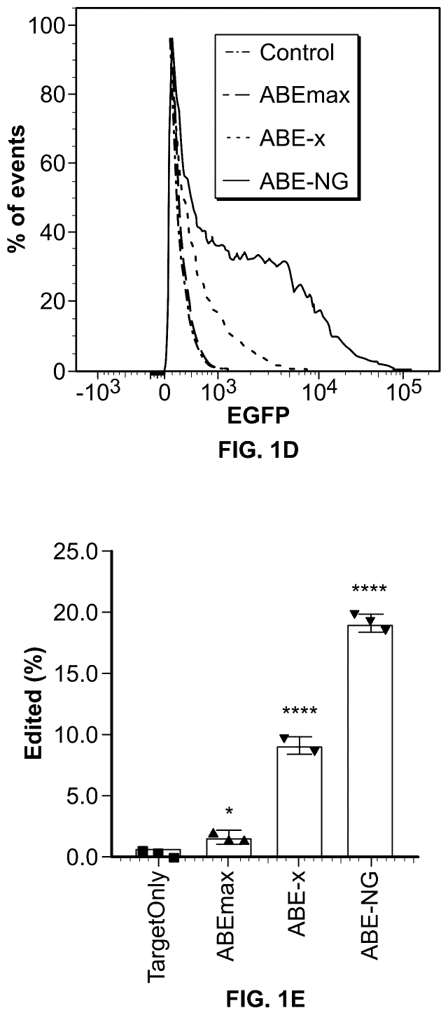

The mdx^{4cv} mouse carries a premature stop codon (CAA-to-TAA) in the exon 53 of Did gene, which disrupts the expression of dystrophin and leads to the development of muscular dystrophy. Targeting the noncoding strand with ABEs can correct this nonsense mutation. However, in the noncoding strand, there is a lack of 5′-NGG sequence at the downstream of this mutation within the suitable editing window, but a 5′-TGT PAM is present with the mutated A located at position 4 in the guide RNA (gRNA) (FIG. 1a), making it feasible to correct the stop codon with the NG-targeting base editors in this widely used mouse model of DMD. A reporter plasmid with the targeting sequence from the mdx^{4cv} mice was first constructed (FIG. 1b). The nonsense mutation in the mdx^{4cv} targeting sequence disrupts the expression of downstream EGFP and successful editing of the nonsense mutation is indicated by the restoration of EGFP expression. As shown in FIG. 1c, transfection with the reporter alone resulted in minimal background fluorescence. Similarly, co-transfection with the reporter, mdx^{4cv}-gRNA and ABEmax failed to restore EGFP expression. However, ABE-NG (based on SpCas9-NG) successfully restored EGFP expression in this reporter assay. In contrast, ABE-x (based on xCas9-3.7) was found to be less efficient in restoring EGFP expression even though xCas9-3.7 was also engineered to target 5′-NG PAM, consistent with previous reports that xCas9-3.7 is generally less efficient than SpCas9-NG. FACS analysis showed that ABE-x and ABE-NG restored EGFP expression in 10% and 20% cells, respectively (FIGS. 1d, 1e). These in vitro studies showed that ABE-NG corrects the nonsense mdx^{4cv} mutation.

### Example 2. Improvements in the Editing Efficiency and Specificity of ABE-NG

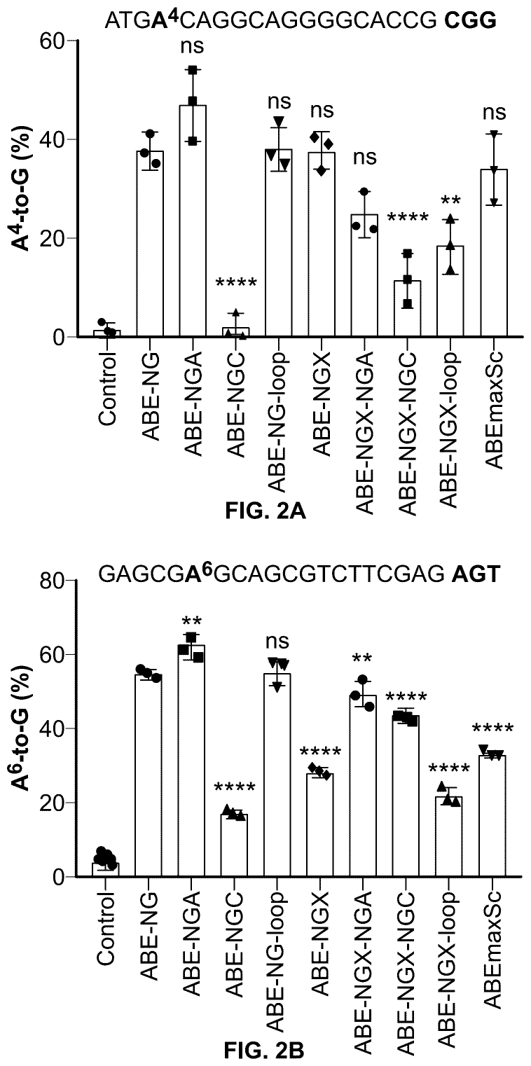

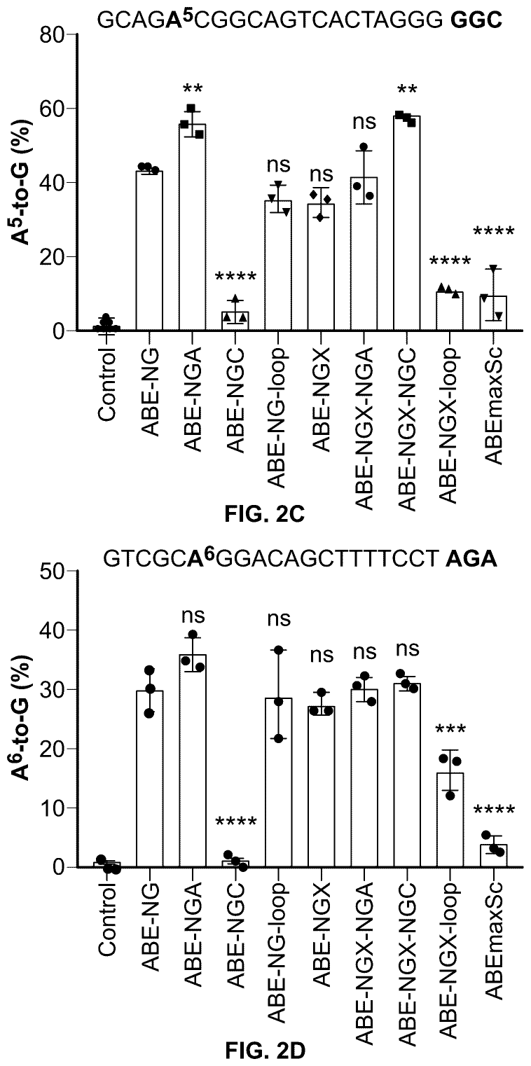

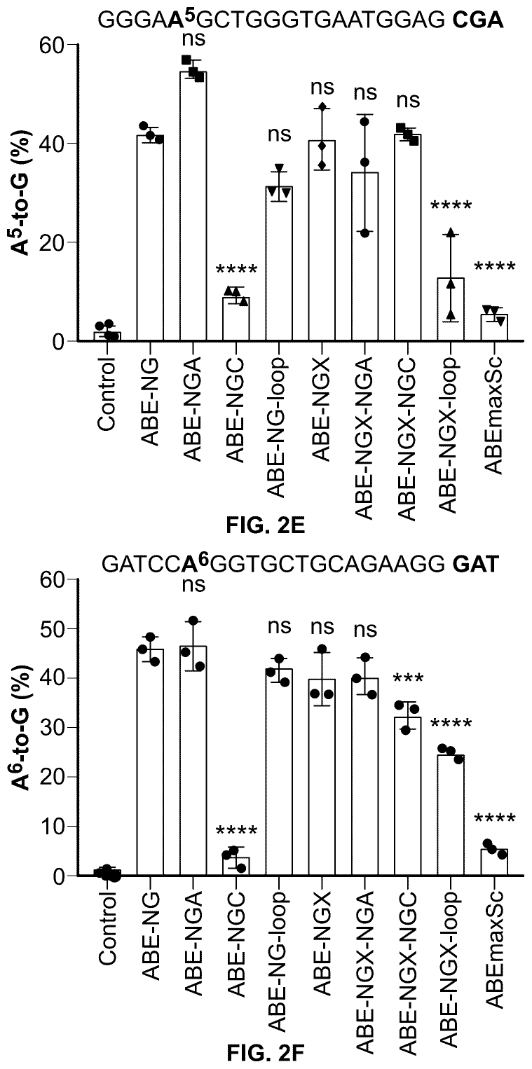

The relative low efficiency of ABE-NG, together with the recently reported off-target RNA editing activity, prompted the re-design of ABE-NG in order to improve the editing efficiency and specificity. First, the targeting efficiency of ABE-NG at the sites with 5′-NG PAM can be improved by optimizing the PAM-interacting domain. The targeting property of ABE-NG can be modified by combining the mutations in SpCas9-NG (R1335V/L1111R/D1135V/G1218R/E1219F/A1322R/T1337R) with other mutations designed to target different PAM sequences such as those in xCas9(3.7) (A262T/R324L/S409I/E480K/E543D/M694I/E1219V), VQR (D1135V/R1335Q/T1337R), VRER (D1135V/G1218R/R1335E/T1337R) and the loop sequence in ScCas9 (amino acids 367-376). Seven new ABE variants were generated with different combinations of the aforementioned variants (Table 1) and compared their base editing activities at six different loci with those of ABE-NG and ABEmaxSC. While all variants except ABE-NGC (containing all NG mutations plus R1335E) performed similarly at the NGG site (FIG. 2a), it was observed that ABE-NGA (carrying all NG mutations plus R1335Q) had a small improvement in editing the NGH sites as compared to ABE-NG (FIGS. 2b-2e). ABE-NGA and ABE-NGX-NGC (carrying the xCas9(3.7) mutations, NG mutations and R1335E) worked equally well at the NGC site (FIG. 2c). The ABE-NG and ABE-NGA also edited the site containing a 5′-GAT PAM with high efficiency (FIG. 2f), with similar efficiency as compared to the ABE-NGX variant carrying both the xCas9(3.7) mutations and ABE-NG mutations, which was previously reported to have the broadest targeting scope active in plant. However, the efficiency of ABE-NGX at the NGH sites was lower than ABE-NGA (FIG. 2b). Since ABE-NGA is generally superior to other variants tested at NGH sites, ABE-NGA was chosen for further in vitro and in vivo studies.

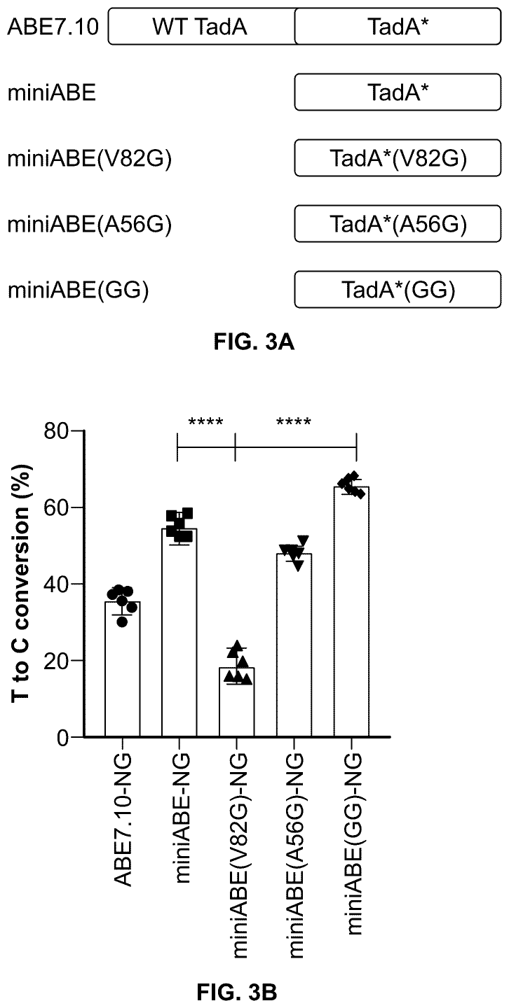

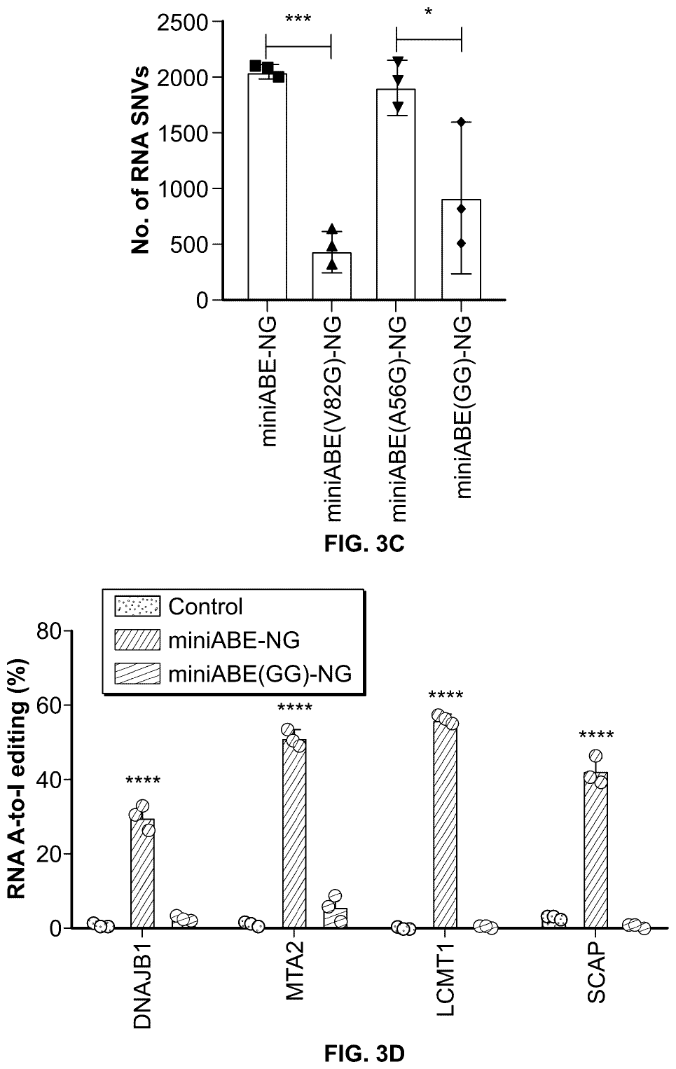

Previous studies showed that the deaminase domain in the ABEs can elicit transcriptome-wide RNA off-target editing activity, and that the off-target RNA editing activity can be substantially reduced by removing the WT ecTadA domain and mutating the evolved ecTadA domain. The dimeric adenine deaminase domain (ecTadA-ecTadA*) in ABE-NG was replaced with the originally evolved ecTadA* monomer or its high-fidelity version (ecTadA*-V82G) (FIG. 3a) in order to minimize the off-target RNA editing activity. The miniABE-NG (the monomeric TadA* fused with SpCas9-NG nickase) performed slightly better at the mdx^{4cv} target site as compared to ABE7.10-NG (FIG. 3b). However, the on-target DNA editing activity of miniABE(V82G)-NG was remarkably reduced by over 50% when compared to ABE7.10-NG (FIG. 3b). Then the next experiment attempted to improve the on-target DNA editing efficiency of the high-fidelity miniABE(V82G)-NG without compromising its low off-target RNA editing activity. The V82G is one of the 26 amino acid residue positions in ecTadA that reside near the enzymatic pocket around the substrate tRNA, inferred from the S. aureus TadA-tRNA co-crystal structure. The V82G mutation does not only affect the non-specific affinity to RNA substrates, but can also reduce its affinity to the DNA substrates. It was noticed that the A56G mutation (which is also inferred to lie near the enzymatic pocket around the tRNA substrate) had higher on-target DNA editing activity without affecting the off-target RNA editing activity as compared to miniABEmax. Installing the A56G mutation into miniABE(V82G)-NG can improve its on-target DNA editing activity without compromising its off-target RNA editing profile. Indeed, it was observed that adding the A56G mutation into miniABE(V82G)-NG (named miniABE(GG)-NG) completely restored its on-target DNA editing activity (FIG. 3b).

RNA-seq was used to compare the transcriptome-wide off-target RNA editing activities of miniABE(GG)-NG to other ABE variants in mouse Neuro2a cells. These studies were performed in triplicate. Edited RNA adenines were identified from RNA-seq experiments by filtering out background editing observed with read-count-matched controls. MiniABE-NG and miniABE(A56G)-NG induced much higher numbers of adenine editing as compared to miniABE(V82G)-NG or miniABE(GG)-NG (FIG. 3c). There was no statistical difference between miniABE(V82G)-NG and miniABE(GG)-NG. To further verify the off-target RNA editing activity of miniABE((G)-NG, four RT-PCR amplicons were amplified and sequenced, which were shown to be highly modified by ABEmax in human cells. Transfection of HEK293 cells with miniABE-NG induced high levels of A-to-I RNA editing in all these transcripts (FIG. 3d); however, such A-to-I RNA editing was essentially eliminated in cells transfected with miniABE(GG)-NG (FIG. 3d). Taken together, these results showed that miniABE(GG)-NG does not only have increased DNA editing activity, but also inherits the high fidelity of miniABE(V82G)-NG. Hereafter, the improved ABE-NG carrying the miniABE(GG) domain and Cas9-NGA nickase was referred to as iABE-NGA.

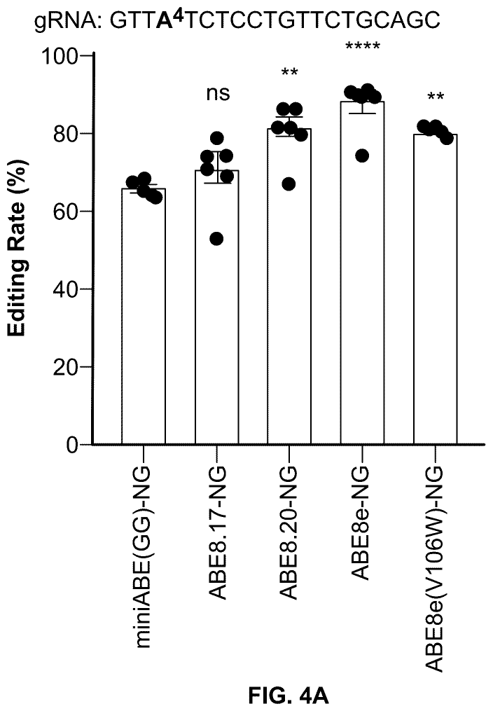

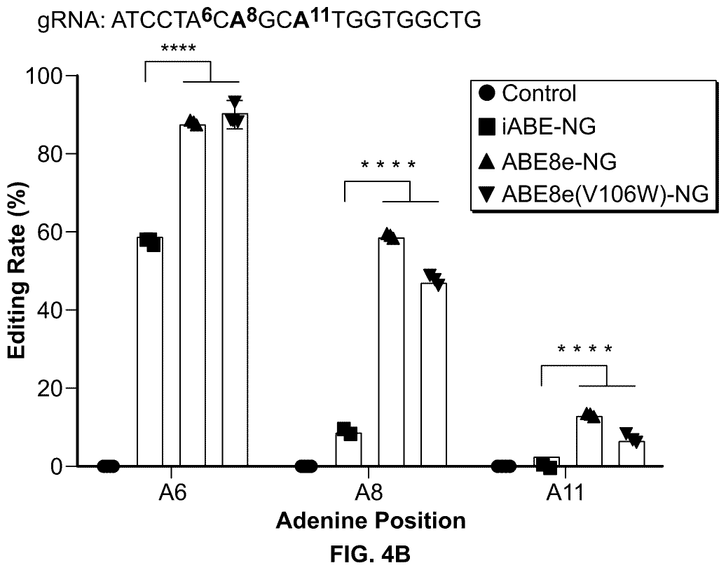

A new generation of ABEs were developed through directed evolution, namely, ABE8s (such as ABE8.17 and ABE8.20) and ABESe. To directly compare miniABE(GG) with ABE8.17, ABE8.20 and ABESe, each of them was fused with SpCas9-NG and tested their activities for editing the mdx^{4cv} target site using the reporter assay in Neuro2A cells. All these editors showed above 60% editing efficiency with the ABESe-NG exhibiting the highest activity (FIG. 4a). ABE8e can have also increased bystander activity than miniABE(GG). Since the mdx^{4cv} target site has no extra adenine within the editing window, the bystander editing activity of ABESe and miniABE(GG) was compared by testing their performance to edit a nonsense mutation in human DYSF gene (encoding dysferlin) that causes limb girdle muscular dystrophy. The target A⁶ was edited with ˜58% and 88% efficiency by iABE-NG and ABESe-NG, respectively (FIG. 4b). The two bystander adenines at positions 8 and 11 were also edited at substantially higher (quantitative numbers) rates by ABESe-NG than miniABE(GG)-NG. Even the ABESe with V106W mutation still displayed very high bystander editing activity at position 8. Thus, for in vivo applications that require not only efficiency but also precision, the use of miniABE(GG) was recommended.

### Example 3. Intein-Split Allows Efficient Assembly of Full-Length ABE-NG and Editing

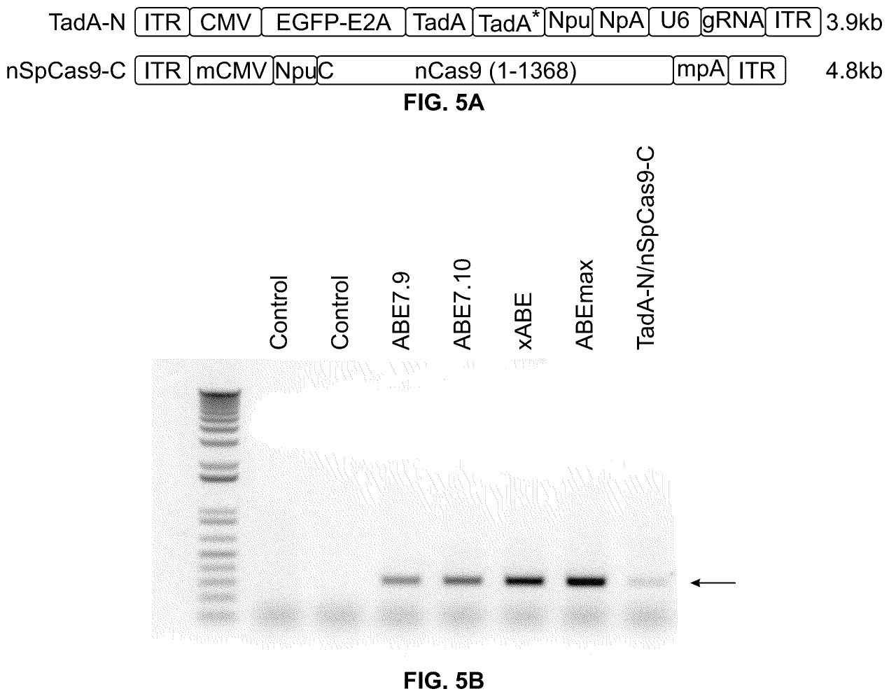

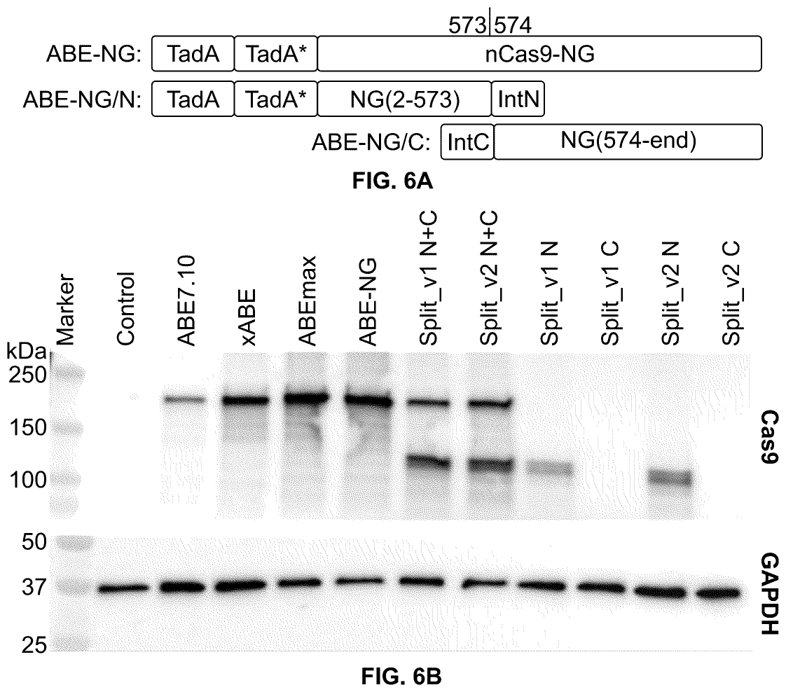

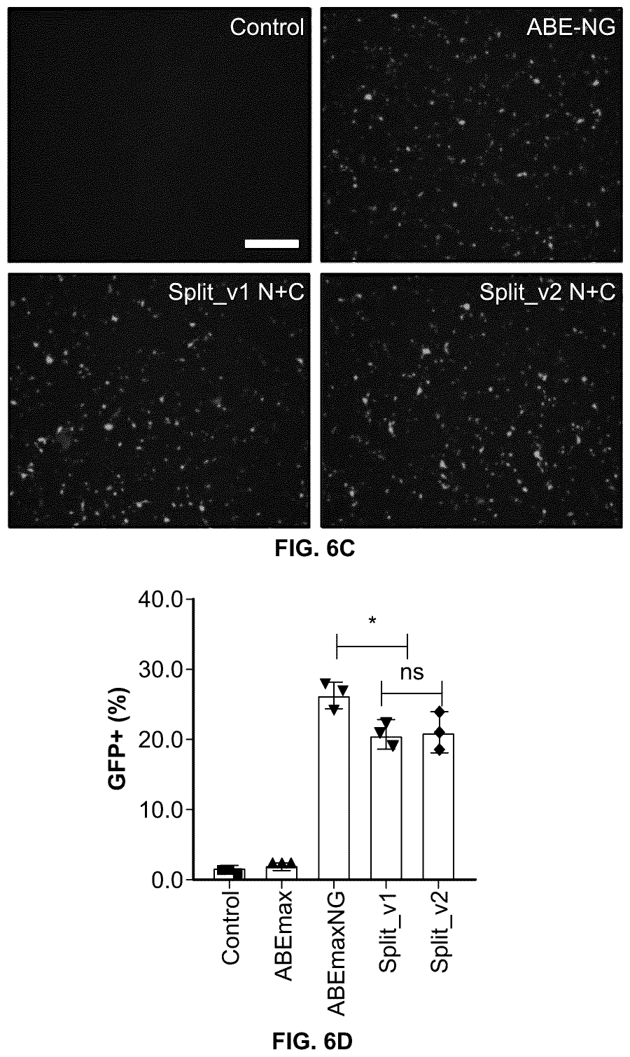

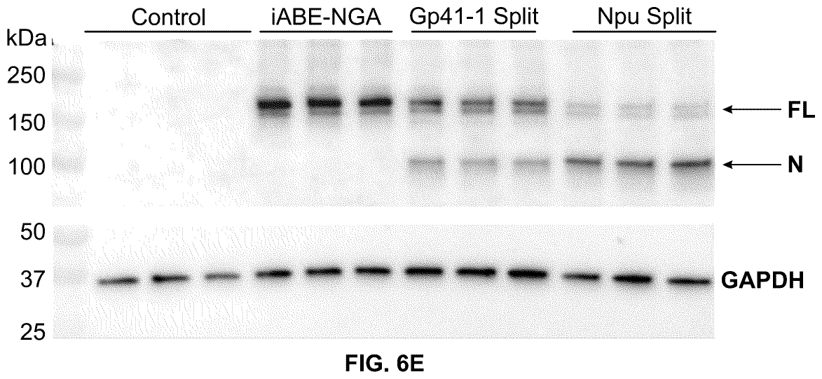

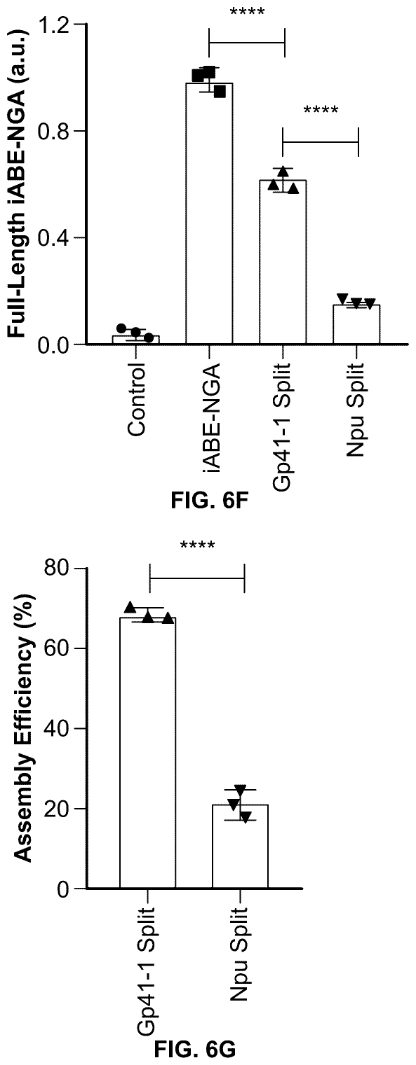

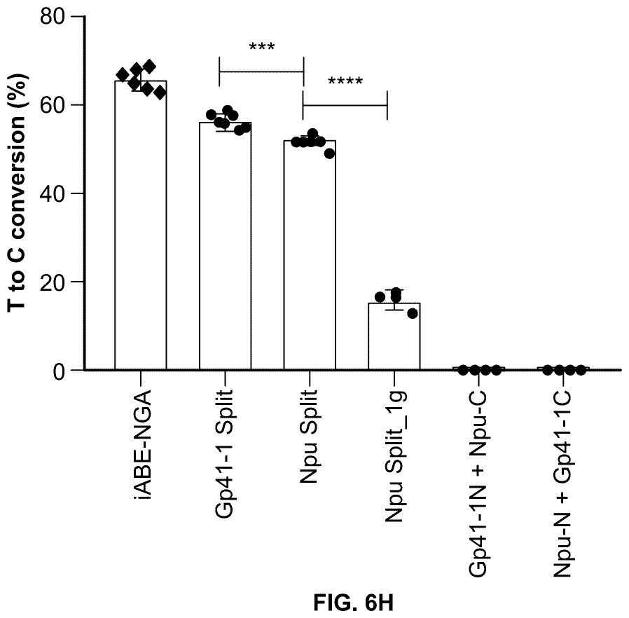

The large size of the ABE-NG and other base editors poses a major challenge for viral packaging and in vivo delivery. A dual trans-splicing adeno-associated virus (AAV) approach was used to deliver ABE and a dual protein trans-splicing (PTS) approach using the split-intein moiety from Nostoc punctiforme (Npu) was used to deliver CBE. The PTS approach was adopted to deliver ABE. The ABE was split between the ecTad-ecTadA* and the Cas9 nickase with Npu intein moieties, and this split renders low editing efficiency (FIG. 5). To improve the editing efficiency of the split ABE, the amino acid position 573 and 574 of the Cas9 nickase were chose as the splitting site because previous studies showed that 573/574 split Cas9 exhibited near the full-length Cas9 activity. Moreover, split at this site would produce a roughly equal size of the two halves for AAV packaging (FIG. 4a). the split ABE can be further improved by using inteins with fast rate of PTS. Two inteins were selected with the remarkably fast rate of PTS: Cfa (t½=20 s at 30° C.) and Gp41-1 (t_{1/2}=5 s at 37° C.), which are ˜2.5-fold and ˜10-fold faster than the rate reported for the Npu DnaE intein (t_{1/2}=50 s at 37° C.), respectively. Transfection of both split versions into HEK293 cells resulted in robust expression of full-length ABEs as detected by the anti-Cas9 antibody (FIG. 6b), although the expression level was generally lower than the ABEmax but higher than the original ABE7.10. Co-transfection with the split ABE-NG, mdx^{4cv}-gRNA and the mdx^{4cv} reporter restored EGFP expression to a similar level as the full-length ABE-NG (FIG. 6c). There was no significant difference between the Cfa and Gp41-1 intein splits (FIG. 6d). The Gp41-1 version was chosen for further studies.

It was shown that the Npu intein split of ABE worked well in vitro and in vivo. The Gp41-1 split and Npu split was directly compared. While both the Gp41-1 split and Npu split allowed the assembly of full-length iABE-NGA, Western blotting analysis showed that the Gp41-1 split rendered significantly more full-length iABE-NGA protein as compared to the Npu split (FIGS. 6e,6f). The assembly efficiency (as measured by the percentage of the full-length band) of the Gp41-1 split was about 70%, while the Npu split resulted in only 21% (FIG. 6g). To further compare the editing efficiency of the Gp41-1 split and Npu split, the T-to-C conversion of the mdx^{4cv} stop codon was quantified quantified in Neuro2A cells using the reporter assay. As compared to the full-length iABE-NGA, the Gp41-1 split and Npu split retained about 85.5% and 78.8% of its activity, respectively (FIG. 6h). The difference in the editing efficiency between the Gp41-1 split and Npu split was small but statistically significant (p=0.009) (FIG. 6h). In the Gp41-1 split and Npu split shown herein, each half carries a U6-gRNA expression cassette, while only the C-terminal half of the Npu split reported by the Liu group carries the U6-gRNA expression cassette. To test if the double U6-gRNA cassette has higher editing activity than a single U6-gRNA cassette, the U6-gRNA cassette was removed from the N-terminal construct of the Npu split used herein, which resulted in greatly reduced editing (FIG. 6h), indicating that the gRNA dosage is a limiting factor for efficient Cas9-mediated editing. Moreover, to test the specificity of intein-mediated assembly of iABE-NGA, the N and C-terminal fragments of the Gp41-1 and Npu splits were swapped, and observed no editing (FIG. 6h), indicating that the intein-mediated protein splicing and assembly of full-length iABE-NGA are required for efficient editing.

### Example 4. Systematic Delivery of AAV9-iNG Leads to Widespread Dystrophin Restoration

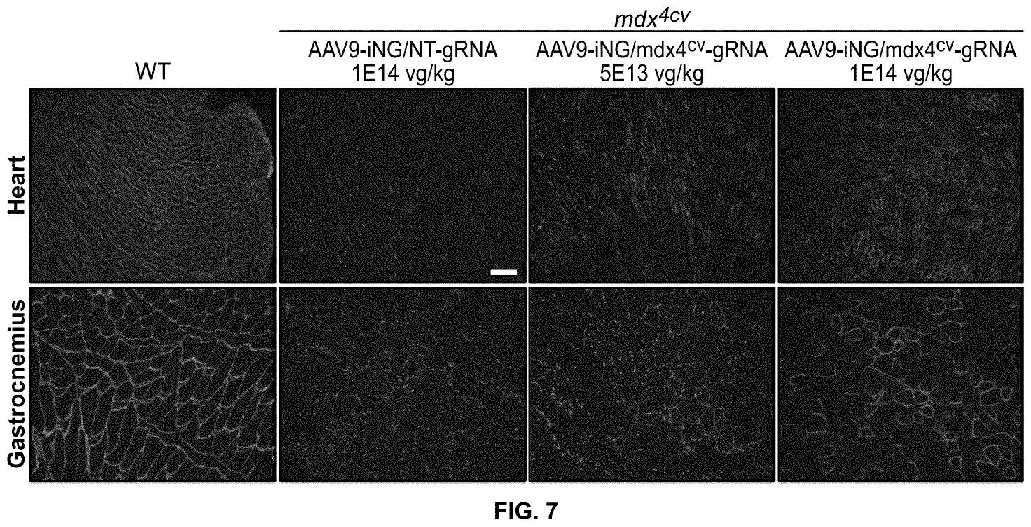

The two Gp41-1 intein split halves of the iABE-NGA were packaged into AAV9 (hereafter referred to as AAV9-iNG) and tested if in vivo delivery of iABE-NGA could correct the mutation in mdx^{4cv} mice. A truncated MHCK7 promoter was used to drive the expression of two halves of iABE-NGA. A preliminary testing of two dosages (a total of 5×10¹³ or 1×10¹⁴ vg/kg, 1:1 of the N and C-terminal half) showed that the higher dose appeared to increase the dystrophin-positive myocytes in the mdx^{4cv} mouse heart (FIG. 7). The higher dose (1×10¹⁴ vg/kg, 1:1 of the N and C-terminal half) was chosen for the rest of the study. In addition, the study also showed that injection of AAV9-iNG carrying a non-targeting gRNA failed to induce dystrophin rescue (FIG. 7).

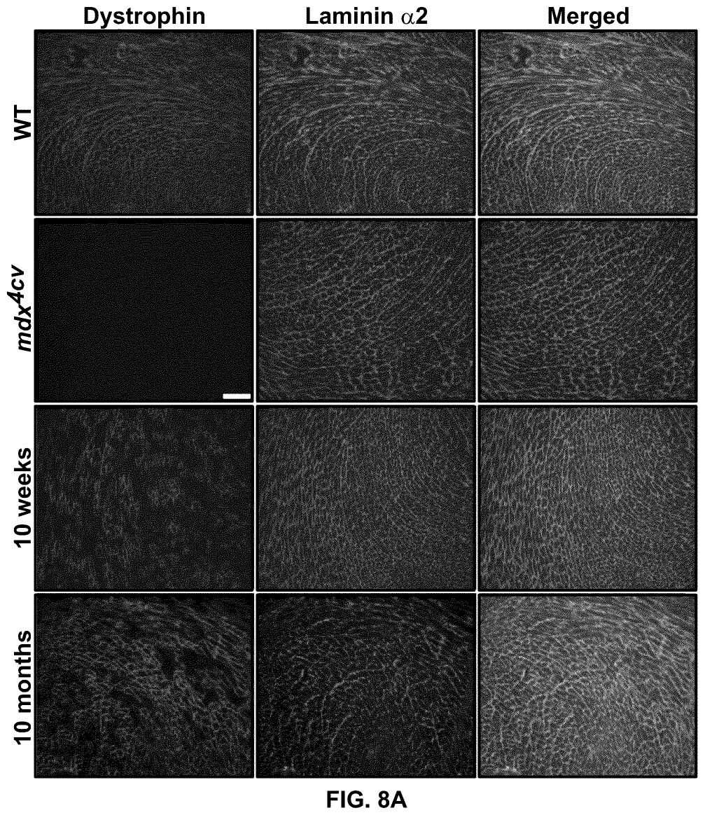

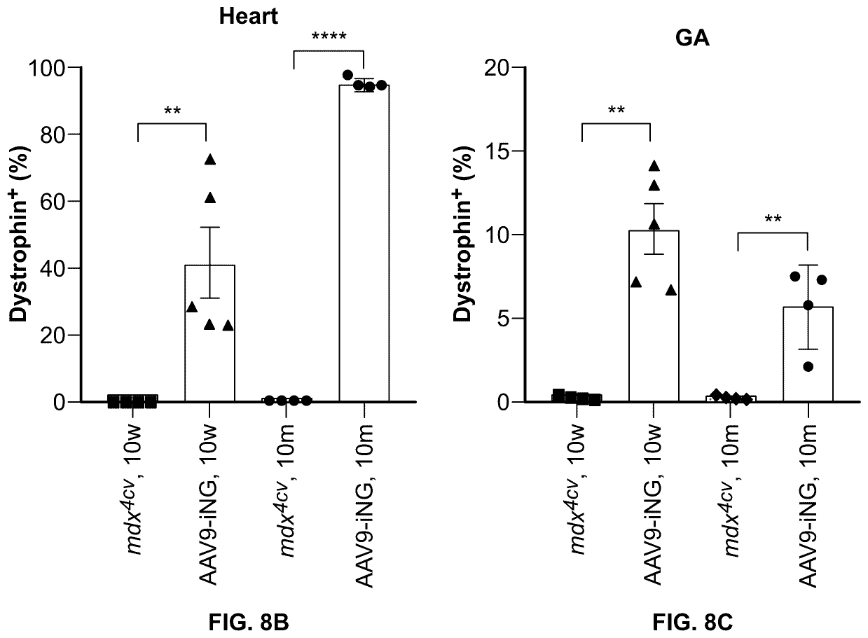

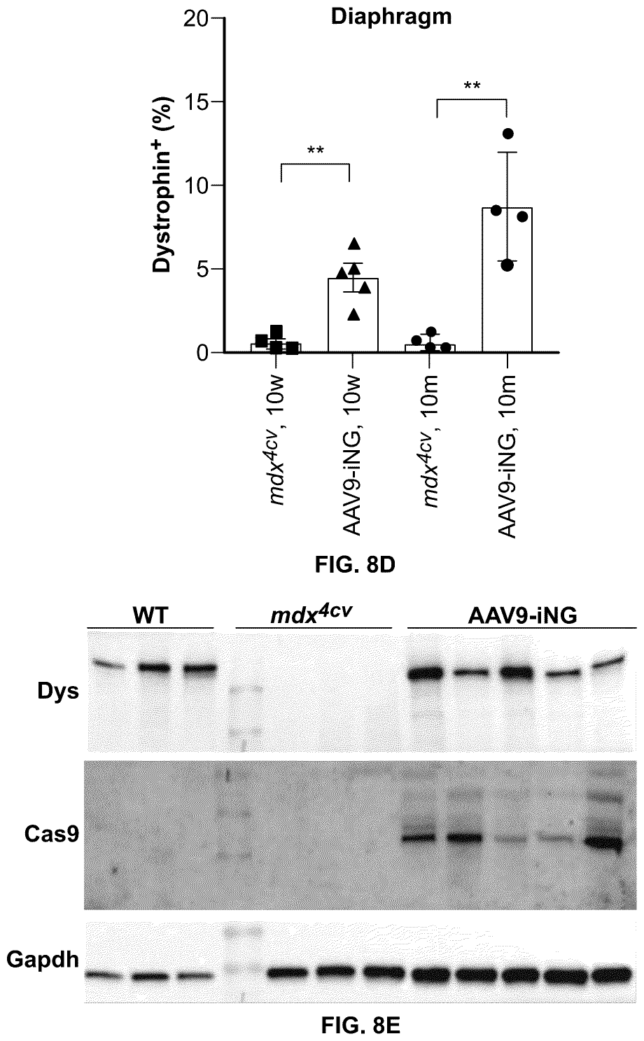

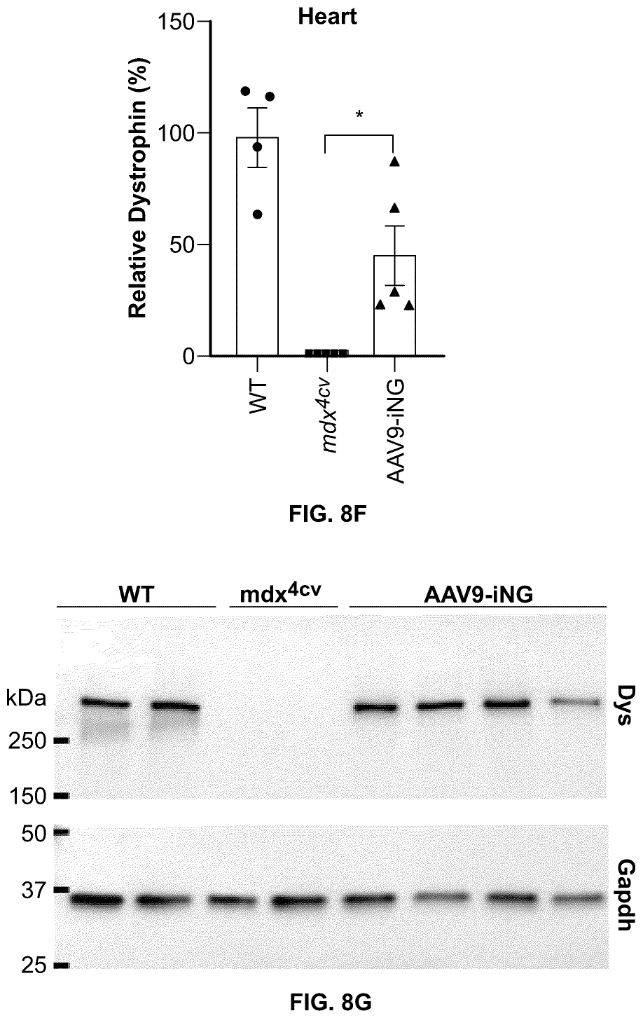

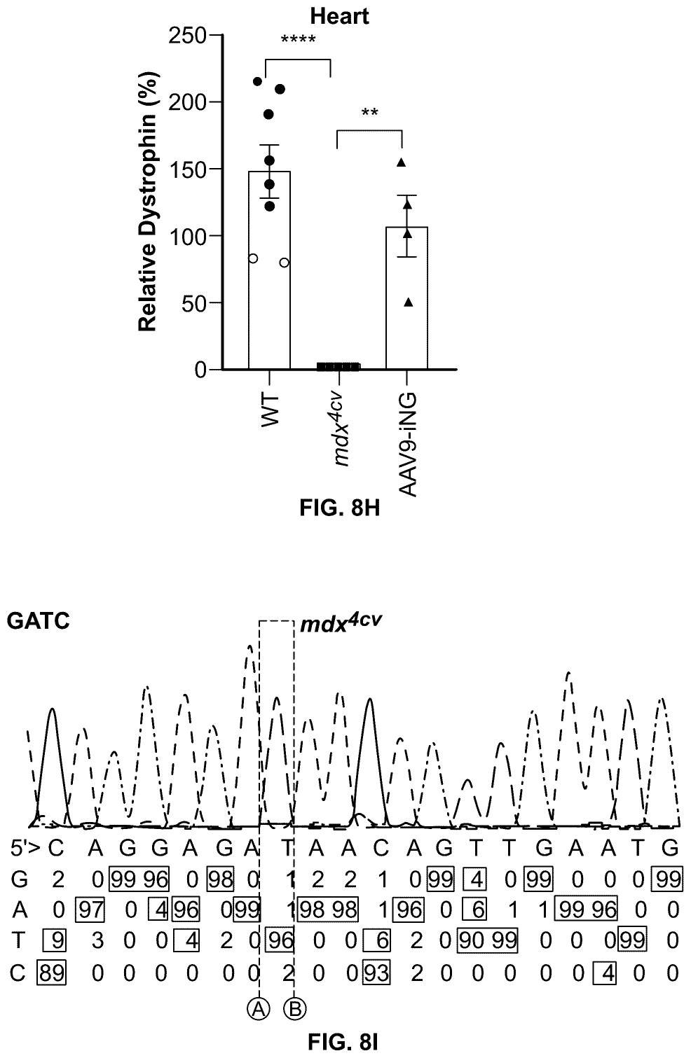

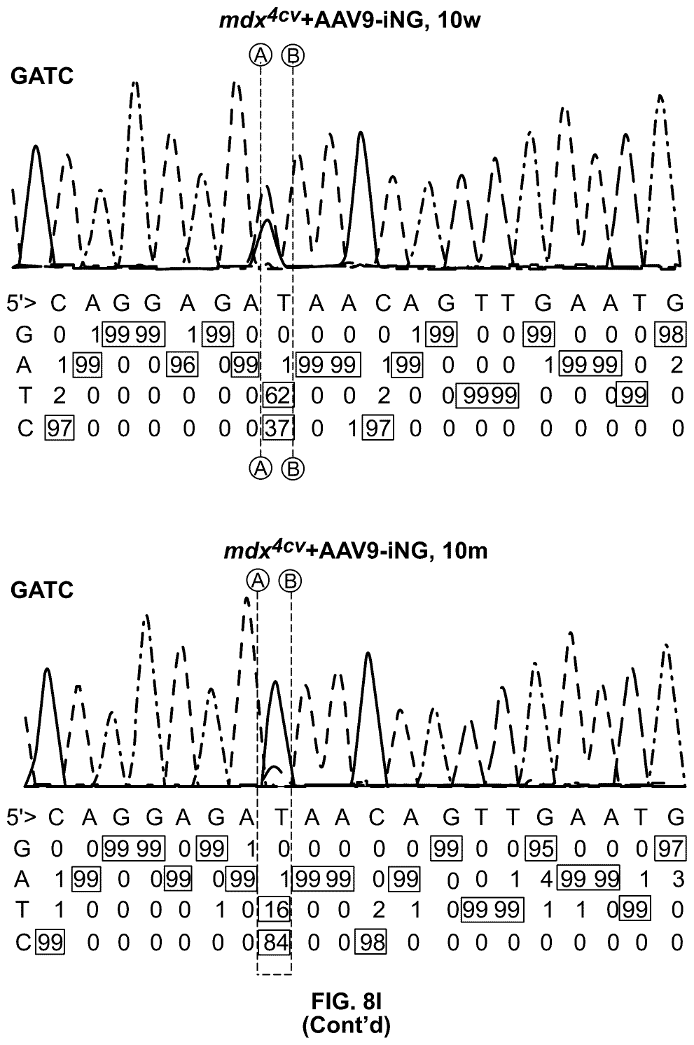

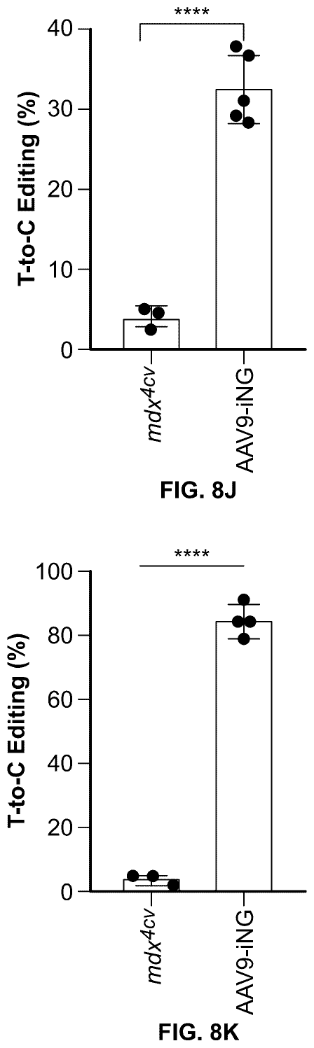

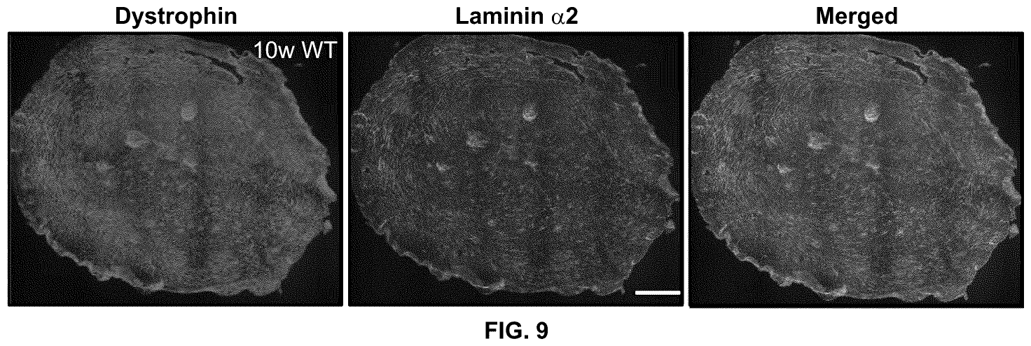

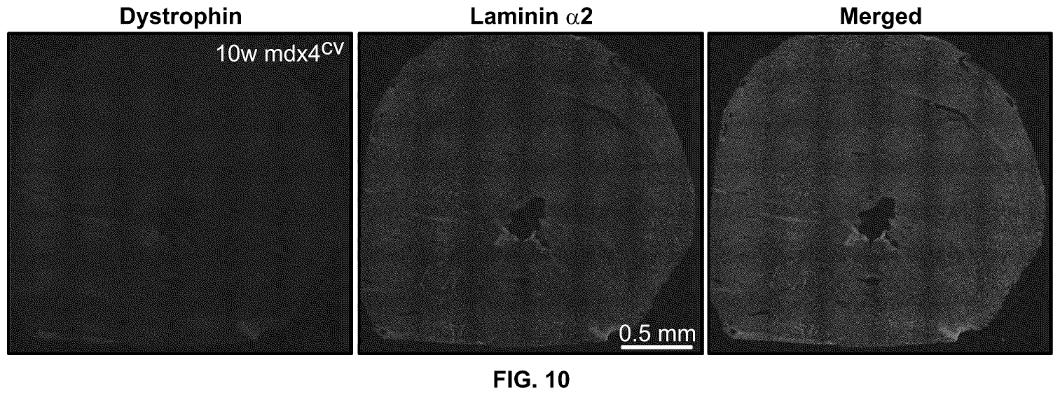

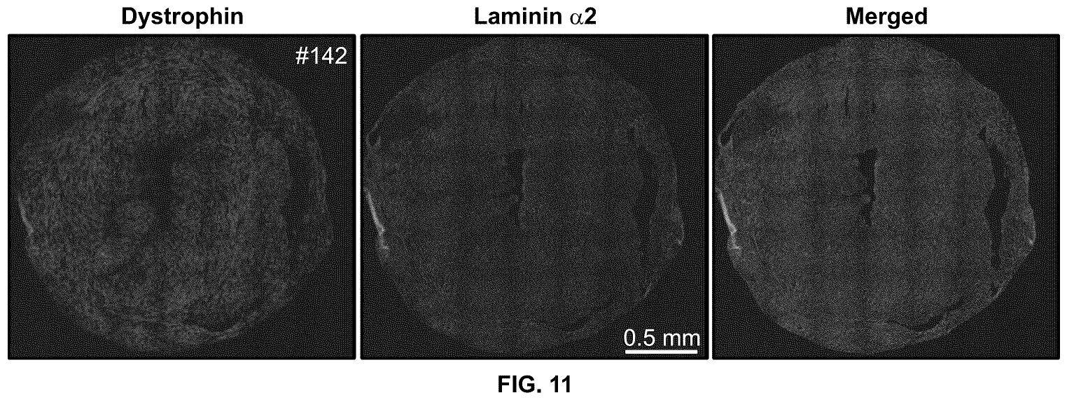

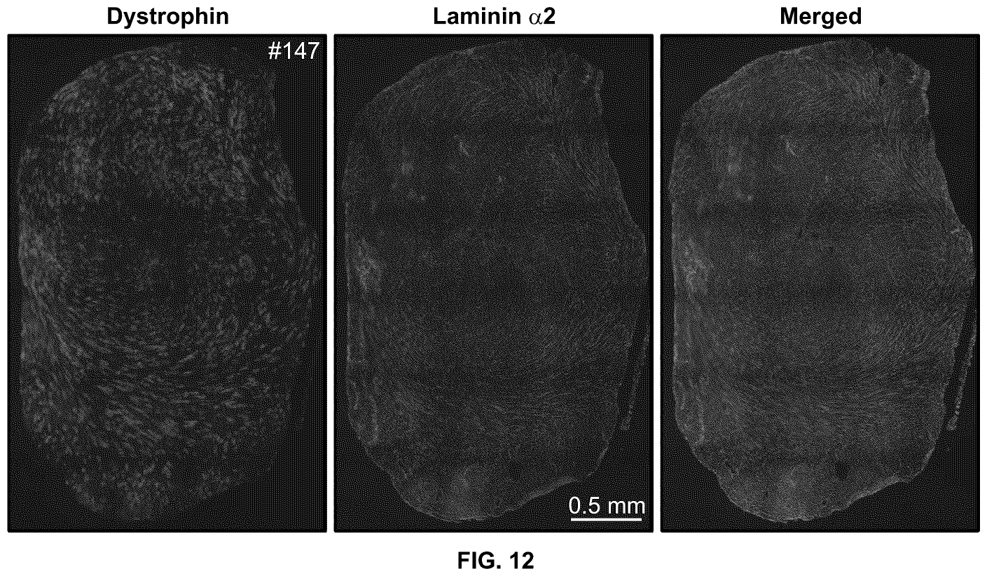

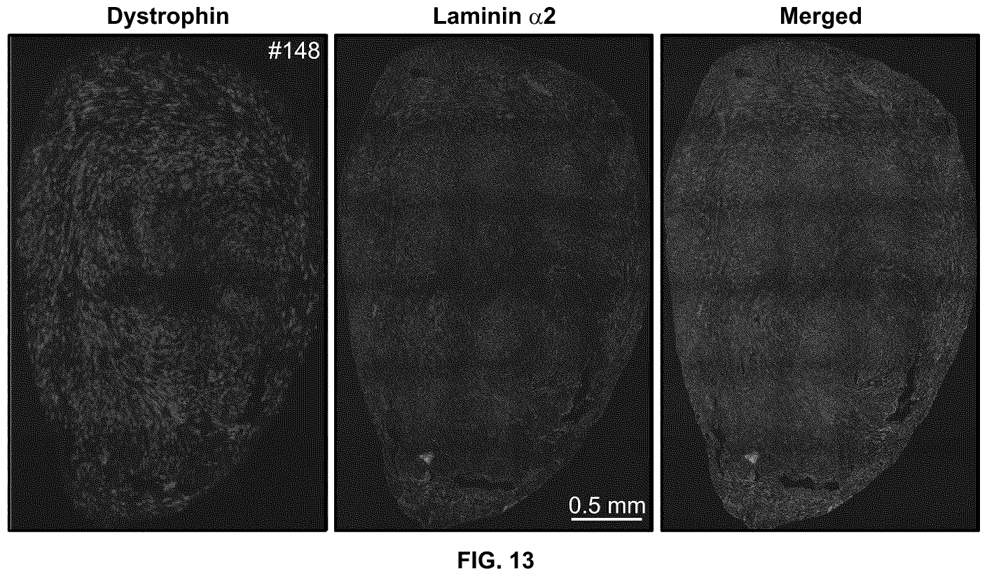

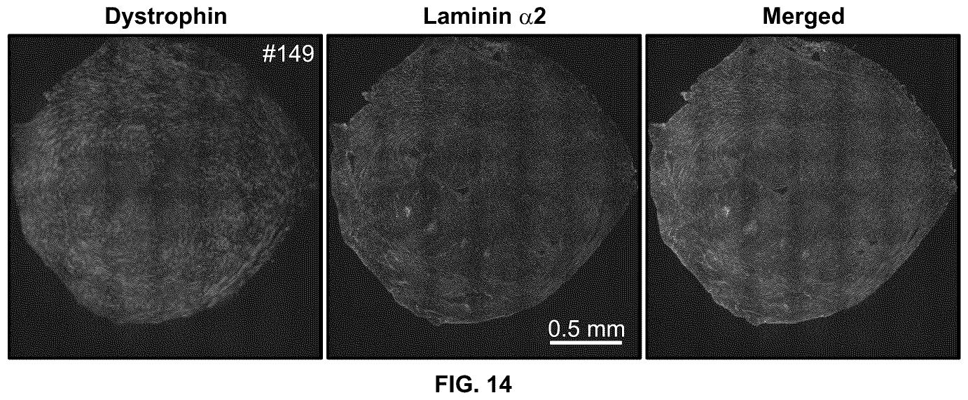

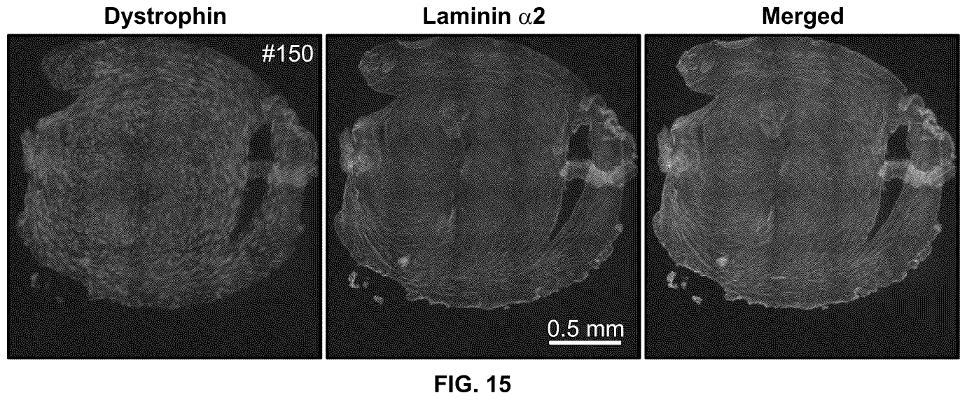

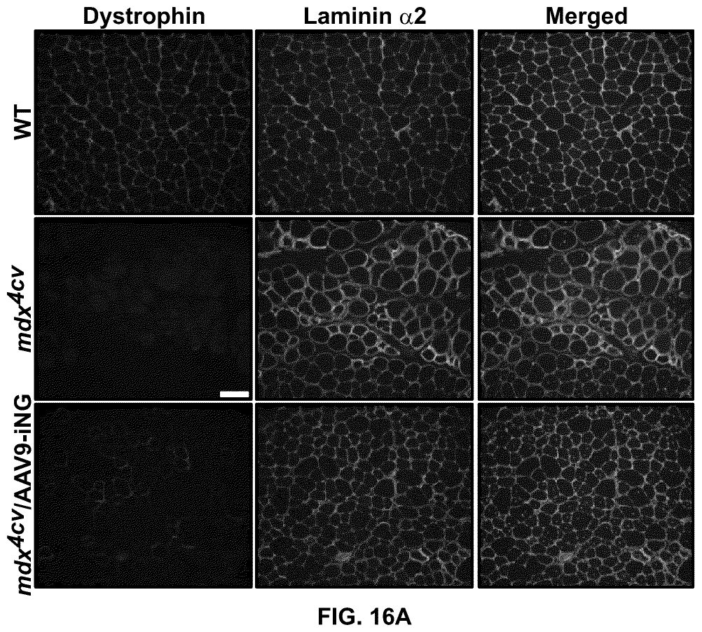

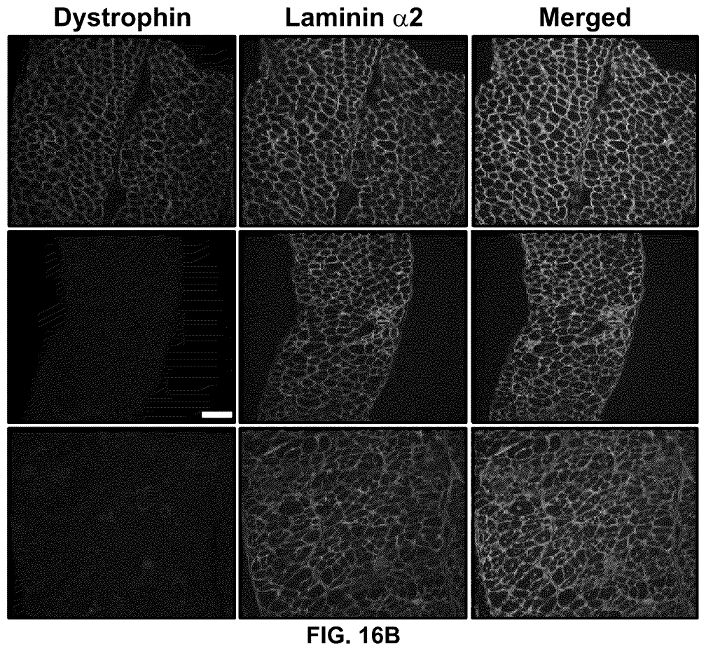

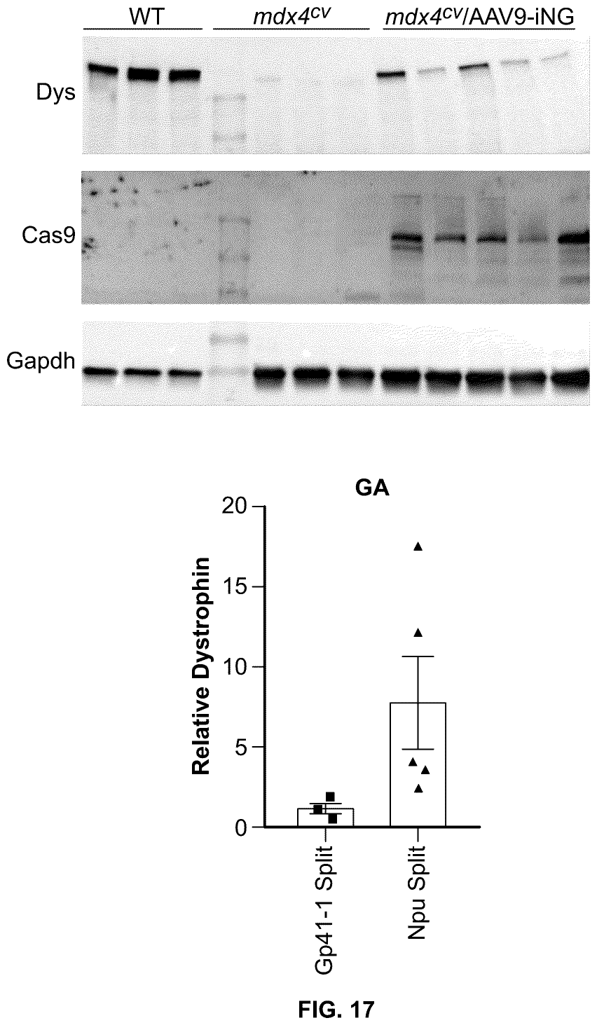

A cohort of nine mdx^{4cv} mice were treated with AAV9-iNG (a total of 1×10¹⁴ vg/kg, 1:1 of the N and C-terminal half) through a single tail vein injection at 5 weeks of age. A subset of the mice was sacrificed at 5 weeks after AAV9-iNG administration. Dystrophin was found to be widely rescued in mdx^{4cv} heart (FIG. 8a and FIGS. 9-15). Quantification of the entire heart sections showed that 41.9±10.5% cardiomyocytes of mdx^{4cv} mice became dystrophin positive at 10 weeks of age after systematic AAV9-iNG treatment (N=5) while the control mdx^{4cv} hearts were essentially dystrophin negative (0.03±0.02%, N=4) (FIG. 8b). Dystrophin was also rescued in skeletal muscles (diaphragm and gastrocnemius) of mdx^{4cv} mice treated with AAV9-iNG, albeit the recovery was less efficient as compared to that in the heart (FIGS. 8c, 8d, and FIG. 16). Western blot analysis showed that dystrophin was rescued in mdx^{4cv} mouse heart to 45.9±11.7% of the WT level following systemic AAV9-iNG treatment (FIGS. 8e, 8f). Consistent with the immunofluorescence data, Western blot showed dystrophin was restored to about 8.0±2.6% of the WT level in the gastrocnemius muscle of mdx^{4cv} mice (FIG. 17).

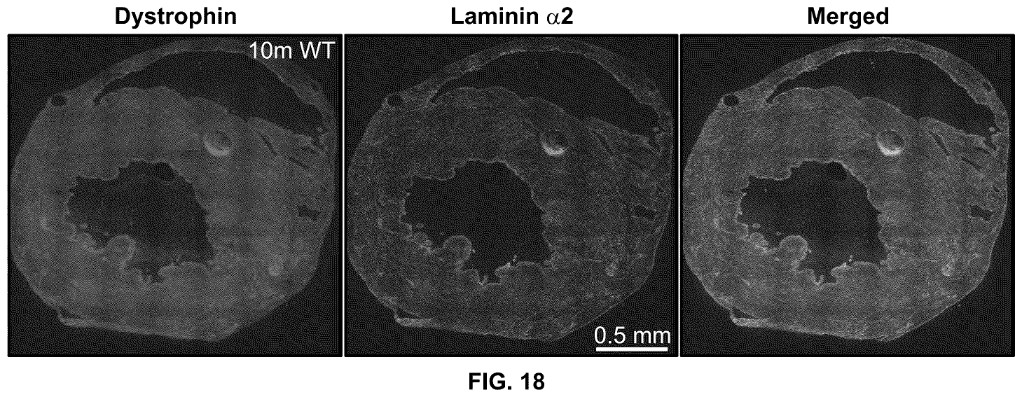

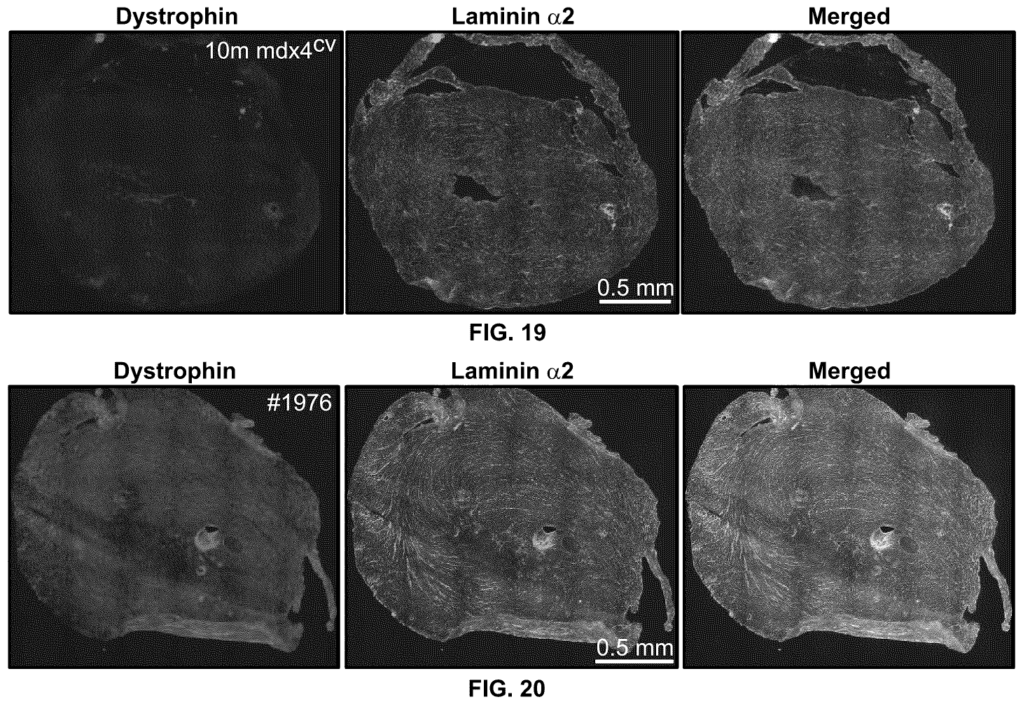

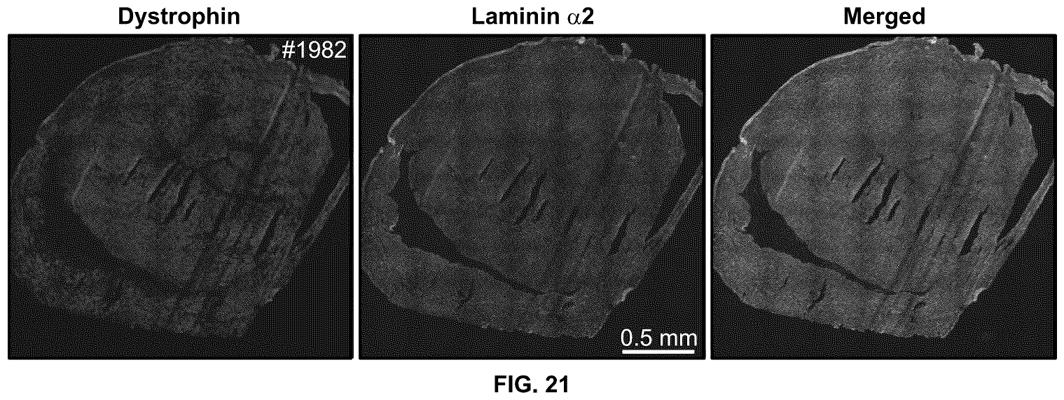

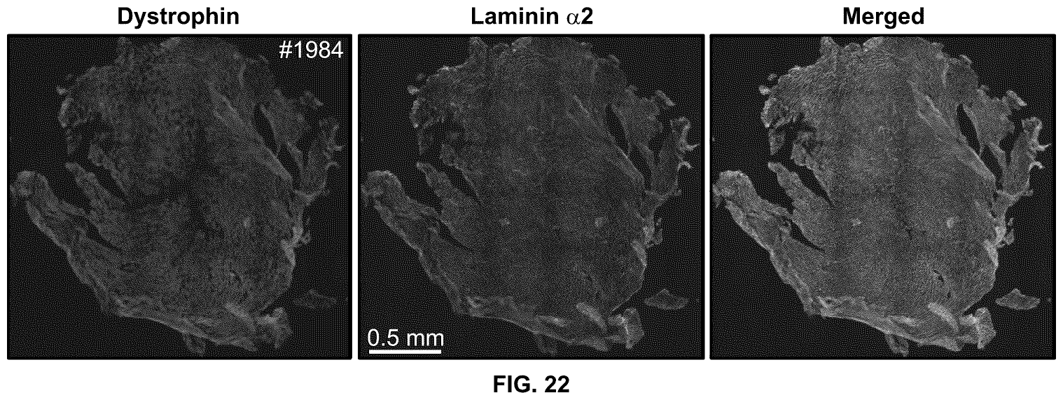

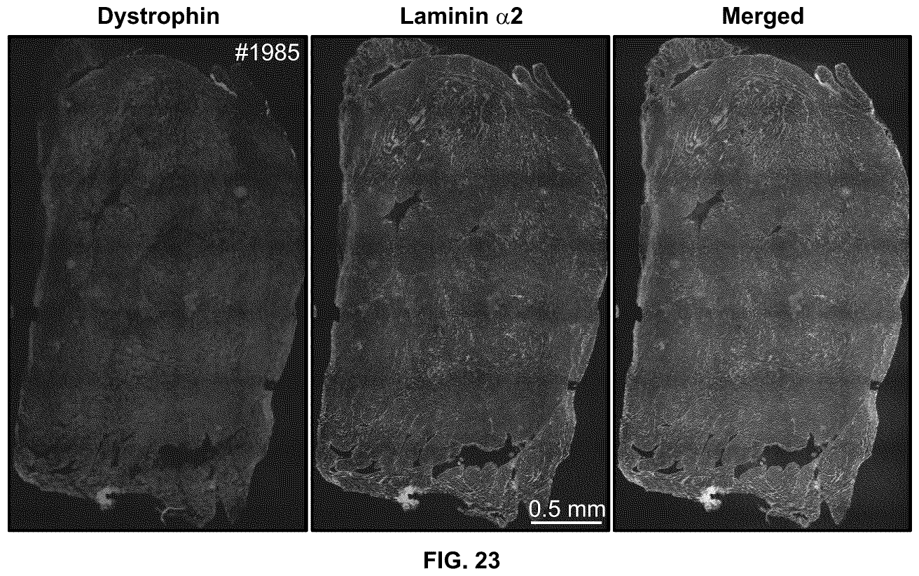

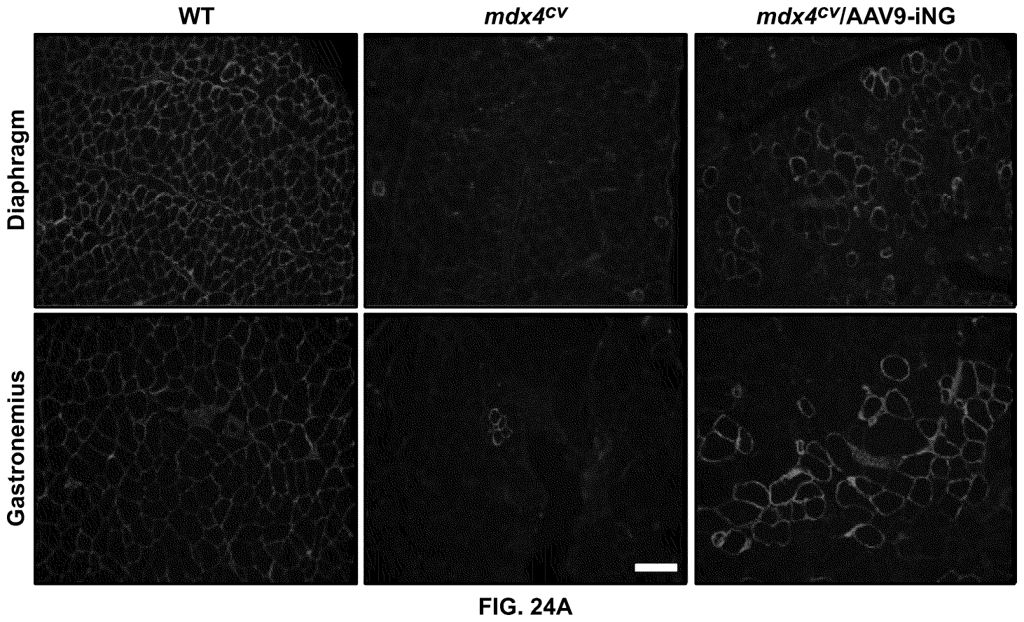

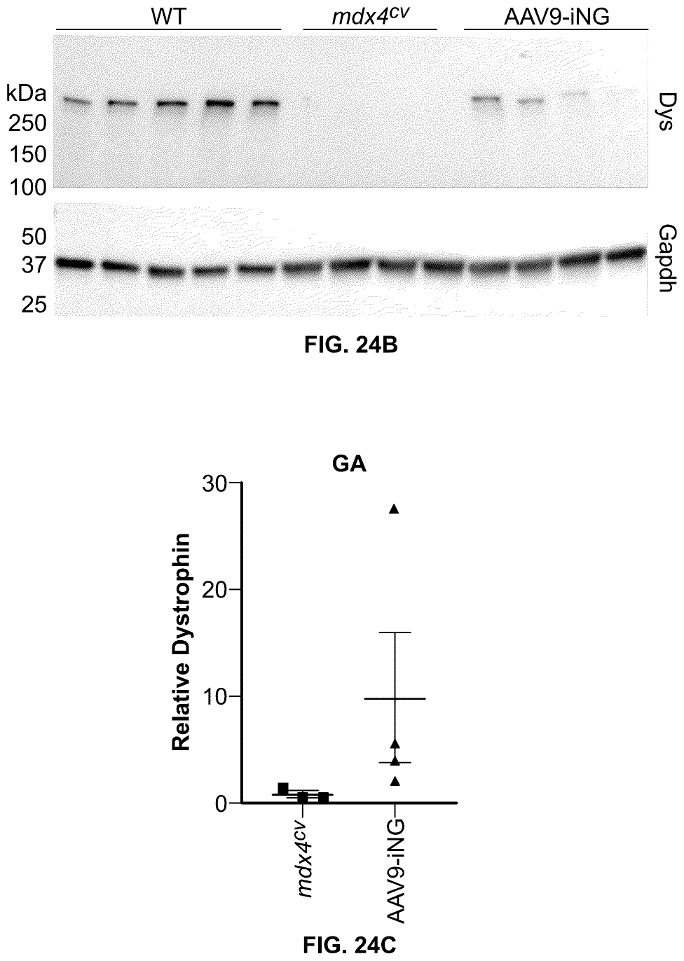

A group of mdx^{4cv} mice treated with intravenous administration of AAV9-iNG at 5 weeks of age were kept for 10 months to study the long-term impact of systemic ABE editing therapy. A near complete dystrophin restoration was observed in the hearts of all four treated mdx^{4cv} mice at 10 months of age (FIGS. 8a, 8b and FIGS. 18-23). Dystrophin was also rescued in the skeletal muscles of these older animals with a similar percentage of dystrophin-positive muscle fibers as analyzed at the 10 weeks of age (FIGS. 5c, 5d and FIG. 24). Western blot analysis showed near WT levels of dystrophin expression in the hearts of the 10-month-old mdx^{4cv} mice treated with AAV9-iNG (FIGS. 8g, 8h).

The heart and muscle tissues contain many different types of cells, which makes it challenging to precisely determine the DNA editing efficiency in myocytes. To estimate the editing efficiency of the Did gene, the total RNA was extracted from the heart tissues treated with or without AAV9-iNG, amplified the target region by RT-PCR, and analyzed the resulting amplicons by Sanger sequencing and BEAT program. The AAV9-iNG treated mdx^{4cv} hearts showed an average 32.6±2.0% T-to-C editing at 10 weeks of age (FIGS. 8i, 8j) and 84.6±2.6% at 10 months of age (FIGS. 8i, 8k).

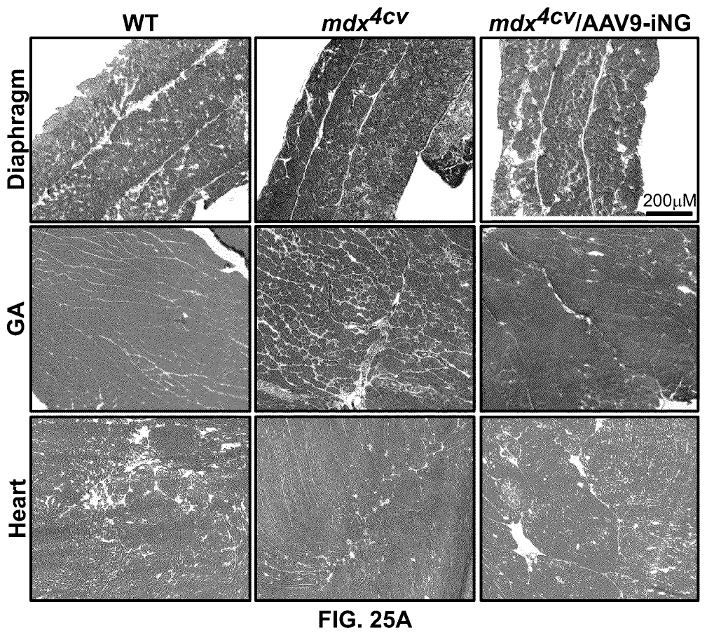

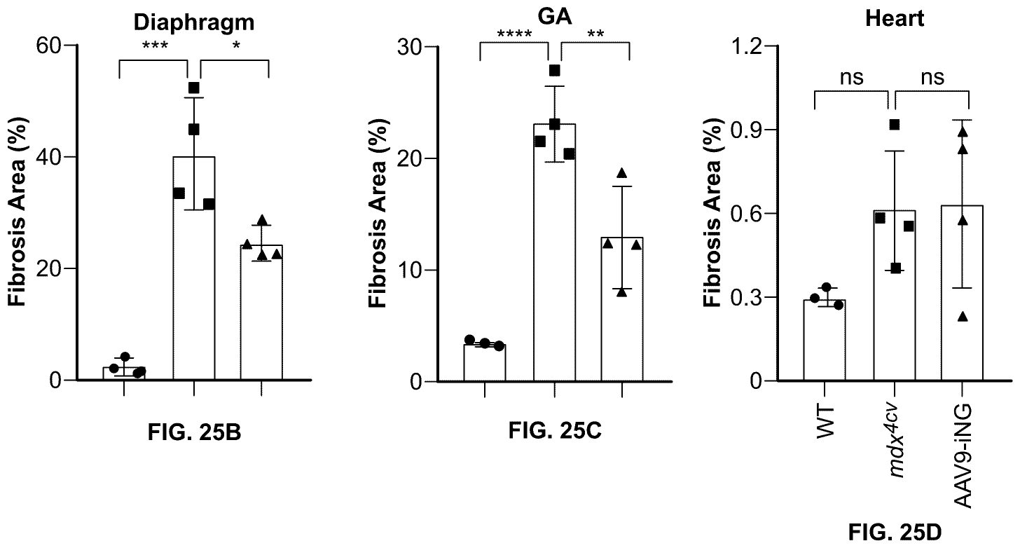

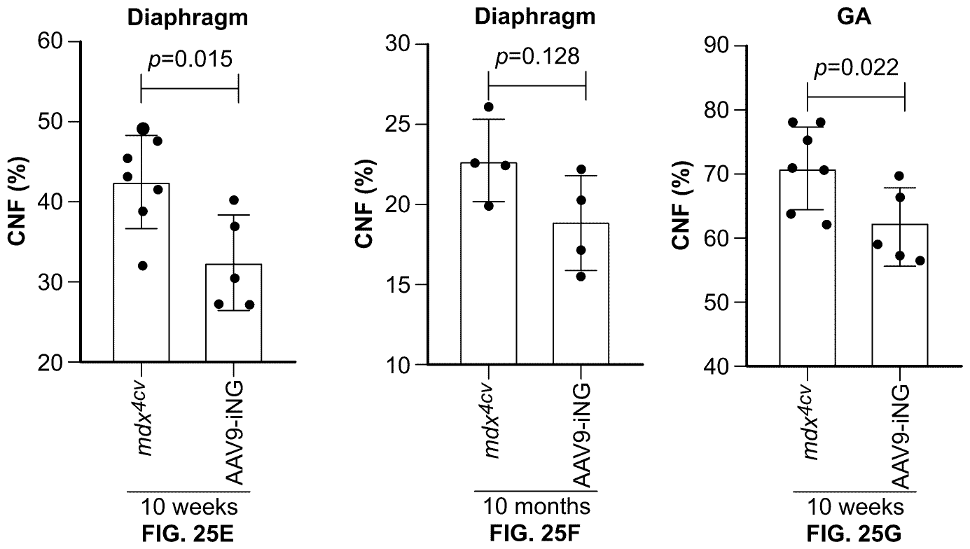

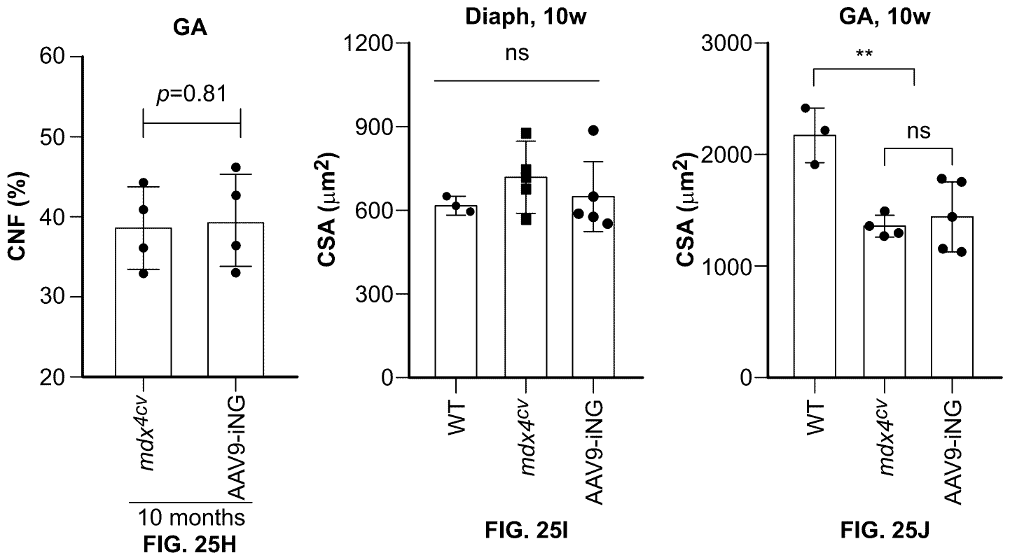

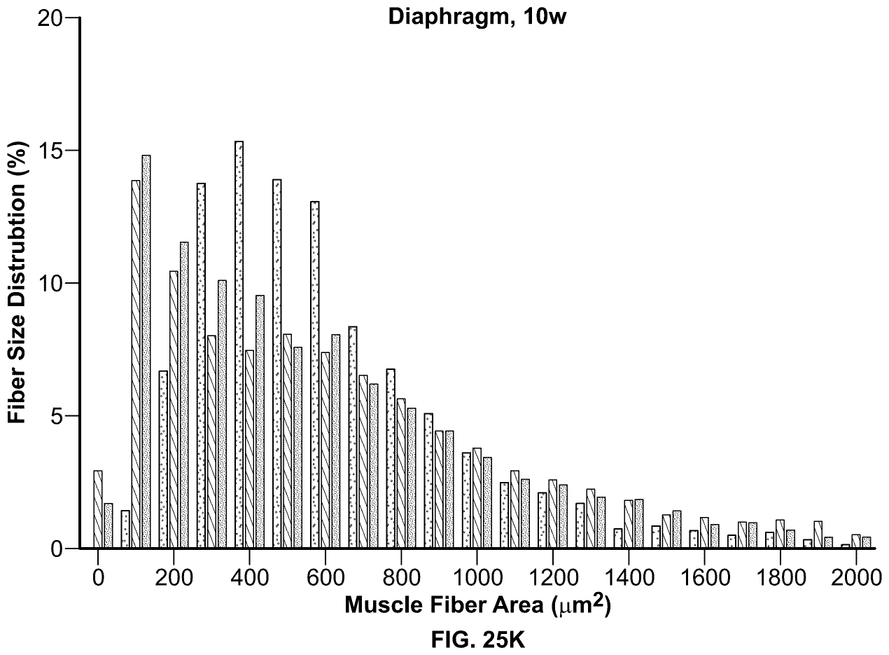

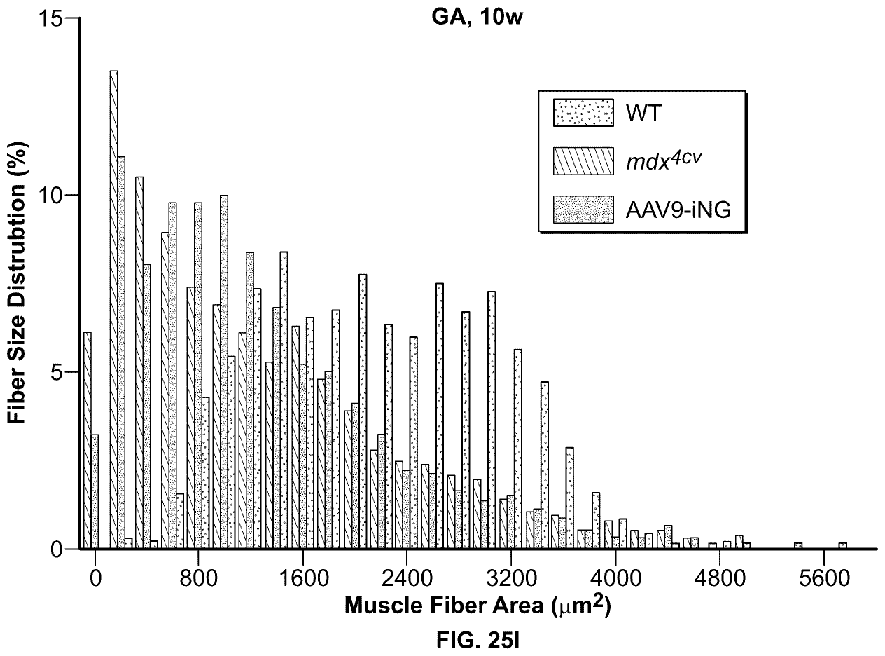

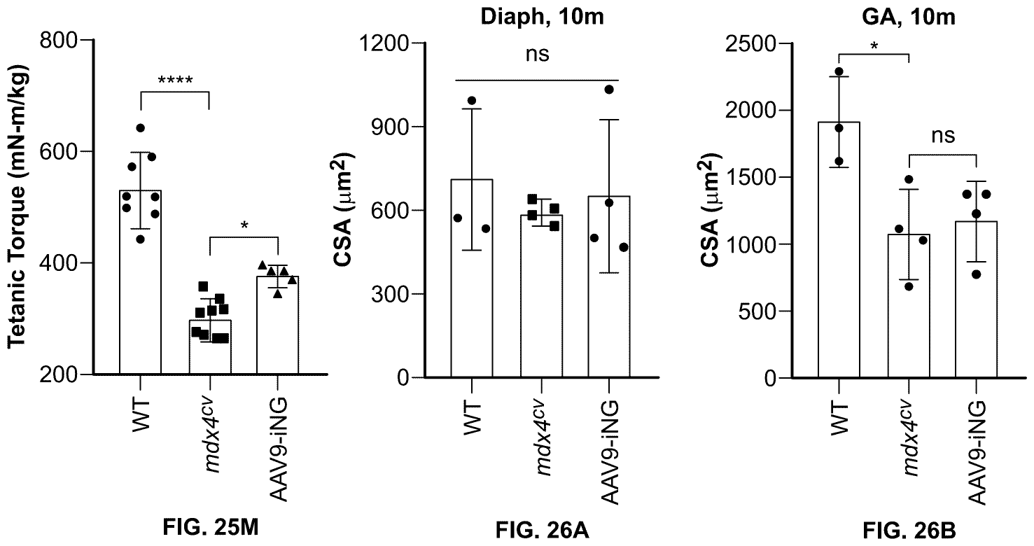

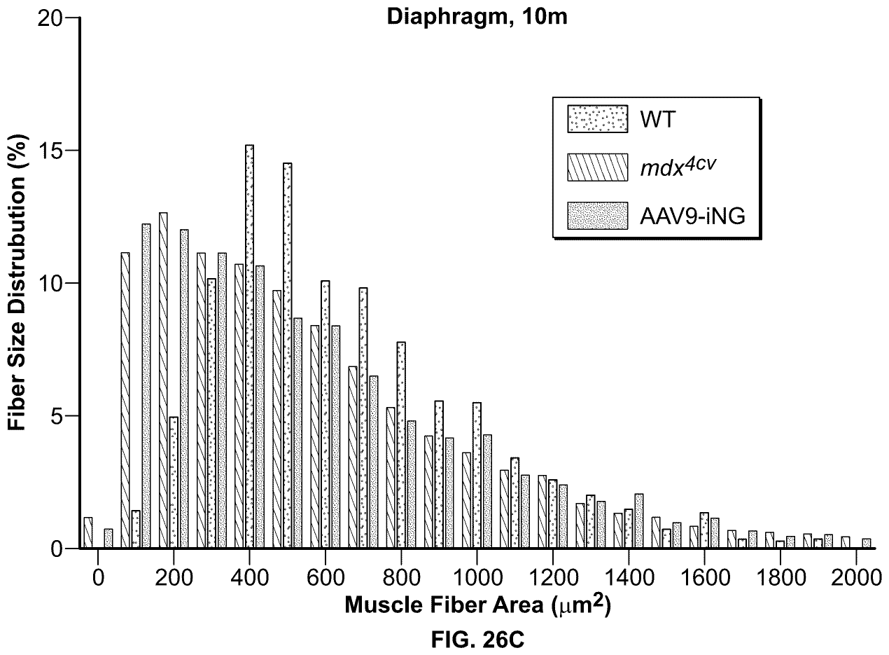

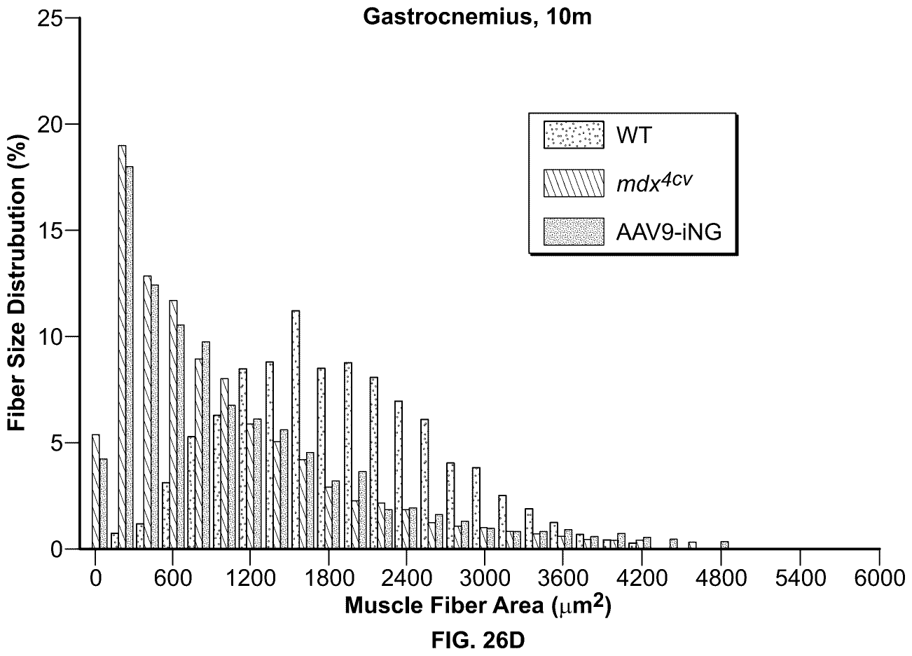

Repeated cycles of muscle degeneration and regeneration in muscular dystrophy result in muscle fibrosis. To examine if systemic AAV9-iNG delivery can improve the histopathology of mdx^{4cv} mice, Trichrome staining was performed in 10-month-old mice. As compared to WT mice, the mdx^{4cv} mice showed significantly elevated fibrosis in both diaphragm and gastrocnemius muscles and the fibrotic areas in these muscles were significantly reduced in the mdx^{4cv} mice treated with AAV9-iNG (FIGS. 25a-25c). Consistent with previous studies that the mdx mice do not develop overt cardiomyopathy before one year old, there were no significant changes in cardiac fibrosis in mdx^{4cv} mice with or without AAV9-iNG treatment at 10 months of age as compared to the WT controls (FIG. 25d). AAV9-iNG treatment also significantly reduced the percentage of centrally nucleated fibers (CNF) in both diaphragm and gastrocnemius muscles at 10 weeks of age (FIGS. 25e, 25g). By 10 months of age, the effects of AAV9-iNG treatments on CNF were blunted (FIGS. 25f, 25h). Although a significant difference in cross-sectional area (CSA) of muscle fibers was not observe following AAV9-iNG treatment (FIGS. 25i, 25j, and FIGS. 26a, 26b), the AAV9-iNG treatment appeared to shift the fiber size distribution towards those of the WT muscles (FIGS. 25k, 25l, and FIGS. 26c, 26d), particularly in gastrocnemius muscles at weeks of age.

To test if systemic AAV9-iNG treatment can improve the muscle function, the muscle contractility was measured using an in vivo muscle test system. Maximum plantarflexion tetanic torque was measured during supramaximal electric stimulation of the tibial nerve at 150 Hz. While the mdx^{4cv} mice produced significantly reduced torque as compared to the WT controls, systemic delivery of AAV9-iNG significantly increased the tetanic torque in mdx^{4cv} mice (FIG. 25m).

### Example 5. The Safety Profile and Off-Target Activity of AAV9-iNG Treatment

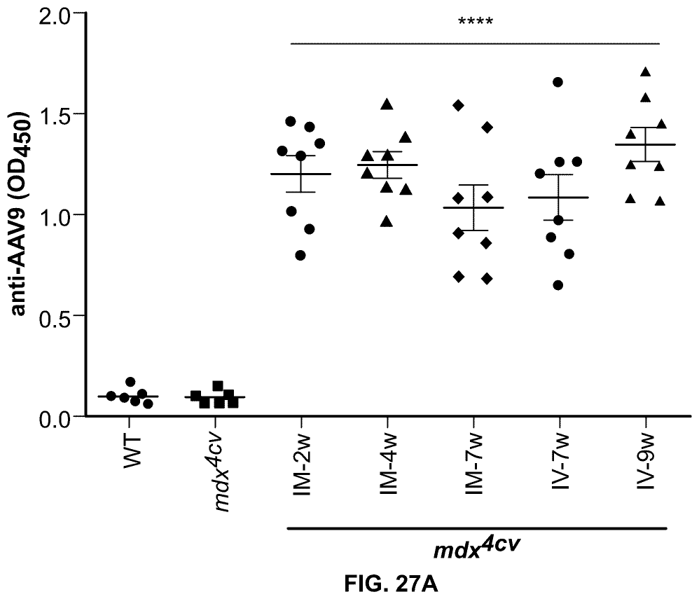

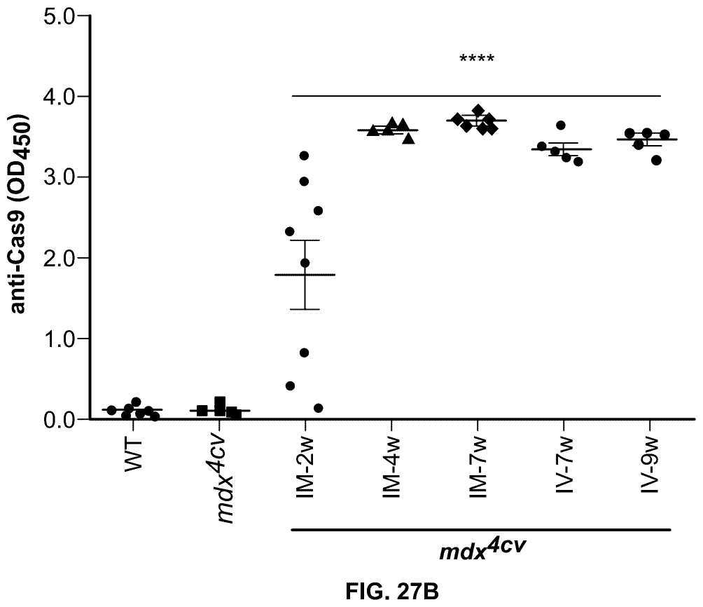

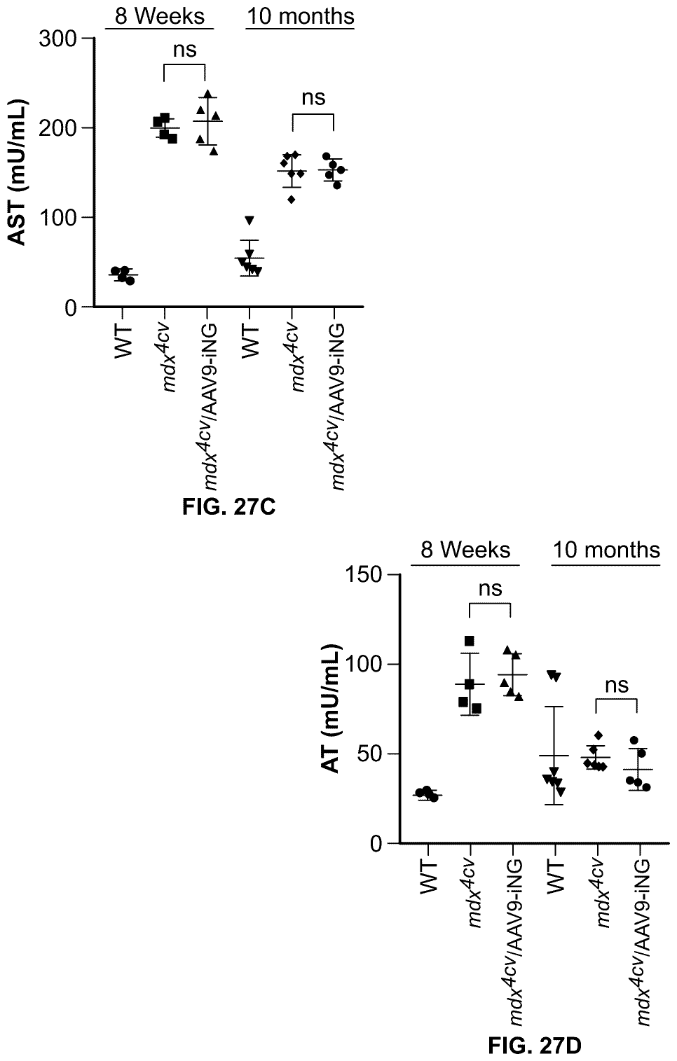

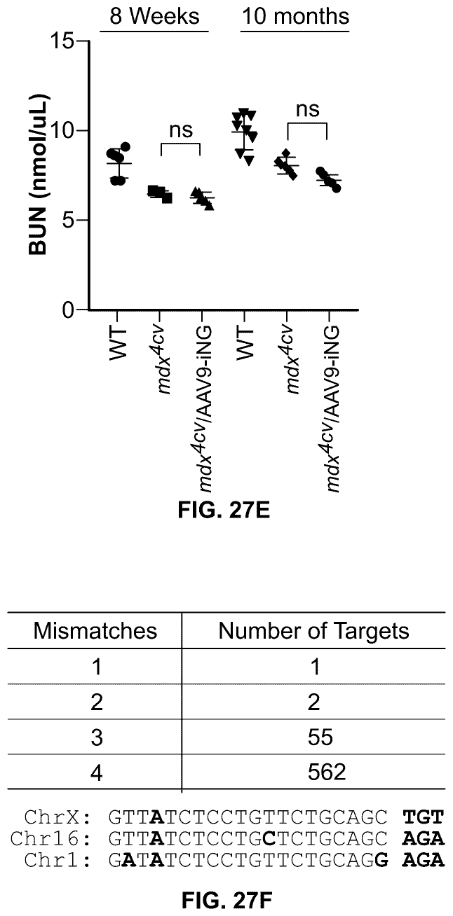

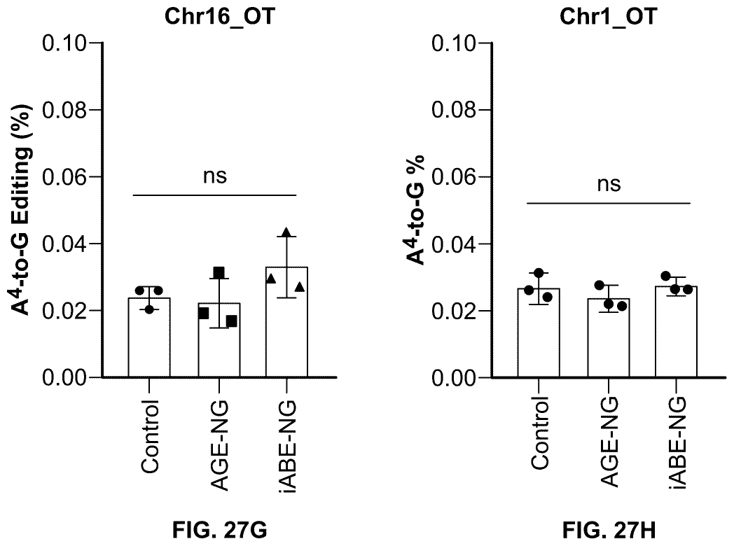

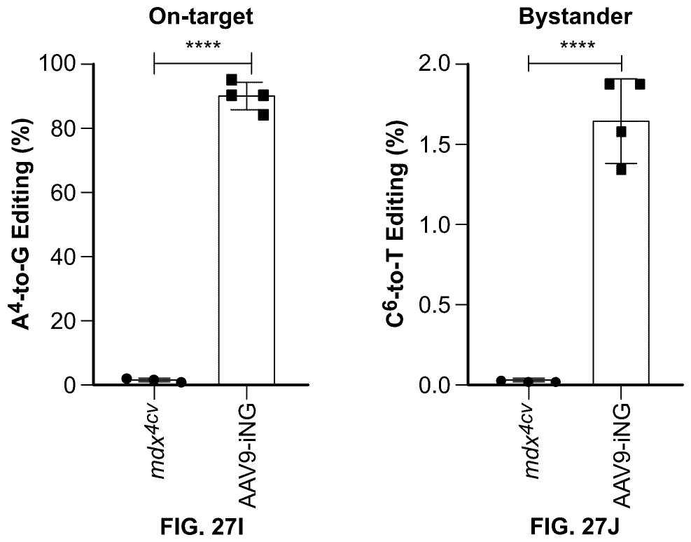

Previous studies showed that AAV-mediated delivery of CRISPR/Cas9 into neonatal mice resulted in humoral immune responses to AAV capsid but not Cas9. In contrast, AAV-mediated delivery of CRISPR/Cas9 into adult mice evoked robust anti-Cas9 immunity. Serum samples were collected to analyze the host immune responses to the AAV9 capsid and the base editor iABE-NGA. Intramuscular injection of AAV9-iNG into 5-6 weeks old mdx^{4cv} mice produced robust anti-AAV9 capsid (FIG. 27a) and anti-Cas9 antibodies (FIG. 27b) at 2 weeks after injection. The anti-AAV9 titers were similar at different time points from 2 to 7 weeks post intramuscular injection and from 7 to 9 weeks post intravenous injection (FIG. 27a). The anti-Cas9 antibody titers showed a large variation among mice at 2 weeks after intramuscular injection, but all increased to peak by 4 weeks (FIG. 27b).

The liver toxicity of AAV9-iNG treatment was examined by measuring serum aspartate aminotransferase (AST) and alanine aminotransferase (ALT), and kidney toxicity by measuring blood urine nitrogen (BUN). As compared to WT mice, the mdx^{4cv} mice showed elevated AST (FIG. 27c) and ALT (FIG. 27d). However, treatment of mdx^{4cv} mice with AAV9-iNG did not further increase the serum levels of AST and ALT at either 8 weeks or 10 months of age. Measurement of BUN did not find significant changes in the treated or untreated mdx^{4cv} mice (FIG. 27e).

One concern with ABE-mediated gene correction is the off-target activities such as gRNA mismatch tolerance, bystander editing, and off-target RNA editing. Previous studies showed that ABE can tolerate 1-2 mismatches between the gRNA and its target sites. Prediction by Cas-OFFinder showed that one site on chromosome 16 (Chr16_OT) has only one mismatch, two other sites have two mismatches and 55 sites have three mismatches (FIG. 27f). The Chr16_OT differs from the mdx^{4cv} target sequence by only one Cat position 12. Neuro-2a cells were transfected with ABE-NG or iABE-NG plus the gRNA, amplified the Chr16_OT by PCR and subjected the amplicon to next generation sequencing (NGS). As shown in FIG. 27g, no significant editing of the A4 in either ABE-NG or iABE-NG transfected cells was observed. Similarly, the off-target site on chromosome 1 (Chr1_OT) was analyzed, which differs from the mdx^{4cv} target sequence by an A at position 2 and a G at position 20. Again, it was found that ABE-NG or iABE-NG did not edit the A4 at Chr1_OT (FIG. 27h).

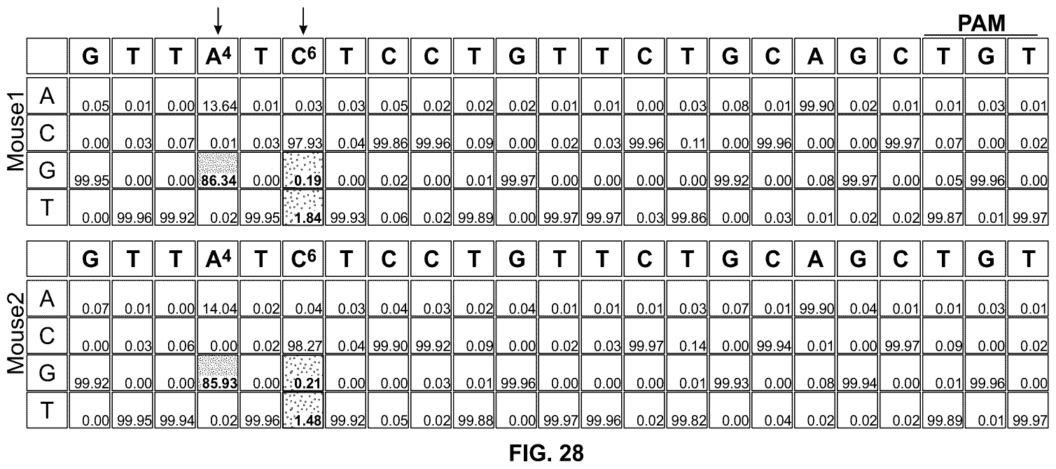

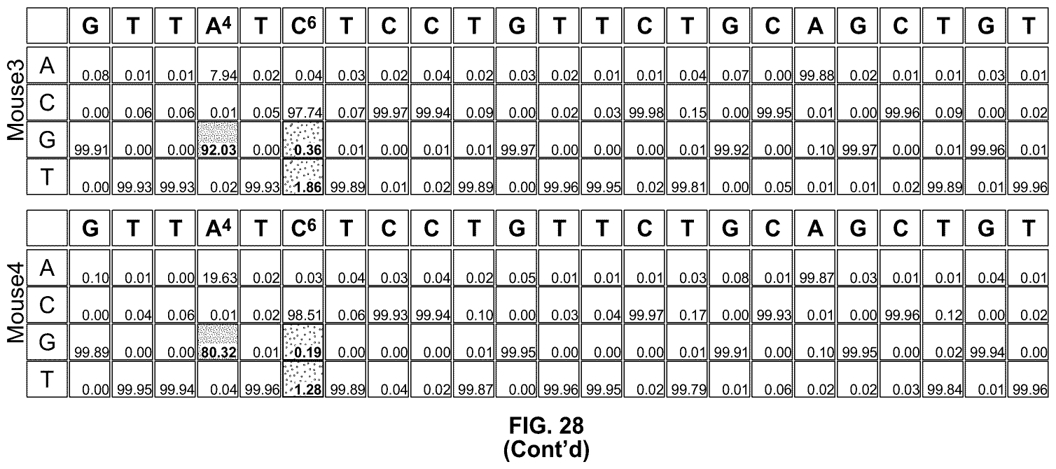

Next, the bystander editing at the on-target mdx^{4cv} locus was analyzed in the mice treated with AAV9-iNG. Since the 10-month treated mouse hearts showed a high level of dystrophin rescue, the on-target editing efficiency was first determined in these mouse hearts by NGS. As mouse hearts contain multiple different cell types, analysis of the genomic DNA PCR products can significantly underestimate the editing efficiency. To verify this, NGS of the genomic DNA PCR products was performed from two mouse hearts receiving AAV9-iNG and exhibiting high dystrophin rescue, and an up to 11% edits at A4 was detected. Thus, the RT-PCR products were sequenced to estimate the editing efficiency at the on-target mdx^{4cv} locus. The A at position 4 (corresponding to the T within the premature stop codon in the coding strand) was converted to G with high efficiency from all four mouse hearts (FIG. 28). On average, 86.2±2.4% A-to-G conversion was measured (FIG. 27i). At the mdx^{4cv} target site, there was only one A within the editing window of 4-8, disallowing us to analyze the bystander A-to-G editing at this particular site. Another type of undesired ABE-mediated genome edits at an on-target locus is ABE-dependent cytosine-to-uracil conversion resulting in C.G to T.A mutation at that site. It was found that C6 at the mdx^{4cv} target site was edited above background with an average efficiency of 1.6±0.1% (FIG. 27j).

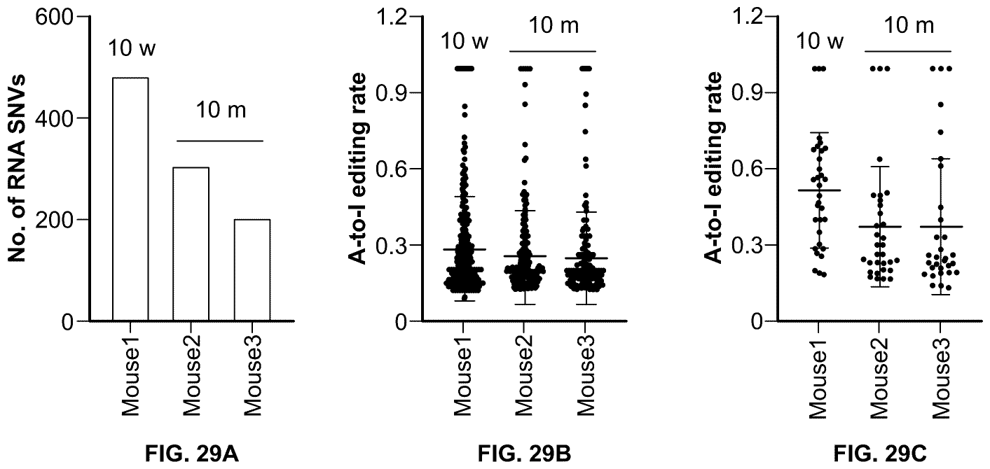

Finally, RNA-seq was performed to characterize the transcriptome-wide RNA off-target RNA editing induced by AAV9-iNG in the mdx^{4cv} mouse heart samples. After filtering the confident variants from control mdx^{4cv} heart samples, a few hundred RNA editing events were found in the three AAV9-iNG treated heart samples with only 32 shared by all of them (FIG. 29).

The present study has improved the split ABE-NG for AAV-mediated in vivo delivery by engineering a new NG PAM-interacting domain variant, a new adenine deaminase domain with higher on-target DNA editing efficiency without compromising the high fidelity of ABE-V82G, and a Gp41-1 intein split that mediates higher efficiency of protein splicing and editing. Together, these improvements allowed to achieve widespread dystrophin rescue and functional improvement in dystrophic mice. The editing efficiency in the heart was extraordinarily high in mdx^{4cv} mice following systemic delivery of AAV9-iNG and over 90% of cardiomyocytes were corrected to express dystrophin in mdx^{4cv} hearts at 10 months of age after a single intravenous administration of AAV9-iNG at 5 weeks old. There was no obvious toxicity detected following AAV9-iNG treatment, despite the host immune response to the AAV9 capsid and ABE. This has tremendous implication for base correction of genetic cardiomyopathies.

It was shown herein that the ecTadA* domain with the V82G mutation had significantly reduced on-target DNA editing activity as compared to the original ecTadA*. By adding the A56G mutation into the V82G variant of ecTadA*, the on-target DNA editing activity was dramatically improved without compromising the high fidelity of the V82G variant in terms of its low off-target RNA editing activity. Although the A56G_V82G variant was less efficient than the recently reported ABE8e, ABESe had also significantly higher bystander editing activity than the A56G_V82G variant. It is essential to consider both the on-target DNA editing efficiency and the bystander DNA editing as well as off-target RNA editing activity for in vivo applications. The editors have high on-target DNA editing but induce minimal bystander DNA editing and off-target RNA editing events. The A56G_V82G variant offers a balance between the editing efficiency and the editing precision.

The iABE-NG and iABE-NGA can be broadly applied to correct DMD mutations and many other disease-causing mutations. Analysis of the ClinVar database showed that over 100 of the 174 total G>A or T>C point mutations for DMD can be targeted for repair by at least one of the ABEs (iABE-NGA). Describe all other cases including targeting the splicing sites for exon skipping and other diseases.

The recent advances in engineering Cas9 variants with non-G PAM further increases targeting capacity. Moreover, the ABE editing can be designed to induce skipping of mutant exons via targeting the canonical splicing donor or acceptor, thus further broadening the applicability of ABE editing therapy for a larger population of DMD.

The mice at ten months after AAV9-iNG delivery showed significantly higher dystrophin rescue than the mice at 10 weeks after the treatment. One explanation is that the DMD cardiomyocytes with restored dystrophin expression can gain advantage for selective survival and regeneration during the development stages after delivery of AAV9-iNG. Additionally, transduced cardiomyocyte-derived extracellular vesicles can deliver genetic materials such as transcripts encoding iABE-NG into proximal un-transduced cardiomyocytes and confer base editing in those cells.

This study has also shown that systemic delivery of AAV9-iNG resulted in dystrophin restoration in skeletal muscles and functional improvement. As compared to cardiomyocytes, the editing efficiency in skeletal muscles was substantially lower. This can be attributed to the observation that AAV9 has higher tropism towards cardiomyocytes than skeletal muscles. However, other mechanisms can also be responsible for the lower editing efficiency in skeletal muscles. For example, the dystrophic and inflammatory microenvironment in skeletal muscles can pose further constrains on AAV9 delivery and base editing. In addition, targeting muscle satellite cells can be required to improve the overall editing outcomes in skeletal muscle as they are constantly activated to replace injured skeletal muscle in DMD. Although AAV9 has been shown to transduce muscle satellite cells, the efficiency is relatively low. Moreover, the use of a muscle-specific promoter can further reduce the base editing in muscle satellite cells in the present study.

Improvements in these examples show exceptionally high editing efficiency in comparison to other approaches. First, the intein used in the current study (Gp41-1) has superfast kinetics, which allows more efficient assembly of full-length ABE (FIG. 6). Second, each half of the AAVs carries a gRNA-expressing cassette in the present study, while in the other study, gRNA is present in only the C-terminal half of the Npu intein split constructs. The data (FIG. 6h) showed that the gRNA dosage affects the editing efficiency. Third, the promoters used in these studies were also different, which can drive different expression levels of ABE in heart tissues. Finally, the intrinsic difference in the gRNAs and ABE variants can have impacts on the overall editing outcomes. Thus, the exceptionally high editing efficiency achieved in adult dystrophic mice indicates that the AAV9-iNG vectors disclosed herein are useful for clinical applications.

### Example 6. Methods

Mice. Mice (C57BL/6J and B6Ros.Cg-Dmd^{mdx-4cv}/J) were purchased from the Jackson Laboratory and maintained at The Ohio State University Laboratory Animal Resources in accordance with animal use guidelines. All the experimental procedures were approved by the Animal Care, Use, and Review Committee of the Ohio State University.

Plasmid construction. The pCMV-ABE7.10, pCMV-ABE-xCas9(3.7) and pCMV-ABEmax were obtained from Addgene. The NG mutations were introduced by fusion PCR of pCMV-ABEmax and subcloned into pCMV-ABEmax to make pCMV-ABEmaxNG. The A56G and V82G mutations were introduced into TadA* domain by fusion PCR and cloned into pCMV-ABEmaxNG to generate pCMV-iABEmaxNG. The CfaN minigene was synthesized by IDTdna and fused at the amino acid 573 of SpCas9-max through PCR amplification. The TadA-TadA*-SpCas9max (2-573)-CfaN fragment was PCR amplified and subcloned into pAAV under the control of meCMV promoter to generate pAAV-ABEmaxN-temp. The hU6 promoter with mdx^{4cv}-targeting gRNA was PCR amplified and cloned into pAAV-ABEmaxN-temp to make pAAV-ABEmaxN. The CfaC fused with SpCas9max (574-end) was generated by PCR and cloned into pAAV-ABEmaxN-temp to make pAAV-ABEmaxC. Similarly, pAAV-ABEmaxN2 and pAAV-ABEmaxC2NG with the Gp41-1 intein, and pAAV-ABEmaxN3 and pAAV-ABEmaxC3NG with the Npu intein were constructed. The mdx^{4cv} gRNA and other gRNA oligos (listed in Table 2) were annealed and ligated into plenti-ogRNA. The mdx^{4cv} reporter oligos were annealed and ligated into pLKO-puro-2A-mdx^{4cv}-EGFP. All plasmids used in this study are listed in Table 3.

Generation of AAV particles. AAV vectors were produced at the viral vector core of the Nationwide Children's Hospital as previously described. The Gp41-1 intein split of iABE-NGA and the gRNA targeting mdx^{4cv} mutation (GTTaTCTCCTGTTCTGCAGC TGT (SEQ ID NO: 621); note: the underlined PAM sequences were not included in the gRNA) or a non-targeting gRNA (GTTTaTGTCACCAGAGTAAC (SEQ ID NO: 571), the different nucleotides are highlighted in blue) expression cassettes were packaged into AAV9 capsid using the standard triple transfection protocol. A quantitative PCR-based titration method was used to determine an encapsulated vector genome titer utilizing a Prism 7500 Fast Taqman detector system (PE Applied Biosystems Grand Island, NY USA). The following primers/probes were used: 5′-GGATTTCCAAGTCTCCACCC-3′ (SEQ ID NO: 630) and 5′-TCCCACCGTACACGCCTAC-3′ (SEQ ID NO: 631) for titering AAV9-NG, and AAV9-iNG was titered using digital droplet PCR. Titers are expressed as DNase resistant particles per ml (DRP/ml) and rAAV titers used for injection in mice were 8.9×10¹² DRP/ml (AAV9-NG) and 3.0×10¹³ DRP/ml (AAV9-iNG).

Cell culture and transfection. HEK293 cells were cultured in Dulbecco's modified eagle's medium (DMEM) (Corning, Manassas, VA) containing 10% fetal bovine serum (FBS) and 1% 100× penicillin-streptomycin (10,000 U/ml. Invitrogen). Cells were plated in 6-well plates and transfected with the 2 μg plasmids (0.5 μg reporter, 0.75 μg gRNA and 0.75 μg ABE) per well unless specified otherwise by polyethylenimine (PEI) as previously described.

Flow cytometry. At 72 hour post transfection. HEK293 cells transfected with ABE plasmids were collected from 6-well plate and analyzed on Becton Dickinson LSR II (BD Biosciences) to determine GFP-positive cells. A total of 100.000 cell events were collected and data analysis was performed using the FlowJo software (Tree Star, Ashland, OR, USA).

Intramuscular and intravenous administration of AAV9 particles. AAV9-iNG viral particles (2×10¹¹ vg, 25 μl) were injected into the right gastrocnemius compartment of the male mdx^{4cv} mice at 5-6 weeks of age or day 3. For systematic delivery, the male mdx^{4cv} mice at 5-6 weeks of age were administered with AAV9-NG, AAV9-iNG or AAV9-GFP viral particles (1×10¹⁴ vg/kg) via tail vein injection.

Serological analysis. Blood samples were collected at various time points after intramuscular or intravenous injection. The blood samples were allowed to clot for 15 min to 30 min and centrifuged at 5000 rpm for 10 min in room temperature. The supernatant was collected as serum and stored at −80° C. for the biochemical assays. Measurement of ALT (BioVision Incorporated), AST (BioVision Incorporated), BUN (Arbor Assays, Michigan, USA) and cardiac Troponin I (Life Diagnostics, Inc) were performed according to the manufacturer's protocols.

Antibody ELISA. Antibodies against AAV9 and SpCas9 were detected by adapting previously published protocols. In brief, recombinant AAV9 (2×10⁹ vg/well) and SpCas9 protein (0.27 μg/well) were diluted in 1× Coating Buffer A (BioLegend) and used to coat a 96-well Nunc MaxiSorp plate. Proteins were incubated overnight at 4° C. to adsorb to the plate. Plates were washed four times 5 min each with PBS plus 0.05% Tween-20 and then blocked with 1× Assay Diluent A (BioLegend) for 1 h at room temperature. The anti-AAV2 (A20, cat. #03-65155, American Research Products, Inc) and anti-SpCas9 antibody (Diagenode C15310258) was used as positive control for detection of anti-AAV9 and anti-SpCas9 antibodies, respectively. Serum samples were added in 1:50 dilution and plates were incubated for 2 h at room temperature with shaking. Plates were washed four times 5 min each and 100 μl of blocking solution containing goat anti-mouse IgG (Sigma 1:3,000) was added to each well and incubated at 1h at room temperature. Plates were washed four times 5 min each, 100 μl of freshly mixed TMB Substrate Solution (BioLegend) was added to each well, and incubated in the dark for 20 min. The reaction was stopped by adding 100 μl 2N H₂SO₄ Stop Solution. Optical density at 450 nm was measured with a plate reader.

Muscle contractility measurements. At 5 weeks after intramuscular AAV9-NG or intravenous AAV9-iNG injection, muscle contractility was measured using an in vivo muscle test system (AuroraScientific Inc). Mice were anesthetized with 3% (w/v) isoflurane and anesthesia was maintained by 1.5% isoflurane (w/v) during muscle contractility measurement. Maximum plantarflexion tetanic torque was measured during a train of supramaximal electric stimulations of the tibial nerve (pulse frequency 150 Hz, pulse duration 0.2 ms).

Histopathological assessment of tissues. Mice were sacrificed at various time points, and tissues (heart, lung, diaphragm, spleen, kidney, liver, quadriceps and gastrocnemius) were harvested for histological, histochemical, biochemical and molecular analyses. For immunohistological examinations, tissues were embedded in optimal cutting temperature (OCT, Sakura Finetek, Netherlands) compound and snap-frozen in cold isopentane for cryosectioning. The tissues were stored at −80° C. and processed for biochemical analysis and histology assessment. Frozen cryosections (7 μm) were fixed with 4% paraformaldehyde for 15 minutes at room temperature. After washing with PBS, the slides were blocked with 3% BSA for 1 hour. The slides were incubated with primary antibodies against dystrophin (ab15277, 1:100, Abcam) and laminin-α2 (ALX-804-190-C100, 1:100, Enzo) at 4° C. for 1 hour. After that, the slides were washed extensively with PBS and incubated with secondary antibodies (Alexa Fluor 488 goat anti-rat IgG, Invitrogen, Carlsbad, CA or Alexa Fluor 568 donkey anti-rabbit IgG, Invitrogen) for 1 hour at room temperature. The slides were sealed with VECTASHIELD Antifade Mounting Medium with DAPI (Vector Laboratory, Burlingame, CA). All images were taken under a Nikon Ti-E fluorescence microscope (magnification 200×) (Nikon, Melville, NY). Laminin-α2-positive and dystrophin-positive muscle fibers were counted using NIS-Elements AR version 4.50 (Nikon, Melville, NY). The amount of dystrophin positive muscle fibers is represented as a percentage of total laminin-α2-positive muscle fibers.

For trichrome staining, Masson's 2000 Trichrome Kit was used (American MasterTech, Lodi, CA). The muscle and heart sections were fixed with 4% paraformaldehyde for 1 hour at room temperature. After washing with PBS, the tissue sections were stained with Masson's trichrome reagent following the manufacturer's instruction.

Western blot analysis. Mouse tissues from mdx^{4cv} mice treated with or without AAV9-NG or AAV9-iNG were lysed with cold RIPA buffer supplemented with protease inhibitors and extracted protein samples were separated by SDS-PAGE (BioRad, 4-15%) and transferred onto Nitrocellulose membranes (0.45 μm). The rabbit polyclonal anti-dystrophin (E2660, 1:500, Spring Bioscience, Pleasanton, CA), rabbit polyclonal anti-Cas9 (C15310258-100, 1:1000, Diagenode, Denville, NJ) and rabbit monoclonal anti-Gapdh (#2118, 1:2000, Cell Signaling Technology, Danvers, MA) antibodies were used for immunoblotting analysis. HRP conjugated goat anti-mouse (1:4000) and goat anti-rabbit (1:4000) secondary antibodies were obtained from Cell Signaling Technology, Danvers, MA. The membranes were developed using ECL western blotting substrate (Pierce Biotechnology, Rockford, IL) and scanned by ChemiDoc XRS+ system (BioRad, Hercules, CA). Western blots were quantified using Image Lab 6.0.1 software (Bio-Rad Laboratories, Hercules, CA) according to the manufacturer's instruction.

Extraction of genomic DNA and total RNA, PCR and Sanger sequencing. Genomic DNA from mouse tissues and cultured HEK293 cells were extracted using DNeasy Blood & Tissue Kit (Qiagen, Germantown, MD). Total RNA was extracted from mouse tissues and HEK293 cells using Quick-RNA MiniPrep Kit (ZYMO Research, Irvine, CA). Five μg of treated RNA was used as template for first-strand cDNA synthesis by using RevertAid RT Reverse Transcription Kit (Life Technologies, Carlsbad, CA). Aliquots of the RT product were used for RT-PCR analysis of dystrophin editing. PCR reactions were carried out with 100 ng genomic DNA or cDNA in the GoTaq Master Mix (Promega) according to the manufacturer's instruction. The primers used for RT-PCR of the reporter genes and PCR of endogenous loci were listed in Table 2. The PCR products were purified using the Wizard SV Gel and PCR Clean-up System (Promega). Purified genomic DNA and RT PCR products (100 ng) were subjected to Sanger sequencing at the Genomics Shared Resource of the Ohio State University Comprehensive Cancer Center. The sequencing data were analyzed by BEAT program.

Targeted deep sequencing. The on-target and off-target loci were first amplified by genomic DNA PCR and/or RT-PCR using gene-specific primers with Illumina adapters (primers are provided in Table 4). The first PCR products were purified using a commercial purification kit (Promega, Madison, WI, USA), diluted, pooled, and subjected to a second round PCR with primers including the index sequences. The final PCR products were electrophoresed on an agarose gel, showing a single sharp peak. The quality and quantity were assayed using an Agilent Bioanalyzer 2100 (Genomics Shared Resource, Ohio State University Comprehensive Cancer Center). The purified amplicons were pooled and sent for sequencing using a MiSeq nano-scale flow cell (Paired-end 300 base-pair reads) at The Genomics Services Laboratory of Nationwide Children's Hospital. The FASTQ files were analyzed using CRISPResso2 with default parameters.

RNA-seq experiments. RNA library preparation was performed using NEBNext® Ultra™ II Directional (stranded) RNA Kit for Illumina (NEB #E7760L, New England Biolabs) with an initial input of 100 ng ng extracted RNA per sample, measured using Qubit RNA HS reagents (#Q32852 Invitrogen) for fragmentation, cDNA synthesis and amplification. Depletion of ribosomal RNA (rRNA) was carried out with NEBNext rRNA Depletion Kit (human, mouse, rat) from New England Biolabs (#E6310X). NEBNext Multiplex oligos indexes kits (E7335L, E7500L and E7710L) from New England Biolabs were used to barcode each library following the manufacturer protocol. RNA-seq libraries were examined using an Agilent 2100 Bioanalyzer and a High Sensitivity DNA kit (Agilent Technologies, Inc). RNA-seq libraries were sequenced on Novaseq SP Paired-End 150 bp format at The Genomics Services Laboratory of Nationwide Children's Hospital.

RNA sequence variant calling and variant filtering. Illumina paired-end fastq sequencing reads were processed according to GATK Best Practices for RNA-seq variant calling. In brief, reads were aligned to the mouse mm10 reference genome using STAR version 1.5.2 in two-pass mode with the parameters implemented by the ENCODE project. Picard tools (version 2.19.0) was then applied to sort and mark duplicates of the mapped BAM files. The refined BAM files were subject to split reads that spanned splice junctions, local realignment, base recalibration and variant calling with SplitNCigarReads, IndelRealigner, BaseRecalibrator and HaplotypeCaller tools from GATK (version 4.1.2.0), respectively. Known variants in dbSNP version 142 were used during base quality recalibration. From all called variants, downstream analyses focused solely on single-nucleotide variants (SNVs) on canonical (1-22, X, Y and M) chromosomes. To identify variants with high confidence, clusters of at least five SNVs were filtered that were within a window of 35 bases and variants with Fisher strand values >30.0, qual by depth values <2.0 and sequencing depth <10. Base edits labelled as A-to-I comprise A-to-I edits called on the positive strand as well as T-to-C edits sourced from the negative strand, since the RNAs were converted into cDNA before sequencing, both the nucleotide and its complementary base can be sequenced. Results obtained with this pipeline can underestimate the actual number of RNA edits occurring in cells because of the high stringency of the variant calling pipeline and potential under-representation of intronic and intergenic RNA in our experiments.

Any confident variants found in wild-type Neuro2a cells were considered to be SNPs and were filtered out from the base-editor-transfected groups for off-target analysis. Similarly, any confident variants found in control mdx^{4cv} heart samples were filtered out from the AAV9-iNG group for off-target analysis. The editing rate was calculated as the number of mutated reads divided by the sequencing depth for each site.

ClinVar database analysis. The ClinVar data was converted into a tab-delimited flat file. A python script (clinvar.py) was written to process the tab-delimited flat file of ClinVar data.

Statistical analysis. The data were expressed as mean±S.E.M. and analyzed with GraphPad Prism 8.0.1 software (San Diego, USA). Statistical significance was determined using one-way ANOVA followed by Bonferroni post hoc-tests for multiple groups or student's t-test for two groups. A P value of less than 0.05 is regarded as significant.

Data availability. The sequencing data have been deposited in the NCBI SRA under project accession numbers (PRJNA673243).

### Example 7. Use of the System for Treatment of Spinal Muscular Atrophy

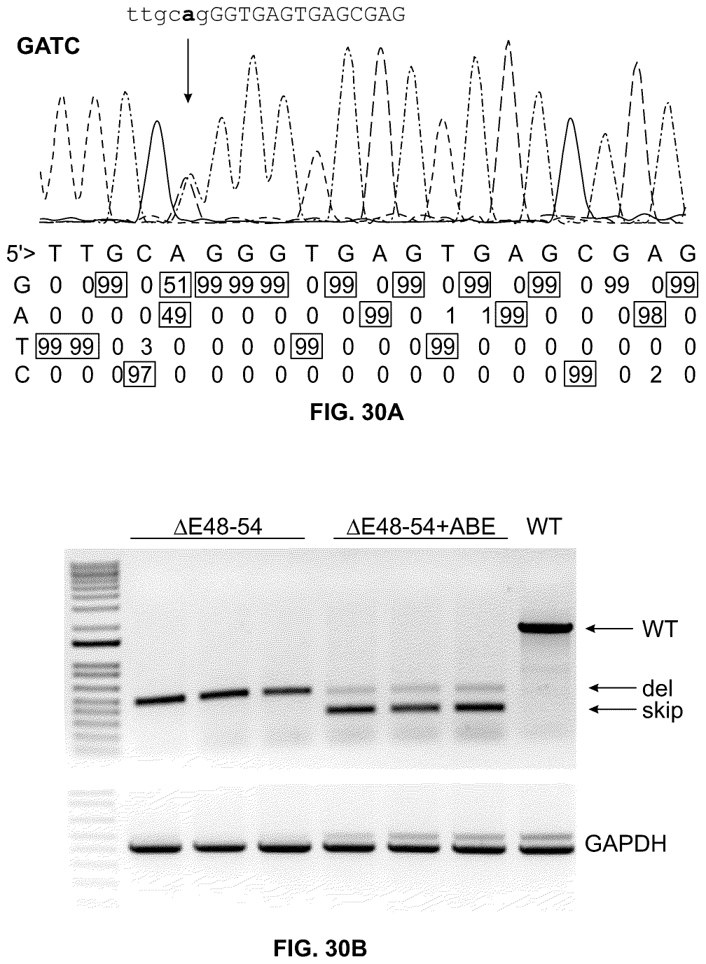

The iABE-NGA system can be broadly used to rescue dystrophin expression in DMD and treat other conditions, in addition to correcting point mutations in DMD and dysferlinopathy. For example, by targeting the splice sites, iABE-NGA can mediate exon skipping and rescue dystrophin expression in a human induced pluripotent stem cell-derived cardiomyocytes with a large deletion from exon 48 through 54 in DMD gene (FIG. 30).

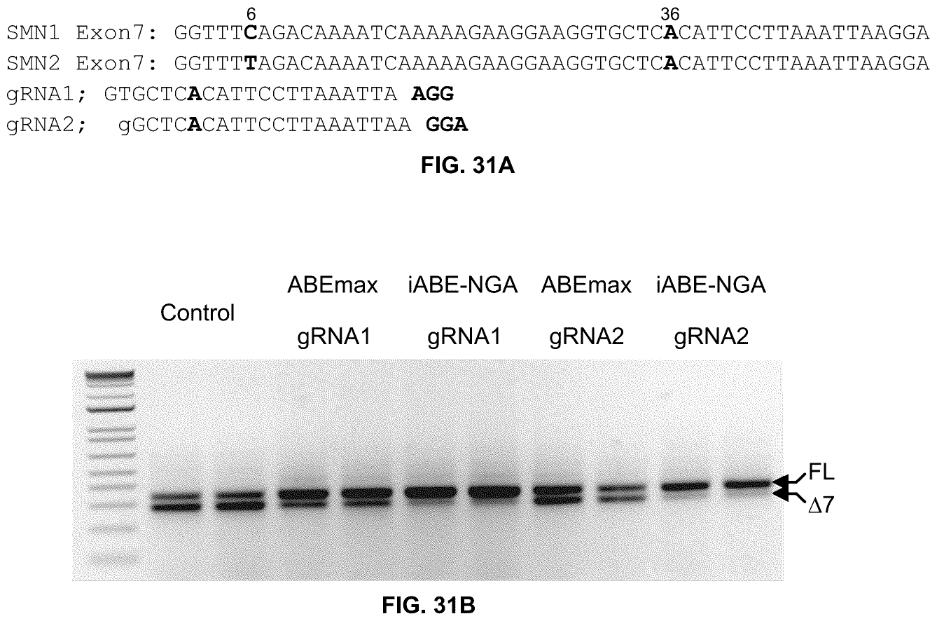

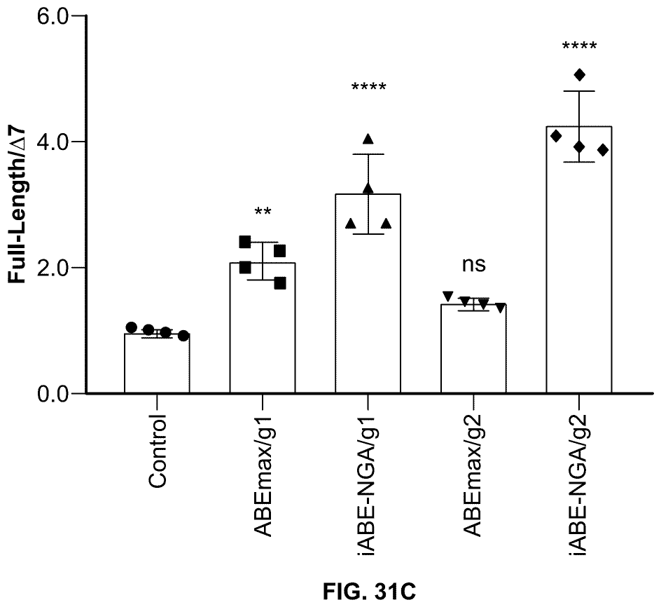

Mutations in the survival motor neuron 1 (SMN1) gene cause spinal muscular atrophy (SMA). There is a paralogous gene SMN2 in human, present in almost all SMA patients. The SMN2 is different from SMN1 in exon 7, with position 6 converted from C to T in SMN2, which results in the skipping of exon 7 in SMN2 mRNA and non-functional SMN2 protein. Using iABE-NGA-mediated editing of A36 showed that the exon 7 of SMN2 was spliced in (FIG. 31), and thus this can be a therapy for SMA.

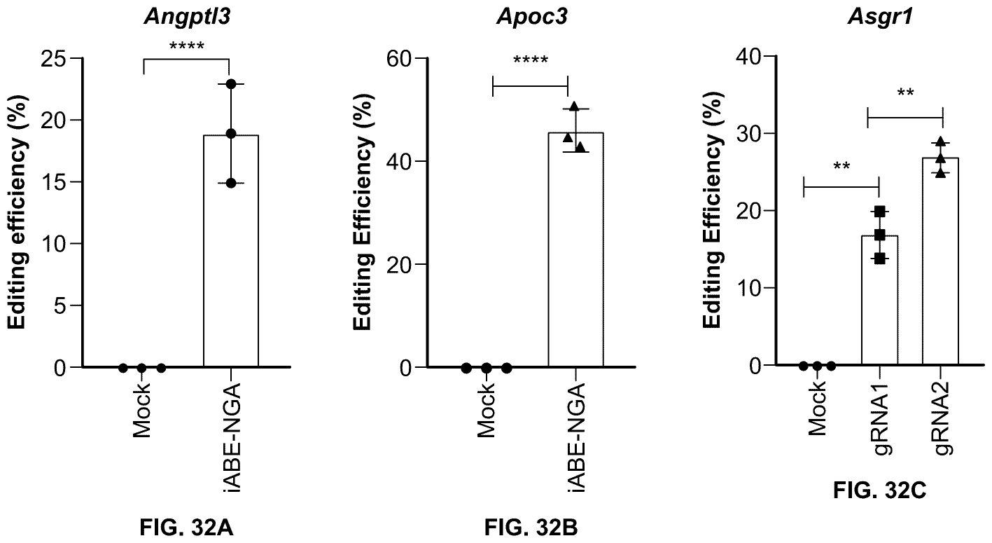

Moreover, by targeting the splice sites, one can generate loss-of-functions in the therapeutic targets such as those involved in the regulation of plasma cholesterol levels. Previous studies identified a number of protective loss-of-functions in genes such as ANGPTL3, APOC3 and ASGR1, which reduce the plasma low cholesterol levels and renders cardioprotection. iABE-NGA can install such loss-of-function mutations in the aforementioned genes, which can be used as a one-time cure for dyslipidemia (FIG. 32).

TABLE 1
List of ABE variants engineered in this study.

| Name | Description |
|---|---|
| ABE-NG | ABEmax with SpCas9-NG mutations R1335V/ |
| | L1111R/D1135V/G1218R/E1219F/A1322R/T1337R |
| ABE-NGA | ABE-NG with R1335Q mutation |
| ABE-NGC | ABE-NG with R1335E mutation |
| ABE-NG-loop | ABE-NG with the loop sequence from ScCas9 |
| | (amino acids 367-376) inserted |
| ABE-NGX | ABE-NG with A262T/R324L/S409I/E480K/ |
| | E543D/M694I mutations |
| ABE-NGX-NGA | ABE-NGX with R1335Q |
| ABE-NGX-NGC | ABE-NGX with R1335E |
| ABE-NGX-loop | ABE-NGX with the loop sequence from ScCas9 |
| | (amino acids 367-376) inserted |
| ABEmaxSc | ABEmax with SpCas9 nickase replaced with |
| | ScCas9 nickase |
| ABE-NGm | ABE-NG with the dimeric TadA-TadA* replaced |
| | with monomeric TadA* containing two additional |
| | mutations A56G and V82G |
| iABE-NGA | ABE-NGA with the dimeric TadA-TadA* replaced |
| | with monomeric TadA* containing two additional |
| | mutations A56G and V82G |

TABLE 2
List of gRNA target sequences and primers for PCR in this study.

| Name | Sequence | SEQ ID NOs |
|---|---|---|
| Mdx4cv-gRNA | GTTATCTCCTGTTCTGCAGC | SEQ ID NO: 570 |
| NT-gRNA | GTTTATGTCACCAGAGTAAC | SEQ ID NO: 571 |
| mDMD-i52-F | GAGGTAATAGAGCCAAGCCCT | SEQ ID NO: 572 |
| mDMD-i53-R | GCAAGAATTCCACTTTTCACTTCCT | SEQ ID NO: 573 |
| mDMD-E51-F | CTGTCATCTCCAAACTAGAAATGC | SEQ ID NO: 574 |
| mDMD-E55-R | GCAGCCTCTTGCTCACTTACTC | SEQ ID NO: 575 |
| S1-gRNA | GATGACAGGCAGGGGCACCG | SEQ ID NO: 576 |
| S1-F | TTCCAGTGGTTCAATGGTCA | SEQ ID NO: 577 |
| S1-R | CTTTCAACCCGAACGGAGAC | SEQ ID NO: 578 |
| VEGFA-S5-gRNA | GAGCGAGCAGCGTCTTCGAG | SEQ ID NO: 579 |
| VEGFA-S12-gRNA | GCAGACGGCAGTCACTAGGG | SEQ ID NO: 580 |
| VEGFA-S14-gRNA | GGGAAGCTGGGTGAATGGAG | SEQ ID NO: 581 |
| VEGFA-F | AGCTGTTTGGGAGGTCAGAA | SEQ ID NO: 582 |
| VEGFA-R | AGGGAGCAGGAAAGTGAGGT | SEQ ID NO: 583 |
| Site13-gRNA | GTCGCAGGACAGCTTTTCCT | SEQ ID NO: 584 |
| Site13-F | TGTAGCTACGCCTGTGATGG | SEQ ID NO: 585 |
| Site13-R | TGCCCTGAGATCTTTTCCTC | SEQ ID NO: 586 |
| FANCF-gRNA | GATCCAGGTGCTGCAGAAGG | SEQ ID NO: 587 |
| FANCF-F | CTCTTGCCTCCACTGGTTGT | SEQ ID NO: 588 |
| FANCF-R | TCGGTAGGATGCCCTACATC | SEQ ID NO: 589 |
| Q623X-gRNA | ATCCTACAGCATGGTGGCTG | SEQ ID NO: 590 |
| Puro-F | AGTGGTCTCCGGAAACCTCCGCGCCCC | SEQ ID NO: 591 |
| | GCAAC | |
| GFP-R | TCCTTGAAGAAGATGGTGCG | SEQ ID NO: 592 |

TABLE 3
List of plasmids used in this study.

| ID | Name | Description |
|---|---|---|
| pXL-0570 | pCMV_ABEmax (Addgene # | Expressing ABEmax; used in |
| | 112095) | FIG. 1c-e; FIG. 4b, d; |
| | | Suppl. FIG. S2 |
| pXL-0550 | pCMV_xCas9_3.7_-ABE_7.10 | Expressing ABE-x; used in |
| | (Addgene #108382) | FIG. 1c-e; FIG. 6b; FIG. 5. |
| pXL-0645 | pCMV_ABEmaxNG | Expressing ABE-NG; used in |
| | | FIG. 1c-e; FIG. 2a-f; FIG. 3b; |
| | | FIG. 6b-6d; |
| pXL-0752 | pLKO-puro-2A-mdx4cv-GFP | mdx^{4cv} reporter; used in |
| | | FIG. 1c-e; FIG. 6c, 6d, h; FIG. 4. |
| pXL-0631 | pLenti-puro-OgRNA_mdxE53 | gRNA targeting mdx^{4cv} mutation; |
| | | used in FIG. 1c-e; FIG. 3b-d; FIG. |
| | | 6c-6h; FIG. 27g, 27h; FIG. 4. |
| pXL-0858 | pCMV_ABEmaxNG-NGA | Expressing ABE-NGA; used |
| | | in FIG. 2a-f. |
| pXL-0869 | pCMV_ABE-NGC | Expressing ABE-NGC; used |
| | | in FIG. 2a-f. |
| pXL-0872 | pCMV_ABE-NG-loop | Expression ABE-NG-loop; |
| | | used in FIG. 2a-f. |
| pXL-0712 | pCMV-ABEmaxNGX | Expressing ABE-NGX; |
| | | used in FIG. 2a-f. |
| pXL-0868 | pCMV-ABEmaxNGX-NGA | Expressing ABE-NGX-NGA; |
| | | used in FIG. 2a-f. |
| pXL-0875 | pCMV-ABEmaxNGX-NGC | Expressing ABE-NGX-NGC; |
| | | used in FIG. 2a-f. |
| pXL-0877 | pCMV-ABEmaxNGX-loop | Expressing ABE-NGX-loop; |
| | | used in FIG. 2a-f. |
| pXL-0723 | pCMV-ABEmaxSC | Expressing ABEmaxSc; |
| | | used in FIG. 2a-f. |
| pXL-0627 | pLenti-puro-S1OgRNA | S1 targeting gRNA; |
| | | used in FIG. 2a. |
| pZC0009 | pLenti-Q2440X-ogRNA | Non-targeting gRNA; |
| | | used in FIG. 7. |
| pXL-0796 | pLenti-VEGFA-S5 | gRNA targeting VEGFA Site5; |
| | | used in FIG. 2b. |
| pXL-0797 | pLenti-VEGFA-S12 | gRNA targeting VEGFA Site 12; |
| | | used in FIG. 2c. |
| pXL-0798 | pLenti-VEGFA-S14 | gRNA targeting VEGFA Site14; |
| | | used in FIG. 2d. |
| pXL-0800 | pLenti-Site13 | gRNA targeting Site13; |
| | | used in FIG. 2e. |
| pXL-0801 | pLenti-FANCF-ogRNA | gRNA targeting FANCF; |
| | | used in FIG. 2f. |
| pZC0103 | pCMV_miniABE_NG(V82G) | Expressing miniABE(82G)-NG; |
| | | used in FIG. 3b, 3c. |
| pZC0104 | pCMV_miniABE-NG | Expressing miniABE-NG; |
| | | used in FIG. 3b-3d. |
| pXL-0853 | pCMV_miniABEmax- | Expressing miniABE(A56G)-NG; |
| | NG(A56G) | used in FIG. 3b, 3c. |
| pXL-0854 | pCMV_miniABEmaxNG(GG) | Expressing miniABE(GG)-NG; |
| | | used in FIG. 3b-3d, FIG. 4. |
| pXL-0420 | pCMV_ABE7.10 (Addgene | Expressing ABE7.10; used |
| | #102919) | in FIG. 6b, FIG. 5 |
| pXL-0670 | pBac-rAAV-ABEmaxN-E53 | Expressing Cfa Split_N of |
| | OgRNA | ABE and mdx4cv gRNA; used |
| | | in FIG. 6b-6d. |
| pXL-0671 | pBac-rAAV-ABEmaxC-NG-E53 | Expressing Cfa Split_C of |
| | OgRNA | ABE-NG and mdx4cv gRNA; used |
| | | in FIG. 6b-6d. |
| pXL-0672 | pBac-rAAV-ABEmaxN2-E53 | Expressing Gp41-1 Split_N of |
| | OgRNA | ABE and mdx4cv gRNA; used |
| | | in FIG. 6b-6d. |
| pXL-0673 | pBac-rAAV-ABEmaxC2-NG- | Expressing Gp41-1 Split_C of |
| | E53 OgRNA | ABE-NG and mdx4cv gRNA; used |
| | | in FIG. 6b-6d. |
| pZC0117 | pX601-mhCMV- ABEmaxNGA- | Expressing Npu Split_C of |
| | C3-E53ogRNA | iABE-NGA and mdx4cv gRNA; |
| | | used in FIG. 6e-6h. |
| pZC0118 | pX601-mhCMV-miniABEmax- | Expressing Npu Split_N of |
| | N3-E53ogRNA | iABE-NGA and mdx4cv gRNA; |
| | | used in FIG. 6e-6h. |
| pZC0119 | pX601-mhCMV-miniABEmax- | Expressing Npu Split_N of |
| | N3-zeo | iABE-NGA; used in FIG. 6e-6h. |
| pZC0031 | pX601-mhCMV-miniABEmax- | Expressing Gp41-1 Split_N of |
| | N2-E53ogRNA | iABE-NGA and mdx4cv gRNA; |
| | | used in FIG. 6e-6h. |
| pZC0033 | pX601-mhCMV-ABEmaxNGA- | Expressing Gp41-1 Split_C of |
| | C2-E53ogRNA | iABE-NGA and mdx4cv gRNA; |
| | | used in FIG. 6e-6h. |
| pXL-0855 | pX601-MHP1- | Expressing Gp41-1 Split_N of |
| | miniABEmaxNG-N2-(GG) | iABE-NGA and mdx4cv gRNA, |
| | | MHP1 promoter; used for AAV9 |
| | | production in FIG. 8, 27. |
| pXL-0706 | pX601-MHP1-ABEmaxC2-NG- | Expressing Gp41-1 Split_C of |
| | E53 ogRNA | iABE-NGA and mdx4cv gRNA, |
| | | MHP1 promoter; used for AAV9 |
| | | production in FIG. 8, 27. |
| pYZ1059 | pCMV_NG-ABE8e | Expressing ABE8e-NG; |
| | | used in FIG. 4 |
| pZC0105 | pCMV_NG-ABE8.17 | Expressing ABE8.17-NG; |
| | | used in FIG. 4 |
| pZC0106 | pCMV_NG-ABE8.20 | Expressing ABE8.20-NG; |
| | | used in FIG. 4 |
| pZC0111 | pCMV_SpG-ABE8e(V106W) | Expressing ABE8e-SpG(V106W); |
| | | used in FIG. 4 |
| pPW-0007 | pLKO-puro-2A-Q623X-GFP | Q623X reporter; used |
| | | in FIG. 4b. |
| pPW-0010 | pLenti-Q623X-ogRNA | Q623X targeting gRNA; |
| | | used in FIG. 4b. |
| pXL-0419 | pCMV-ABE7.9 (Addgene | Expressing ABE7.9; |
| | #102918) | used in FIG. 8. |
| pXL-0419 | pCMV-ABE7.9 (Addgene | Expressing ABE7.9; |
| | #102918) | used in FIG. 8. |
| pYG9010 | pBac-rAAV-IntC-SpCas9n | Expressing Npu_N-SpCas9 |
| | | nickase; used in FIG. 8. |
| pYG9011 | pBac-rAAV-ABE7.10v3 | Expressing |
| | | GFP-TadA-TadA*-Npu_C; |
| | | used in FIG. 8. |

TABLE 4
List of primers used for NGS in this study.

| Name | Sequence | |
|---|---|---|
| Mdx4cv- | ACACTCTTTCCCTACACGACGCTCTTCCGATCTGAACTCAT | SEQ ID |
| E52-F | TACTGCTGCCCAGA | NO: 593 |
| Mdx4cc- | GTGACTGGAGTTCAGACGTGTGCTCTTCCGATCGACCTGTT | SEQ ID |
| E53-R | CGGCTTCTTCCTTA | NO: 594 |
| Mdx4cv- | ACACTCTTTCCCTACACGACGCTCTTCCGATCTAAATTTCC | SEQ ID |
| i52-F | ACTGTCTTCTCTTGAGT | NO: 595 |
| Mdx4cv- | GTGACTGGAGTTCAGACGTGTGGTCTTCCGATCGCTTGCCT | SEQ ID |
| i53-R | CTGACCTGTCCTAT | NO: 596 |
| mChr16OT- | ACACTCTTTCCCTACACGACGCTCTTCCGATCTGTGACTAG | SEQ ID |
| F | GGGCAAAGCAAGAT | NO: 597 |
| mChr16OT- | GTGACTGGAGTTCAGACGTGTGCTCTTCCGATCCTTCCAAA | SEQ ID |
| R | CTTTCTGCCCATTC | NO: 598 |
| mChr1OT- | ACACTCTTTCCCTACACGACGCTCTTCCGATCTAACACAGC | SEQ ID |
| F | GTGCTCTTTCCTTAC | NO: 599 |
| mChr1OT- | GTGACTGGAGTTCAGACGTGTGCTCTTCCGATCGTTCAGAA | SEQ ID |
| R | GAACATCCCGTTGAC | NO: 600 |
| NGS- | AATGATACGGCGACCACCGAGATCTACACTCTTTCCCTACA | SEQ ID |
| final-F | CGAC | NO: 601 |
| NGS- | CAAGCAGAAGACGGCATACGAGATCTTGTAGTGACTGGAG | SEQ ID |
| final-R1 | TTCAGACGT | NO: 602 |
| NGS- | CAAGCAGAAGACGGCATACGAGATCAGATCGTGACTGGA | SEQ ID |
| final-R2 | GTTCAGACGT | NO: 603 |
| NGS- | CAAGCAGAAGACGGCATACGAGATCCGTCCGTGACTGGAG | SEQ ID |
| final-R3 | TTCAGACGT | NO: 604 |
| NGS- | CAAGCAGAAGACGGCATACGAGATATGTCAGTGACTGGAG | SEQ ID |
| final-R4 | TTCAGACGT | NO: 605 |
| NGS- | CAAGCAGAAGACGGCATACGAGAT GTCCGC | SEQ ID |
| final-R5 | GTGACTGGAGTTCAGACGT | NO: 606 |
| NGS- | CAAGCAGAAGACGGCATACGAGAT TTAGGC | SEQ ID |
| final-R6 | GTGACTGGAGTTCAGACGT | NO: 607 |
| NGS- | CAAGCAGAAGACGGCATACGAGAT CGATGT | SEQ ID |
| final-R7 | GTGACTGGAGTTCAGACGT | NO: 608 |
| NGS- | CAAGCAGAAGACGGCATACGAGAT TGACCA | SEQ ID |
| final-R8 | GTGACTGGAGTTCAGACGT | NO: 609 |
| NGS- | CAAGCAGAAGACGGCATACGAGAT AGTCAA | SEQ ID |
| final-R9 | GTGACTGGAGTTCAGACGT | NO: 610 |
| NGS- | CAAGCAGAAGACGGCATACGAGAT AGTTCC | SEQ ID |
| final-R10 | GTGACTGGAGTTCAGACGT | NO: 611 |
| NGS- | CAAGCAGAAGACGGCATACGAGAT GATCAG | SEQ ID |
| final-R11 | GTGACTGGAGTTCAGACGT | NO: 612 |
| NGS- | CAAGCAGAAGACGGCATACGAGAT ACAGTG | SEQ ID |
| final-R12 | GTGACTGGAGTTCAGACGT | NO: 613 |
| NGS- | CAAGCAGAAGACGGCATACGAGAT TATACT | SEQ ID |
| final-R13 | GTGACTGGAGTTCAGACGT | NO: 614 |
| NGS- | CAAGCAGAAGACGGCATACGAGAT CAACAA | SEQ ID |
| final-R14 | GTGACTGGAGTTCAGACGT | NO: 615 |
| NGS- | CAAGCAGAAGACGGCATACGAGAT GTTGTT | SEQ ID |
| final-R15 | GTGACTGGAGTTCAGACGT | NO: 616 |
| NGS- | CAAGCAGAAGACGGCATACGAGAT TCGGTT | SEQ ID |
| final-R16 | GTGACTGGAGTTCAGACGT | NO: 617 |
| NGS- | CAAGCAGAAGACGGCATACGAGAT AGTATT | SEQ ID |
| final-R17 | GTGACTGGAGTTCAGACGT | NO: 618 |
| NGS- | CAAGCAGAAGACGGCATACGAGAT TCTTGT | SEQ ID |
| final-R18 | GTGACTGGAGTTCAGACGT | NO: 619 |

TABLE 5

| | | | | | | | re- | | |
|---|---|---|---|---|---|---|---|---|---|
| | | | | | vari- | vari- | pair | refer- | sgRNA_re- |
| chr | pos | hgvs_c | hgvs_p | all_traits | ants | ant type | editor | ence_seq | pair |
| X | 31173588 | NM_004020.3:c.2843 + | NP_003997.1:p.Gln3427Ter | Dilated | G > A | intron | ABE | SEQ ID | SEQ ID |
| | | 5081C > T | | cardiomy- | | variant | | NO: 48 | NO: 222; |
| | | | | opathy | | | | | SEQ ID |
| | | | | 3B; Becker | | | | | NO: 223 |
| | | | | muscular | | | | | |
| | | | | dystrophy; | | | | | |
| | | | | Duchenne | | | | | |
| | | | | muscular | | | | | |
| | | | | dystrophy | | | | | |
| X | 32464674 | NM_004006.2:c.3188G > | NP_003997.1:p.Trp1063Ter | Duchenne | C > T | nonsense | ABE | SEQ ID | SEQ ID |
| | | A | | muscular | | | | NO: 49 | NO: 224; |
| | | | | dystrophy; | | | | | SEQ ID |
| | | | | DUCHENNE | | | | | NO: 225; |
| | | | | MUSCULAR | | | | | SEQ ID |
| | | | | DYSTROPHY | | | | | NO: 226; |
| | | | | | | | | | SEQ ID |
| | | | | | | | | | NO: 227 |
| X | 32217037 | NM_004006.2:c.6317G > | NP_003997.1:p.Trp2106Ter | not provided; | C > T | nonsense | ABE | SEQ ID | SEQ ID |
| | | A | | Not Provided | | | | NO: 50 | NO: 228; |
| | | | | | | | | | SEQ ID |
| | | | | | | | | | NO: 229 |
| X | 32362826 | NM_004006.2:c.5287C > | NP_003997.1:p.Arg1763Ter | Dilated | G > A | nonsense | ABE | SEQ ID | SEQ ID |
| | | T | | cardiomy- | | | | NO: 51 | NO: 230; |
| | | | | opathy | | | | | SEQ ID |
| | | | | 3B; Becker | | | | | NO: 231; |
| | | | | muscular | | | | | SEQ ID |
| | | | | dystrophy; | | | | | NO: 232 |
| | | | | Duchenne | | | | | |
| | | | | muscular | | | | | |
| | | | | dystrophy | | | | | |
| X | 31178668 | NM_004006.2:c.10223 + | NP_004010.1:p.Thr340= | Dilated | C > T | synon- | ABE | SEQ ID | SEQ ID |
| | | 1G > A | | cardiomy- | | ymous | | NO: 52 | NO: 233; |
| | | | | opathy | | variant | | | SEQ ID |
| | | | | 3B; Becker | | | | | NO: 234; |
| | | | | muscular | | | | | SEQ ID |
| | | | | dystrophy; | | | | | NO: 235; |
| | | | | Duchenne | | | | | SEQ ID |
| | | | | muscular | | | | | NO: 236 |
| | | | | dystrophy | | | | | |
| X | 31180423 | NM_004006.2:c.10033C > | NP_003997.1:p.Arg3345Ter | Dilated | G > A | nonsense | ABE | SEQ ID | SEQ ID |
| | | T | | cardiomy- | | | | NO: 53 | NO: 237; |
| | | | | opathy | | | | | SEQ ID |
| | | | | 3B; Becker | | | | | NO: 238 |
| | | | | muscular | | | | | |
| | | | | dystrophy; | | | | | |
| | | | | Duchenne | | | | | |
| | | | | muscular | | | | | |
| | | | | dystrophy; | | | | | |
| | | | | not provided | | | | | |
| X | 31478163 | NM_004006.2:c.8880G > | NP_003997.1:p.Trp2960Ter | not specified | C > T | nonsense | ABE | SEQ ID | SEQ ID |
| | | A | | | | | | NO: 54 | NO: 239 |
| X | 31479043 | NM_004006.2:c.8608C > | NP_003997.1:p.Arg2870Ter | Dilated | G > A | nonsense | ABE | SEQ ID | SEQ ID |
| | | T | | cardiomy- | | | | NO: 55 | NO: 240 |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32697947 | NM_004006.2:c.883C > | NP_003997.1:p.Arg295Ter | Becker | G > A | nonsense | ABE | SEQ ID | SEQ ID |
| | | T | | muscular | | | | NO: 56 | NO: 241; |
| | | | | dystrophy; | | | | | SEQ ID |
| | | | | Duchenne | | | | | NO: 242; |
| | | | | muscular | | | | | SEQ ID |
| | | | | dystrophy; | | | | | NO: 243; |
| | | | | Dilated | | | | | SEQ ID |
| | | | | cardiomy- | | | | | NO: 244; |
| | | | | opathy 3B | | | | | SEQ ID |
| | | | | | | | | | NO: 245 |
| X | 32573766 | NM_004006.2:c.1683G > | NP_003997.1:p.Trp561Ter | Duchenne | C > T | nonsense | ABE | SEQ ID | SEQ ID |
| | | A | | muscular | | | | NO: 57 | NO: 246; |
| | | | | dystrophy; | | | | | SEQ ID |
| | | | | Duchenne | | | | | NO: 247; |
| | | | | muscular | | | | | SEQ ID |
| | | | | dystrophy | | | | | NO: 248; |
| | | | | | | | | | SEQ ID |
| | | | | | | | | | NO: 249 |
| X | 32484918 | NM_004006.2:c.2803 + | | Dilated | C > T | splice | ABE | SEQ ID | SEQ ID |
| | | 1G > A | | cardiomy- | | donor | | NO: 58 | NO: 250 |
| | | | | opathy | | variant | | | |
| | | | | 3B; Dilated | | | | | |
| | | | | cardiomy- | | | | | |
| | | | | opathy 3B | | | | | |
| X | 32573744 | NM_004006.2:c.1704 + | | Becker | C > T | splice | ABE | SEQ ID | SEQ ID |
| | | 1G > A | | muscular | | donor | | NO: 59 | NO: 251; |
| | | | | dystrophy; | | variant | | | SEQ ID |
| | | | | Duchenne | | | | | NO: 252; |
| | | | | muscular | | | | | SEQ ID |
| | | | | dystrophy; | | | | | NO: 253; |
| | | | | not specified | | | | | SEQ ID |
| | | | | | | | | | NO: 254 |
| X | 32816541 | NM_004006.2:c.457C > | NP_003997.1:p.Gln153Ter | Duchenne | G > A | nonsense | ABE | SEQ ID | SEQ ID |
| | | T | | muscular | | | | NO: 60 | NO: 255; |
| | | | | dystrophy | | | | | SEQ ID |
| | | | | | | | | | NO: 256; |
| | | | | | | | | | SEQ ID |
| | | | | | | | | | NO: 257; |
| | | | | | | | | | SEQ ID |
| | | | | | | | | | NO: 258 |
| X | 32348501 | NM_004006.2:c.5353C > | NP_003997.1:p.Gln1785Ter | Dilated | G > A | nonsense | ABE | SEQ ID | SEQ ID |
| | | T | | cardiomy- | | | | NO: 61 | NO: 259; |
| | | | | opathy | | | | | SEQ ID |
| | | | | 3B; Dilated | | | | | NO: 260; |
| | | | | cardiomy- | | | | | SEQ ID |
| | | | | opathy 3B | | | | | NO: 261 |
| X | 32411811 | NM_004006.2:c.4174C > | NP_003997.1:p.Gln1392Ter | Duchenne | G > A | nonsense | ABE | SEQ ID | SEQ ID |
| | | T | | muscular | | | | NO: 62 | NO: 262; |
| | | | | dystrophy; | | | | | SEQ ID |
| | | | | Becker | | | | | NO: 263; |
| | | | | muscular | | | | | SEQ ID |
| | | | | dystrophy | | | | | NO: 264; |
| | | | | | | | | | SEQ ID |
| | | | | | | | | | NO: 265; |
| | | | | | | | | | SEQ ID |
| | | | | | | | | | NO: 266 |
| X | 31182861 | NM_004006.2:c.9851G > | NP_003997.1:p.Trp3284Ter | Dilated | C > T | nonsense | ABE | SEQ ID | SEQ ID |
| | | A | | cardiomy- | | | | NO: 63 | NO: 267; |
| | | | | opathy | | | | | SEQ ID |
| | | | | 3B; Dilated | | | | | NO: 268 |
| | | | | cardiomy- | | | | | |
| | | | | opathy 3B | | | | | |
| X | 32809577 | NM_004006.2:c.565C > | NP_003997.1:p.Gln189Ter | Duchenne | G > A | nonsense | ABE | SEQ ID | SEQ ID |
| | | T | | muscular | | | | NO: 64 | NO: 269 |
| | | | | dystrophy; | | | | | |
| | | | | Becker | | | | | |
| | | | | muscular | | | | | |
| | | | | dystrophy | | | | | |
| X | 32216981 | NM_004006.2:c.6373C > | NP_003997.1:p.Gln2125Ter | Duchenne | G > A | nonsense | ABE | SEQ ID | SEQ ID |
| | | T | | muscular | | | | NO: 65 | NO: 270 |
| | | | | dystrophy; | | | | | |
| | | | | DUCHENNE | | | | | |
| | | | | MUSCULAR | | | | | |
| | | | | DYSTROPHY; | | | | | |
| | | | | Dilated | | | | | |
| | | | | cardiomy- | | | | | |
| | | | | opathy | | | | | |
| | | | | 3B; Becker | | | | | |
| | | | | muscular | | | | | |
| | | | | dystrophy | | | | | |
| X | 32573812 | NM_004006.2:c.1637G > | NP_003997.1:p.Trp546Ter | Motor delay; | C > T | nonsense | ABE | SEQ ID | SEQ ID |
| | | A | | Muscle | | | | NO: 66 | NO: 271; |
| | | | | weakness; | | | | | SEQ ID |
| | | | | Muscle | | | | | NO: 272; |
| | | | | cramps; EMG | | | | | SEQ ID |
| | | | | abnormality; | | | | | NO: 273; |
| | | | | EMG; | | | | | SEQ ID |
| | | | | myopathic | | | | | NO: 274; |
| | | | | abnormalities; | | | | | SEQ ID |
| | | | | Calf muscle | | | | | NO: 275 |
| | | | | hypertrophy | | | | | |
| X | 31507314 | NM_004006.2:c.8357G > | NP_003997.1:p.Trp2786Ter | Duchenne | C > T | nonsense | ABE | SEQ ID | SEQ ID |
| | | A | | muscular | | | | NO: 67 | NO: 276; |
| | | | | dystrophy; | | | | | SEQ ID |
| | | | | Duchenne | | | | | NO: 277; |
| | | | | muscular | | | | | SEQ ID |
| | | | | dystrophy | | | | | NO: 278; |
| | | | | | | | | | SEQ ID |
| | | | | | | | | | NO: 279; |
| | | | | | | | | | SEQ ID |
| | | | | | | | | | NO: 280 |
| X | 32463458 | NM_004006.2:c.3413G > | NP_003997.1:p.Trp1138Ter | Becker | C > T | nonsense | ABE | SEQ ID | SEQ ID |
| | | A | | muscular | | | | NO: 68 | NO: 281; |
| | | | | dystrophy | | | | | SEQ ID |
| | | | | | | | | | NO: 282; |
| | | | | | | | | | SEQ ID |
| | | | | | | | | | NO: 283 |
| X | 31223071 | NM_004006.2:c.9337C > | NP_003997.1:p.Arg3113Ter | Dilated | G > A | nonsense | ABE | SEQ ID | SEQ ID |
| | | T | | cardiomy- | | | | NO: 69 | NO: 284; |
| | | | | opathy | | | | | SEQ ID |
| | | | | 3B; Becker | | | | | NO: 285 |
| | | | | muscular | | | | | |
| | | | | dystrophy; | | | | | |
| | | | | Duchenne | | | | | |
| | | | | muscular | | | | | |
| | | | | dystrophy; not | | | | | |
| | | | | provided; Not | | | | | |
| | | | | Provided | | | | | |
| X | 31929602 | NM_004006.2:c.6906G > | NP_003997.1:p.Trp2302Ter | Dilated | C > T | 5 prime | ABE | SEQ ID | SEQ ID |
| | | A | | cardiomy- | | UTR | | NO: 70 | NO: 286 |
| | | | | opathy | | variant | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32595765 | NM_004006.2:c.1594C > | NP_003997.1:p.Gln532Ter | Duchenne | G > A | nonsense | ABE | SEQ ID | SEQ ID |
| | | T | | muscular | | | | NO: 71 | NO: 287; |
| | | | | dystrophy | | | | | SEQ ID |
| | | | | | | | | | NO: 288; |
| | | | | | | | | | SEQ ID |
| | | | | | | | | | NO: 289; |
| | | | | | | | | | SEQ ID |
| | | | | | | | | | NO: 290 |
| X | 32573834 | NM_004006.2:c.1615C > | NP_003997.1:p.Arg539Ter | Dilated | G > A | nonsense | ABE | SEQ ID | SEQ ID |
| | | T | | cardiomy- | | | | NO: 72 | NO: 291; |
| | | | | opathy | | | | | SEQ ID |
| | | | | 3B; Becker | | | | | NO: 292; |
| | | | | muscular | | | | | SEQ ID |
| | | | | dystrophy; | | | | | NO: 293; |
| | | | | Duchenne | | | | | SEQ ID |
| | | | | muscular | | | | | NO: 294; |
| | | | | dystrophy | | | | | SEQ ID |
| | | | | | | | | | NO: 295 |
| X | 31679565 | NM_004006.2:c.7682G > | NP_003997.1:p.Trp2561Ter | Dilated | C > T | nonsense | ABE | SEQ ID | SEQ ID |
| | | A | | cardiomy- | | | | NO: 73 | NO: 296; |
| | | | | opathy | | | | | SEQ ID |
| | | | | 3B; Becker | | | | | NO: 297 |
| | | | | muscular | | | | | |
| | | | | dystrophy; | | | | | |
| | | | | Duchenne | | | | | |
| | | | | muscular | | | | | |
| | | | | dystrophy | | | | | |
| X | 31679564 | NM_004006.2:c.7683G > | NP_003997.1:p.Trp2561Ter | Dilated | C > T | nonsense | ABE | SEQ ID | SEQ ID |
| | | A | | cardiomy- | | | | NO: 74 | NO: 298 |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32485057 | NM_004006.2:c.2665C > | NP_003997.1:p.Arg889Ter | Duchenne | G > A | nonsense | ABE | SEQ ID | SEQ ID |
| | | T | | muscular | | | | NO: 75 | NO: 299; |
| | | | | dystrophy | | | | | SEQ ID |
| | | | | | | | | | NO: 300; |
| | | | | | | | | | SEQ ID |
| | | | | | | | | | NO: 301; |
| | | | | | | | | | SEQ ID |
| | | | | | | | | | NO: 302; |
| | | | | | | | | | SEQ ID |
| | | | | | | | | | NO: 303 |
| X | 32849781 | NM_004006.2:c.133C > | NP_003997.1:p.Gln45Ter | Duchenne | G > A | 5 prime | ABE | SEQ ID | SEQ ID |
| | | T | | muscular | | UTR | | NO: 76 | NO: 304; |
| | | | | dystrophy; | | variant | | | SEQ ID |
| | | | | Becker | | | | | NO: 305; |
| | | | | muscular | | | | | SEQ ID |
| | | | | dystrophy | | | | | NO: 306; |
| | | | | | | | | | SEQ ID |
| | | | | | | | | | NO: 307 |
| X | 32501767 | NM_004006.2:c.2368C > | NP_003997.1:p.Gln790Ter | Duchenne | G > A | nonsense | ABE | SEQ ID | SEQ ID |
| | | T | | muscular | | | | NO: 77 | NO: 308; |
| | | | | dystrophy; | | | | | SEQ ID |
| | | | | Duchenne | | | | | NO: 309; |
| | | | | muscular | | | | | SEQ ID |
| | | | | dystrophy | | | | | NO: 310 |
| X | 32545250 | NM_004006.2:c.2077C > | NP_003997.1:p.Gln693Ter | Duchenne | G > A | nonsense | ABE | SEQ ID | SEQ ID |
| | | T | | muscular | | | | NO: 78 | NO: 311; |
| | | | | dystrophy | | | | | SEQ ID |
| | | | | | | | | | NO: 312; |
| | | | | | | | | | SEQ ID |
| | | | | | | | | | NO: 313 |
| X | 31679429 | NM_004006.2:c.7818G > | NP_003997.1:p.Trp2606Ter | Dilated | C > T | nonsense | ABE | SEQ ID | SEQ ID |
| | | A | | cardiomy- | | | | NO: 79 | NO: 314; |
| | | | | opathy | | | | | SEQ ID |
| | | | | 3B; Dilated | | | | | NO: 315 |
| | | | | cardiomy- | | | | | |
| | | | | opathy 3B | | | | | |
| X | 31679492 | NM_004006.2:c.7755G > | NP_003997.1:p.Trp2585Ter | Duchenne | C > T | nonsense | ABE | SEQ ID | SEQ ID |
| | | A | | muscular | | | | NO: 80 | NO: 316; |
| | | | | dystrophy; | | | | | SEQ ID |
| | | | | Duchenne | | | | | NO: 317; |
| | | | | muscular | | | | | SEQ ID |
| | | | | dystrophy | | | | | NO: 318; |
| | | | | | | | | | SEQ ID |
| | | | | | | | | | NO: 319; |
| | | | | | | | | | SEQ ID |
| | | | | | | | | | NO: 320 |
| X | 31875331 | NM_004006.2:c.6955C > | NP_003997.1:p.Gln2319Ter | Duchenne | G > A | 5 prime | ABE | SEQ ID | SEQ ID |
| | | T | | muscular | | UTR | | NO: 81 | NO: 321 |
| | | | | dystrophy; | | variant | | | |
| | | | | DUCHENNE | | | | | |
| | | | | MUSCULAR | | | | | |
| | | | | DYSTROPHY | | | | | |
| X | 32342105 | NM_004006.2:c.5917C > | NP_003997.1:p.Gln1973Ter | Duchenne | G > A | nonsense | ABE | SEQ ID | SEQ ID |
| | | T | | muscular | | | | NO: 82 | NO: 322 |
| | | | | dystrophy; | | | | | |
| | | | | Duchenne | | | | | |
| | | | | muscular | | | | | |
| | | | | dystrophy | | | | | |
| X | 31774193 | NM_004006.2:c.7310 − | | Becker | C > T | splice | ABE | SEQ ID | SEQ ID |
| | | 1G > A | | muscular | | acceptor | | NO: 83 | NO: 323; |
| | | | | dystrophy; | | variant | | | SEQ ID |
| | | | | Duchenne | | | | | NO: 324 |
| | | | | muscular | | | | | |
| | | | | dystrophy | | | | | |
| X | 31177932 | NM_004006.2:c.10262C > | NP_003997.1:p.Ala3421Val | Becker | G > A | 500B | ABE | SEQ ID | SEQ ID |
| | | T | | muscular | | down- | | NO: 84 | NO: 325; |
| | | | | dystrophy; | | stream | | | SEQ ID |
| | | | | BECKER | | variant | | | NO: 326 |
| | | | | MUSCULAR | | | | | |
| | | | | DYSTROPHY; | | | | | |
| | | | | not specified | | | | | |
| X | 31658123 | NM_004006.2:c.7894C > | NP_003997.1:p.Gln2632Ter | Dilated | G > A | nonsense | ABE | SEQ ID | SEQ ID |
| | | T | | cardiomy- | | | | NO: 85 | NO: 327 |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32468683 | NM_004006.2:c.2977C > | NP_003997.1:p.Gln993Ter | Dilated | G > A | nonsense | ABE | SEQ ID | SEQ ID |
| | | T | | cardiomy- | | | | NO: 86 | NO: 328; |
| | | | | opathy | | | | | SEQ ID |
| | | | | 3B; Dilated | | | | | NO: 329 |
| | | | | cardiomy- | | | | | |
| | | | | opathy 3B | | | | | |
| X | 32699111 | NM_004006.2:c.831 + | | Duchenne | C > T | splice | ABE | SEQ ID | SEQ ID |
| | | 1G > A | | muscular | | donor | | NO: 87 | NO: 330; |
| | | | | dystrophy | | variant | | | SEQ ID |
| | | | | | | | | | NO: 331; |
| | | | | | | | | | SEQ ID |
| | | | | | | | | | NO: 332 |
| X | 32411772 | NM_004006.2:c.4213C > | NP_003997.1:p.Gln1405Ter | Duchenne | G > A | nonsense | ABE | SEQ ID | SEQ ID |
| | | T | | muscular | | | | NO: 88 | NO: 333; |
| | | | | dystrophy; | | | | | SEQ ID |
| | | | | DUCHENNE | | | | | NO: 334 |
| | | | | MUSCULAR | | | | | |
| | | | | DYSTROPHY | | | | | |
| X | 31178721 | NM_004006.2:c.10171C > | NP_003997.1:p.Arg3391Ter | Dilated | G > A | nonsense | ABE | SEQ ID | SEQ ID |
| | | T | | cardiomy- | | | | NO: 89 | NO: 335; |
| | | | | opathy | | | | | SEQ ID |
| | | | | 3B; Becker | | | | | NO: 336 |
| | | | | muscular | | | | | |
| | | | | dystrophy; | | | | | |
| | | | | Duchenne | | | | | |
| | | | | muscular | | | | | |
| | | | | dystrophy; not | | | | | |
| | | | | provided; Not | | | | | |
| | | | | Provided | | | | | |
| X | 31478983 | NM_004006.2:c.8668G > | NP_003997.1:p.Glu2890Lys | Dilated | C > T | missense | ABE | SEQ ID | SEQ ID |
| | | A | | cardiomy- | | variant | | NO: 90 | NO: 337; |
| | | | | opathy | | | | | SEQ ID |
| | | | | 3B; Dilated | | | | | NO: 338 |
| | | | | cardiomy- | | | | | |
| | | | | opathy 3B | | | | | |
| X | 31180369 | NM_004006.2:c.10086 + | | Dilated | C > T | splice | ABE | SEQ ID | SEQ ID |
| | | 1G > A | | cardiomy- | | donor | | NO: 91 | NO: 339; |
| | | | | opathy | | variant | | | SEQ ID |
| | | | | 3B; Becker | | | | | NO: 340; |
| | | | | muscular | | | | | SEQ ID |
| | | | | dystrophy; | | | | | NO: 341 |
| | | | | Duchenne | | | | | |
| | | | | muscular | | | | | |
| | | | | dystrophy | | | | | |
| X | 32364647 | NM_004006.2:c.5089C > | NP_003997.1:p.Gln1697Ter | Duchenne | G > A | nonsense | ABE | SEQ ID | SEQ ID |
| | | T | | muscular | | | | NO: 92 | NO: 342 |
| | | | | dystrophy; | | | | | |
| | | | | Becker | | | | | |
| | | | | muscular | | | | | |
| | | | | dystrophy | | | | | |
| X | 31496892 | NM_004006.2:c.8443C > | NP_003997.1:p.Gln2815Ter | Dilated | G > A | nonsense | ABE | SEQ ID | SEQ ID |
| | | T | | cardiomy- | | | | NO: 93 | NO: 343; |
| | | | | opathy 3B; | | | | | SEQ ID |
| | | | | Duchenne | | | | | NO: 344; |
| | | | | muscular | | | | | SEQ ID |
| | | | | dystrophy | | | | | NO: 345; |
| | | | | | | | | | SEQ ID |
| | | | | | | | | | NO: 346 |
| X | 32411868 | NM_004006.2:c.4117C > | NP_003997.1:p.Gln1373Ter | Dilated | G > A | nonsense | ABE | SEQ ID | SEQ ID |
| | | T | | cardiomy- | | | | NO: 94 | NO: 347; |
| | | | | opathy | | | | | SEQ ID |
| | | | | 3B; Becker | | | | | NO: 348 |
| | | | | muscular | | | | | |
| | | | | dystrophy; | | | | | |
| | | | | Duchenne | | | | | |
| | | | | muscular | | | | | |
| | | | | dystrophy | | | | | |
| X | 31819974 | NM_004006.2:c.7309 + | | Dilated | C > T | splice | ABE | SEQ ID | SEQ ID |
| | | 1G > A | | cardiomy- | | donor | | NO: 95 | NO: 349 |
| | | | | opathy | | variant | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 31444621 | NM_004006.2:c.8944C > | NP_003997.1:p.Arg2982Ter | Duchenne | G > A | nonsense | ABE | SEQ ID | SEQ ID |
| | | T | | muscular | | | | NO: 96 | NO: 350; |
| | | | | dystrophy; | | | | | SEQ ID |
| | | | | DUCHENNE | | | | | NO: 351; |
| | | | | MUSCULAR | | | | | SEQ ID |
| | | | | DYSTROPHY; | | | | | NO: 352; |
| | | | | Dilated | | | | | SEQ ID |
| | | | | cardiomy- | | | | | NO: 353 |
| | | | | opathy | | | | | |
| | | | | 3B; Becker | | | | | |
| | | | | muscular | | | | | |
| | | | | dystrophy | | | | | |
| X | 31627852 | NM_004006.2:c.8038C > | NP_003997.1:p.Arg2680Ter | Duchenne | G > A | nonsense | ABE | SEQ ID | SEQ ID |
| | | T | | muscular | | | | NO: 97 | NO: 354; |
| | | | | dystrophy; | | | | | SEQ ID |
| | | | | Becker | | | | | NO: 355; |
| | | | | muscular | | | | | SEQ ID |
| | | | | dystrophy | | | | | NO: 356; |
| | | | | | | | | | SEQ ID |
| | | | | | | | | | NO: 357 |
| X | 32501833 | NM_004006.2:c.2302C > | NP_003997.1:p.Arg768Ter | Duchenne | G > A | nonsense | ABE | SEQ ID | SEQ ID |
| | | T | | muscular | | | | NO: 98 | NO: 358; |
| | | | | dystrophy; | | | | | SEQ ID |
| | | | | DUCHENNE | | | | | NO: 359; |
| | | | | MUSCULAR | | | | | SEQ ID |
| | | | | DYSTROPHY; | | | | | NO: 360 |
| | | | | Dilated | | | | | |
| | | | | cardiomy- | | | | | |
| | | | | opathy 3B | | | | | |
| X | 32365049 | NM_004006.2:c.4996C > | NP_003997.1:p.Arg1666Ter | Dilated | G > A | nonsense | ABE | SEQ ID | SEQ ID |
| | | T | | cardiomy- | | | | NO: 99 | NO: 361; |
| | | | | opathy | | | | | SEQ ID |
| | | | | 3B; not | | | | | NO: 362 |
| | | | | provided; | | | | | |
| | | | | Duchenne | | | | | |
| | | | | muscular | | | | | |
| | | | | dystrophy | | | | | |
| X | 32345999 | NM_004006.2:c.5530C > | NP_003997.1:p.Arg1844Ter | Dilated | G > A | nonsense | ABE | SEQ ID | SEQ ID |
| | | T | | cardiomy- | | | | NO: 100 | NO: 363; |
| | | | | opathy | | | | | SEQ ID |
| | | | | 3B; Becker | | | | | NO: 364; |
| | | | | muscular | | | | | SEQ ID |
| | | | | dystrophy; | | | | | NO: 365; |
| | | | | Duchenne | | | | | SEQ ID |
| | | | | muscular | | | | | NO: 366 |
| | | | | dystrophy | | | | | |
| X | 32468573 | NM_004006.2:c.3087G > | NP_003997.1:p.Trp1029Ter | Dilated | C > T | nonsense | ABE | SEQ ID | SEQ ID |
| | | A | | cardiomy- | | | | NO: 101 | NO: 367; |
| | | | | opathy | | | | | SEQ ID |
| | | | | 3B; Dilated | | | | | NO: 368 |
| | | | | cardiomy- | | | | | |
| | | | | opathy 3B | | | | | |
| X | 32518098 | NM_004006.2:c.2202G > | NP_003997.1:p.Trp734Ter | Duchenne | C > T | nonsense | ABE | SEQ ID | SEQ ID |
| | | A | | muscular | | | | NO: 102 | NO: 369; |
| | | | | dystrophy | | | | | SEQ ID |
| | | | | | | | | | NO: 370; |
| | | | | | | | | | SEQ ID |
| | | | | | | | | | NO: 371; |
| | | | | | | | | | SEQ ID |
| | | | | | | | | | NO: 372 |
| X | 31820055 | NM_004006.2:c.7229G > | NP_003997.1:p.Trp2410Ter | Dilated | C > T | 5 prime | ABE | SEQ ID | SEQ ID |
| | | A | | cardiomy- | | UTR | | NO: 103 | NO: 373; |
| | | | | opathy | | variant | | | SEQ ID |
| | | | | 3B; Dilated | | | | | NO: 374 |
| | | | | cardiomy- | | | | | |
| | | | | opathy 3B | | | | | |
| X | 32650983 | NM_004006.2:c.961 − | | Dilated | G > A | intron | ABE | SEQ ID | SEQ ID |
| | | 5831C > T | | cardiomy- | | variant | | NO: 104 | NO: 375; |
| | | | | opathy 3B; | | | | | SEQ ID |
| | | | | Duchenne | | | | | NO: 376; |
| | | | | muscular | | | | | SEQ ID |
| | | | | dystrophy | | | | | NO: 377; |
| | | | | | | | | | SEQ ID |
| | | | | | | | | | NO: 378; |
| | | | | | | | | | SEQ ID |
| | | | | | | | | | NO: 379 |
| X | 31478129 | NM_004006.2:c.8914C > | NP_003997.1:p.Gln2972Ter | Duchenne | G > A | nonsense | ABE | SEQ ID | SEQ ID |
| | | T | | muscular | | | | NO: 105 | NO: 380; |
| | | | | dystrophy | | | | | SEQ ID |
| | | | | | | | | | NO: 381; |
| | | | | | | | | | SEQ ID |
| | | | | | | | | | NO: 382; |
| | | | | | | | | | SEQ ID |
| | | | | | | | | | NO: 383; |
| | | | | | | | | | SEQ ID |
| | | | | | | | | | NO: 384; |
| | | | | | | | | | SEQ ID |
| | | | | | | | | | NO: 385 |
| X | 32389605 | NM_004006.2:c.4414C > | NP_003997.1:p.Gln1472Ter | Duchenne | G > A | nonsense | ABE | SEQ ID | SEQ ID |
| | | T | | muscular | | | | NO: 106 | NO: 386; |
| | | | | dystrophy; | | | | | SEQ ID |
| | | | | DUCHENNE | | | | | NO: 387; |
| | | | | MUSCULAR | | | | | SEQ ID |
| | | | | DYSTROPHY | | | | | NO: 388; |
| | | | | | | | | | SEQ ID |
| | | | | | | | | | NO: 389 |
| X | 32595855 | NM_004006.2:c.1504C > | NP_003997.1:p.Gln502Ter | Duchenne | G > A | nonsense | ABE | SEQ ID | SEQ ID |
| | | T | | muscular | | | | NO: 107 | NO: 390 |
| | | | | dystrophy | | | | | |
| X | 32699219 | NM_004006.2:c.724C > | NP_003997.1:p.Gln242Ter | Duchenne | G > A | nonsense | ABE | SEQ ID | SEQ ID |
| | | T | | muscular | | | | NO: 108 | NO: 391; |
| | | | | dystrophy; | | | | | SEQ ID |
| | | | | DUCHENNE | | | | | NO: 392; |
| | | | | MUSCULAR | | | | | SEQ ID |
| | | | | DYSTROPHY | | | | | NO: 393; |
| | | | | | | | | | SEQ ID |
| | | | | | | | | | NO: 394 |
| X | 33211304 | NM_004006.2:c.9G > | NP_003997.1:p.Trp3Ter | Becker | C > T | nonsense | ABE | SEQ ID | SEQ ID |
| | | A | | muscular | | | | NO: 109 | NO: 395; |
| | | | | dystrophy; | | | | | SEQ ID |
| | | | | BECKER | | | | | NO: 396; |
| | | | | MUSCULAR | | | | | SEQ ID |
| | | | | DYSTROPHY; | | | | | NO: 397 |
| | | | | Dilated | | | | | |
| | | | | cardiomy- | | | | | |
| | | | | opathy 3B; | | | | | |
| | | | | Duchenne | | | | | |
| | | | | muscular | | | | | |
| | | | | dystrophy; not | | | | | |
| | | | | provided; Not | | | | | |
| | | | | Provided | | | | | |
| X | 32454833 | NM_004006.2:c.3433 − | | Dilated | C > T | splice | ABE | SEQ ID | SEQ ID |
| | | 1G > A | | cardiomy- | | acceptor | | NO: 110 | NO: 398; |
| | | | | opathy | | variant | | | SEQ ID |
| | | | | 3B; Dilated | | | | | NO: 399; |
| | | | | cardiomy- | | | | | SEQ ID |
| | | | | opathy 3B | | | | | NO: 400; |
| | | | | | | | | | SEQ ID |
| | | | | | | | | | NO: 401; |
| | | | | | | | | | SEQ ID |
| | | | | | | | | | NO: 402 |
| X | 32472252 | NM_004006.2:c.2861G > | NP_003997.1:p.Trp954Ter | Duchenne | C > T | nonsense | ABE | SEQ ID | SEQ ID |
| | | A | | muscular | | | | NO: 111 | NO: 403; |
| | | | | dystrophy | | | | | SEQ ID |
| | | | | | | | | | NO: 404; |
| | | | | | | | | | SEQ ID |
| | | | | | | | | | NO: 405 |
| X | 32389614 | NM_004006.2:c.4405C > | NP_003997.1:p.Gln1469Ter | Dilated | G > A | nonsense | ABE | SEQ ID | SEQ ID |
| | | T | | cardiomy- | | | | NO: 112 | NO: 406; |
| | | | | opathy | | | | | SEQ ID |
| | | | | 3B; Dilated | | | | | NO: 407; |
| | | | | cardiomy- | | | | | SEQ ID |
| | | | | opathy 3B | | | | | NO: 408 |
| X | 32438240 | NM_004006.2:c.4071 + | | Duchenne | C > T | splice | ABE | SEQ ID | SEQ ID |
| | | 1G > A | | muscular | | donor | | NO: 113 | NO: 409; |
| | | | | dystrophy | | variant | | | SEQ ID |
| | | | | | | | | | NO: 410; |
| | | | | | | | | | SEQ ID |
| | | | | | | | | | NO: 411; |
| | | | | | | | | | SEQ ID |
| | | | | | | | | | NO: 412; |
| | | | | | | | | | SEQ ID |
| | | | | | | | | | NO: 413; |
| | | | | | | | | | SEQ ID |
| | | | | | | | | | NO: 414 |
| X | 32491492 | NM_004006.2:c.2407C > | NP_003997.1:p.Gln803Ter | Duchenne | G > A | nonsense | ABE | SEQ ID | SEQ ID |
| | | T | | muscular | | | | NO: 114 | NO: 415 |
| | | | | dystrophy; | | | | | |
| | | | | Duchenne | | | | | |
| | | | | muscular | | | | | |
| | | | | dystrophy | | | | | |
| X | 31206581 | NM_004006.2:c.9649 + | | Duchenne | C > T | splice | ABE | SEQ ID | SEQ ID |
| | | 1G > A | | muscular | | donor | | NO: 115 | NO: 416; |
| | | | | dystrophy | | variant | | | SEQ ID |
| | | | | | | | | | NO: 417; |
| | | | | | | | | | SEQ ID |
| | | | | | | | | | NO: 418 |
| X | 32390175 | NM_004006.2:c.4240C > | NP_003997.1:p.Gln1414Ter | Duchenne | G > A | nonsense | ABE | SEQ ID | SEQ ID |
| | | T | | muscular | | | | NO: 116 | NO: 419; |
| | | | | dystrophy; | | | | | SEQ ID |
| | | | | not specified | | | | | NO: 420; |
| | | | | | | | | | SEQ ID |
| | | | | | | | | | NO: 421; |
| | | | | | | | | | SEQ ID |
| | | | | | | | | | NO: 422; |
| | | | | | | | | | SEQ ID |
| | | | | | | | | | NO: 423 |
| X | 32491387 | NM_004006.2:c.2512C > | NP_003997.1:p.Gln838Ter | Duchenne | G > A | nonsense | ABE | SEQ ID | SEQ ID |
| | | T | | muscular | | | | NO: 117 | NO: 424; |
| | | | | dystrophy; | | | | | SEQ ID |
| | | | | Becker | | | | | NO: 425; |
| | | | | muscular | | | | | SEQ ID |
| | | | | dystrophy | | | | | NO: 426; |
| | | | | | | | | | SEQ ID |
| | | | | | | | | | NO: 427 |
| X | 32342154 | NM.004006.2:c.5868G > | NP_003997.1:p.Trp1956Ter | Duchenne | C > T | nonsense | ABE | SEQ ID | SEQ ID |
| | | A | | muscular | | | | NO: 118 | NO: 428; |
| | | | | dystrophy; | | | | | SEQ ID |
| | | | | Becker | | | | | NO: 429; |
| | | | | muscular | | | | | SEQ ID |
| | | | | dystrophy | | | | | NO: 430; |
| | | | | | | | | | SEQ ID |
| | | | | | | | | | NO: 431 |
| X | 32816509 | NM_004006.2:c.489G > | NP_003997.1:p.Trp163Ter | Dilated | C > T | nonsense | ABE | SEQ ID | SEQ ID |
| | | A | | cardiomy- | | | | NO: 119 | NO: 432; |
| | | | | opathy | | | | | SEQ ID |
| | | | | 3B; Dilated | | | | | NO: 433 |
| | | | | cardiomy- | | | | | |
| | | | | opathy 3B | | | | | |
| X | 32454685 | NM_004006.2:c.3580C > | NP_003997.1:p.Gln1194Ter | Dilated | G > A | nonsense | ABE | SEQ ID | SEQ ID |
| | | T | | cardiomy- | | | | NO: 120 | NO: 434; |
| | | | | opathy | | | | | SEQ ID |
| | | | | 3B; Becker | | | | | NO: 435 |
| | | | | muscular | | | | | |
| | | | | dystrophy; | | | | | |
| | | | | Duchenne | | | | | |
| | | | | muscular | | | | | |
| | | | | dystrophy | | | | | |
| X | 32573529 | NM_004006.2:c.1812 + | | Becker | C > T | splice | ABE | SEQ ID | SEQ ID |
| | | 1G > A | | muscular | | donor | | NO: 121 | NO: 436 |
| | | | | dystrophy; | | variant | | | |
| | | | | Muscular | | | | | |
| | | | | dystrophy, | | | | | |
| | | | | Becker; | | | | | |
| | | | | Duchenne | | | | | |
| | | | | muscular | | | | | |
| | | | | dystrophy; not | | | | | |
| | | | | provided; Not | | | | | |
| | | | | Provided | | | | | |
| X | 31223046 | NM_004006.2:c.9361 + | | Dilated | C > T | splice | ABE | SEQ ID | SEQ ID |
| | | 1G > A | | cardiomy- | | donor | | NO: 122 | NO: 437 |
| | | | | opathy | | variant | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32545310 | NM_004006.2:c.2017C > | NP_003997.1:p.Gln673Ter | Duchenne | G > A | nonsense | ABE | SEQ ID | SEQ ID |
| | | T | | muscular | | | | NO: 123 | NO: 438; |
| | | | | dystrophy; | | | | | SEQ ID |
| | | | | DUCHENNE | | | | | NO: 439 |
| | | | | MUSCULAR | | | | | |
| | | | | DYSTROPHY | | | | | |
| X | 32343232 | NM_004006.2:c.5641C > | NP_003997.1:p.Gln1881Ter | Duchenne | G > A | nonsense | ABE | SEQ ID | SEQ ID |
| | | T | | muscular | | | | NO: 124 | NO: 440; |
| | | | | dystrophy; | | | | | SEQ ID |
| | | | | Duchenne | | | | | NO: 441; |
| | | | | muscular | | | | | SEQ ID |
| | | | | dystrophy | | | | | NO: 442; |
| | | | | | | | | | SEQ ID |
| | | | | | | | | | NO: 443 |
| X | 31679430 | NM_004006.2:c.7817G > | NP_003997.1:p.Trp2606Ter | Duchenne | C > T | nonsense | ABE | SEQ ID | SEQ ID |
| | | A | | muscular | | | | NO: 125 | NO: 444 |
| | | | | dystrophy; | | | | | |
| | | | | Duchenne | | | | | |
| | | | | muscular | | | | | |
| | | | | dystrophy | | | | | |
| X | 32809559 | NM_004006.2:c.583C > | NP_003997.1:p.Arg195Ter | Dilated | G > A | nonsense | ABE | SEQ ID | SEQ ID |
| | | T | | cardiomy- | | | | NO: 126 | NO: 445; |
| | | | | opathy | | | | | SEQ ID |
| | | | | 3B; Becker | | | | | NO: 446; |
| | | | | muscular | | | | | SEQ ID |
| | | | | dystrophy; | | | | | NO: 447; |
| | | | | Duchenne | | | | | SEQ ID |
| | | | | muscular | | | | | NO: 448 |
| | | | | dystrophy; not | | | | | |
| | | | | provided; Not | | | | | |
| | | | | Provided | | | | | |
| X | 32573786 | NM_004006.2:c.1663C > | NP 003997.1:p.Gln555Ter | Duchenne | G > A | nonsense | ABE | SEQ ID | SEQ ID |
| | | T | | muscular | | | | NO: 127 | NO: 449; |
| | | | | dystrophy; | | | | | SEQ ID |
| | | | | Duchenne | | | | | NO: 450; |
| | | | | muscular | | | | | SEQ ID |
| | | | | dystrophy | | | | | NO: 451; |
| | | | | | | | | | SEQ ID |
| | | | | | | | | | NO: 452 |
| X | 31507313 | NM_004006.2:c.8358G > | NP_003997.1:p.Trp2786Ter | Dilated | C > T | nonsense | ABE | SEQ ID | SEQ ID |
| | | A | | cardiomy- | | | | NO: 128 | NO: 453; |
| | | | | opathy | | | | | SEQ ID |
| | | | | 3B; Dilated | | | | | NO: 454; |
| | | | | cardiomy- | | | | | SEQ ID |
| | | | | opathy 3B | | | | | NO: 455; |
| | | | | | | | | | SEQ ID |
| | | | | | | | | | NO: 456; |
| | | | | | | | | | SEQ ID |
| | | | | | | | | | NO: 457; |
| | | | | | | | | | SEQ ID |
| | | | | | | | | | NO: 458 |
| X | 32491463 | NM_004006.2:c.2436G > | NP_003997.1:p.Trp812Ter | Dilated | C > T | nonsense | ABE | SEQ ID | SEQ ID |
| | | A | | cardiomy- | | | | NO: 129 | NO: 459; |
| | | | | opathy | | | | | SEQ ID |
| | | | | 3B; Dilated | | | | | NO: 460; |
| | | | | cardiomy- | | | | | SEQ ID |
| | | | | opathy 3B | | | | | NO: 461 |
| X | 31658118 | NM_004006.2:c.7899G > | NP_003997.1:p.Trp2633Ter | not specified | C > T | nonsense | ABE | SEQ ID | SEQ ID |
| | | A | | | | | | NO: 130 | NO: 462; |
| | | | | | | | | | SEQ ID |
| | | | | | | | | | NO: 463; |
| | | | | | | | | | SEQ ID |
| | | | | | | | | | NO: 464; |
| | | | | | | | | | SEQ ID |
| | | | | | | | | | NO: 465; |
| | | | | | | | | | SEQ ID |
| | | | | | | | | | NO: 466 |
| X | 32448495 | NM_004006.2:c.3747G > | NP_003997.1:p.Trp1249Ter | Duchenne | C > T | nonsense | ABE | SEQ ID | SEQ ID |
| | | A | | muscular | | | | NO: 131 | NO: 467; |
| | | | | dystrophy | | | | | SEQ ID |
| | | | | | | | | | NO: 468; |
| | | | | | | | | | SEQ ID |
| | | | | | | | | | NO: 469; |
| | | | | | | | | | SEQ ID |
| | | | | | | | | | NO: 470; |
| | | | | | | | | | SEQ ID |
| | | | | | | | | | NO: 471 |
| X | 31348571 | NM_004006.2:c.9148C > | NP_003997.1:p.Gln3050Ter | not provided; | G > A | nonsense | ABE | SEQ ID | SEQ ID |
| | | T | | Not Provided | | | | NO: 132 | NO: 472; |
| | | | | | | | | | SEQ ID |
| | | | | | | | | | NO: 473; |
| | | | | | | | | | SEQ ID |
| | | | | | | | | | NO: 474; |
| | | | | | | | | | SEQ ID |
| | | | | | | | | | NO: 475 |
| X | 32485072 | NM_004006.2:c.2650C > | NP_003997.1:p.Gln884Ter | Dilated | G > A | nonsense | ABE | SEQ ID | SEQ ID |
| | | T | | cardiomy- | | | | NO: 133 | NO: 476; |
| | | | | opathy | | | | | SEQ ID |
| | | | | 3B; Becker | | | | | NO: 477; |
| | | | | muscular | | | | | SEQ ID |
| | | | | dystrophy; | | | | | NO: 478; |
| | | | | Duchenne | | | | | SEQ ID |
| | | | | muscular | | | | | NO: 479 |
| | | | | dystrophy; | | | | | |
| | | | | not provided | | | | | |
| X | 32463444 | NM_004006.2:c.3427C > | NP_003997.1:p.Gln1143Ter | Duchenne | G > A | nonsense | ABE | SEQ ID | SEQ ID |
| | | T | | muscular | | | | NO: 134 | NO: 480; |
| | | | | dystrophy; | | | | | SEQ ID |
| | | | | Duchenne | | | | | NO: 481; |
| | | | | muscular | | | | | SEQ ID |
| | | | | dystrophy | | | | | NO: 482 |
| X | 32565742 | NM_004006.2:c.1952G > | NP 003997.1:p.Trp651Ter | Duchenne | C > T | nonsense | ABE | SEQ ID | SEQ ID |
| | | A | | muscular | | | | NO: 135 | NO: 483; |
| | | | | dystrophy; | | | | | SEQ ID |
| | | | | DUCHENNE | | | | | NO: 484; |
| | | | | MUSCULAR | | | | | SEQ ID |
| | | | | DYSTROPHY; | | | | | NO: 485; |
| | | | | Dilated | | | | | SEQ ID |
| | | | | cardiomy- | | | | | NO: 486 |
| | | | | opathy 3B | | | | | |
| X | 31147421 | NM_004006.2:c.10651C > | NP_003997.1p.Gln3551Ter | not provided; | G > A | nonsense | ABE | SEQ ID | |
| | | T | | Not Provided | | | | NO: 136 | |
| X | 31178700 | NM_004006.2:c.10192C > | NP_003997.1:p.Gln3398Ter | Dilated | G > A | nonsense | ABE | SEQ ID | |
| | | T | | cardiomy- | | | | NO: 137 | |
| | | | | opathy | | | | | |
| | | | | 3B; Dilated | | | | | |
| | | | | cardiomy- | | | | | |
| | | | | opathy 3B | | | | | |
| X | 31178751 | NM_004006.2:c.10141C > | NP_003997.1:p.Arg3381Ter | Duchenne | G > A | nonsense | ABE | SEQ ID | |
| | | T | | muscular | | | | NO: 138 | |
| | | | | dystrophy; | | | | | |
| | | | | DUCHENNE | | | | | |
| | | | | MUSCULAR | | | | | |
| | | | | DYSTROPHY; | | | | | |
| | | | | Dilated | | | | | |
| | | | | cardiomy- | | | | | |
| | | | | opathy | | | | | |
| | | | | 3B; not | | | | | |
| | | | | provided; Not | | | | | |
| | | | | Provided | | | | | |
| X | 31180437 | NM_004006.2:c.10019G > | NP_003997.1:p.Cys3340Tyr | Duchenne | C > T | missense | ABE | SEQ ID | |
| | | A | | muscular | | variant | | NO: 139 | |
| | | | | dystrophy, | | | | | |
| | | | | mental | | | | | |
| | | | | retardation, | | | | | |
| | | | | and absence | | | | | |
| | | | | of erg | | | | | |
| | | | | b-wave; | | | | | |
| | | | | DUCHENNE | | | | | |
| | | | | MUSCULAR | | | | | |
| | | | | DYSTROPHY, | | | | | |
| | | | | MENTAL | | | | | |
| | | | | RETARDA- | | | | | |
| | | | | TION, AND | | | | | |
| | | | | ABSENCE OF | | | | | |
| | | | | ERG B-WAVE | | | | | |
| X | 31182784 | NM_004006.2:c.9928C > | NP_003997.1:p.Gln3310Ter | Duchenne | G > A | nonsense | ABE | SEQ ID | |
| | | T | | muscular | | | | NO: 140 | |
| | | | | dystrophy | | | | | |
| X | 31209497 | NM_004006.2:c.9563 + | | Duchenne | C > T | splice | ABE | SEQ ID | |
| | | 1G > A | | muscular | | donor | | NO: 141 | |
| | | | | dystrophy | | variant | | | |
| X | 31223062 | NM_004006.2:c.9346C > | NP_003997.1:p.Gln3116Ter | Dilated | G > A | nonsense | ABE | SEQ ID | |
| | | T | | cardiomy- | | | | NO: 142 | |
| | | | | opathy | | | | | |
| | | | | 3B; Dilated | | | | | |
| | | | | cardiomy- | | | | | |
| | | | | opathy 3B | | | | | |
| X | 31478330 | NM_004006.2:c.8713C > | NP_003997.1:p.Arg2905Ter | Duchenne | G > A | nonsense | ABE | SEQ ID | |
| | | T | | muscular | | | | NO: 143 | |
| | | | | dystrophy; | | | | | |
| | | | | DUCHENNE | | | | | |
| | | | | MUSCULAR | | | | | |
| | | | | DYSTROPHY; | | | | | |
| | | | | Dilated | | | | | |
| | | | | cardiomy- | | | | | |
| | | | | opathy | | | | | |
| | | | | 3B; Becker | | | | | |
| | | | | muscular | | | | | |
| | | | | dystrophy | | | | | |
| X | 31478995 | NM_004006.2:c.8656C > | NP_003997.1:p.Gln2886Ter | Dilated | G > A | nonsense | ABE | SEQ ID | |
| | | T | | cardiomy- | | | | NO: 144 | |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 31627681 | NM_004006.2:c.82.09C > | NP_003997.1:p.Gln2737Ter | Duchenne | G > A | nonsense | ABE | SEQ ID | |
| | | T | | muscular | | | | NO: 145 | |
| | | | | dystrophy | | | | | |
| X | 31679575 | NM_004006.2:c.7672C > | NP_003997.1:p.Gln2558Ter | not provided; | G > A | nonsense | ABE | SEQ ID | |
| | | T | | Duchenne | | | | NO: 146 | |
| | | | | muscular | | | | | |
| | | | | dystrophy | | | | | |
| X | 31836729 | NM_004006.2:c.7189C > | NP_003997.1:p.Gln2.397Ter | Dilated | G > A | 5 prime | ABE | SEQ ID | |
| | | T | | cardiomy- | | UTR | | NO: 147 | |
| | | | | opathy | | variant | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 31929718 | NM_004006.2:c.6790C > | NP_003997.1:p.Gln2264Ter | Duchenne | G > A | 5 prime | ABE | SEQ ID | |
| | | T | | muscular | | UTR | | NO: 148 | |
| | | | | dystrophy; | | variant | | | |
| | | | | DUCHENNE | | | | | |
| | | | | MUSCULAR | | | | | |
| | | | | DYSTROPHY | | | | | |
| X | 32287536 | NM_004006.2:c.6283C > | NP_003997.1:p.Arg2095Ter | Dilated | G > A | nonsense | ABE | SEQ ID | |
| | | T | | cardiomy- | | | | NO: 149 | |
| | | | | opathy | | | | | |
| | | | | 3B; Dilated | | | | | |
| | | | | cardiomy- | | | | | |
| | | | | opathy 3B | | | | | |
| X | 32287596 | NM_004006.2:c.6223C > | NP_003997.1:p.Gln2075Ter | Duchenne | G > A | nonsense | ABE | SEQ ID | |
| | | T | | muscular | | | | NO: 150 | |
| | | | | dystrophy | | | | | |
| X | 32287680 | NM_004006.2:c.6139C > | NP_003997.1:p.Gln2047Ter | Elevated | G > A | nonsense | ABE | SEQ ID | |
| | | T | | serum | | | | NO: 151 | |
| | | | | creatine | | | | | |
| | | | | phosphokinase | | | | | |
| X | 32287702 | NM_004006.2:c.6118 − | | Duchenne | C > T | splice | ABE | SEQ ID | |
| | | 1G > A | | muscular | | acceptor | | NO: 152 | |
| | | | | dystrophy | | variant | | | |
| X | 32342171 | NM_004006.2:c.5851C > | NP_003997.1:p.Gln1951Ter | not provided; | G > A | nonsense | ABE | SEQ ID | |
| | | T | | not provided | | | | NO: 153 | |
| X | 32345975 | NM_004006.2:c.5554C > | NP_003997.1:p.Gln1852Ter | Dilated | G > A | nonsense | ABE | SEQ ID | |
| | | T | | cardiomy- | | | | NO: 154 | |
| | | | | opathy | | | | | |
| | | | | 3B; Dilated | | | | | |
| | | | | cardiomy- | | | | | |
| | | | | opathy 3B | | | | | |
| X | 32345978 | NM_004006.2:c.5551C > | NP_003997.1:p.Gln1851Ter | Duchenne | G > A | nonsense | ABE | SEQ ID | |
| | | T | | muscular | | | | NO: 155 | |
| | | | | dystrophy; | | | | | |
| | | | | DUCHENNE | | | | | |
| | | | | MUSCULAR | | | | | |
| | | | | DYSTROPHY; | | | | | |
| | | | | Duchenne | | | | | |
| | | | | muscular | | | | | |
| | | | | dystrophy | | | | | |
| X | 32346023 | NM_004006.2:c.5506C > | NP_003997.1:p.Gln1836Ter | Duchenne | G > A | nonsense | ABE | SEQ ID | |
| | | T | | muscular | | | | NO: 156 | |
| | | | | dystrophy | | | | | |
| X | 32364602 | NM_004006.2:c.5134C > | NP_003997.1:p.Gln1712Ter | Dilated | G > A | nonsense | ABE | SEQ ID | |
| | | T | | cardiomy- | | | | NO: 157 | |
| | | | | opathy | | | | | |
| | | | | 3B; Dilated | | | | | |
| | | | | cardiomy- | | | | | |
| | | | | opathy 3B | | | | | |
| X | 32364605 | NM_004006.2:c.5131C > | XP_003997.1:p.Gln1711Ter | Duchenne | G > A | nonsense | ABE | SEQ ID | |
| | | T | | muscular | | | | NO: 158 | |
| | | | | dystrophy; | | | | | |
| | | | | Duchenne | | | | | |
| | | | | muscular | | | | | |
| | | | | dystrophy | | | | | |
| X | 32365175 | NM_004006.2:c.4870C > | NP_003997.1:p.Gln1624Ter | Duchenne | G > A | nonsense | ABE | SEQ ID | |
| | | T | | muscular | | | | NO: 159 | |
| | | | | dystrophy | | | | | |
| X | 32389536 | NM_004006.2:c.4483C > | NP_003997.1:p.Gln1495Ter | Duchenne | G > A | nonsense | ABE | SEQ ID | |
| | | T | | muscular | | | | NO: 160 | |
| | | | | dystrophy | | | | | |
| X | 32389644 | NM_004006.2:c.4375C > | NP_003997.1:p.Arg1459Ter | Dilated | G > A | nonsense | ABE | SEQ ID | |
| | | T | | cardiomy- | | | | NO: 161 | |
| | | | | opathy 3B; | | | | | |
| | | | | Duchenne | | | | | |
| | | | | muscular | | | | | |
| | | | | dystrophy | | | | | |
| X | 32454778 | NM_004006.2:c.3487C > | NP_003997.1:p.Gln1163Ter | not provided; | G > A | nonsense | ABE | SEQ ID | |
| | | T | | Not Provided | | | | NO: 162 | |
| X | 32463438 | NM_004006.2:c.3432 + | | Dilated | C > T | splice | ABE | SEQ ID | |
| | | 1G > A | | cardiomy- | | donor | | NO: 163 | |
| | | | | opathy | | variant | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32464585 | NM_004006.2:c.3276 + | | Dilated | C > T | splice | ABE | SEQ ID | |
| | | 1G > A | | cardiomy- | | donor | | NO: 164 | |
| | | | | opathy | | variant | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32464603 | NM_004006.2:c.3259C > | XP_003997.1:p.Gln1087Ter | not provided; | G > A | nonsense | ABE | SEQ ID | |
| | | T | | Not Provided | | | | NO: 165 | |
| X | 32468509 | NM_004006.2:c.3151C > | NP_003997.1:p.Arg1051Ter | Dilated | G > A | nonsense | ABE | SEQ ID | |
| | | T | | cardiomy- | | | | NO: 166 | |
| | | | | opathy 3B; | | | | | |
| | | | | Duchenne | | | | | |
| | | | | muscular | | | | | |
| | | | | dystrophy; | | | | | |
| | | | | Becker | | | | | |
| | | | | muscular | | | | | |
| | | | | dystrophy | | | | | |
| X | 32468704 | NM_004006.2:c.2956C > | XP_003997.1:p.Gln986Ter | Duchenne | G > A | nonsense | ABE | SEQ ID | |
| | | T | | muscular | | | | NO: 167 | |
| | | | | dystrophy; | | | | | |
| | | | | Becker | | | | | |
| | | | | muscular | | | | | |
| | | | | dystrophy; | | | | | |
| | | | | Dilated | | | | | |
| | | | | cardiomy- | | | | | |
| | | | | opathy | | | | | |
| | | | | 3B; not | | | | | |
| | | | | provided; Not | | | | | |
| | | | | Provided | | | | | |
| X | 32472310 | NM_004006.2:c.2804 − | | Dilated | C > T | splice | ABE | SEQ ID | |
| | | 1G > A | | cardiomy- | | acceptor | | NO: 168 | |
| | | | | opathy | | variant | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32484925 | NM_004006.2:c.2797C > | NP_003997.1:p.Gln933Ter | Duchenne | G > A | nonsense | ABE | SEQ ID | |
| | | T | | muscular | | | | NO: 169 | |
| | | | | dystrophy; | | | | | |
| | | | | Duchenne | | | | | |
| | | | | muscular | | | | | |
| | | | | dystrophy | | | | | |
| X | 32484964 | NM_004006.2:c.2758C > | NP_003997.1:p.Gln920Ter | Dilated | G > A | nonsense | ABE | SEQ ID | |
| | | T | | cardiomy- | | | | NO: 170 | |
| | | | | opathy | | | | | |
| | | | | 3B; Dilated | | | | | |
| | | | | cardiomy- | | | | | |
| | | | | opathy 3B | | | | | |
| X | 32491276 | NM_004006.2:c.2622 + | | Dilated | C > T | splice | ABE | SEQ ID | |
| | | 1G > A | | cardiomy- | | donor | | NO: 171 | |
| | | | | opathy | | variant | | | |
| | | | | 3B; Dilated | | | | | |
| | | | | cardiomy- | | | | | |
| | | | | opathy 3B | | | | | |
| X | 32491344 | NM_004006.2:c.2555G > | NP_003997.1:p.Trp852Ter | not provided; | C > T | nonsense | ABE | SEQ ID | |
| | | A | | Not Provided | | | | NO: 172 | |
| X | 32491480 | NM_004006.2:c.2419C > | NP_003997.1:p.Gln807Ter | Dilated | G > A | nonsense | ABE | SEQ ID | |
| | | T | | cardiomy- | | | | NO: 173 | |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32501803 | NM_004006.2:c.2332C > | NP_003997.1:p.Gln778Ter | Dilated | G > A | nonsense | ABE | SEQ ID | |
| | | T | | cardiomy- | | | | NO: 174 | |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32545158 | NM_004006.2:c.2168 + | | not provided; | C > T | splice | ABE | SEQ ID | |
| | | 1G > A | | Not Provided; | | donor | | NO: 175 | |
| | | | | Duchenne | | variant | | | |
| | | | | muscular | | | | | |
| | | | | dystrophy | | | | | |
| X | 32545190 | NM_004006.2:c.2137C > | NP_003997.1:p.Gln713Ter | Dilated | G > A | nonsense | ABE | SEQ ID | |
| | | T | | cardiomy- | | | | NO: 176 | |
| | | | | opathy | | | | | |
| | | | | 3B; Dilated | | | | | |
| | | | | cardiomy- | | | | | |
| | | | | opathy 3B | | | | | |
| X | 32565704 | NM_004006.2:c.1990C > | NP_003997.1:p.Gln664Ter | Dilated | G > A | nonsense | ABE | SEQ ID | |
| | | T | | cardiomy- | | | | NO: 177 | |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32565782 | NM_004006.2:c.1912C > | NP_003997.1:p.Gln638Ter | Duchenne | G > A | nonsense | ABE | SEQ ID | |
| | | T | | muscular | | | | NO: 178 | |
| | | | | dystrophy; | | | | | |
| | | | | Becker | | | | | |
| | | | | muscular | | | | | |
| | | | | dystrophy | | | | | |
| X | 32573796 | NM_004006.2:c.1653G > | NP_003997.1:p.Trp551Ter | Duchenne | C > T | nonsense | ABE | SEQ ID | |
| | | A | | muscular | | | | NO: 179 | |
| | | | | dystrophy; | | | | | |
| | | | | not provided | | | | | |
| X | 32595870 | NM_004006.2:c.1489C > | NP_003997.1:p.Gln497Ter | Duchenne | G > A | nonsense | ABE | SEQ ID | |
| | | T | | muscular | | | | NO: 180 | |
| | | | | dystrophy; | | | | | |
| | | | | DUCHENNE | | | | | |
| | | | | MUSCULAR | | | | | |
| | | | | DYSTROPHY; | | | | | |
| | | | | Duchenne | | | | | |
| | | | | muscular | | | | | |
| | | | | dystrophy | | | | | |
| X | 32614320 | NM_004006.2:c.1465C > | NP_003997.1:p.Gln489Ter | Dilated | G > A | nonsense | ABE | SEQ ID | |
| | | T | | cardiomy- | | | | NO: 181 | |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32614397 | NM_004006.2:c.1388G > | NP_003997.1:p.Trp463Ter | Duchenne | C > T | nonsense | ABE | SEQ ID | |
| | | A | | muscular | | | | NO: 182 | |
| | | | | dystrophy; | | | | | |
| | | | | Duchenne | | | | | |
| | | | | muscular | | | | | |
| | | | | dystrophy | | | | | |
| X | 32644131 | NM_004006.2:c.1331 + | | Duchenne | C > T | splice | ABE | SEQ ID | |
| | | 1G > A | | muscular | | donor | | NO: 183 | |
| | | | | dystrophy; | | variant | | | |
| | | | | Duchenne | | | | | |
| | | | | muscular | | | | | |
| | | | | dystrophy | | | | | |
| X | 32644139 | NM_004006.2:c.1324C > | NP_003997.1:p.Gln442Ter | Duchenne | G > A | nonsense | ABE | SEQ ID | |
| | | T | | muscular | | | | NO: 184 | |
| | | | | dystrophy; | | | | | |
| | | | | Duchenne | | | | | |
| | | | | muscular | | | | | |
| | | | | dystrophy | | | | | |
| X | 32644202 | NM_004006.2:c.1261C > | NP_003997.1:p.Gln421Ter | Dilated | G > A | nonsense | ABE | SEQ ID | |
| | | T | | cardiomy- | | | | NO: 185 | |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32645020 | NM_004006.2:c.1093C > | NP_003997.1:p.Gln365Ter | Duchenne | G > A | nonsense | ABE | SEQ ID | |
| | | T | | muscular | | | | NO: 186 | |
| | | | | dystrophy; | | | | | |
| | | | | Becker | | | | | |
| | | | | muscular | | | | | |
| | | | | dystrophy | | | | | |
| | 32809492 | NM_004006.2:c.649 + | | Dilated | C > T | splice | ABE | SEQ ID | |
| | | 1G > A | | cardiomy- | | donor | | NO: 187 | |
| | | | | opathy | | variant | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 32816641 | NM.004006.2:c.358 − | | Becker | C > T | splice | ABE | SEQ ID | |
| | | 1G > A | | muscular | | acceptor | | NO: 188 | |
| | | | | dystrophy | | variant | | | |
| X | 32823297 | NM_004006.2:c.355C > | NP_003997.1:p.Gln119Ter | Duchenne | G > A | 5 prime | ABE | SEQ ID | |
| | | T | | muscular | | UTR | | NO: 189 | |
| | | | | dystrophy; | | variant | | | |
| | | | | Duchenne | | | | | |
| | | | | muscular | | | | | |
| | | | | dystrophy | | | | | |
| X | 32849727 | NM_004006.2:c.186 + | | Duchenne | C > T | splice | ABE | SEQ ID | |
| | | 1G > A | | muscular | | donor | | NO: 190 | |
| | | | | dystrophy | | variant | | | |
| X | 32849736 | NM_004006.2:c.178C > | NP_003997.1:p.Gln60Ter | Duchenne | G > A | 5 prime | ABE | SEQ ID | |
| | | T | | muscular | | UTR | | NO: 191 | |
| | | | | dystrophy; | | variant | | | |
| | | | | DUCHENNE | | | | | |
| | | | | MUSCULAR | | | | | |
| | | | | DYSTROPHY; | | | | | |
| | | | | not provided | | | | | |
| X | 33020138 | NM_004006.2:c.93 + | | Becker | C > T | splice | ABE | SEQ ID | |
| | | 1G > A | | muscular | | donor | | NO: 192 | |
| | | | | dystrophy; | | variant | | | |
| | | | | Duchenne | | | | | |
| | | | | muscular | | | | | |
| | | | | dystrophy | | | | | |
| X | 32595756 | NM_004006.2:c.1602 + | | not provided; | C > T | splice | ABE | SEQ ID | SEQ ID |
| | | 1G > A | | Not Provided | | donor | | NO: 193 | NO: 487 |
| | | | | | | variant | | | |
| X | 31206668 | NM_004006.2:c.9564 − | | Dilated | C > T | splice | ABE | SEQ ID | SEQ ID |
| | | 1G > A | | cardiomy- | | acceptor | | NO: 194 | NO: 488; |
| | | | | opathy | | variant | | | SEQ ID |
| | | | | 3B; Becker | | | | | NO: 489; |
| | | | | muscular | | | | | SEQ ID |
| | | | | dystrophy; | | | | | NO: 490; |
| | | | | Duchenne | | | | | SEQ ID |
| | | | | muscular | | | | | NO: 491 |
| | | | | dystrophy | | | | | |
| X | 32217062 | NM_004006.2:c.6292C > | NP_003997.1:p.Arg2098Ter | Duchenne | G > A | nonsense | ABE | SEQ ID | SEQ ID |
| | | T | | muscular | | | | NO: 195 | NO: 492 |
| | | | | dystrophy; | | | | | |
| | | | | DUCHENNE | | | | | |
| | | | | MUSCULAR | | | | | |
| | | | | DYSTROPHY; | | | | | |
| | | | | Dilated | | | | | |
| | | | | cardiomy- | | | | | |
| | | | | opathy 3B | | | | | |
| X | 32644314 | NM_004006.2:c.1150 − | | Duchenne | C > T | splice | ABE | SEQ ID | SEQ ID |
| | | 1G > A | | muscular | | acceptor | | NO: 196 | NO: 493; |
| | | | | dystrophy; not | | variant | | | SEQ ID |
| | | | | provided; Not | | | | | NO: 494; |
| | | | | Provided | | | | | SEQ ID |
| | | | | | | | | | NO: 495 |
| X | 32849821 | NM_004006.2:c.94 − | | Duchenne | C > T | splice | ABE | SEQ ID | SEQ ID |
| | | 1G > A | | muscular | | acceptor | | NO: 197 | NO: 496 |
| | | | | dystrophy; | | variant | | | |
| | | | | Duchenne | | | | | |
| | | | | muscular | | | | | |
| | | | | dystrophy | | | | | |
| X | 31496871 | NM_004006.2:c.8464C > | NP_003997.1:p.Gln2822Ter | Dilated | G > A | nonsense | ABE | SEQ ID | SEQ ID |
| | | T | | cardiomy- | | | | NO: 198 | NO: 497; |
| | | | | opathy | | | | | SEQ ID |
| | | | | 3B; Dilated | | | | | NO: 498; |
| | | | | cardiomy- | | | | | SEQ ID |
| | | | | opathy 3B | | | | | NO: 499 |
| X | 32380626 | NM_004006.2:c.4729C > | NP_003997.1:p.Arg1577Ter | Duchenne | G > A | nonsense | ABE | SEQ ID | SEQ ID |
| | | T | | muscular | | | | NO: 199 | NO: 500; |
| | | | | dystrophy; not | | | | | SEQ ID |
| | | | | provided; Not | | | | | NO: 501 |
| | | | | Provided | | | | | |
| X | 32645052 | NM_004006.2:c.1061G > | NP_003997.1:p.Trp354Ter | Duchenne | C > T | nonsense | ABE | SEQ ID | SEQ ID |
| | | A | | muscular | | | | NO: 200 | NO: 502; |
| | | | | dystrophy | | | | | SEQ ID |
| | | | | | | | | | NO: 503; |
| | | | | | | | | | SEQ ID |
| | | | | | | | | | NO: 504; |
| | | | | | | | | | SEQ ID |
| | | | | | | | | | NO: 505 |
| X | 32491414 | NM_004006.2:c.2485C > | NP_003997.1:p.Gln829Ter | not specified | G > A | nonsense | ABE | SEQ ID | SEQ ID |
| | | T | | | | | | NO: 201 | NO: 506; |
| | | | | | | | | | SEQ ID |
| | | | | | | | | | NO: 507; |
| | | | | | | | | | SEQ ID |
| | | | | | | | | | NO: 508; |
| | | | | | | | | | SEQ ID |
| | | | | | | | | | NO: 509; |
| | | | | | | | | | SEQ ID |
| | | | | | | | | | NO: 510 |
| X | 31169519 | NM_004020.3:c.2843 + | NP_003997.1:p.Gln3493Ter | Duchenne | G > A | intron | ABE | SEQ ID | SEQ ID |
| | | 9150C > T | | muscular | | variant | | NO: 202 | NO: 511; |
| | | | | dystrophy; | | | | | SEQ ID |
| | | | | Becker | | | | | NO: 512; |
| | | | | muscular | | | | | SEQ ID |
| | | | | dystrophy | | | | | NO: 513; |
| | | | | | | | | | SEQ ID |
| | | | | | | | | | NO: 514 |
| X | 32364704 | NM_004006.2:c.5032C > | NP_003997.1:p.Gln1678Ter | Duchenne | G > A | nonsense | ABE | SEQ ID | SEQ ID |
| | | T | | muscular | | | | NO: 203 | NO: 515; |
| | | | | dystrophy | | | | | SEQ ID |
| | | | | | | | | | NO: 516 |
| X | 32545295 | NM_004006.2:c.2032C > | NP_003997.1:p.Gln678Ter | Dilated | G > A | nonsense | ABE | SEQ ID | SEQ ID |
| | | T | | cardiomy- | | | | NO: 204 | NO: 517; |
| | | | | opathy | | | | | SEQ ID |
| | | | | 3B; Becker | | | | | NO: 518; |
| | | | | muscular | | | | | SEQ ID |
| | | | | dystrophy; | | | | | NO: 519; |
| | | | | Duchenne | | | | | SEQ ID |
| | | | | muscular | | | | | NO: 520; |
| | | | | dystrophy | | | | | SEQ ID |
| | | | | | | | | | NO: 521; |
| | | | | | | | | | SEQ ID |
| | | | | | | | | | NO: 522; |
| | | | | | | | | | SEQ ID |
| | | | | | | | | | NO: 523 |
| X | 31178784 | NM.004006.2:c.10108C > | NP_003997.1:p.Arg3370Ter | Duchenne | G > A | nonsense | ABE | SEQ ID | SEQ ID |
| | | T | | muscular | | | | NO: 205 | NO: 524; |
| | | | | dystrophy; | | | | | SEQ ID |
| | | | | DUCHENNE | | | | | NO: 525; |
| | | | | MUSCULAR | | | | | SEQ ID |
| | | | | DYSTROPHY; | | | | | NO: 526; |
| | | | | Dilated | | | | | SEQ ID |
| | | | | cardiomy- | | | | | NO: 527 |
| | | | | opathy | | | | | |
| | | | | 3B; Becker | | | | | |
| | | | | muscular | | | | | |
| | | | | dystrophy | | | | | |
| X | 32342123 | NM_004006.2:c.5899C > | NP_003997.1:p.Arg1967Ter | Duchenne | G > A | nonsense | ABE | SEQ ID | SEQ ID |
| | | T | | muscular | | | | NO: 206 | NO: 528; |
| | | | | dystrophy; | | | | | SEQ ID |
| | | | | DUCHENNE | | | | | NO: 529 |
| | | | | MUSCULAR | | | | | |
| | | | | DYSTROPHY; | | | | | |
| | | | | Dilated | | | | | |
| | | | | cardiomy- | | | | | |
| | | | | opathy | | | | | |
| | | | | 3B; Becker | | | | | |
| | | | | muscular | | | | | |
| | | | | dystrophy | | | | | |
| X | 31729736 | NM_004006.2:c.7555G > | NP_003997.1:p.Asp2519Asn | not provided; | C > T | missense | ABE | SEQ ID | SEQ ID |
| | | A | | Not Provided; | | variant | | NO: 207 | NO: 530; |
| | | | | Duchenne | | | | | SEQ ID |
| | | | | muscular | | | | | NO: 531; |
| | | | | dystrophy | | | | | SEQ ID |
| | | | | | | | | | NO: 532 |
| X | 33174335 | NM_004006.2:c.31 + | | Duchenne | C > T | intron | ABE | SEQ ID | SEQ ID |
| | | 36947G > A | | muscular | | variant | | NO: 208 | NO: 533; |
| | | | | dystrophy; | | | | | SEQ ID |
| | | | | Becker | | | | | NO: 534 |
| | | | | muscular | | | | | |
| | | | | dystrophy; | | | | | |
| | | | | Dilated | | | | | |
| | | | | cardiomy- | | | | | |
| | | | | opathy 3B | | | | | |
| X | 32472247 | NM_004006.2:c.2866C > | NP_003997.1:p.Gln956Ter | Dilated | G > A | nonsense | ABE | SEQ ID | SEQ ID |
| | | T | | cardiomy- | | | | NO: 209 | NO: 535; |
| | | | | opathy | | | | | SEQ ID |
| | | | | 3B; Dilated | | | | | NO: 536; |
| | | | | cardiomy- | | | | | SEQ ID |
| | | | | opathy 3B | | | | | NO: 537; |
| | | | | | | | | | SEQ ID |
| | | | | | | | | | NO: 538 |
| X | 32389496 | NM_004006.2:c.4518 + | | Dilated | C > T | intron | ABE | SEQ ID | SEQ ID |
| | | 5G > A | | cardiomy- | | variant | | NO: 210 | NO: 539; |
| | | | | opathy 3B; | | | | | SEQ ID |
| | | | | Duchenne | | | | | NO: 540; |
| | | | | muscular | | | | | SEQ ID |
| | | | | dystrophy | | | | | NO: 541 |
| X | 32438372 | NM_004006.2:c.3940C > | NP_003997.1:p.Arg1314Ter | Becker | G > A | nonsense | ABE | SEQ ID | SEQ ID |
| | | T | | muscular | | | | NO: 211 | NO: 542; |
| | | | | dystrophy; | | | | | SEQ ID |
| | | | | BECKER | | | | | NO: 543 |
| | | | | MUSCULAR | | | | | |
| | | | | DYSTROPHY; | | | | | |
| | | | | not provided; | | | | | |
| | | | | Not Provided; | | | | | |
| | | | | Duchenne | | | | | |
| | | | | muscular | | | | | |
| | | | | dystrophy | | | | | |
| X | 31206663 | NM_004006.2:c.9568C > | NP_003997.1:p.Arg3190Ter | Duchenne | G > A | nonsense | ABE | SEQ ID | SEQ ID |
| | | T | | muscular | | | | NO: 212 | NO: 544; |
| | | | | dystrophy; | | | | | SEQ ID |
| | | | | DUCHENNE | | | | | NO: 545; |
| | | | | MUSCULAR | | | | | SEQ ID |
| | | | | DYSTROPHY; | | | | | NO: 546; |
| | | | | Dilated | | | | | SEQ ID |
| | | | | cardiomy- | | | | | NO: 547 |
| | | | | opathy | | | | | |
| | | | | 3B; Becker | | | | | |
| | | | | muscular | | | | | |
| | | | | dystrophy | | | | | |
| X | 31729634 | NM_004006.2:c.7657C > | NP_003997.1:p.Arg2553Ter | Dilated | G > A | nonsense | ABE | SEQ ID | SEQ ID |
| | | T | | cardiomy- | | | | NO: 213 | NO: 548 |
| | | | | opathy | | | | | |
| | | | | 3B; Becker | | | | | |
| | | | | muscular | | | | | |
| | | | | dystrophy; | | | | | |
| | | | | Duchenne | | | | | |
| | | | | muscular | | | | | |
| | | | | dystrophy | | | | | |
| X | 31507280 | NM_004006.2:c.8390 + | | not provided; | C > T | splice | ABE | SEQ ID | SEQ ID |
| | | 1G > A | | Not Provided | | donor | | NO: 214 | NO: 549; |
| | | | | | | variant | | | SEQ ID |
| | | | | | | | | | NO: 550; |
| | | | | | | | | | SEQ ID |
| | | | | | | | | | NO: 551; |
| | | | | | | | | | SEQ ID |
| | | | | | | | | | NO: 552 |
| X | 32823316 | NM_004006.2:c.336G > | NP_003997.1:p.Trp112Ter | Dilated | C > T | 5 prime | ABE | SEQ ID | SEQ ID |
| | | A | | cardiomy- | | UTR | | NO: 215 | NO: 553; |
| | | | | opathy | | variant | | | SEQ ID |
| | | | | 3B; Becker | | | | | NO: 554; |
| | | | | muscular | | | | | SEQ ID |
| | | | | dystrophy; | | | | | NO: 555 |
| | | | | Duchenne | | | | | |
| | | | | muscular | | | | | |
| | | | | dystrophy | | | | | |
| X | 32816565 | NM_004006.2:c.433C > | NP_003997.1:p.Arg145Ter | Duchenne | G > A | nonsense | ABE | SEQ ID | SEQ ID |
| | | T | | muscular | | | | NO: 216 | NO: 556; |
| | | | | dystrophy; | | | | | SEQ ID |
| | | | | DUCHENNE | | | | | NO: 557; |
| | | | | MUSCULAR | | | | | SEQ ID |
| | | | | DYSTROPHY; | | | | | NO: 558; |
| | | | | Dilated | | | | | SEQ ID |
| | | | | cardiomy- | | | | | NO: 559 |
| | | | | opathy | | | | | |
| | | | | 3B; not | | | | | |
| | | | | provided; Not | | | | | |
| | | | | Provided; | | | | | |
| | | | | Becker | | | | | |
| | | | | muscular | | | | | |
| | | | | dystrophy | | | | | |
| X | 32463576 | NM_004006.2:c.3295C > | NP_003997.1:p.Gln1099Ter | Dilated | G > A | nonsense | ABE | SEQ ID | SEQ ID |
| | | T | | cardiomy- | | | | NO: 217 | NO: 560; |
| | | | | opathy | | | | | SEQ ID |
| | | | | 3B; Becker | | | | | NO: 561 |
| | | | | muscular | | | | | |
| | | | | dystrophy; | | | | | |
| | | | | Duchenne | | | | | |
| | | | | muscular | | | | | |
| | | | | dystrophy | | | | | |
| X | 32844794 | NM_004006.2:c.253C > | NP_003997.1:p.Gln85Ter | Duchenne | G > A | 5 prime | ABE | SEQ ID | SEQ ID |
| | | T | | muscular | | UTR | | NO: 218 | NO: 562 |
| | | | | dystrophy; | | variant | | | |
| | | | | DUCHENNE | | | | | |
| | | | | MUSCULAR | | | | | |
| | | | | DYSTROPHY | | | | | |
| X | 32468539 | NM_004006.2:c.312C > | NP_003997.1:p.Gln1041Ter | Duchenne | G > A | nonsense | ABE | SEQ ID | SEQ ID |
| | | T | | muscular | | | | NO: 219 | NO: 563; |
| | | | | dystrophy; | | | | | SEQ ID |
| | | | | DUCHENNE | | | | | NO: 564; |
| | | | | MUSCULAR | | | | | SEQ ID |
| | | | | DYSTROPHY; | | | | | NO: 565; |
| | | | | Dilated | | | | | SEQ ID |
| | | | | cardiomy- | | | | | NO: 566 |
| | | | | opathy 3B | | | | | |
| X | 32390103 | NM_004006.2:c.4312C > | NP_003997.1:p.Gln1438Ter | Duchenne | G > A | nonsense | ABE | SEQ ID | SEQ ID |
| | | T | | muscular | | | | NO: 220 | NO: 567; |
| | | | | dystrophy | | | | | SEQ ID |
| | | | | | | | | | NO: 568 |
| X | 32342264 | NM_004006.2:c.575C > | NP_003997.1:p.Gln1920Ter | not provided; | G > A | nonsense | ABE | SEQ ID | SEQ ID |
| | | T | | Not Provided | | | | NO: 221 | NO: 569 |

SEQUENCES
(pCMV-ABEmaxNG)
SEQ ID NO: 1
ATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCC
ATTGACGCAAATGGGCGGTAGGCGTGTACGGTGGGAGGTCTATATAAGCAGAGCTG
GTTTAGTGAACCGTCAGATCCGCTAGAGATCCGCGGCCGCTAATACGACTCACTAT
AGGGAGAGCCGCCACCATGAAACGGACAGCCGACGGAAGCGAGTTCGAGTCACCA
AAGAAGAAGCGGAAAGTCTCTGAAGTCGAGTTTAGCCACGAGTATTGGATGAGGCA
CGCACTGACCCTGGCAAAGCGAGCATGGGATGAAAGAGAAGTCCCCGTGGGCGCC
GTGCTGGTGCACAACAATAGAGTGATCGGAGAGGGATGGAACAGGCCAATCGGCC
GCCACGACCCTACCGCACACGCAGAGATCATGGCACTGAGGCAGGGAGGCCTGGTC
ATGCAGAATTACCGCCTGATCGATGCCACCCTGTATGTGACACTGGAGCCATGCGT
GATGTGCGCAGGAGCAATGATCCACAGCAGGATCGGAAGAGTGGTGTTCGGAGCA
CGGGACGCCAAGACCGGCGCAGCAGGCTCCCTGATGGATGTGCTGCACCACCCCGG
CATGAACCACCGGGTGGAGATCACAGAGGGAATCCTGGCAGACGAGTGCGCCGCC
CTGCTGAGCGATTTCTTTAGAATGCGGAGACAGGAGATCAAGGCCCAGAAGAAGGC
ACAGAGCTCCACCGACTCTGGAGGATCTAGCGGAGGATCCTCTGGAAGCGAGACAC
CAGGCACAAGCGAGTCCGCCACACCAGAGAGCTCCGGCGGCTCCTCCGGAGGATCC
TCTGAGGTGGAGTTTTCCCACGAGTACTGGATGAGACATGCCCTGACCCTGGCCAA
GAGGGCACGCGATGAGAGGGAGGTGCCTGTGGGAGCCGTGCTGGTGCTGAACAAT
AGAGTGATCGGCGAGGGCTGGAACAGAGCCATCGGCCTGCACGACCCAACAGCCC
ATGCCGAAATTATGGCCCTGAGACAGGGCGGCCTGGTCATGCAGAACTACAGACTG
ATTGACGCCACCCTGTACGTGACATTCGAGCCTTGCGTGATGTGCGCCGGCGCCATG
ATCCACTCTAGGATCGGCCGCGTGGTGTTTGGCGTGAGGAACGCAAAAACCGGCGC
CGCAGGCTCCCTGATGGACGTGCTGCACTACCCCGGCATGAATCACCGCGTCGAAA
TTACCGAGGGAATCCTGGCAGATGAATGTGCCGCCCTGCTGTGCTATTTCTTTCGGA
TGCCTAGACAGGTGTTCAATGCTCAGAAGAAGGCCCAGAGCTCCACCGACTCCGGA
GGATCTAGCGGAGGCTCCTCTGGCTCTGAGACACCTGGCACAAGCGAGAGCGCAAC
ACCTGAAAGCAGCGGGGGCAGCAGCGGGGGGTCAGACAAGAAGTACAGCATCGGC
CTGGCCATCGGCACCAACTCTGTGGGCTGGGCCGTGATCACCGACGAGTACAAGGT
GCCCAGCAAGAAATTCAAGGTGCTGGGCAACACCGACCGGCACAGCATCAAGAAG
AACCTGATCGGAGCCCTGCTGTTCGACAGCGGCGAAACAGCCGAGGCCACCCGGCT
GAAGAGAACCGCCAGAAGAAGATACACCAGACGGAAGAACCGGATCTGCTATCTG
CAAGAGATCTTCAGCAACGAGATGGCCAAGGTGGACGACAGCTTCTTCCACAGACT
GGAAGAGTCCTTCCTGGTGGAAGAGGATAAGAAGCACGAGCGGCACCCCATCTTCG
GCAACATCGTGGACGAGGTGGCCTACCACGAGAAGTACCCCACCATCTACCACCTG
AGAAAGAAACTGGTGGACAGCACCGACAAGGCCGACCTGCGGCTGATCTATCTGGC
CCTGGCCCACATGATCAAGTTCCGGGGCCACTTCCTGATCGAGGGCGACCTGAACC
CCGACAACAGCGACGTGGACAAGCTGTTCATCCAGCTGGTGCAGACCTACAACCAG
CTGTTCGAGGAAAACCCCATCAACGCCAGCGGCGTGGACGCCAAGGCCATCCTGTC
TGCCAGACTGAGCAAGAGCAGACGGCTGGAAAATCTGATCGCCCAGCTGCCCGGCG
AGAAGAAGAATGGCCTGTTCGGAAACCTGATTGCCCTGAGCCTGGGCCTGACCCCC
AACTTCAAGAGCAACTTCGACCTGGCCGAGGATGCCAAACTGCAGCTGAGCAAGGA
CACCTACGACGACGACCTGGACAACCTGCTGGCCCAGATCGGCGACCAGTACGCCG
ACCTGTTTCTGGCCGCCAAGAACCTGTCCGACGCCATCCTGCTGAGCGACATCCTGA
GAGTGAACACCGAGATCACCAAGGCCCCCCTGAGCGCCTCTATGATCAAGAGATAC
GACGAGCACCACCAGGACCTGACCCTGCTGAAAGCTCTCGTGCGGCAGCAGCTGCC
TGAGAAGTACAAAGAGATTTTCTTCGACCAGAGCAAGAACGGCTACGCCGGCTACA
TTGACGGCGGAGCCAGCCAGGAAGAGTTCTACAAGTTCATCAAGCCCATCCTGGAA
AAGATGGACGGCACCGAGGAACTGCTCGTGAAGCTGAACAGAGAGGACCTGCTGC
GGAAGCAGCGGACCTTCGACAACGGCAGCATCCCCCACCAGATCCACCTGGGAGAG
CTGCACGCCATTCTGCGGCGGCAGGAAGATTTTTACCCATTCCTGAAGGACAACCG
GGAAAAGATCGAGAAGATCCTGACCTTCCGCATCCCCTACTACGTGGGCCCTCTGG
CCAGGGGAAACAGCAGATTCGCCTGGATGACCAGAAAGAGCGAGGAAACCATCAC
CCCCTGGAACTTCGAGGAAGTGGTGGACAAGGGCGCTTCCGCCCAGAGCTTCATCG
AGCGGATGACCAACTTCGATAAGAACCTGCCCAACGAGAAGGTGCTGCCCAAGCAC
AGCCTGCTGTACGAGTACTTCACCGTGTATAACGAGCTGACCAAAGTGAAATACGT
GACCGAGGGAATGAGAAAGCCCGCCTTCCTGAGCGGCGAGCAGAAAAAGGCCATC
GTGGACCTGCTGTTCAAGACCAACCGGAAAGTGACCGTGAAGCAGCTGAAAGAGG
ACTACTTCAAGAAAATCGAGTGCTTCGACTCCGTGGAAATCTCCGGCGTGGAAGAT
CGGTTCAACGCCTCCCTGGGCACATACCACGATCTGCTGAAAATTATCAAGGACAA
GGACTTCCTGGACAATGAGGAAAACGAGGACATTCTGGAAGATATCGTGCTGACCC
TGACACTGTTTGAGGACAGAGAGATGATCGAGGAACGGCTGAAAACCTATGCCCAC
CTGTTCGACGACAAAGTGATGAAGCAGCTGAAGCGGCGGAGATACACCGGCTGGG
GCAGGCTGAGCCGGAAGCTGATCAACGGCATCCGGGACAAGCAGTCCGGCAAGAC
AATCCTGGATTTCCTGAAGTCCGACGGCTTCGCCAACAGAAACTTCATGCAGCTGAT
CCACGACGACAGCCTGACCTTTAAAGAGGACATCCAGAAAGCCCAGGTGTCCGGCC
AGGGCGATAGCCTGCACGAGCACATTGCCAATCTGGCCGGCAGCCCCGCCATTAAG
AAGGGCATCCTGCAGACAGTGAAGGTGGTGGACGAGCTCGTGAAAGTGATGGGCC
GGCACAAGCCCGAGAACATCGTGATCGAAATGGCCAGAGAGAACCAGACCACCCA
GAAGGGACAGAAGAACAGCCGCGAGAGAATGAAGCGGATCGAAGAGGGCATCAA
AGAGCTGGGCAGCCAGATCCTGAAAGAACACCCCGTGGAAAACACCCAGCTGCAG
AACGAGAAGCTGTACCTGTACTACCTGCAGAATGGGCGGGATATGTACGTGGACCA
GGAACTGGACATCAACCGGCTGTCCGACTACGATGTGGACCATATCGTGCCTCAGA
GCTTTCTGAAGGACGACTCCATCGACAACAAGGTGCTGACCAGAAGCGACAAGAAC
CGGGGCAAGAGCGACAACGTGCCCTCCGAAGAGGTCGTGAAGAAGATGAAGAACT
ACTGGCGGCAGCTGCTGAACGCCAAGCTGATTACCCAGAGAAAGTTCGACAATCTG
ACCAAGGCCGAGAGAGGCGGCCTGAGCGAACTGGATAAGGCCGGCTTCATCAAGA
GACAGCTGGTGGAAACCCGGCAGATCACAAAGCACGTGGCACAGATCCTGGACTCC
CGGATGAACACTAAGTACGACGAGAATGACAAGCTGATCCGGGAAGTGAAAGTGA
TCACCCTGAAGTCCAAGCTGGTGTCCGATTTCCGGAAGGATTTCCAGTTTTACAAAG
TGCGCGAGATCAACAACTACCACCACGCCCACGACGCCTACCTGAACGCCGTCGTG
GGAACCGCCCTGATCAAAAAGTACCCTAAGCTGGAAAGCGAGTTCGTGTACGGCGA
CTACAAGGTGTACGACGTGCGGAAGATGATCGCCAAGAGCGAGCAGGAAATCGGC
AAGGCTACCGCCAAGTACTTCTTCTACAGCAACATCATGAACTTTTTCAAGACCGAG
ATTACCCTGGCCAACGGCGAGATCCGGAAGCGGCCTCTGATCGAGACAAACGGCGA
AACCGGGGAGATCGTGTGGGATAAGGGCCGGGATTTTGCCACCGTGCGGAAAGTGC
TGAGCATGCCCCAAGTGAATATCGTGAAAAAGACCGAGGTGCAGACAGGCGGCTTC
AGCAAAGAGTCTATCCgGCCCAAGAGGAACAGCGATAAGCTGATCGCCAGAAAGA
AGGACTGGGACCCTAAGAAGTACGGCGGCTTCGtCAGCCCCACCGTGGCCTATTCTG
TGCTGGTGGTGGCCAAAGTGGAAAAGGGCAAGTCCAAGAAACTGAAGAGTGTGAA
AGAGCTGCTGGGGATCACCATCATGGAAAGAAGCAGCTTCGAGAAGAATCCCATCG
ACTTTCTGGAAGCCAAGGGCTACAAAGAAGTGAAAAAGGACCTGATCATCAAGCTG
CCTAAGTACTCCCTGTTCGAGCTGGAAAACGGCCGGAAGAGAATGCTGGCCTCTGC
CcGCtttCTGCAGAAGGGAAACGAACTGGCCCTGCCCTCCAAATATGTGAACTTCCTG
TACCTGGCCAGCCACTATGAGAAGCTGAAGGGCTCCCCCGAGGATAATGAGCAGAA
ACAGCTGTTTGTGGAACAGCACAAGCACTACCTGGACGAGATCATCGAGCAGATCA
GCGAGTTCTCCAAGAGAGTGATCCTGGCCGACGCTAATCTGGACAAAGTGCTGTCC
GCCTACAACAAGCACCGGGATAAGCCCATCAGAGAGCAGGCCGAGAATATCATCC
ACCTGTTTACCCTGACCAATCTGGGAGCCCCTcggGCCTTCAAGTACTTTGACACCAC
CATCGACCGGAAGgtGTACcggAGCACCAAAGAGGTGCTGGACGCCACCCTGATCCA
CCAGAGCATCACCGGCCTGTACGAGACACGGATCGACCTGTCTCAGCTGGGAGGTG
ACTCTGGCGGCTCAAAAAGAACCGCCGACGGCAGCGAATTCGAGCCCAAGAAGAA
GAGGAAAGTCTAACCGGTCATCATCACCATCACCATTGAGTTTAAACCCGCTGATC
AGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGC
TCTATGGCTTCTGAGGCGGAAAGAACCAGCTGGGGCTCGATACCGTCGACCTCTAG
CTAGAGCTTGGCGTAATCATGGTCATAGCTGTTTCCTGTGTGAAATTGTTATCCGCT
CACAATTCCACACAACATACGAGCCGGAAGCATAAAGTGTAAAGCCTAGGGTGCCT
AATGAGTGAGCTAACTCACATTAATTGCGTTGCGCTCACTGCCCGCTTTCCAGTCGG
GAAACCTGTCGTGCCAGCTGCATTAATGAATCGGCCAACGCGCGGGGAGAGGCGGT
TTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAA
TCAGGGGATAACGCAGGAAAGAACATGTGAGCAAAAGGCCAGCAAAAGGCCAGGA
ACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTTCTCATAG
CTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGGCCTAAC
TACGGCTACACTAGAAGAACAGTATTTGGTATCTGCGCTCTGCTGAAGCCAGTTACC
TTCGGAAAAAGAGTTGGTAGCTCTTGATCCGGCAAACAAACCACCGCTGGTAGCGG
TGGTTTTTTTGTTTGCAAGCAGCAGATTACGCGCAGAAAAAAAGGATCTCAAGAAG
ATCCTTTGATCTTTTCTACGGGGTCTGACACTCAGTGGAACGAAAACTCACGTTAAG
GGATTTTGGTCATGAGATTATCAAAAAGGATCTTCACCTAGATCCTTTTAAATTAAA
AATGAAGTTTTAAATCAATCTAAAGTATATATGAGTAAACTTGGTCTGACAGTTACC
AATGCTTAATCAGTGAGGCACCTATCTCAGCGATCTGTCTATTTCGTTCATCCATAG
TTGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGCCAGTTAA
TAGTTTGCGCAACGTTGTTGCCATTGCTACAGGCATCGTGGTGTCACGCTCGTCGTT
TGGTATGGCTTCATTCAGCTCCGGTTCCCAACGATCAAGGCGAGTTACATGATCCCC
CATGTTGTGCAAAAAAGCGGTTAGCTCCTTCGGTCCTCCGATCGTTGTCAGAAGTAA
GTTGGCCGCAGTGTTATCACTCATGGTTATGGCAGCACTGCATAATTCTCTTACTGT
CATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATA
CCGCGCCACATAGCAGAACTTTAAAAGTGCTCATCATTGGAAAACGTTCTTCGGGG
CGAAAACTCTCAAGGATCTTACCGCTGTTGAGATCCAGTTCGATGTAACCCACTCGT
GCACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCA
CATTTCCCCGAAAAGTGCCACCTGACGTCGACGGATCGGGAGATCGATCTCCCGAT
CCCCTAGGGTCGACTCTCAGTACAATCTGCTCTGATGCCGCATAGTTAAGCCAGTAT
CTGCTCCCTGCTTGTGTGTTGGAGGTCGCTGAGTAGTGCGCGAGCAAAATTTAAGCT
ACAACAAGGCAAGGCTTGACCGACAATTGCATGAAGAATCTGCTTAGGGTTAGGCG
TTTTGCGCTGCTTCGCGATGTACGGGCCAGATATACGCGTTGACATTGATTATTGAC
TAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAA
GTGTATC
(pCMV-ABEmaxNG)
SEQ ID NO: 2
ATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCC
ATTGACGCAAATGGGCGGTAGGCGTGTACGGTGGGAGGTCTATATAAGCAGAGCTG
GTTTAGTGAACCGTCAGATCCGCTAGAGATCCGCGGCCGCTAATACGACTCACTAT
AGGGAGAGCCGccaccatggctagcATGAAACGGACAGCCGACGGAAGCGAGTTCGAGTC
ACCAAAGAAGAAGCGGAAAGTCGGATCCTCTGAGGTGGAGTTTTCCCACGAGTACT
GGATGAGACATGCCCTGACCCTGGCCAAGAGGGCACGCGATGAGAGGGAGGTGCC
TGTGGGAGCCGTGCTGGTGCTGAACAATAGAGTGATCGGCGAGGGCTGGAACAGA
GCCATCGGCCTGCACGACCCAACAGgCCATGCCGAAATTATGGCCCTGAGgCAGGG
CGGCCTGGTCATGCAGAACTACAGACTGATTGACGCCACCCTGTACGgGACATTCGA
GCCTTGCGTGATGTGCGCCGGCGCCATGATCCACTCTAGGATCGGCCGCGTGGTGTT
TGGCGTGAGGAACGCAAAAACCGGCGCCGCAGGCTCCCTGATGGACGTGCTGCACT
ACCCCGGCATGAATCACCGCGTCGAAATTACCGAGGGAATCCTGGCAGATGAATGT
GCCGCCCTGCTGTGCTATTTCTTTCGGATGCCTAGACAGGTGTTCAATGCTCAGAAG
AAGGCCCAGAGCTCCACCGACTCCGGAGGATCTAGCGGAGGCTCCTCTGGCTCTGA
GACACCTGGCACAAGCGAGAGCGCAACACCTGAAAGCAGCGGGGGCAGCAGCGGG
GGGTCAGACAAGAAGTACAGCATCGGCCTGGCCATCGGCACCAACTCTGTGGGCTG
GGCCGTGATCACCGACGAGTACAAGGTGCCCAGCAAGAAATTCAAGGTGCTGGGCA
ACACCGACCGGCACAGCATCAAGAAGAACCTGATCGGAGCCCTGCTGTTCGACAGC
GGCGAAACAGCCGAGGCCACCCGGCTGAAGAGAACCGCCAGAAGAAGATACACCA
GACGGAAGAACCGGATCTGCTATCTGCAAGAGATCTTCAGCAACGAGATGGCCAAG
GTGGACGACAGCTTCTTCCACAGACTGGAAGAGTCCTTCCTGGTGGAAGAGGATAA
GAAGCACGAGCGGCACCCCATCTTCGGCAACATCGTGGACGAGGTGGCCTACCACG
AGAAGTACCCCACCATCTACCACCTGAGAAAGAAACTGGTGGACAGCACCGACAA
GGCCGACCTGCGGCTGATCTATCTGGCCCTGGCCCACATGATCAAGTTCCGGGGCC
ACTTCCTGATCGAGGGCGACCTGAACCCCGACAACAGCGACGTGGACAAGCTGTTC
ATCCAGCTGGTGCAGACCTACAACCAGCTGTTCGAGGAAAACCCCATCAACGCCAG
CGGCGTGGACGCCAAGGCCATCCTGTCTGCCAGACTGAGCAAGAGCAGACGGCTGG
AAAATCTGATCGCCCAGCTGCCCGGCGAGAAGAAGAATGGCCTGTTCGGAAACCTG
ATTGCCCTGAGCCTGGGCCTGACCCCCAACTTCAAGAGCAACTTCGACCTGGCCGA
GGATGCCAAACTGCAGCTGAGCAAGGACACCTACGACGACGACCTGGACAACCTGC
TGGCCCAGATCGGCGACCAGTACGCCGACCTGTTTCTGGCCGCCAAGAACCTGTCC
GACGCCATCCTGCTGAGCGACATCCTGAGAGTGAACACCGAGATCACCAAGGCCCC
CCTGAGCGCCTCTATGATCAAGAGATACGACGAGCACCACCAGGACCTGACCCTGC
TGAAAGCTCTCGTGCGGCAGCAGCTGCCTGAGAAGTACAAAGAGATTTTCTTCGAC
CAGAGCAAGAACGGCTACGCCGGCTACATTGACGGCGGAGCCAGCCAGGAAGAGT
TCTACAAGTTCATCAAGCCCATCCTGGAAAAGATGGACGGCACCGAGGAACTGCTC
GTGAAGCTGAACAGAGAGGACCTGCTGCGGAAGCAGCGGACCTTCGACAACGGCA
GCATCCCCCACCAGATCCACCTGGGAGAGCTGCACGCCATTCTGCGGCGGCAGGAA
GATTTTTACCCATTCCTGAAGGACAACCGGGAAAAGATCGAGAAGATCCTGACCTT
CCGCATCCCCTACTACGTGGGCCCTCTGGCCAGGGGAAACAGCAGATTCGCCTGGA
TGACCAGAAAGAGCGAGGAAACCATCACCCCCTGGAACTTCGAGGAAGTGGTGGA
CAAGGGCGCTTCCGCCCAGAGCTTCATCGAGCGGATGACCAACTTCGATAAGAACC
TGCCCAACGAGAAGGTGCTGCCCAAGCACAGCCTGCTGTACGAGTACTTCACCGTG
TATAACGAGCTGACCAAAGTGAAATACGTGACCGAGGGAATGAGAAAGCCCGCCTT
CCTGAGCGGCGAGCAGAAAAAGGCCATCGTGGACCTGCTGTTCAAGACCAACCGGA
AAGTGACCGTGAAGCAGCTGAAAGAGGACTACTTCAAGAAAATCGAGTGCTTCGAC
TCCGTGGAAATCTCCGGCGTGGAAGATCGGTTCAACGCCTCCCTGGGCACATACCA
CGATCTGCTGAAAATTATCAAGGACAAGGACTTCCTGGACAATGAGGAAAACGAGG
ACATTCTGGAAGATATCGTGCTGACCCTGACACTGTTTGAGGACAGAGAGATGATC
GAGGAACGGCTGAAAACCTATGCCCACCTGTTCGACGACAAAGTGATGAAGCAGCT
GAAGCGGCGGAGATACACCGGCTGGGGCAGGCTGAGCCGGAAGCTGATCAACGGC
ATCCGGGACAAGCAGTCCGGCAAGACAATCCTGGATTTCCTGAAGTCCGACGGCTT
CGCCAACAGAAACTTCATGCAGCTGATCCACGACGACAGCCTGACCTTTAAAGAGG
ACATCCAGAAAGCCCAGGTGTCCGGCCAGGGCGATAGCCTGCACGAGCACATTGCC
AATCTGGCCGGCAGCCCCGCCATTAAGAAGGGCATCCTGCAGACAGTGAAGGTGGT
GGACGAGCTCGTGAAAGTGATGGGCCGGCACAAGCCCGAGAACATCGTGATCGAA
ATGGCCAGAGAGAACCAGACCACCCAGAAGGGACAGAAGAACAGCCGCGAGAGA
ATGAAGCGGATCGAAGAGGGCATCAAAGAGCTGGGCAGCCAGATCCTGAAAGAAC
ACCCCGTGGAAAACACCCAGCTGCAGAACGAGAAGCTGTACCTGTACTACCTGCAG
AATGGGCGGGATATGTACGTGGACCAGGAACTGGACATCAACCGGCTGTCCGACTA
CGATGTGGACCATATCGTGCCTCAGAGCTTTCTGAAGGACGACTCCATCGACAACA
AGGTGCTGACCAGAAGCGACAAGAACCGGGGCAAGAGCGACAACGTGCCCTCCGA
AGAGGTCGTGAAGAAGATGAAGAACTACTGGCGGCAGCTGCTGAACGCCAAGCTG
ATTACCCAGAGAAAGTTCGACAATCTGACCAAGGCCGAGAGAGGCGGCCTGAGCG
AACTGGATAAGGCCGGCTTCATCAAGAGACAGCTGGTGGAAACCCGGCAGATCACA
AAGCACGTGGCACAGATCCTGGACTCCCGGATGAACACTAAGTACGACGAGAATGA
CAAGCTGATCCGGGAAGTGAAAGTGATCACCCTGAAGTCCAAGCTGGTGTCCGATT
TCCGGAAGGATTTCCAGTTTTACAAAGTGCGCGAGATCAACAACTACCACCACGCC
CACGACGCCTACCTGAACGCCGTCGTGGGAACCGCCCTGATCAAAAAGTACCCTAA
GCTGGAAAGCGAGTTCGTGTACGGCGACTACAAGGTGTACGACGTGCGGAAGATGA
TCGCCAAGAGCGAGCAGGAAATCGGCAAGGCTACCGCCAAGTACTTCTTCTACAGC
AACATCATGAACTTTTTCAAGACCGAGATTACCCTGGCCAACGGCGAGATCCGGAA
GCGGCCTCTGATCGAGACAAACGGCGAAACCGGGGAGATCGTGTGGGATAAGGGC
CGGGATTTTGCCACCGTGCGGAAAGTGCTGAGCATGCCCCAAGTGAATATCGTGAA
AAAGACCGAGGTGCAGACAGGCGGCTTCAGCAAAGAGTCTATCCgGCCCAAGAGGA
ACAGCGATAAGCTGATCGCCAGAAAGAAGGACTGGGACCCTAAGAAGTACGGCGG
CTTCGtCAGCCCCACCGTGGCCTATTCTGTGCTGGTGGTGGCCAAAGTGGAAAAGGG
CAAGTCCAAGAAACTGAAGAGTGTGAAAGAGCTGCTGGGGATCACCATCATGGAA
AGAAGCAGCTTCGAGAAGAATCCCATCGACTTTCTGGAAGCCAAGGGCTACAAAGA
AGTGAAAAAGGACCTGATCATCAAGCTGCCTAAGTACTCCCTGTTCGAGCTGGAAA
ACGGCCGGAAGAGAATGCTGGCCTCTGCCcGCtttCTGCAGAAGGGAAACGAACTGGC
CCTGCCCTCCAAATATGTGAACTTCCTGTACCTGGCCAGCCACTATGAGAAGCTGAA
GGGCTCCCCCGAGGATAATGAGCAGAAACAGCTGTTTGTGGAACAGCACAAGCACT
ACCTGGACGAGATCATCGAGCAGATCAGCGAGTTCTCCAAGAGAGTGATCCTGGCC
GACGCTAATCTGGACAAAGTGCTGTCCGCCTACAACAAGCACCGGGATAAGCCCAT
CAGAGAGCAGGCCGAGAATATCATCCACCTGTTTACCCTGACCAATCTGGGAGCCC
CTcggGCCTTCAAGTACTTTGACACCACCATCGACCGGAAGgtGTACcggAGCACCAAA
GAGGTGCTGGACGCCACCCTGATCCACCAGAGCATCACCGGCCTGTACGAGACACG
GATCGACCTGTCTCAGCTGGGAGGTGACTCTGGCGGCTCAAAAAGAACCGCCGACG
GCAGCGAATTCGAGCCCAAGAAGAAGAGGAAAGTCTAACCGGTCATCATCACCATC
ACCATTGAGTTTAAACCCGCTGATCAGCCTCGACTGTGCCTTCTAGTTGCCAGCCAT
CTGTTGTTTGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGC
AGGCATGCTGGGGATGCGGTGGGCTCTATGGCTTCTGAGGCGGAAAGAACCAGCTG
GGGCTCGATACCGTCGACCTCTAGCTAGAGCTTGGCGTAATCATGGTCATAGCTGTT
TCCTGTGTGAAATTGTTATCCGCTCACAATTCCACACAACATACGAGCCGGAAGCAT
AAAGTGTAAAGCCTAGGGTGCCTAATGAGTGAGCTAACTCACATTAATTGCGTTGC
GCTCACTGCCCGCTTTCCAGTCGGGAAACCTGTCGTGCCAGCTGCATTAATGAATCG
GCCAACGCGCGGGGAGAGGCGGTTTGCGTATTGGGCGCTCTTCCGCTTCCTCGCTCA
CTGACTCGCTGCGCTCGGTCGTTCGGCTGCGGCGAGCGGTATCAGCTCACTCAAAG
GCGGTAATACGGTTATCCACAGAATCAGGGGATAACGCAGGAAAGAACATGTGAG
CAAAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTC
GGGAAGCGTGGCGCTTTCTCATAGCTCACGCTGTAGGTATCTCAGTTCGGTGTAGGT
CGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACT
GGCAGCAGCCACTGGTAACAGGATTAGCAGAGCGAGGTATGTAGGCGGTGCTACA
GAGTTCTTGAAGTGGTGGCCTAACTACGGCTACACTAGAAGAACAGTATTTGGTAT
CTGCGCTCTGCTGAAGCCAGTTACCTTCGGAAAAAGAGTTGGTAGCTCTTGATCCGG
CAAACAAACCACCGCTGGTAGCGGTGGTTTTTTTGTTTGCAAGCAGCAGATTACGCG
CAGAAAAAAAGGATCTCAAGAAGATCCTTTGATCTTTTCTACGGGGTCTGACACTC
AGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATAT
GAGTAAACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATAACTACGA
TACGGGAGGGCTTACCATCTGGCCCCAGTGCTGCAATGATACCGCGAGACCCACGC
TCACCGGCTCCAGATTTATCAGCAATAAACCAGCCAGCCGGAAGGGCCGAGCGCAG
AAGTGGTCCTGCAACTTTATCCGCCTCCATCCAGTCTATTAATTGTTGCCGGGAAGC
TAGAGTAAGTAGTTCGCCAGTTAATAGTTTGCGCAACGTTGTTGCCATTGCTACAGG
CATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTT
GCCCGGCGTCAATACGGGATAATACCGCGCCACATAGCAGAACTTTAAAAGTGCTC
ATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAA
TAAGGGCGACACGGAAATGTTGAATACTCATACTCTTCCTTTTTCAATATTATTGAA
GCATTTATCAGGGTTATTGTCTCATGAGCGGATACATATTTGAATGTATTTAGAAAA
ATAAACAAATAGGGGTTCCGCGCACATTTCCCCGAAAAGTGCCACCTGACGTCGAC
GGATCGGGAGATCGATCTCCCGATCCCCTAGGGTCGACTCTCAGTACAATCTGCTCT
GATGCCGCATAGTTAAGCCAGTATCTGCTCCCTGCTTGTGTGTTGGAGGTCGCTGAG
TAGTGCGCGAGCAAAATTTAAGCTACAACAAGGCAAGGCTTGACCGACAATTGCAT
GAAGAATCTGCTTAGGGTTAGGCGTTTTGCGCTGCTTCGCGATGTACGGGCCAGATA
TACGCGTTGACATTGATTATTGACTAGTTATTAATAGTAATCAATTACGGGGTCATT
AGTTCATAGCCCATATATGGAGTTCCGCGTTACATAACTTACGGTAAATGGCCCGCC
TGGCTGACCGCCCAACGACCCCCGCCCATTGACGTCAATAATGACGTATGTTCCCAT
AGTAACGCCAATAGGGACTTTCCATTGACGTCAATGGGTGGAGTATTTACGGTAAA
CTGCCCACTTGGCAGTACATCAAGTGTATC
(pAAV-meCMV-ABEmax-N)
SEQ ID NO: 3
aaagtagccgaagatgacggtttgtcacatggagttggcaggatgtttgattaaaaacataacaggaagaaaaatgccccgctg
tgggcggacaaaatagttgggaactgggaggggtggaaatggagtttttaaggattatttagggaagagtgacaaaatagatgggaactg
ggtgtagcgtcgtaagctaatacgaaaattaaaaatgacaaaatagtttggaactagatttcacttatctggttcggatctcctaggctcaagca
gtgatcagatccagacatgataagatacattgatgagtttggacaaaccacaactagaatgcagtgaaaaaaatgctttatttgtgaaatttgtg
atgctattgctttatttgtaaccattataagctgcaataaacaagttaacaacaacaattccatgtcctgcaggcagctgcgcgctcgctcgctc
actgaggccgcccgggcgtcgggcgacctttggtcgcccggcctcagtgagcgagcgagcgcgcagagagggagtggccaactccat
cactaggggttcctgcggcctctagactcgagCGCGTGATGAGAGCAGCCACTACGGGTCTAGGCTGC
CCATGTAAGGAGGCAAGGCCTGGGGACACCCGAGATGCCTGGTTATAATTAACCCA
GACATGTGGCTGCCCCCCCCCCCCCAACACCTGCTGCCTGCTAAAAATAACCCTGTC
CCTGGTGGccctgcatgcccACTCACGGGGATTTCCAAGTCTCCACCCCATTGACGTCAAT
GGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAG
AGCTGGTTTAGTGAACCGTCAGATCcgccaccATGgctagcATGAAACGGACAGCCGACG
GAAGCGAGTTCGAGTCACCAAAGAAGAAGCGGAAAGTCTCTGAAGTCGAGTTTAGC
CACGAGTATTGGATGAGGCACGCACTGACCCTGGCAAAGCGAGCATGGGATGAAA
GAGAAGTCCCCGTGGGCGCCGTGCTGGTGCACAACAATAGAGTGATCGGAGAGGG
ATGGAACAGGCCAATCGGCCGCCACGACCCTACCGCACACGCAGAGATCATGGCAC
TGAGGCAGGGAGGCCTGGTCATGCAGAATTACCGCCTGATCGATGCCACCCTGTAT
GTGACACTGGAGCCATGCGTGATGTGCGCAGGAGCAATGATCCACAGCAGGATCGG
AAGAGTGGTGTTCGGAGCACGGGACGCCAAGACCGGCGCAGCAGGCTCCCTGATG
GATGTGCTGCACCACCCCGGCATGAACCACCGGGTGGAGATCACAGAGGGAATCCT
GGCAGACGAGTGCGCCGCCCTGCTGAGCGATTTCTTTAGAATGCGGAGACAGGAGA
TCAAGGCCCAGAAGAAGGCACAGAGCTCCACCGACTCTGGAGGATCTAGCGGAGG
ATCCTCTGGAAGCGAGACACCAGGCACAAGCGAGTCCGCCACACCAGAGAGCTCCG
GCGGCTCCTCCGGAGGATCCTCTGAGGTGGAGTTTTCCCACGAGTACTGGATGAGA
CATGCCCTGACCCTGGCCAAGAGGGCACGCGATGAGAGGGAGGTGCCTGTGGGAG
CCGTGCTGGTGCTGAACAATAGAGTGATCGGCGAGGGCTGGAACAGAGCCATCGGC
CTGCACGACCCAACAGCCCATGCCGAAATTATGGCCCTGAGACAGGGCGGCCTGGT
CATGCAGAACTACAGACTGATTGACGCCACCCTGTACGTGACATTCGAGCCTTGCGT
GATGTGCGCCGGCGCCATGATCCACTCTAGGATCGGCCGCGTGGTGTTTGGCGTGA
GGAACGCAAAAACCGGCGCCGCAGGCTCCCTGATGGACGTGCTGCACTACCCCGGC
ATGAATCACCGCGTCGAAATTACCGAGGGAATCCTGGCAGATGAATGTGCCGCCCT
GCTGTGCTATTTCTTTCGGATGCCTAGACAGGTGTTCAATGCTCAGAAGAAGGCCCA
GAGCTCCACCGACTCCGGAGGATCTAGCGGAGGCTCCTCTGGCTCTGAGACACCTG
GCACAAGCGAGAGCGCAACACCTGAAAGCAGCGGGGGCAGCAGCGGGGGGTCAGA
CAAGAAGTACAGCATCGGCCTGGCCATCGGCACCAACTCTGTGGGCTGGGCCGTGA
TCACCGACGAGTACAAGGTGCCCAGCAAGAAATTCAAGGTGCTGGGCAACACCGAC
CGGCACAGCATCAAGAAGAACCTGATCGGAGCCCTGCTGTTCGACAGCGGCGAAAC
AGCCGAGGCCACCCGGCTGAAGAGAACCGCCAGAAGAAGATACACCAGACGGAAG
AACCGGATCTGCTATCTGCAAGAGATCTTCAGCAACGAGATGGCCAAGGTGGACGA
CAGCTTCTTCCACAGACTGGAAGAGTCCTTCCTGGTGGAAGAGGATAAGAAGCACG
AGCGGCACCCCATCTTCGGCAACATCGTGGACGAGGTGGCCTACCACGAGAAGTAC
CCCACCATCTACCACCTGAGAAAGAAACTGGTGGACAGCACCGACAAGGCCGACCT
GCGGCTGATCTATCTGGCCCTGGCCCACATGATCAAGTTCCGGGGCCACTTCCTGAT
CGAGGGCGACCTGAACCCCGACAACAGCGACGTGGACAAGCTGTTCATCCAGCTGG
TGCAGACCTACAACCAGCTGTTCGAGGAAAACCCCATCAACGCCAGCGGCGTGGAC
GCCAAGGCCATCCTGTCTGCCAGACTGAGCAAGAGCAGACGGCTGGAAAATCTGAT
CGCCCAGCTGCCCGGCGAGAAGAAGAATGGCCTGTTCGGAAACCTGATTGCCCTGA
GCCTGGGCCTGACCCCCAACTTCAAGAGCAACTTCGACCTGGCCGAGGATGCCAAA
CTGCAGCTGAGCAAGGACACCTACGACGACGACCTGGACAACCTGCTGGCCCAGAT
CGGCGACCAGTACGCCGACCTGTTTCTGGCCGCCAAGAACCTGTCCGACGCCATCCT
GCTGAGCGACATCCTGAGAGTGAACACCGAGATCACCAAGGCCCCCCTGAGCGCCT
CTATGATCAAGAGATACGACGAGCACCACCAGGACCTGACCCTGCTGAAAGCTCTC
GTGCGGCAGCAGCTGCCTGAGAAGTACAAAGAGATTTTCTTCGACCAGAGCAAGAA
CGGCTACGCCGGCTACATTGACGGCGGAGCCAGCCAGGAAGAGTTCTACAAGTTCA
TCAAGCCCATCCTGGAAAAGATGGACGGCACCGAGGAACTGCTCGTGAAGCTGAAC
AGAGAGGACCTGCTGCGGAAGCAGCGGACCTTCGACAACGGCAGCATCCCCCACCA
GATCCACCTGGGAGAGCTGCACGCCATTCTGCGGCGGCAGGAAGATTTTTACCCAT
TCCTGAAGGACAACCGGGAAAAGATCGAGAAGATCCTGACCTTCCGCATCCCCTAC
TACGTGGGCCCTCTGGCCAGGGGAAACAGCAGATTCGCCTGGATGACCAGAAAGAG
CGAGGAAACCATCACCCCCTGGAACTTCGAGGAAGTGGTGGACAAGGGCGCTTCCG
CCCAGAGCTTCATCGAGCGGATGACCAACTTCGATAAGAACCTGCCCAACGAGAAG
GTGCTGCCCAAGCACAGCCTGCTGTACGAGTACTTCACCGTGTATAACGAGCTGAC
CAAAGTGAAATACGTGACCGAGGGAATGAGAAAGCCCGCCTTCCTGAGCGGCGAG
CAGAAAAAGGCCATCGTGGACCTGCTGTTCAAGACCAACCGGAAAGTGACCGTGAA
GCAGCTGAAAGAGGACTACTTCAAGAAAATCGAGTGTCTCAGTTATGACACCGAAA
TCCTGACAGTCGAGTATGGATTCTGCCGATCGGCAAGATTGTGGAGgAGAGAATTG
AATGTACGGTCTATAcgGTCGACAAgAATGGTtCgTCTACACCCAACCAaTTGCTCAA
TGGCATaATCGAGGGGAGCAGGAGGTGTTTGAGTATTGCCTGGAGGACGGGTCAATC
ATTAGAGCTACAAAGGACCATAAGTTTATGACAacCGATGGTCAAATGCTGCCGATA
GATGAAATATTCGAAAGGGgACTGGATCTTAAGCaAGTCGATggCCTTCCAaacTAgtAg
aattcctagagctcgctgatcagcctcgactgtgccttctagttgccagccatctgttgtttgcccctcccccgtgccttccttgaccctggaag
gtgccactcccactgtcctttcctaataaaatgaggaaattgcatcgcattgtctgagtaggtgtcattctattctggggggtggggtggggca
ggacagcaagggggaggattgggaagagaatagcaggcatgctggggaggtaccgagggcctatttcccatgattccttcatatttgcat
atacgatacaaggctgttagagagataattggaattaatttgactgtaaacacaaagatattagtacaaaatacgtgacgtagaaagtaataat
ttcttgggtagtttgcagttttaaaattatgttttaaaatggactatcatatgcttaccgtaacttgaaagtatttcgatttcttggctttatatatc
ttGTGGAAAGGACGAAACACCGGTTATCTCCTGTTCTGCAGCGTTTcAGAGCTAtgctgGAA
AcagcaTAGCAAGTTgAAATAAGGCTAGTCCGTTATCAACTTGAAAAAGTGGCACCGA
GTCGGTGCTTTTTTgcggccgcaggaacccctagtgatggagttggccactccctctctgcgcgctcgctcgctcactgaggc
cgggcgaccaaaggtogcccgacgcccgggctttgcccgggcggcctcagtgagcgagogagcgcgcagctgcctgcaggggcgcc
tgtaccgggagatgggggaggctaactgaaacacggaaggagacaataccggaaggaacccgcgctatgacggcaataaaaagacag
aataaaacgcacgggtgttgggtcgtttgttcataaacgcggggttcggtcccagggctggcactctgtcgataccccaccgagaccccatt
gggaccaatacgcccgcgtttcttccttttccccaccccaacccccaagttcgggtgaaggcccagggctcgcagccaacgtcggggcg
gcaagccctgccatagccactacgggtacgtaggccaaccactagaactatagctagagtcctgggcgaacaaacgatgctcgccttcca
gaaaaccgaggatgcgaaccacttcatccggggtcagcaccaccggcaagcgccgcgacggccgaggtctaccgatctcctgaagcca
gggcagatccgtgcacagcaccttgccgtagaagaacagcaaggccgccaatgcctgacgatgcgtggagaccgaaaccttgcgctcg
ttcgccagccaggacagaaatgcctcgacttcgctgctgcccaaggttgccgggtgacgcacaccgtggaaacggatgaaggcacgaa
cccagttgacataagcctgttcggttcgtaaactgtaatgcaagtagcgtatgcgctcacgcaactggtccagaaccttgaccgaacgcagc
ggtggtaacggcgcagtggcggttttcatggcttgttatgactgtttttttgtacagtctatgcctcgggcatccaagcagcaagcgcgttacg
ccgtgggtcgatgtttgatgttatggagcagcaacgatgttacgcagcagcaacgatgttacgcagcagggcagtcgccctaaaacaaagt
taggtggctcaagtatgggcatcattcgcacatgtaggctcggccctgaccaagtcaaatccatgcgggctgctcttgatcttttcggtcgtg
agttcggagacgtagccacctactcccaacatcagccggactccgattacctcgggaacttgctccgtagtaagacattcatcgcgcttgct
gccttcgaccaagaagcggttgttggcgctctcgcggcttacgttctgcccaggtttgagcagccgcgtagtgagatctatatctatgatctc
gcagtctccggcgagcaccggaggcagggcattgccaccgcgctcatcaatctcctcaagcatgaggccaacgcgcttggtgcttatgtg
atctacgtgcaagcagattacggtgacgatcccgcagtggctctctatacaaagttgggcatacgggaagaagtgatgcactttgatatcga
cccaagtaccgccacctaacaattcgttcaagccgagatcggcttcccggccgcggagttgttcggtaaattgtcacaacgccgcgaatat
agtctttaccatgcccttggccacgcccctctttaatacgacgggcaatttgcacttcagaaaatgaagagtttgctttagccataacaaaagtc
cagtatgctttttcacagcataactggactgatttcagtttacaactattctgtctagtttaagactttattgtcatagtttagatctattttgttca
gtttaagactttattgtccgcccacacccgcttacgcagggcatccatttattactcaaccgtaaccgattttgccaggttacgcggctggtctgcgg
tgtgaaataccgcacagatgcgtaaggagaaaataccgcatcaggcgctcttccgcttcctcgctcactgactcgctgcgctcggtcgttcg
gctgcggcgagcggtatcagctcactcaaaggcggtaatacggttatccacagaatcaggggataacgcaggaaagaacatgtgagcaa
aaggccagcaaaaggccaggaaccgtaaaaaggccgcgttgctggcgtttttccataggctccgcccccctgacgagcatcacaaaaat
cgacgctcaagtcagaggtggcgaaacccgacaggactataaagataccaggcgtttccccctggaagctccctcgtgcgctctcctgttc
cgaccctgccgcttaccggatacctgtccgcctttctcccttcgggaagcgtggcgctttctcaatgctcacgctgtaggtatctcagttcggt
gtaggtcgttcgctccaagctgggctgtgtgcacgaaccccccgttcagcccgaccgctgcgccttatccggtaactatcgtcttgagtcca
acccggtaagacacgacttatcgccactggcagcagccactggtaacaggattagcagagcgaggtatgtaggcggtgctacagagttct
tgaagtggtggcctaactacggctacactagaaggacagtatttggtatctgcgctctgctgaagccagttaccttcggaaaaagagttggta
gctcttgatccggcaaacaaaccaccgctggtagcggtggtttttttgtttgcaagcagcagattacgcgcagaaaaaaaggatctcaagaa
gatcctttgatcttttctacggggtctgacgctcagtggaacgaaaactcacgttaagggattttggtcatgagattatcaaaaaggatcttcac
ctagatccttttaaattaaaaatgaagttttaaatcaatctaaagtatatatgagtaaacttggtctgacagttaccaatgcttaatcagtgaggca
cctatctcagcgatctgtctatttcgttcatccatagttgcctgactccccgtcgtgtagataactacgatacgggagggcttaccatctggccc
cagtgctgcaatgataccgcgagacccacgctcaccggctccagatttatcagcaataaaccagccagccggaagggccgagcgcaga
agtggtcctgcaactttatccgcctccatccagtctattaattgttgccgggaagctagagtaagtagttcgccagttaatagtttgcgcaacgt
tgttgccattgctacaggcatcgtggtgtcacgctcgtcgtttggtatggcttcattcagctccggttcccaacgatcaaggcgagttacatgat
cccccatgttgtgcaaaaaagcggttagctccttcggtcctccgatcgttgtcagaagtaagttggccgcagtgttatcactcatggttatggc
agcactgcataattctcttactgtcatgccatccgtaagatgcttttctgtgactggtgagtactcaaccaagtcattctgagaatagtgtatgcg
gcgaccgagttgctcttgcccggcgtcaatacgggataataccgcgccacatagcagaactttaaaagtgctcatcattggaaaacgttcttc
ggggcgaaaactctcaaggatcttaccgctgttgagatccagttcgatgtaacccactcgtgcacccaactgatcttcagcatcttttactttca
ccagcgtttctgggtgagcaaaaacaggaaggcaaaatgccgcaaaaaagggaataagggcgacacggaaatgttgaatactcatactct
tcctttttcaatattattgaagcatttatcagggttattgtctcatgagcggatacatatttgaatgtatttagaaaaataaacaaataggggttccg
cgcacatttccccgaaaagtgccacctgaaattgtaaacgttaatattttgttaaaattcgcgttaaatttttgttaaatcagctcattttttaaccaa
taggccgaaatcggcaaaatcccttataaatcaaaagaatagaccgagatagggttgagtgttgttccagtttggaacaagagtccactatta
aagaacgtggactccaacgtcaaagggcgaaaaaccgtctatcagggcgatggcccactacgtgaaccatcaccctaatcaagttttttgg
ggtcgaggtgccgtaaagcactaaatcggaaccctaaagggagcccccgatttagagcttgacggggaaagccggcgaacgtggcgag
aaaggaagggaagaaagcgaaaggagcgggcgctagggcgctggcaagtgtagcggtcacgctgcgcgtaaccaccacacccgccg
cgcttaatgcgccgctacagggcgcgtcccattcgccattcaggctgcaaataagcgttgatattcagtcaattacaaacattaataacgaag
agatgacagaaaaattttcattctgtgacagagaa
(pAAV-ABEmaxNG-C)
SEQ ID NO: 4
aaagtagccgaagatgacggtttgtcacatggagttggcaggatgtttgattaaaaacataacaggaagaaaaatgccccgctg
tgggcggacaaaatagttgggaactgggaggggtggaaatggagtttttaaggattatttagggaagagtgacaaaatagatgggaactg
ggtgtagcgtcgtaagctaatacgaaaattaaaaatgacaaaatagtttggaactagatttcacttatctggttcggatctcctaggctcaagca
gtgatcagatccagacatgataagatacattgatgagtttggacaaaccacaactagaatgcagtgaaaaaaatgctttatttgtgaaatttgtg
atgctattgctttatttgtaaccattataagctgcaataaacaagttaacaacaacaattccatgtcctgcaggcagctgcgcgctcgctcgctc
actgaggccgcccgggcgtcgggcgacctttggtcgcccggcctcagtgagcgagcgagcgcgcagagagggagtggccaactccat
cactaggggttcctgcggcctctagactcgagCGCGTGATGAGAGCAGCCACTACGGGTCTAGGCTGC
CCATGTAAGGAGGCAAGGCCTGGGGACACCCGAGATGCCTGGTTATAATTAACCCA
GACATGTGGCTGCCCCCCCCCCCCCAACACCTGCTGCCTGCTAAAAATAACCCTGTC
CCTGGTGGccctgcatgcccACTCACGGGGATTTCCAAGTCTCCACCCCATTGACGTCAAT
GGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAG
AGCTGGTTTAGTGAACCGTCAGATCcgccaccATGgTcAAgATTatCAgcCGCAAATcCTTG
GGGAcACAGAATGTATATGACATCGGCGTGGAAaaGGATCACAATTTTctgCTGAAGA
ATGGTcTTgTTGCTtccAAtTGCTTCGACTCCGTGGAAATCTCCGGCGTGGAAGATCGG
TTCAACGCCTCCCTGGGCACATACCACGATCTGCTGAAAATTATCAAGGACAAGGA
CTTCCTGGACAATGAGGAAAACGAGGACATTCTGGAAGATATCGTGCTGACCCTGA
CACTGTTTGAGGACAGAGAGATGATCGAGGAACGGCTGAAAACCTATGCCCACCTG
TTCGACGACAAAGTGATGAAGCAGCTGAAGCGGCGGAGATACACCGGCTGGGGCA
GGCTGAGCCGGAAGCTGATCAACGGCATCCGGGACAAGCAGTCCGGCAAGACAAT
CCTGGATTTCCTGAAGTCCGACGGCTTCGCCAACAGAAACTTCATGCAGCTGATCCA
CGACGACAGCCTGACCTTTAAAGAGGACATCCAGAAAGCCCAGGTGTCCGGCCAGG
GCGATAGCCTGCACGAGCACATTGCCAATCTGGCCGGCAGCCCCGCCATTAAGAAG
GGCATCCTGCAGACAGTGAAGGTGGTGGACGAGCTCGTGAAAGTGATGGGCCGGC
ACAAGCCCGAGAACATCGTGATCGAAATGGCCAGAGAGAACCAGACCACCCAGAA
GGGACAGAAGAACAGCCGCGAGAGAATGAAGCGGATCGAAGAGGGCATCAAAGA
GCTGGGCAGCCAGATCCTGAAAGAACACCCCGTGGAAAACACCCAGCTGCAGAAC
GAGAAGCTGTACCTGTACTACCTGCAGAATGGGCGGGATATGTACGTGGACCAGGA
ACTGGACATCAACCGGCTGTCCGACTACGATGTGGACCATATCGTGCCTCAGAGCTT
TCTGAAGGACGACTCCATCGACAACAAGGTGCTGACCAGAAGCGACAAGAACCGG
GGCAAGAGCGACAACGTGCCCTCCGAAGAGGTCGTGAAGAAGATGAAGAACTACT
GGCGGCAGCTGCTGAACGCCAAGCTGATTACCCAGAGAAAGTTCGACAATCTGACC
AAGGCCGAGAGAGGCGGCCTGAGCGAACTGGATAAGGCCGGCTTCATCAAGAGAC
AGCTGGTGGAAACCCGGCAGATCACAAAGCACGTGGCACAGATCCTGGACTCCCGG
ATGAACACTAAGTACGACGAGAATGACAAGCTGATCCGGGAAGTGAAAGTGATCA
CCCTGAAGTCCAAGCTGGTGTCCGATTTCCGGAAGGATTTCCAGTTTTACAAAGTGC
GCGAGATCAACAACTACCACCACGCCCACGACGCCTACCTGAACGCCGTCGTGGGA
ACCGCCCTGATCAAAAAGTACCCTAAGCTGGAAAGCGAGTTCGTGTACGGCGACTA
CAAGGTGTACGACGTGCGGAAGATGATCGCCAAGAGCGAGCAGGAAATCGGCAAG
GCTACCGCCAAGTACTTCTTCTACAGCAACATCATGAACTTTTTCAAGACCGAGATT
ACCCTGGCCAACGGCGAGATCCGGAAGCGGCCTCTGATCGAGACAAACGGCGAAA
CCGGGGAGATCGTGTGGGATAAGGGCCGGGATTTTGCCACCGTGCGGAAAGTGCTG
AGCATGCCCCAAGTGAATATCGTGAAAAAGACCGAGGTGCAGACAGGCGGCTTCA
GCAAAGAGTCTATCCgGCCCAAGAGGAACAGCGATAAGCTGATCGCCAGAAAGAA
GGACTGGGACCCTAAGAAGTACGGCGGCTTCGtCAGCCCCACCGTGGCCTATTCTGT
GCTGGTGGTGGCCAAAGTGGAAAAGGGCAAGTCCAAGAAACTGAAGAGTGTGAAA
GAGCTGCTGGGGATCACCATCATGGAAAGAAGCAGCTTCGAGAAGAATCCCATCGA
CTTTCTGGAAGCCAAGGGCTACAAAGAAGTGAAAAAGGACCTGATCATCAAGCTGC
CTAAGTACTCCCTGTTCGAGCTGGAAAACGGCCGGAAGAGAATGCTGGCCTCTGCCC
GCttCTGCAGAAGGGAAACGAACTGGCCCTGCCCTCCAAATATGTGAACTTCCTGTA
CCTGGCCAGCCACTATGAGAAGCTGAAGGGCTCCCCCGAGGATAATGAGCAGAAAC
AGCTGTTTGTGGAACAGCACAAGCACTACCTGGACGAGATCATCGAGCAGATCAGC
GAGTTCTCCAAGAGAGTGATCCTGGCCGACGCTAATCTGGACAAAGTGCTGTCCGC
CTACAACAAGCACCGGGATAAGCCCATCAGAGAGCAGGCCGAGAATATCATCCACC
TGTTTACCCTGACCAATCTGGGAGCCCCTcggGCCTTCAAGTACTTTGACACCACCAT
CGACCGGAAGgtGTACcggAGCACCAAAGAGGTGCTGGACGCCACCCTGATCCACCA
GAGCATCACCGGCCTGTACGAGACACGGATCGACCTGTCTCAGCTGGGAGGTGACT
CTGGCGGCTCAAAAAGAACCGCCGACGGCAGCGAATTCGAGCCCAAGAAGAAGAG
GAAAGTCtaacTAgtAgaattcctagagctcgctgatcagcctcgactgtgccttctagttgccagccatctgttgtttgcccctccc
ccgtgccttccttgaccctggaaggtgccactcccactgt.cctttcctaataaaatgaggaaattgcatcgcattgtctgagtaggtgtcattct
attctggggggtggggtggggcaggacagcaagggggaggattgggaagagaatagcaggcatgctggggaggtaccgagggccta
tttcccatgattccttcatatttgcatatacgatacaaggctgttagagagataattggaattaatttgactgtaaacacaaagatattagtacaaa
atacgtgacgtagaaagtaataatttcttgggtagtttgcagttttaaaattatgttttaaaatggactatcatatgcttaccgtaacttgaaagtatt
tcgatttcttggctttatatatcttGTGGAAAGGACGAAACACCGGTTATCTCCTGTTCTGCAGCGTT
TcAGAGCTAtgctgGAAAcagcaTAGCAAGTTgAAATAAGGCTAGTCCGTTATCAACTTGA
AAAAGTGGCACCGAGTCGGTGCTTTTTTgcggccgcaggaacccctagtgatggagttggccactccctctctg
cgcgctcgctcgctcactgaggccgggcgaccaaaggtcgcccgacgcccgggctttgcccgggcggcctcagtgagcgagcgagcg
cgcagctgcctgcaggggcgcctgtaccgggagatgggggaggctaactgaaacacggaaggagacaataccggaaggaacccgcg
ctatgacggcaataaaaagacagaataaaacgcacgggtgttgggtcgtttgttcataaacgcggggttcggtcccagggctggcactctg
tcgataccccaccgagaccccattgggaccaatacgcccgcgtttcttccttttccccaccccaacccccaagttcgggtgaaggcccagg
gctcgcagccaacgtcggggcggcaagccctgccatagccactacgggtacgtaggccaaccactagaactatagctagagtcctgggc
gaacaaacgatgctcgccttccagaaaaccgaggatgcgaaccacttcatccggggtcagcaccaccggcaagcgccgcgacggccg
aggtctaccgatctcctgaagccagggcagatccgtgcacagcaccttgccgtagaagaacagcaaggccgccaatgcctgacgatgcg
tggagaccgaaaccttgcgctcgttcgccagccaggacagaaatgcctcgacttcgctgctgcccaaggttgccgggtgacgcacaccgt
ggaaacggatgaaggcacgaacccagttgacataagcctgttcggttcgtaaactgtaatgcaagtagcgtatgcgctcacgcaactggtc
cagaaccttgaccgaacgcagcggtggtaacggcgcagtggcggttttcatggcttgttatgactgtttttttgtacagtctatgcctcgggca
tccaagcagcaagcgcgttacgccgtgggtcgatgtttgatgttatggagcagcaacgatgttacgcagcagcaacgatgttacgcagcag
ggcagtcgccctaaaacaaagttaggtggctcaagtatgggcatcattcgcacatgtaggctcggccctgaccaagtcaaatccatgcggg
ctgctcttgatcttttcggtcgtgagttcggagacgtagccacctactcccaacatcagccggactccgattacctcgggaacttgctccgtag
taagacattcatcgcgcttgctgccttcgaccaagaagcggttgttggcgctctcgcggcttacgttctgcccaggtttgagcagccgcgta
gtgagatctatatctatgatctcgcagtctccggcgagcaccggaggcagggcattgccaccgcgctcatcaatctcctcaagcatgaggc
caacgcgcttggtgcttatgtgatctacgtgcaagcagattacggtgacgatcccgcagtggctctctatacaaagttgggcatacgggaag
aagtgatgcactttgatatcgacccaagtaccgccacctaacaattcgttcaagccgagatcggcttcccggccgcggagttgttcggtaaa
ttgtcacaacgccgcgaatatagtctttaccatgcccttggccacgcccctctttaatacgacgggcaatttgcacttcagaaaatgaagagtt
tgctttagccataacaaaagtccagtatgctttttcacagcataactggactgatttcagtttacaactattctgtctagtttaagactttattgtcat
agtttagatctattttgttcagtttaagactttattgtccgcccacacccgcttacgcagggcatccatttattactcaaccgtaaccgattttgcca
ggttacgcggctggtctgcggtgtgaaataccgcacagatgcgtaaggagaaaataccgcatcaggcgctcttccgcttcctcgctcactg
actcgctgcgctcggtcgttcggctgcggcgagcggtatcagctcactcaaaggcggtaatacggttatccacagaatcaggggataacg
caggaaagaacatgtgagcaaaaggccagcaaaaggccaggaaccgtaaaaaggccgcgttgctggcgtttttccataggctccgcccc
cctgacgagcatcacaaaaatcgacgctcaagtcagaggtggcgaaacccgacaggactataaagataccaggcgtttccccctggaag
ctccctcgtgcgctctcctgttccgaccctgccgcttaccggatacctgtccgcctttctcccttcgggaagcgtggcgctttctcaatgctcac
gctgtaggtatctcagttcggtgtaggtcgttcgctccaagctgggctgtgtgcacgaaccccccgttcagcccgaccgctgcgccttatcc
ggtaactatcgtcttgagtccaacccggtaagacacgacttatcgccactggcagcagccactggtaacaggattagcagagcgaggtatg
taggcggtgctacagagttcttgaagtggtggcctaactacggctacactagaaggacagtatttggtatctgcgctctgctgaagccagtta
ccttcggaaaaagagttggtagctcttgatccggcaaacaaaccaccgctggtagcggtggtttttttgtttgcaagcagcagattacgcgca
gaaaaaaaggatctcaagaagatcctttgatcttttctacggggtctgacgctcagtggaacgaaaactcacgttaagggattttggtcatga
gattatcaaaaaggatcttcacctagatccttttaaattaaaaatgaagttttaaatcaatctaaagtatatatgagtaaacttggtctgacagttac
caatgcttaatcagtgaggcacctatctcagcgatctgtctatttcgttcatccatagttgcctgactccccgtcgtgtagataactacgatacg
ggagggcttaccatctggccccagtgctgcaatgataccgcgagacccacgctcaccggctccagatttatcagcaataaaccagccagc
cggaagggccgagcgcagaagtggtcctgcaactttatccgcctccatccagtctattaattgttgccgggaagctagagtaagtagttcgc
cagttaatagtttgcgcaacgttgttgccattgctacaggcatcgtggtgtcacgctcgtcgtttggtatggcttcattcagctccggttcccaa
cgatcaaggcgagttacatgatcccccatgttgtgcaaaaaagcggttagctccttcggtcctccgatcgttgtcagaagtaagttggccgca
gtgttatcactcatggttatggcagcactgcataattctcttactgtcatgccatccgtaagatgcttttctgtgactggtgagtactcaaccaagt
cattctgagaatagtgtatgcggcgaccgagttgctcttgcccggcgtcaatacgggataataccgcgccacatagcagaactttaaaagtg
ctcatcattggaaaacgttcttcggggcgaaaactctcaaggatcttaccgctgttgagatccagttcgatgtaacccactcgtgcacccaact
gatcttcagcatcttttactttcaccagcgtttctgggtgagcaaaaacaggaaggcaaaatgccgcaaaaaagggaataagggcgacacg
gaaatgttgaatactcatactcttcctttttcaatattattgaagcatttatcagggttattgtctcatgagcggatacatatttgaatgtatttaga
aaaataaacaaataggggttccgcgcacatttccccgaaaagtgccacctgaaattgtaaacgttaatattttgttaaaattcgcgttaaatttttgt
taaatcagctcattttttaaccaataggccgaaatcggcaaaatcccttataaatcaaaagaatagaccgagatagggttgagtgttgttccagt
ttggaacaagagtccactattaaagaacgtggactccaacgtcaaagggcgaaaaaccgtctatcagggcgatggcccactacgtgaacc
atcaccctaatcaagttttttggggtcgaggtgccgtaaagcactaaatcggaaccctaaagggagcccccgatttagagcttgacgggga
aagccggcgaacgtggcgagaaaggaagggaagaaagcgaaaggagcgggcgctagggcgctggcaagtgtagcggtcacgctgc
gcgtaaccaccacacccgccgcgcttaatgcgccgctacagggcgcgtcccattcgccattcaggctgcaaataagcgttgatattcagtc
aattacaaacattaataacgaagagatgacagaaaaattttcattctgtgacagagaa
(pX601-meCMV-ABEmax-N2)
SEQ ID NO: 5
cctgcaggcagctgcgcgctcgctcgctcactgaggccgcccgggcgtcgggcgacctttggtcgcccggcctcagtgagc
gagcgagcgcgcagagagggagtggccaactccatcactaggggttcctgcggcctctagactcgagCGCGTGATGAGAG
CAGCCACTACGGGTCTAGGCTGCCCATGTAAGGAGGCAAGGCCTGGGGACACCCGA
GATGCCTGGTTATAATTAACCCAGACATGTGGCTGCCCCCCCCCCCCCAACACCTGC
TGCCTGCTAAAAATAACCCTGTCCCTGGTGGccCtgcatgcccACTCACGGGGATTTCCAA
GTCTCCACCCCATTGACGTCAATGGGAGTTTGTTTTGGCACCAAAATCAACGGGACT
TTCCAAAATGTCGTAACAACTCCGCCCCATTGACGCAAATGGGCGGTAGGCGTGTA
CGGTGGGAGGTCTATATAAGCAGAGCTGGTTTAGTGAACCGTCAGATCcgccAcCATGg
ctagcATGAAACGGACAGCCGACGGAAGCGAGTTCGAGTCACCAAAGAAGAAGCGGA
AAGTCTCTGAAGTCGAGTTTAGCCACGAGTATTGGATGAGGCACGCACTGACCCTG
GCAAAGCGAGCATGGGATGAAAGAGAAGTCCCCGTGGGCGCCGTGCTGGTGCACA
ACAATAGAGTGATCGGAGAGGGATGGAACAGGCCAATCGGCCGCCACGACCCTAC
CGCACACGCAGAGATCATGGCACTGAGGCAGGGAGGCCTGGTCATGCAGAATTACC
GCCTGATCGATGCCACCCTGTATGTGACACTGGAGCCATGCGTGATGTGCGCAGGA
GCAATGATCCACAGCAGGATCGGAAGAGTGGTGTTCGGAGCACGGGACGCCAAGA
CCGGCGCAGCAGGCTCCCTGATGGATGTGCTGCACCACCCCGGCATGAACCACCGG
GTGGAGATCACAGAGGGAATCCTGGCAGACGAGTGCGCCGCCCTGCTGAGCGATTT
CTTTAGAATGCGGAGACAGGAGATCAAGGCCCAGAAGAAGGCACAGAGCTCCACC
GACTCTGGAGGATCTAGCGGAGGATCCTCTGGAAGCGAGACACCAGGCACAAGCG
AGTCCGCCACACCAGAGAGCTCCGGCGGCTCCTCCGGAGGATCCTCTGAGGTGGAG
TTTTCCCACGAGTACTGGATGAGACATGCCCTGACCCTGGCCAAGAGGGCACGCGA
TGAGAGGGAGGTGCCTGTGGGAGCCGTGCTGGTGCTGAACAATAGAGTGATCGGCG
AGGGCTGGAACAGAGCCATCGGCCTGCACGACCCAACAGCCCATGCCGAAATTATG
GCCCTGAGACAGGGCGGCCTGGTCATGCAGAACTACAGACTGATTGACGCCACCCT
GTACGTGACATTCGAGCCTTGCGTGATGTGCGCCGGCGCCATGATCCACTCTAGGAT
CGGCCGCGTGGTGTTTGGCGTGAGGAACGCAAAAACCGGCGCCGCAGGCTCCCTGA
TGGACGTGCTGCACTACCCCGGCATGAATCACCGCGTCGAAATTACCGAGGGAATC
CTGGCAGATGAATGTGCCGCCCTGCTGTGCTATTTCTTTCGGATGCCTAGACAGGTG
TTCAATGCTCAGAAGAAGGCCCAGAGCTCCACCGACTCCGGAGGATCTAGCGGAGG
CTCCTCTGGCTCTGAGACACCTGGCACAAGCGAGAGCGCAACACCTGAAAGCAGCG
GGGGCAGCAGCGGGGGGTCAGACAAGAAGTACAGCATCGGCCTGGCCATCGGCAC
CAACTCTGTGGGCTGGGCCGTGATCACCGACGAGTACAAGGTGCCCAGCAAGAAAT
TCAAGGTGCTGGGCAACACCGACCGGCACAGCATCAAGAAGAACCTGATCGGAGC
CCTGCTGTTCGACAGCGGCGAAACAGCCGAGGCCACCCGGCTGAAGAGAACCGCCA
GAAGAAGATACACCAGACGGAAGAACCGGATCTGCTATCTGCAAGAGATCTTCAGC
AACGAGATGGCCAAGGTGGACGACAGCTTCTTCCACAGACTGGAAGAGTCCTTCCT
GGTGGAAGAGGATAAGAAGCACGAGCGGCACCCCATCTTCGGCAACATCGTGGAC
GAGGTGGCCTACCACGAGAAGTACCCCACCATCTACCACCTGAGAAAGAAACTGGT
GGACAGCACCGACAAGGCCGACCTGCGGCTGATCTATCTGGCCCTGGCCCACATGA
TCAAGTTCCGGGGCCACTTCCTGATCGAGGGCGACCTGAACCCCGACAACAGCGAC
GTGGACAAGCTGTTCATCCAGCTGGTGCAGACCTACAACCAGCTGTTCGAGGAAAA
CCCCATCAACGCCAGCGGCGTGGACGCCAAGGCCATCCTGTCTGCCAGACTGAGCA
AGAGCAGACGGCTGGAAAATCTGATCGCCCAGCTGCCCGGCGAGAAGAAGAATGG
CCTGTTCGGAAACCTGATTGCCCTGAGCCTGGGCCTGACCCCCAACTTCAAGAGCA
ACTTCGACCTGGCCGAGGATGCCAAACTGCAGCTGAGCAAGGACACCTACGACGAC
GACCTGGACAACCTGCTGGCCCAGATCGGCGACCAGTACGCCGACCTGTTTCTGGC
CGCCAAGAACCTGTCCGACGCCATCCTGCTGAGCGACATCCTGAGAGTGAACACCG
AGATCACCAAGGCCCCCCTGAGCGCCTCTATGATCAAGAGATACGACGAGCACCAC
CAGGACCTGACCCTGCTGAAAGCTCTCGTGCGGCAGCAGCTGCCTGAGAAGTACAA
AGAGATTTTCTTCGACCAGAGCAAGAACGGCTACGCCGGCTACATTGACGGCGGAG
CCAGCCAGGAAGAGTTCTACAAGTTCATCAAGCCCATCCTGGAAAAGATGGACGGC
ACCGAGGAACTGCTCGTGAAGCTGAACAGAGAGGACCTGCTGCGGAAGCAGCGGA
CCTTCGACAACGGCAGCATCCCCCACCAGATCCACCTGGGAGAGCTGCACGCCATT
CTGCGGCGGCAGGAAGATTTTTACCCATTCCTGAAGGACAACCGGGAAAAGATCGA
GAAGATCCTGACCTTCCGCATCCCCTACTACGTGGGCCCTCTGGCCAGGGGAAACA
GCAGATTCGCCTGGATGACCAGAAAGAGCGAGGAAACCATCACCCCCTGGAACTTC
GAGGAAGTGGTGGACAAGGGCGCTTCCGCCCAGAGCTTCATCGAGCGGATGACCAA
CTTCGATAAGAACCTGCCCAACGAGAAGGTGCTGCCCAAGCACAGCCTGCTGTACG
AGTACTTCACCGTGTATAACGAGCTGACCAAAGTGAAATACGTGACCGAGGGAATG
AGAAAGCCCGCCTTCCTGAGCGGCGAGCAGAAAAAGGCCATCGTGGACCTGCTGTT
CAAGACCAACCGGAAAGTGACCGTGAAGCAGCTGAAAGAGGACTACTTCAAGAAA
ATCGAGTGTTTGGATCTGAAAACGCAAGTTCAAACGCCACAGGGTATGAAAGAAAT
ATCCAATATACAGGTCGGCGATCTCGTCTTGTCTAACACTGGCTATAACGAGGTGCT
GAATGTATTTCCAAAAAGCAAGAAAAAAAGTTACAAGATAACTCTGGAAGATGGA
AAAGAAATTATCTGTTCTGAGGAGCATCTGTTTCCGACCCAAACAGGGGAGATGAA
TATCAGTGGCGGTCTCAAAGAGGGTATGTGTTTGTATGTCAAGGaataactagtagaattcctag
agctcgctgatcagcctcgactgtgccttctagttgccagccatctgttgtttgcccctcccccgtgccttccttgaccctggaaggtgccact
cccactgtcctttcctaataaaatgaggaaattgcatcgcattgtctgagtaggtgtcattctattctggggggtggggtggggcaggacagc
aagggggaggattgggaagagaatagcaggcatgctggggaggtaccgagggcctatttcccatgattccttcatatttgcatatacgata
caaggctgttagagagataattggaattaatttgactgtaaacacaaagatattagtacaaaatacgtgacgtagaaagtaataatttcttgggt
agtttgcagttttaaaattatgttttaaaatggactatcatatgcttaccgtaacttgaaagtatttcgatttcttggctttatatatcttGTGGAA
AGGACGAAACACCGGTTATCTCCTGTTCTGCAGCGTTTcAGAGCTAtgctgGAAAcagcaT
AGCAAGTTgAAATAAGGCTAGTCCGTTATCAACTTGAAAAAGTGGCACCGAGTCGG
TGCTTTTTTgcggccgcaggaacccctagtgatggagttggccactccctctctgcgcgctcgctcgctcactgaggccgggcga
ccaaaggtcgcccgacgcccgggctttgcccgggcggcctcagtgagcgagcgagcgcgcagctgcctgcaggggcgcctgatgcg
gtattttctccttacgcatctgtgcggtatttcacaccgcatacgtcaaagcaaccatagtacgcgccctgtagcggcgcattaagcgcggcg
ggtgtggtggttacgcgcagcgtgaccgctacacttgccagcgccctagcgcccgctcctttcgctttcttcccttcctttctcgccacgttcg
ccggctttccccgtcaagctctaaatcgggggctccctttagggttccgatttagtgctttacggcacctcgaccccaaaaaacttgatttggg
tgatggttcacgtagtgggccatcgccctgatagacggtttttcgccctttgacgttggagtccacgttctttaatagtggactcttgttccaaac
tggaacaacactcaaccctatctcgggctattcttttgatttataagggattttgccgatttcggcctattggttaaaaaatgagctgatttaacaa
aaatttaacgcgaattttaacaaaatattaacgtttacaattttatggtgcactctcagtacaatctgctctgatgccgcatagttaagccagccc
cgacacccgccaacacccgctgacgcgccctgacgggcttgtctgctcccggcatccgcttacagacaagctgtgaccgtctccgggag
ctgcatgtgtcagaggttttcaccgtcatcaccgaaacgcgcgagacgaaagggcctcgtgatacgcctatttttataggttaatgtcatgata
ataatggtttcttagacgtcaggtggcacttttcggggaaatgtgcgcggaacccctatttgtttatttttctaaatacattcaaatatgtatccgct
catgagacaataaccctgataaatgcttcaataatattgaaaaaggaagagtatgagtattcaacatttccgtgtcgcccttattcccttttttgcg
gcattttgccttcctgtttttgctcacccagaaacgctggtgaaagtaaaagatgctgaagatcagttgggtgcacgagtgggttacatcgaa
ctggatctcaacagcggtaagatccttgagagttttcgccccgaagaacgttttccaatgatgagcacttttaaagttctgctatgtggcgcgg
tattatcccgtattgacgccgggcaagagcaactcggtcgccgcatacactattctcagaatgacttggttgagtactcaccagtcacagaaa
agcatcttacggatggcatgacagtaagagaattatgcagtgctgccataaccatgagtgataacactgcggccaacttacttctgacaacg
atcggaggaccgaaggagctaaccgcttttttgcacaacatgggggatcatgtaactcgccttgatcgttgggaaccggagctgaatgaag
ccataccaaacgacgagcgtgacaccacgatgcctgtagcaatggcaacaacgttgcgcaaactattaactggcgaactacttactctagc
ttcccggcaacaattaatagactggatggaggcggataaagttgcaggaccacttctgcgctcggcccttccggctggctggtttattgctg
ataaatctggagccggtgagcgtggaagccgcggtatcattgcagcactggggccagatggtaagccctcccgtatcgtagttatctacac
gacggggagtcaggcaactatggatgaacgaaatagacagatcgctgagataggtgcctcactgattaagcattggtaactgtcagacca
agtttactcatatatactttagattgatttaaaacttcatttttaatttaaaaggatctaggtgaagatcctttttgataatctcatgaccaaaatcc
cttaacgtgagttttcgttccactgagcgtcagaccccgtagaaaagatcaaaggatcttcttgagatcctttttttctgcgcgtaatctgctgcttgc
aaacaaaaaaaccaccgctaccagcggtggtttgtttgccggatcaagagctaccaactctttttccgaaggtaactggcttcagcagagcg
cagataccaaatactgtccttctagtgtagccgtagttaggccaccacttcaagaactctgtagcaccgcctacatacctcgctctgctaatcc
tgttaccagtggctgctgccagtggcgataagtcgtgtcttaccgggttggactcaagacgatagttaccggataaggcgcagcggtcggg
ctgaacggggggttcgtgcacacagcccagcttggagcgaacgacctacaccgaactgagatacctacagcgtgagctatgagaaagc
gccacgcttcccgaagggagaaaggcggacaggtatccggtaagcggcagggtcggaacaggagagcgcacgagggagcttccagg
gggaaacgcctggtatctttatagtcctgtcgggtttcgccacctctgacttgagcgtcgatttttgtgatgctcgtcaggggggcggagccta
tggaaaaacgccagcaacgcggcctttttacggttcctggccttttgctggccttttgctcacatgt
(pX601-meCMV-ABEmaxC2NG)
SEQ ID NO: 6
cctgcaggcagctgcgcgctcgctcgctcactgaggccgcccgggcgtcgggcgacctttggtcgcccggcctcagtgagc
gagcgagcgcgcagagagggagtggccaactccatcactaggggttcctgcggcctctagactcgagCGCGTGATGAGAG
CAGCCACTACGGGTCTAGGCTGCCCATGTAAGGAGGCAAGGCCTGGGGACACCCGA
GATGCCTGGTTATAATTAACCCAGACATGTGGCTGCCCCCCCCCCCCCAACACCTGC
TGCCTGCTAAAAATAACCCTGTCCCTGGTGGccctgcatgcccACTCACGGGGATTTCCAA
GTCTCCACCCCATTGACGTCAATGGGAGTTTGTTTTGGCACCAAAATCAACGGGACT
TTCCAAAATGTCGTAACAACTCCGCCCCATTGACGCAAATGGGCGGTAGGCGTGTA
CGGTGGGAGGTCTATATAAGCAGAGCTGGTTTAGTGAACCGTCAGATCgctagccaccAT
GATGCTCAAGAAGATCCTCAAGATTGAAGAGTTGGACGAGCGCGAGCTTATAGACA
TAGAAGTCAGTGGTAATCACCTTTTCTACGCAAATGACATTTTGACTCACAACTCCT
CTTCAGACGTTTGCTTCGACTCCGTGGAAATCTCCGGCGTGGAAGATCGGTTCAACG
CCTCCCTGGGCACATACCACGATCTGCTGAAAATTATCAAGGACAAGGACTTCCTG
GACAATGAGGAAAACGAGGACATTCTGGAAGATATCGTGCTGACCCTGACACTGTT
TGAGGACAGAGAGATGATCGAGGAACGGCTGAAAACCTATGCCCACCTGTTCGACG
ACAAAGTGATGAAGCAGCTGAAGCGGCGGAGATACACCGGCTGGGGCAGGCTGAG
CCGGAAGCTGATCAACGGCATCCGGGACAAGCAGTCCGGCAAGACAATCCTGGATT
TCCTGAAGTCCGACGGCTTCGCCAACAGAAACTTCATGCAGCTGATCCACGACGAC
AGCCTGACCTTTAAAGAGGACATCCAGAAAGCCCAGGTGTCCGGCCAGGGCGATAG
CCTGCACGAGCACATTGCCAATCTGGCCGGCAGCCCCGCCATTAAGAAGGGCATCC
TGCAGACAGTGAAGGTGGTGGACGAGCTCGTGAAAGTGATGGGCCGGCACAAGCC
CGAGAACATCGTGATCGAAATGGCCAGAGAGAACCAGACCACCCAGAAGGGACAG
AAGAACAGCCGCGAGAGAATGAAGCGGATCGAAGAGGGCATCAAAGAGCTGGGCA
GCCAGATCCTGAAAGAACACCCCGTGGAAAACACCCAGCTGCAGAACGAGAAGCT
GTACCTGTACTACCTGCAGAATGGGCGGGATATGTACGGGACCAGGAACTGGACA
TCAACCGGCTGTCCGACTACGATGTGGACCATATCGTGCCTCAGAGCTTTCTGAAGG
ACGACTCCATCGACAACAAGGTGCTGACCAGAAGCGACAAGAACCGGGGCAAGAG
CGACAACGTGCCCTCCGAAGAGGTCGTGAAGAAGATGAAGAACTACTGGCGGCAG
CTGCTGAACGCCAAGCTGATTACCCAGAGAAAGTTCGACAATCTGACCAAGGCCGA
GAGAGGCGGCCTGAGCGAACTGGATAAGGCCGGCTTCATCAAGAGACAGCTGGTG
GAAACCCGGCAGATCACAAAGCACGTGGCACAGATCCTGGACTCCCGGATGAACAC
TAAGTACGACGAGAATGACAAGCTGATCCGGGAAGTGAAAGTGATCACCCTGAAGT
CCAAGCTGGTGTCCGATTTCCGGAAGGATTTCCAGTTTTACAAAGTGCGCGAGATCA
ACAACTACCACCACGCCCACGACGCCTACCTGAACGCCGTCGTGGGAACCGCCCTG
ATCAAAAAGTACCCTAAGCTGGAAAGCGAGTTCGTGTACGGCGACTACAAGGTGTA
CGACGTGCGGAAGATGATCGCCAAGAGCGAGCAGGAAATCGGCAAGGCTACCGCC
AAGTACTTCTTCTACAGCAACATCATGAACTTTTTCAAGACCGAGATTACCCTGGCC
AACGGCGAGATCCGGAAGCGGCCTCTGATCGAGACAAACGGCGAAACCGGGGAGA
TCGTGTGGGATAAGGGCCGGGATTTTGCCACCGTGCGGAAAGTGCTGAGCATGCCC
CAAGTGAATATCGTGAAAAAGACCGAGGTGCAGACAGGCGGCTTCAGCAAAGAGT
CTATCCgGCCCAAGAGGAACAGCGATAAGCTGATCGCCAGAAAGAAGGACTGGGAC
CCTAAGAAGTACGGCGGCTTCGtCAGCCCCACCGTGGCCTATTCTGTGCTGGTGGTG
GCCAAAGTGGAAAAGGGCAAGTCCAAGAAACTGAAGAGTGTGAAAGAGCTGCTGG
GGATCACCATCATGGAAAGAAGCAGCTTCGAGAAGAATCCCATCGACTTTCTGGAA
GCCAAGGGCTACAAAGAAGTGAAAAAGGACCTGATCATCAAGCTGCCTAAGTACTC
CCTGTTCGAGCTGGAAAACGGCCGGAAGAGAATGCTGGCCTCTGCCcGCtttCTGCAG
AAGGGAAACGAACTGGCCCTGCCCTCCAAATATGTGAACTTCCTGTACCTGGCCAG
CCACTATGAGAAGCTGAAGGGCTCCCCCGAGGATAATGAGCAGAAACAGCTGTTTG
TGGAACAGCACAAGCACTACCTGGACGAGATCATCGAGCAGATCAGCGAGTTCTCC
AAGAGAGTGATCCTGGCCGACGCTAATCTGGACAAAGTGCTGTCCGCCTACAACAA
GCACCGGGATAAGCCCATCAGAGAGCAGGCCGAGAATATCATCCACCTGTTTACCC
TGACCAATCTGGGAGCCCCTcggGCCTTCAAGTACTTTGACACCACCATCGACCGGA
AGgtGTACcggAGCACCAAAGAGGTGCTGGACGCCACCCTGATCCACCAGAGCATCA
CCGGCCTGTACGAGACACGGATCGACCTGTCTCAGCTGGGAGGTGACTCTGGCGGC
TCAAAAAGAACCGCCGACGGCAGCGAATTCGAGCCCAAGAAGAAGAGGAAAGTCta
acTAgtAgaattcctagagctcgctgatcagcctcgactgtgccttctagttgccagccatctgttgtttgcccctcccccgtgccttccttg
accctggaaggtgccactcccactgtcctttcctaataaaatgaggaaattgcatcgcattgtctgagtaggtgtcattctattctggggggtg
gggtggggcaggacagcaagggggaggattgggaagagaatagcaggcatgctggggaggtaccgagggcctatttcccatgattcct
tcatatttgcatatacgatacaaggctgttagagagataattggaattaatttgactgtaaacacaaagatattagtacaaaatacgtgacgtag
aaagtaataatttcttgggtagtttgcagttttaaaattatgttttaaaatggactatcatatgcttaccgtaacttgaaagtatttcgatttcttgg
ctttatatatcttGTGGAAAGGACGAAACACCGGTTATCTCCTGTTCTGCAGCGTTTcAGAGCTA
tgctgGAAAcagcatAGCAAGTTgAAATAAGGCTAGTCCGTTATCAACTTGAAAAAGTGG
CACCGAGTCGGTGCTTTTTTgcggccgcaggaacccctagtgatggagttggccactccctctctgcgcgctcgctcgc
tcactgaggccgggcgaccaaaggtcgcccgacgcccgggctttgcccgggcggcctcagtgagcgagcgagcgcgcagctgcctgc
aggggcgcctgatgcggtattttctccttacgcatctgtgcggtatttcacaccgcatacgtcaaagcaaccatagtacgcgccctgtagcgg
cgcattaagcgcggcgggtgtggtggttacgcgcagcgtgaccgctacacttgccagcgccctagcgcccgctcctttcgctttcttccctt
cctttctcgccacgttcgccggctttccccgtcaagctctaaatcgggggctccctttagggttccgatttagtgctttacggcacctcgaccc
caaaaaacttgatttgggtgatggttcacgtagtgggccatcgccctgatagacggtttttcgccctttgacgttggagtccacgttctttaata
gtggactcttgttccaaactggaacaacactcaaccctatctcgggctattcttttgatttataagggattttgccgatttcggcctattggttaaa
aaatgagctgatttaacaaaaatttaacgcgaattttaacaaaatattaacgtttacaattttatggtgcactctcagtacaatctgctctgatgcc
gcatagttaagccagccccgacacccgccaacacccgctgacgcgccctgacgggcttgtctgctcccggcatccgcttacagacaagct
gtgaccgtctccgggagctgcatgtgtcagaggttttcaccgtcatcaccgaaacgcgcgagacgaaagggcctcgtgatacgcctattttt
ataggttaatgtcatgataataatggtttcttagacgtcaggtggcacttttcggggaaatgtgcgcggaacccctatttgtttatttttctaaatac
attcaaatatgtatccgctcatgagacaataaccctgataaatgcttcaataatattgaaaaaggaagagtatgagtattcaacatttccgtgtc
gcccttattcccttttttgcggcattttgccttcctgtttttgctcacccagaaacgctggtgaaagtaaaagatgctgaagatcagttgggtgca
cgagtgggttacatcgaactggatctcaacagcggtaagatccttgagagttttcgccccgaagaacgttttccaatgatgagcacttttaaa
gttctgctatgtggcgcggtattatcccgtattgacgccgggcaagagcaactcggtcgccgcatacactattctcagaatgacttggttgag
tactcaccagtcacagaaaagcatcttacggatggcatgacagtaagagaattatgcagtgctgccataaccatgagtgataacactgcgg
ccaacttacttctgacaacgatcggaggaccgaaggagctaaccgcttttttgcacaacatgggggatcatgtaactcgccttgatcgttggg
aaccggagctgaatgaagccataccaaacgacgagcgtgacaccacgatgcctgtagcaatggcaacaacgttgcgcaaactattaactg
gcgaactacttactctagcttcccggcaacaattaatagactggatggaggcggataaagttgcaggaccacttctgcgctcggcccttccg
gctggctggtttattgctgataaatctggagccggtgagcgtggaagccgcggtatcattgcagcactggggccagatggtaagccctccc
gtatcgtagttatctacacgacggggagtcaggcaactatggatgaacgaaatagacagatcgctgagataggtgcctcactgattaagcat
tggtaactgtcagaccaagtttactcatatatactttagattgatttaaaacttcatttttaatttaaaaggatctaggtgaagatcctttttgataa
tctcatgaccaaaatcccttaacgtgagttttcgttccactgagcgtcagaccccgtagaaaagatcaaaggatcttcttgagatcctttttttctgcg
cgtaatctgctgcttgcaaacaaaaaaaccaccgctaccagcggtggtttgtttgccggatcaagagctaccaactctttttccgaaggtaact
ggcttcagcagagcgcagataccaaatactgtccttctagtgtagccgtagttaggccaccacttcaagaactctgtagcaccgcctacata
cctcgctctgctaatcctgttaccagtggctgctgccagtggcgataagtcgtgtcttaccgggttggactcaagacgatagttaccggataa
ggcgcagcggtcgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacaggagagcgcac
gagggagcttccagggggaaacgcctggtatctttatagtcctgtcgggtttcgccacctctgacttgagcgtcgatttttgtgatgctcgtca
ggggggcggagcctatggaaaaacgccagcaacgcggcctttttacggttcctggccttttgctggccttttgctcacatgt
(pX601-MHP1-ABEmaxN2)
SEQ ID NO: 7
cctgcaggcagctgcgcgctcgctcgctcactgaggccgcccgggcgtcgggcgacctttggtcgcccggcctcagtgagc
gagcgagcgcgcagagagggagtggccaactccatcactaggggttcctgcggcctctagactcgaggCCCTTCAGATTAA
AAATAACTGAGGTAAGGGCCTGGGTAGGGGAGGTGGTGTGAGACGCTCCTGTCTCT
CCTCTATCTGCCCATCGGCCCTTTGGGGAGGAGGAATGTGCCCAAGGACTAAAAAA
AGGCCATGGAGCCAGAGGGGCGAGGGCAACAGACCTTTCATGGGCAAACCTTGGG
GCCcTGCTGactgtaGATGAGAGCAGCCACTACGGGTCTAGGCTGCCCATGTAAGGAG
GCAAGGCCTGGGGACACCCGAGATGCCTGGTTATAATTAACCCAGACATGTGGCTG
CCCCCCCCCCCCCAACACCTGCTGCCTGCTAAAAATAACCCTGTCCCTGGTGGccCtgc
atgCCCTCCCTGGGGACAGCCCCTCCTGGCTAGTCACACCCTGTAGGCTCCTCTATAT
AACCCAGGGGCACAGGGGCTGCCCtcatttACCACCACCTCCACAGCACAGACAGACA
CTCAGGAGCCAGCccaccatggctagcATGAAACGGACAGCCGACGGAAGCGAGTTCGAGT
CACCAAAGAAGAAGCGGAAAGTCGGATCCTCTGAGGTGGAGTTTTCCCACGAGTAC
TGGATGAGACATGCCCTGACCCTGGCCAAGAGGGCACGCGATGAGAGGGAGGTGC
CTGTGGGAGCCGTGCTGGTGCTGAACAATAGAGTGATCGGCGAGGGCTGGAACAGA
GCCATCGGCCTGCACGACCCAACAGgCCATGCCGAAATTATGGCCCTGAGgCAGGG
CGGCCTGGTCATGCAGAACTACAGACTGATTGACGCCACCCTGTACGgGACATTCGA
GCCTTGCGTGATGTGCGCCGGCGCCATGATCCACTCTAGGATCGGCCGCGTGGTGTT
TGGCGTGAGGAACGCAAAAACCGGCGCCGCAGGCTCCCTGATGGACGTGCTGCACT
ACCCCGGCATGAATCACCGCGTCGAAATTACCGAGGGAATCCTGGCAGATGAATGT
GCCGCCCTGCTGTGCTATTTCTTTCGGATGCCTAGACAGGTGTTCAATGCTCAGAAG
AAGGCCCAGAGCTCCACCGACTCCGGAGGATCTAGCGGAGGCTCCTCTGGCTCTGA
GACACCTGGCACAAGCGAGAGCGCAACACCTGAAAGCAGCGGGGGCAGCAGCGGG
GGGTCAGACAAGAAGTACAGCATCGGCCTGGCCATCGGCACCAACTCTGTGGGCTG
GGCCGTGATCACCGACGAGTACAAGGTGCCCAGCAAGAAATTCAAGGTGCTGGGCA
ACACCGACCGGCACAGCATCAAGAAGAACCTGATCGGAGCCCTGCTGTTCGACAGC
GGCGAAACAGCCGAGGCCACCCGGCTGAAGAGAACCGCCAGAAGAAGATACACCA
GACGGAAGAACCGGATCTGCTATCTGCAAGAGATCTTCAGCAACGAGATGGCCAAG
GTGGACGACAGCTTCTTCCACAGACTGGAAGAGTCCTTCCTGGTGGAAGAGGATAA
GAAGCACGAGCGGCACCCCATCTTCGGCAACATCGTGGACGAGGTGGCCTACCACG
AGAAGTACCCCACCATCTACCACCTGAGAAAGAAACTGGTGGACAGCACCGACAA
GGCCGACCTGCGGCTGATCTATCTGGCCCTGGCCCACATGATCAAGTTCCGGGGCC
ACTTCCTGATCGAGGGCGACCTGAACCCCGACAACAGCGACGTGGACAAGCTGTTC
ATCCAGCTGGTGCAGACCTACAACCAGCTGTTCGAGGAAAACCCCATCAACGCCAG
CGGCGTGGACGCCAAGGCCATCCTGTCTGCCAGACTGAGCAAGAGCAGACGGCTGG
AAAATCTGATCGCCCAGCTGCCCGGCGAGAAGAAGAATGGCCTGTTCGGAAACCTG
ATTGCCCTGAGCCTGGGCCTGACCCCCAACTTCAAGAGCAACTTCGACCTGGCCGA
GGATGCCAAACTGCAGCTGAGCAAGGACACCTACGACGACGACCTGGACAACCTGC
TGGCCCAGATCGGCGACCAGTACGCCGACCTGTTTCTGGCCGCCAAGAACCTGTCC
GACGCCATCCTGCTGAGCGACATCCTGAGAGTGAACACCGAGATCACCAAGGCCCC
CCTGAGCGCCTCTATGATCAAGAGATACGACGAGCACCACCAGGACCTGACCCTGC
TGAAAGCTCTCGTGCGGCAGCAGCTGCCTGAGAAGTACAAAGAGATTTTCTTCGAC
CAGAGCAAGAACGGCTACGCCGGCTACATTGACGGCGGAGCCAGCCAGGAAGAGT
TCTACAAGTTCATCAAGCCCATCCTGGAAAAGATGGACGGCACCGAGGAACTGCTC
GTGAAGCTGAACAGAGAGGACCTGCTGCGGAAGCAGCGGACCTTCGACAACGGCA
GCATCCCCCACCAGATCCACCTGGGAGAGCTGCACGCCATTCTGCGGCGGCAGGAA
GATTTTTACCCATTCCTGAAGGACAACCGGGAAAAGATCGAGAAGATCCTGACCTT
CCGCATCCCCTACTACGTGGGCCCTCTGGCCAGGGGAAACAGCAGATTCGCCTGGA
TGACCAGAAAGAGCGAGGAAACCATCACCCCCTGGAACTTCGAGGAAGTGGTGGA
CAAGGGCGCTTCCGCCCAGAGCTTCATCGAGCGGATGACCAACTTCGATAAGAACC
TGCCCAACGAGAAGGTGCTGCCCAAGCACAGCCTGCTGTACGAGTACTTCACCGTG
TATAACGAGCTGACCAAAGTGAAATACGTGACCGAGGGAATGAGAAAGCCCGCCTT
CCTGAGCGGCGAGCAGAAAAAGGCCATCGTGGACCTGCTGTTCAAGACCAACCGGA
AAGTGACCGTGAAGCAGCTGAAAGAGGACTACTTCAAGAAAATCGAGTGTTTGGAT
CTGAAAACGCAAGTTCAAACGCCACAGGGTATGAAAGAAATATCCAATATACAGGT
CGGCGATCTCGTCTTGTCTAACACTGGCTATAACGAGGTGCTGAATGTATTTCCAAA
AAGCAAGAAAAAAAGTTACAAGATAACTCTGGAAGATGGAAAAGAAATTATCTGT
TCTGAGGAGCATCTGTTTCCGACCCAAACAGGGGAGATGAATATCAGTGGCGGTCT
CAAAGAGGGTATGTGTTTGTATGTCAAGGaataactagtagaattcctagagctcgctgatcagcctcgactgt
gccttctagttgccagccatctgttgtttgcccctcccccgtgccttccttgaccctggaaggtgccactcccactgtcctttcctaataaaatga
ggaaattgcatcgcattgtctgagtaggtgtcattctattctggggggtggggtggggcaggacagcaagggggaggattgggaagaga
atagcaggcatgctggggaggtaccgagggcctatttcccatgattccttcatatttgcatatacgatacaaggctgttagagagataattgg
aattaatttgactgtaaacacaaagatattagtacaaaatacgtgacgtagaaagtaataatttcttgggtagtttgcagttttaaaattatgtttta
aaatggactatcatatgcttaccgtaacttgaaagtatttcgatttcttggctttatatatcttGTGGAAAGGACGAAACACCG
GTTATCTCCTGTTCTGCAGCGTTTcAGAGCTAtgctgGAAAcagcaTAGCAAGTTgAAATA
AGGCTAGTCCGTTATCAACTTGAAAAAGTGGCACCGAGTCGGTGCTTTTTTgcggccgca
ggaacccctagtgatggagttggccactccctctctgcgcgctcgctcgctcactgaggccgggcgaccaaaggtcgcccgacgcccgg
gctttgcccgggcggcctcagtgagcgagcgagcgcgcagctgcctgcaggggcgcctgatgcggtattttctccttacgcatctgtgcg
gtatttcacaccgcatacgtcaaagcaaccatagtacgcgccctgtagcggcgcattaagcgcggcgggtgtggtggttacgcgcagcgt
gaccgctacacttgccagcgccctagcgcccgctcctttcgctttcttcccttcctttctcgccacgttcgccggctttccccgtcaagctctaa
atcgggggctccctttagggttccgatttagtgctttacggcacctcgaccccaaaaaacttgatttgggtgatggttcacgtagtgggccatc
gccctgatagacggtttttcgccctttgacgttggagtccacgttctttaatagtggactcttgttccaaactggaacaacactcaaccctatctc
gggctattcttttgatttataagggattttgccgatttcggcctattggttaaaaaatgagctgatttaacaaaaatttaacgcgaattttaacaaaa
tattaacgtttacaattttatggtgcactctcagtacaatctgctctgatgccgcatagttaagccagccccgacacccgccaacacccgctga
cgcgccctgacgggcttgtctgctcccggcatccgcttacagacaagctgtgaccgtctccgggagctgcatgtgtcagaggttttcaccgt
catcaccgaaacgcgcgagacgaaagggcctcgtgatacgcctatttttataggttaatgtcatgataataatggtttcttagacgtcaggtgg
cacttttcggggaaatgtgcgcggaacccctatttgtttatttttctaaatacattcaaatatgtatccgctcatgagacaataaccctgataaatg
cttcaataatattgaaaaaggaagagtatgagtattcaacatttccgtgt.cgcccttattcccttttttgcggcattttgccttcctgtttttgctc
acccagaaacgctggtgaaagtaaaagatgctgaagatcagttgggtgcacgagtgggttacatcgaactggatctcaacagcggtaagatc
cttgagagttttcgccccgaagaacgttttccaatgatgagcacttttaaagttctgctatgtggcgcggtattatcccgtattgacgccgggca
agagcaactcggtcgccgcatacactattctcagaatgacttggttgagtactcaccagtcacagaaaagcatcttacggatggcatgacag
taagagaattatgcagtgctgccataaccatgagtgataacactgcggccaacttacttctgacaacgatcggaggaccgaaggagctaac
cgcttttttgcacaacatgggggatcatgtaactcgccttgatcgttgggaaccggagctgaatgaagccataccaaacgacgagcgtgac
accacgatgcctgtagcaatggcaacaacgttgcgcaaactattaactggcgaactacttactctagcttcccggcaacaattaatagactgg
atggaggcggataaagttgcaggaccacttctgcgctcggcccttccggctggctggtttattgctgataaatctggagccggtgagcgtg
gaagccgcggtatcattgcagcactggggccagatggtaagccctcccgtatcgtagttatctacacgacggggagtcaggcaactatgg
atgaacgaaatagacagatcgctgagataggtgcctcactgattaagcattggtaactgtcagaccaagtttactcatatatactttagattgat
ttaaaacttcatttttaatttaaaaggatctaggtgaagatcctttttgataatctcatgaccaaaatcccttaacgtgagttttcgttccactgagcg
tcagaccccgtagaaaagatcaaaggatcttcttgagatcctttttttctgcgcgtaatctgctgcttgcaaacaaaaaaaccaccgctaccag
cggtggtttgtttgccggatcaagagctaccaactctttttccgaaggtaactggcttcagcagagcgcagataccaaatactgtccttctagt
gtagccgtagttaggccaccacttcaagaactctgtagcaccgcctacatacctcgctctgctaatcctgttaccagtggctgctgccagtgg
cgataagtcgtgtcttaccgggttggactcaagacgatagttaccggataaggcgcagcggtcgggctgaacggggggttcgtgcacaca
gcccagcttggagcgaacgacctacaccgaactgagatacctacagcgtgagctatgagaaagcgccacgcttcccgaagggagaaag
gcggacaggtatccggtaagcggcagggtcggaacaggagagcgcacgagggagcttccagggggaaacgcctggtatctttatagtc
ctgtcgggtttcgccacctctgacttgagcgtcgatttttgtgatgctcgtcaggggggcggagcctatggaaaaacgccagcaacgcggc
ctttttacggttcctggccttttgctggccttttgctcacatgt
(pX601-MHP1-iABEmaxC2NG: ITR-MHP1-Gp41-1 inteinC-
Cas9NG574-1368-NLS-bGHpA-hU6-mdx4cv_spacer-ogRNA_scaffold-ITR)
SEQ ID NO: 8
cctgcaggcagctgcgcgctcgctcgctcactgaggccgcccgggcgtcgggcgacctttggtcgcccggcctcagtgagc
gagcgagcgcgcagagagggagtggccaactccatcactaggggttcctgcggcctctagactcgaggCCCTTCAGATTAA
AAATAACTGAGGTAAGGGCCTGGGTAGGGGAGGTGGTGTGAGACGCTCCTGTCTCT
CCTCTATCTGCCCATCGGCCCTTTGGGGAGGAGGAATGTGCCCAAGGACTAAAAAA
AGGCCATGGAGCCAGAGGGGCGAGGGCAACAGACCTTTCATGGGCAAACCTTGGG
GCCCTGCTGactgtaGATGAGAGCAGCCACTACGGGTCTAGGCTGCCCATGTAAGGAG
GCAAGGCCTGGGGACACCCGAGATGCCTGGTTATAATTAACCCAGACATGTGGCTG
CCCCCCCCCCCCCAACACCTGCTGCCTGCTAAAAATAACCCTGTCCCTGGTGGccctgc
atgCcCTCCCTGGGGACAGCCCCTCCTGGCTAGTCACACCCTGTAGGCTCCTCTATAT
AACCCAGGGGCACAGGGGCTGCCCtcatttACCACCACCTCCACAGCACAGACAGACA
CTCAGGAGCCAGCtagccaccATGATGCTCAAGAAGATCCTCAAGATTGAAGAGTTGGA
CGAGCGCGAGCTTATAGACATAGAAGTCAGTGGTAATCACCTTTTCTACGCAAATG
ACATTTTGACTCACAACTCCTCTTCAGACGTTTGCTTCGACTCCGTGGAAATCTCCG
GCGTGGAAGATCGGTTCAACGCCTCCCTGGGCACATACCACGATCTGCTGAAAATT
ATCAAGGACAAGGACTTCCTGGACAATGAGGAAAACGAGGACATTCTGGAAGATA
TCGTGCTGACCCTGACACTGTTTGAGGACAGAGAGATGATCGAGGAACGGCTGAAA
ACCTATGCCCACCTGTTCGACGACAAAGTGATGAAGCAGCTGAAGCGGCGGAGATA
CACCGGCTGGGGCAGGCTGAGCCGGAAGCTGATCAACGGCATCCGGGACAAGCAG
TCCGGCAAGACAATCCTGGATTTCCTGAAGTCCGACGGCTTCGCCAACAGAAACTT
CATGCAGCTGATCCACGACGACAGCCTGACCTTTAAAGAGGACATCCAGAAAGCCC
AGGTGTCCGGCCAGGGCGATAGCCTGCACGAGCACATTGCCAATCTGGCCGGCAGC
CCCGCCATTAAGAAGGGCATCCTGCAGACAGTGAAGGTGGTGGACGAGCTCGTGAA
AGTGATGGGCCGGCACAAGCCCGAGAACATCGTGATCGAAATGGCCAGAGAGAAC
CAGACCACCCAGAAGGGACAGAAGAACAGCCGCGAGAGAATGAAGCGGATCGAAG
AGGGCATCAAAGAGCTGGGCAGCCAGATCCTGAAAGAACACCCCGTGGAAAACAC
CCAGCTGCAGAACGAGAAGCTGTACCTGTACTACCTGCAGAATGGGCGGGATATGT
ACGTGGACCAGGAACTGGACATCAACCGGCTGTCCGACTACGATGTGGACCATATC
GTGCCTCAGAGCTTTCTGAAGGACGACTCCATCGACAACAAGGTGCTGACCAGAAG
CGACAAGAACCGGGGCAAGAGCGACAACGTGCCCTCCGAAGAGGTCGTGAAGAAG
ATGAAGAACTACTGGCGGCAGCTGCTGAACGCCAAGCTGATTACCCAGAGAAAGTT
CGACAATCTGACCAAGGCCGAGAGAGGCGGCCTGAGCGAACTGGATAAGGCCGGC
TTCATCAAGAGACAGCTGGTGGAAACCCGGCAGATCACAAAGCACGTGGCACAGAT
CCTGGACTCCCGGATGAACACTAAGTACGACGAGAATGACAAGCTGATCCGGGAAG
TGAAAGTGATCACCCTGAAGTCCAAGCTGGTGTCCGATTTCCGGAAGGATTTCCAGT
TTTACAAAGTGCGCGAGATCAACAACTACCACCACGCCCACGACGCCTACCTGAAC
GCCGTCGTGGGAACCGCCCTGATCAAAAAGTACCCTAAGCTGGAAAGCGAGTTCGT
GTACGGCGACTACAAGGTGTACGACGTGCGGAAGATGATCGCCAAGAGCGAGCAG
GAAATCGGCAAGGCTACCGCCAAGTACTTCTTCTACAGCAACATCATGAACTTTTTC
AAGACCGAGATTACCCTGGCCAACGGCGAGATCCGGAAGCGGCCTCTGATCGAGAC
AAACGGCGAAACCGGGGAGATCGTGTGGGATAAGGGCCGGGATTTTGCCACCGTGC
GGAAAGTGCTGAGCATGCCCCAAGTGAATATCGTGAAAAAGACCGAGGTGCAGAC
AGGCGGCTTCAGCAAAGAGTCTATCCgGCCCAAGAGGAACAGCGATAAGCTGATCG
CCAGAAAGAAGGACTGGGACCCTAAGAAGTACGGCGGCTTCGtCAGCCCCACCGTG
GCCTATTCTGTGCTGGTGGTGGCCAAAGTGGAAAAGGGCAAGTCCAAGAAACTGAA
GAGTGTGAAAGAGCTGCTGGGGATCACCATCATGGAAAGAAGCAGCTTCGAGAAG
AATCCCATCGACTTTCTGGAAGCCAAGGGCTACAAAGAAGTGAAAAAGGACCTGAT
CATCAAGCTGCCTAAGTACTCCCTGTTCGAGCTGGAAAACGGCCGGAAGAGAATGC
TGGCCTCTGCCcGCtttCTGCAGAAGGGAAACGAACTGGCCCTGCCCTCCAAATATGT
GAACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCATC
GAGCAGATCAGCGAGTTCTCCAAGAGAGTGATCCTGGCCGACGCTAATCTGGACAA
AGTGCTGTCCGCCTACAACAAGCACCGGGATAAGCCCATCAGAGAGCAGGCCGAG
AATATCATCCACCTGTTTACCCTGACCAATCTGGGAGCCCCTcggGCCTTCAAGTACT
TTGACACCACCATCGACCGGAAGgtGTACcggAGCACCAAAGAGGTGCTGGACGCCA
CCCTGATCCACCAGAGCATCACCGGCCTGTACGAGACACGGATCGACCTGTCTCAG
CTGGGAGGTGACTCTGGCGGCTCAAAAAGAACCGCCGACGGCAGCGAATTCGAGCC
CAAGAAGAAGAGGAAAGTCtaacTAgtAgaattcctagagctcgctgatcagcctcgactgtgccttctagttgccag
ccatctgttgtttgcccctcccccgtgccttccttgaccctggaaggtgccactcccactgtcctttcctaataaaatgaggaaattgcatcgca
ttgtctgagtaggtgtcattctattctggggggtggggtggggcaggacagcaagggggaggattgggaagagaatagcaggcatgctg
gggaggtaccgagggcctatttcccatgattccttcatatttgcatatacgatacaaggctgttagagagataattggaattaatttgactgtaa
acacaaagatattagtacaaaatacgtgacgtagaaagtaataatttcttgggtagtttgcagttttaaaattatgttttaaaatggactatcatatg
cttaccgtaacttgaaagtatttcgatttcttggctttatatatcttGTGGAAAGGACGAAACACCGGTTATCTCCT
GTTCTGCAGCGTTTcAGAGCTAtgctgGAAAcagcaTAGCAAGTTgAAATAAGGCTAGTCC
GTTATCAACTTGAAAAAGTGGCACCGAGTCGGTGCTTTTTTgcggccgcaggaacccctagtgatg
gagttggccactccctctctgcgcgctcgctcgctcactgaggccgggcgaccaaaggtcgcccgacgcccgggctttgcccgggcgg
cctcagtgagcgagcgagcgcgcagctgcctgcaggggcgcctgatgcggtattttctccttacgcatctgtgcggtatttcacaccgcata
cgtcaaagcaaccatagtacgcgccctgtagcggcgcattaagcgcggcgggtgtggtggttacgcgcagcgtgaccgctacacttgcc
agcgccctagcgcccgctcctttcgctttcttcccttcctttctcgccacgttcgccggctttccccgtcaagctctaaatcgggggctcccttta
gggttccgatttagtgctttacggcacctcgaccccaaaaaacttgatttgggtgatggttcacgtagtgggccatcgccctgatagacggttt
ttcgccctttgacgttggagtccacgttctttaatagtggactcttgttccaaactggaacaacactcaaccctatctcgggctattcttttgattta
taagggattttgccgatttcggcctattggttaaaaaatgagctgatttaacaaaaatttaacgcgaattttaacaaaatattaacgtttacaatttt
atggtgcactctcagtacaatctgctctgatgccgcatagttaagccagccccgacacccgccaacacccgctgacgcgccctgacgggc
ttgtctgctcccggcatccgcttacagacaagctgtgaccgtctccgggagctgcatgtgtcagaggttttcaccgtcatcaccgaaacgcg
cgagacgaaagggcctcgtgatacgcctatttttataggttaatgtcatgataataatggtttcttagacgtcaggtggcacttttcggggaaat
gtgcgcggaacccctatttgtttatttttctaaatacattcaaatatgtatccgctcatgagacaataaccctgataaatgcttcaataatattgaaa
aaggaagagtatgagtattcaacatttccgtgtcgcccttattcccttttttgcggcattttgccttcctgtttttgctcacccagaaacgctggtg
aaagtaaaagatgctgaagatcagttgggtgcacgagtgggttacatcgaactggatctcaacagcggtaagatccttgagagttttcgccc
cgaagaacgttttccaatgatgagcacttttaaagttctgctatgtggcgcggtattatcccgtattgacgccgggcaagagcaactcggtcg
ccgcatacactattctcagaatgacttggttgagtactcaccagtcacagaaaagcatcttacggatggcatgacagtaagagaattatgcag
tgctgccataaccatgagtgataacactgcggccaacttacttctgacaacgatcggaggaccgaaggagctaaccgcttttttgcacaaca
tgggggatcatgtaactcgccttgatcgttgggaaccggagctgaatgaagccataccaaacgacgagcgtgacaccacgatgcctgtag
caatggcaacaacgttgcgcaaactattaactggcgaactacttactctagcttcccggcaacaattaatagactggatggaggcggataaa
gttgcaggaccacttctgcgctcggcccttccggctggctggtttattgctgataaatctggagccggtgagcgtggaagccgcggtatcat
tgcagcactggggccagatggtaagccctcccgtatcgtagttatctacacgacggggagtcaggcaactatggatgaacgaaatagaca
gatcgctgagataggtgcctcactgattaagcattggtaactgtcagaccaagtttactcatatatactttagattgatttaaaacttcatttttaat
ttaaaaggatctaggtgaagatcctttttgataatctcatgaccaaaatcccttaacgtgagttttcgttccactgagcgtcagaccccgtagaaa
agatcaaaggatcttcttgagatcctttttttctgcgcgtaatctgctgcttgcaaacaaaaaaaccaccgctaccagcggtggtttgtttgccg
gatcaagagctaccaactctttttccgaaggtaactggcttcagcagagcgcagataccaaatactgtccttctagtgtagccgtagttaggc
caccacttcaagaactctgtagcaccgcctacatacctcgctctgctaatcctgttaccagtggctgctgccagtggcgataagtcgtgtctta
ccgggttggactcaagacgatagttaccggataaggcgcagcggtcgggctgaacggggggttcgtgcacacagcccagcttggagcg
aacgacctacaccgaactgagatacctacagcgtgagctatgagaaagcgccacgcttcccgaagggagaaaggcggacaggtatccg
gtaagcggcagggtcggaacaggagagcgcacgagggagcttccagggggaaacgcctggtatctttatagtcctgtcgggtttcgcca
cctctgacttgagcgtcgatttttgtgatgctcgtcaggggggcggagcctatggaaaaacgccagcaacgcggcctttttacggttcctgg
ccttttgctggccttttgctcacatgt
(pLenti-mdx^{4cv}-ogRNA: U6 promoter, mdx4cv spacer, optimized gRNA scaffold)
SEQ ID NO: 9
gtcgacggatcgggagatctcccgatcccctatggtgcactctcagtacaatctgctctgatgccgcatagttaagccagtatctg
ctccctgcttgtgtgttggaggtcgctgagtagtgcgcgagcaaaatttaagctacaacaaggcaaggcttgaccgacaattgcatgaagaa
tctgcttagggttaggcgttttgcgctgcttcgcgatgtacgggccagatatacgcgttgacattgattattgactagttattaatagtaatcaatt
acggggtcattagttcatagcccatatatggagttccgcgttacataacttacggtaaatggcccgcctggctgaccgcccaacgacccccg
cccattgacgtcaataatgacgtatgttcccatagtaacgccaatagggactttccattgacgtcaatgggtggagtatttacggtaaactgcc
cacttggcagtacatcaagtgtatcatatgccaagtacgccccctattgacgtcaatgacggtaaatggcccgcctggcattatgcccagtac
atgaccttatgggactttcctacttggcagtacatctacgtattagtcatcgctattaccatggtgatgcggttttggcagtacatcaatgggcgt
ggatagcggtttgactcacggggatttccaagtctccaccccattgacgtcaatgggagtttgttttggcaccaaaatcaacgggactttcca
aaatgtcgtaacaactccgccccattgacgcaaatgggcggtaggcgtgtacggtgggaggtctatataagcagcgcgttttgcctgtactg
ggtctctctggttagaccagatctgagcctgggagctctctggctaactagggaacccactgcttaagcctcaataaagcttgccttgagtgc
ttcaagtagtgtgtgccogtctgttgtgtgactctggtaactagagatccctcagacccttttagtcagtgtggaaaatctctagcagtggogcc
cgaacagggacttgaaagcgaaagggaaaccagaggagctctctcgacgcaggactcggcttgctgaagcgcgcacggcaagaggc
gaggggcggcgactggtgagtacgccaaaaattttgactagcggaggctagaaggagagagatgggtgcgagagcgtcagtattaagc
gggggagaattagatcgcgatgggaaaaaattcggttaaggccagggggaaagaaaaaatataaattaaaacatatagtatgggcaagca
gggagctagaacgattcgcagttaatcctggcctgttagaaacatcagaaggctgtagacaaatactgggacagctacaaccatcccttca
gacaggatcagaagaacttagatcattatataatacagtagcaaccctctattgtgtgcatcaaaggatagagataaaagacaccaaggaag
ctttagacaagatagaggaagagcaaaacaaaagtaagaccaccgcacagcaagcggccgctgatcttcagacctggaggaggagata
tgagggacaattggagaagtgaattatataaatataaagtagtaaaaattgaaccattaggagtagcacccaccaaggcaaagagaagagt
ggtgcagagagaaaaaagagcagtgggaataggagctttgttccttgggttcttgggagcagcaggaagcactatgggcgcagcgtcaa
tgacgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggggatt
tggggttgctctggaaaactcatttgcaccactgctgtgccttggaatgctagttggagtaataaatctctggaacagatttggaatcacacga
cctggatggagtgggacagagaaattaacaattacacaagcttaatacactccttaattgaagaatcgcaaaaccagcaagaaaagaatga
acaagaattattggaattagataaatgggcaagtttgtggaattggtttaacataacaaattggctgtggtatataaaattattcataatgatagta
ggaggcttggtaggtttaagaatagtttttgctgtactttctatagtgaatagagttaggcagggatattcaccattatcgtttcagacccacctc
ccaaccccgaggggacccgacaggcccgaaggaatagaagaagaaggtggagagagagacagagacagatccattcgattagtgaa
cggatcggcactgcgtgcgccaattctgcagacaaatggcagtattcatccacaattttaaaagaaaaggggggattggggggtacagtgc
nggggaaagaatagtagacataatagcaacagacatacaaactaaagaattacaaaaacaaattacaaaaattcaaaattttcgggtttatta
cagggacagcagagatccagtttggttaattagctagcaggtacctgagggcctatttcccatgattccttcatatttgcatatacgatacaag
gctgttagagagataattggaattaatttgactgtaaacacaaagatattagtacaaaatacgtgacgtagaaagtaataatttcttgggtagttt
gcagttttaaaattatgttttaaaatggactatcatatgcttaccgtaacttgaaagtatttcgatttcttggctttatatatcttgtggaaaggacga
aacaccgGTTATCTCCTGTTCTGCAGCGTTTcAGAGCTAtgctgGAAAcagcaTAGCAAGTTgA
AATAAGGCTAGTCCGTTATCAACTTGAAAAAGTGGCACCGAGTCGGTGCTTTTTTgcg
gccgcggatcctgcaaagatggataaagttttaaacagagaggaatctttgcagctaatggaccttctaggtcttgaaaggagtgggaattg
gctccggtgcccgtcagtgggcagagcgcacatcgcccacagtccccgagaagttggggggaggggtcggcaattgatccggtgccta
gagaaggtggcgcggggtaaactgggaaagtgatgtcgtgtactggctccgcctttttcccgagggtgggggagaaccgtatataagtgc
agtagtcgccgtgaacgttctttttcgcaacgggtttgccgccagaacacaggtaagtgccgtgtgtggttcccgcgggcctggcctctttac
gggttatggcccttgcgtgccttgaattacttccactggctgcagtacgtgattcttgatcccgagcttcgggttggaagtgggtgggagagtt
cgaggccttgcgcttaaggagccccttcgcctcgtgcttgagttgaggcctggcctgggcgctggggccgccgcgtgcgaatctggtggc
accttcgcgcctgtctcgctgctttcgataagtctctagccatttaaaatttttgatgacctgctgcgacgctttttttctggcaagatagtcttgta
aatgcgggccaagatctgcacactggtatttcggtttttggggccgcgggcggcgacggggcccgtgcgtcccagcgcacatgttcggc
gaggcggggcctgcgagcgcggccaccgagaatcggacgggggtagtctcaagctggccggcctgctctggtgcctggcctcgcgcc
gccgtgtatcgccccgccctgggcggcaaggctggcccggtcggcaccagttgcgtgagcggaaagatggccgcttcccggccctgct
gcagggagctcaaaatggaggacgcggcgctcgggagagcgggcgggtgagtcacccacacaaaggaaaagggcctttccgtcctca
gccgtcgcttcatgtgactccacggagtaccgggcgccgtccaggcacctcgattagttctcgagcttttggagtacgtcgtctttaggttgg
ggggaggggttttatgcgatggagtttccccacactgagtgggtggagactgaagttaggccagcttggcacttgatgtaattctccttggaa
tttgccctttttgagtttggatcttggttcattctcaagcctcagacagtggttcaaagtttttttcttccatttcaggtgtcgtgatgtacaatggcc
aagttgaccagtgccgttccggtgctcaccgcgcgcgacgtcgccggagcggtcgagttctggaccgaccggctcgggttctcccgggac
ttcgtggaggacgacttcgccggtgtggtccgggacgacgtgaccctgttcatcagcgcggtccaggaccaggtggtgccggacaacac
cctggcctgggtgtgggtgcgcggcctggacgagctgtacgccgagtggtcggaggtcgtgtccacgaacttccgggacgcctccggg
ccggccatgaccgagatcggcgagcagccgtgggggcgggagttcgccctgcgcgacccggccggcaactgcgtgcacttcgtggcc
gaggagcaggactgagaattcgatatcaagcttatcggtaatcaacctctggattacaaaatttgtgaaagattgactggtattcttaactatgtt
gctccttttacgctatgtggatacgctgctttaatgcctttgtatcatgctattgcttccogtatggctttcattttctcctccttgtataaatcctg
gttgctgtctctttatgaggagttgtggcccgttgtcaggcaacgtggcgtggtgtgcactgtgtttgctgacgcaacccccactggttggggcat
tgccaccacctgtcagctcctttccgggactttcgctttccccctccctattgccacggcggaactcatcgccgcctgccttgcccgctgctg
gacaggggctcggctgttgggcactgacaattccgtggtgttgtcggggaaatcatcgtcctttccttggctgctcgcctgtgttgccacctg
gattctgcgcgggacgtccttctgctacgtcccttcggccctcaatccagcggaccttccttcccgcggcctgctgccggctctgcggcctct
tccgcgtcttcgccttcgccctcagacgagtcggatctccctttgggccgcctccccgcatcgataccgtcgacctcgagacctagaaaaac
atggagcaatcacaagtagcaatacagcagctaccaatgctgattgtgcctggctagaagcacaagaggaggaggaggtgggttttccag
tcacacctcaggtacctttaagaccaatgacttacaaggcagctgtagatcttagccactttttaaaagaaaaggggggactggaagggcta
attcactcccaacgaagacaagatatccttgatctgtggatctaccacacacaaggctacttccctgattggcagaactacacaccagggcc
agggatcagatatccactgacctttggatggtgctacaagctagtaccagttgagcaagagaaggtagaagaagccaatgaaggagagaa
cacccgcttgttacaccctgtgagcctgcatgggatggatgacccggagagagaagtattagagtggaggtttgacagccgcctagcattt
catcacatggcccgagagctgcatccggactgtactgggtctctctggttagaccagatctgagcctgggagctctctggctaactaggga
acccactgcttaagcctcaataaagcttgccttgagtgcttcaagtagtgtgtgcccgtctgttgtgtgactctggtaactagagatccctcaga
cccttttagtcagtgtggaaaatctctagcagggcccgtttaaacccgctgatcagcctcgactgtgccttctagttgccagccatctgttgttt
gcccctcccccgtgccttccttgaccctggaaggtgccactcccactgtcctttcctaataaaatgaggaaattgcatcgcattgtctgagtag
gtgtcattctattctggggggtggggtggggcaggacagcaagggggaggattgggaagacaatagcaggcatgctggggatgcggtg
ggctctatggcttctgaggcggaaagaaccagctggggctctagggggtatccccacgcgccctgtagcggcgcattaagcgcggcgg
gtgtggtggttacgcgcagcgtgaccgctacacttgccagcgccctagcgcccgctcctttcgctttcttcccttcctttctcgccacgttcgc
cggctttccccgtcaagctctaaatcgggggctccctttagggttccgatttagtgctttacggcacctcgaccccaaaaaacttgattagggt
gatggttcacgtagtgggccatcgccctgatagacggtttttcgccctttgacgttggagtccacgttctttaatagtggactcttgttccaaact
ggaacaacactcaaccctatctcggtctattcttttgatttataagggattttgccgatttcggcctattggttaaaaaatgagctgatttaacaaa
aatttaacgcgaattaattctgtggaatgtgtgtcagttagggtgtggaaagtccccaggctccccagcaggcagaagtatgcaaagcatgc
atctcaattagtcagcaaccaggtgtggaaagtccccaggctccccagcaggcagaagtatgcaaagcatgcatctcaattagtcagcaac
catagtcccgcccctaactccgcccatcccgcccctaactccgcccagttccgcccattctccgccccatggctgactaattttttttatttatgc
agaggccgaggccgcctctgcctctgagctattccagaagtagtgaggaggcttttttggaggcctaggcttttgcaaaaagctcccggga
gcttgtatatccattttcggatctgatcagcacgtgttgacaattaatcatcggcatagtatatcggcatagtataatacgacaaggtgaggaac
taaaccatggccaagttgaccagtgccgttccggtgctcaccgcgcgcgacgtcgccggagcggtcgagttctggaccgaccggctcgg
gttctcccgggacttcgtggaggacgacttcgccggtgtggtccgggacgacgtgaccctgttcatcagcgcggtccaggaccaggtggt
gccggacaacaccctggcctgggtgtgggtgcgcggcctggacgagctgtacgccgagtggtcggaggtcgtgtccacgaacttccgg
gacgcctccgggccggccatgaccgagatcggcgagcagccgtgggggcgggagttcgccctgcgcgacccggccggcaactgcgt
gcacttcgtggccgaggagcaggactgacacgtgctacgagatttcgattccaccgccgccttctatgaaaggttgggcttcggaatcgttt
tccgggacgccggctggatgatcctccagcgcggggatctcatgctggagttcttcgcccaccccaacttgtttattgcagcttataatggtt
acaaataaagcaatagcatcacaaatttcacaaataaagcatttttttcactgcattctagttgtggtttgtccaaactcatcaatgtatcttatcat
gtctgtataccgtcgacctctagctagagcttggcgtaatcatggtcatagctgtttcctgtgtgaaattgttatccgctcacaattccacacaac
atacgagccggaagcataaagtgtaaagcctggggtgcctaatgagtgagctaactcacattaattgcgttgcgctcactgcccgctttcca
gtcgggaaacctgtcgtgccagctgcattaatgaatcggccaacgcgcggggagaggcggtttgcgtattgggcgctcttccgcttcctcg
ctcactgactcgctgcgctcggtcgttcggctgcggcgagcggtatcagctcactcaaaggcggtaatacggttatccacagaatcagggg
ataacgcaggaaagaacatgtgagcaaaaggccagcaaaaggccaggaaccgtaaaaaggccgcgttgctggcgtttttccataggctc
cgcccccctgacgagcatcacaaaaatcgacgctcaagtcagaggtggcgaaacccgacaggactataaagataccaggcgtttccccc
tggaagctccctcgtgcgctctcctgttccgaccctgccgcttaccggatacctgtccgcctttctcccttcgggaagcgtggcgctttctcat
agctcacgctgtaggtatctcagttcggtgtaggtcgttcgctccaagctgggctgtgtgcacgaaccccccgttcagcccgaccgctgcg
ccttatccggtaactatcgtcttgagtccaacccggtaagacacgacttatcgccactggcagcagccactggtaacaggattagcagagc
gaggtatgtaggcggtgctacagagttcttgaagtggtggcctaactacggctacactagaagaacagtatttggtatctgcgctctgctgaa
gccagttaccttcggaaaaagagttggtagctcttgatccggcaaacaaaccaccgctggtagcggtggtttttttgtttgcaagcagcagat
tacgcgcagaaaaaaaggatctcaagaagatcctttgatcttttctacggggtctgacgctcagtggaacgaaaactcacgttaagggatttt
ggtcatgagattatcaaaaaggatcttcacctagatccttttaaattaaaaatgaagttttaaatcaatctaaagtatatatgagtaaacttggtct
gacagttaccaatgcttaatcagtgaggcacctatctcagcgatctgtctatttcgttcatccatagttgcctgactccccgtcgtgtagataact
acgatacgggagggcttaccatctggccccagtgctgcaatgataccgcgagacccacgctcaccggctccagatttatcagcaataaac
cagccagccggaagggccgagcgcagaagtggtcctgcaactttatccgcctccatccagtctattaattgttgccgggaagctagagtaa
gtagttcgccagttaatagtttgcgcaacgttgttgccattgctacaggcatcgtggtgtcacgctcgtcgtttggtatggcttcattcagctccg
gttcccaacgatcaaggcgagttacatgatcccccatgttgtgcaaaaaagcggttagctccttcggtcctccgatcgttgtcagaagtaagtt
ggccgcagtgttatcactcatggttatggcagcactgcataattctcttactgtcatgccatccgtaagatgcttttctgtgactggtgagtactc
aaccaagtcattctgagaatagtgtatgcggcgaccgagttgctcttgcccggcgtcaatacgggataataccgcgccacatagcagaactt
taaaagtgctcatcattggaaaacgttcttcggggcgaaaactctcaaggatcttaccgctgttgagatccagttcgatgtaacccactcgtgc
acccaactgatcttcagcatcttttactttcaccagcgtttctgggtgagcaaaaacaggaaggcaaaatgccgcaaaaaagggaataagg
gcgacacggaaatgttgaatactcatactcttcctttttcaatattattgaagcatttatcagggttattgtctcatgagcggatacatatttgaatg
tatttagaaaaataaacaaataggggttccgcgcacatttccccgaaaagtgccacctgac
(pLKO-puro-2A-mdx^{4cv}-EGFP)
SEQ ID NO: 10
gggtctctctggttagaccagatctgagcctgggagctctctggctaactagggaacccactgcttaagcctcaataaagcttgc
cttgagtgcttcaagtagtgtgtgcccgtctgttgtgtgactctggtaactagagatccctcagacccttttagtcagtgtggaaaatctctagc
agtggcgcccgaacagggacttgaaagcgaaagggaaaccagaggagctctctcgacgcaggactcggcttgctgaagcgcgcacgg
caagaggcgaggggcggcgactggtgagtacgccaaaaattttgactagcggaggctagaaggagagagatgggtgcgagagogtca
gtattaagcgggggagaattagatcgcgatgggaaaaaattcggttaaggccagggggaaagaaaaaatataaattaaaacatatagtatg
ggcaagcagggagctagaacgattcgcagttaatcctggcctgttagaaacatcagaaggctgtagacaaatactgggacagctacaacc
atcccttcagacaggatcagaagaacttagatcattatataatacagtagcaaccctctattgtgtgcatcaaaggatagagataaaagacac
caaggaagctttagacaagatagaggaagagcaaaacaaaagtaagaccaccgcacagcaagcggccgctgatcttcagacctggagg
aggagatatgagggacaattggagaagtgaattatataaatataaagtagtaaaaattgaaccattaggagtagcacccaccaaggcaaag
agaagagtggtgcagagagaaaaaagagcagtgggaataggagctttgttccttgggttcttgggagcagcaggaagcactatgggcgc
agcgtcaatgacgctgacggtacaggccagacaattattgtctggtatagtgcagcagcagaacaatttgctgagggctattgaggcgcaa
cagcatctgttgcaactcacagtctggggcatcaagcagctccaggcaagaatcctggctgtggaaagatacctaaaggatcaacagctcc
tggggatttggggttgctctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgcaaaaccagcaagaaa
agaatgaacaagaattattggaattagataaatgggcaagtttgtggaattggtttaacataacaaattggctgtggtatataaaattattcataa
tgatagtaggaggcttggtaggtttaagaatagtttttgctgtactttctatagtgaatagagttaggcagggatattcaccattatcgtttcagac
ccacctcccaaccccgaggggacccgacaggcccgaaggaatagaagaagaaggtggagagagagacagagacagatccattcgatt
agtgaacggatctcgacggtatcgatcacgagactagcctcgagacaaatggcagtattcatccacaattttaaaagaaaaggggggattg
gggggtacagtgcaggggaaagaatagtagacataatagcaacagacatacaaactaaagaattacaaaaacaaattacaaaaattcaaa
attttcgggtttattacagggacagcagagatccactttggccgcggTAGTTATTAATAGTAATCAATTACGGGG
TCATTAGTTCATAGCCCATATATGGAGTTCCGCGTTACATAACTTACGGTAAATGGC
CCGCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTAT
TGACGTCAATGACGGTAAATGGCCCGCCTGGCATTATGCCCAGTACATGACCTTATG
GGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGG
ACTTTCCAAAATGTCGTAACAACTCCGCCCCATTGACGCAAATGGGCGGTAGGCGT
GTACGGTGGGAGGTCTATATAAGCAGAGCTGGTTTAGTGAACCGTCAGATCCGCTA
GCCACCATGACCGAGTACAAGCCCACGGTGCGCCTCGCCACCCGCGACGACGTCCC
CAGGGCCGTACGCACCCTCGCCGCCGCGTTCGCCGACTACCCCGCCACGCGCCACA
CCGTCGATCCGGACCGCCACATCGAGCGGGTCACCGAGCTGCAAGAACTCTTCCTC
ACGCGCGTCGGGCTCGACATCGGCAAGGTGTGGGTCGCGGACGACGGCGCCGCGGT
GGCGGTCTGGACCACGCCGGAGAGCGTCGAAGCGGGGGCGGTGTTCGCCGAGATC
GGCCCGCGCATGGCCGAGTTGAGCGGTTCCCGGCTGGCCGCGCAGCAACAGATGGA
AGGCCTCCTGGCGCCGCACCGGCCCAAGGAGCCCGCGTGGTTCCTGGCCACCGTCG
GCGTgTCGCCCGACCACCAGGGCAAGGGTCTGGGCAGCGCCGTCGTGCTCCCCGGA
GTGGAGGCGGCCGAGCGCGCCGGGGTGCCCGCCTTCCTGGAaACCTCCGCGCCCCG
CAACCTCCCCTTCTACGAGCGGCTCGGCTTCACCGTCACCGCCGACGTCGAGGTGCC
CGAAGGACCGCGCACCTGGTGCATGACCCGCAAGCCCGGTGCCCTGTACAAGCAAT
GTACTAACTACGCTTTGTTGAAACTCGCTGGCGATGTTGAAAGTAACCCCGGTCCTg
aattcCAAGAACAGCTGCAGAACAGGAGATAACAGTTGggatccGTGAGCAAGGGCGAG
GAGCTGTTCACCGGGGTGGTGCCCATCCTGGTCGAGCTGGACGGCGACGTAAACGG
CCACAAGTTCAGCGTGTCCGGCGAGGGCGAGGGCGATGCCACCTACGGCAAGCTGA
CCCTGAAGTTCATCTGCACCACCGGCAAGCTGCCCGTGCCCTGGCCCACCCTCGTGA
CCACCCTGACCTACGGCGTGCAGTGCTTCAGCCGCTACCCCGACCACATGAAGCAG
CACGACTTCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGGCGACA
CCCTGGTGAACCGCATCGAGCTGAAGGGCATCGACTTCAAGGAGGACGGCAACATC
CTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGAC
GGCAGCGTGCAGCTCGCCGACCACTACCAGCAGAACACCCCCATCGGCGACGGCCC
CGTGCTGCTGCCCGACAACCACTACCTGAGCACCCAGTCCGCCCTGAGCAAAGACC
CCAACGAGAAGCGCGATCACATGGTCCTGCTGGAGTTCGTGACCGCCGCCGGGATC
ACTCTCGGCATGGACGAGCTGTACAAGtaaGgtacctttaagaccaatgacttacaaggcagctgtagatcttag
ccactttttaaaagaaaaggggggactggaagggctaattcactcccaacgaagacaagatctgctttttgcttgtactgggtctctctggtta
gaccagatctgagcctgggagctctctggctaactagggaacccactgcttaagcctcaataaagcttgccttgagtgcttcaagtagtgtgt
gcccgtctgttgtgtgactctggtaactagagatccctcagacccttttagtcagtgtggaaaatctctagcagtagtagttcatgtcatcttatta
ttcagtatttataacttgcaaagaaatgaatatcagagagtgagaggaacttgtttattgcagcttataatggttacaaataaagcaatagcatca
caaatttcacaaataaagcatttttttcactgcattctagtigiggttigtccaaactcatcaatgtatcttatcatgtctggctctagctatcccgcc
cctaactccgcccatcccgcccctaactccgcccagttccgcccattctccgccccatggctgactaattttttttatttatgcagaggccgag
gccgcctcggcctctgagctattccagaagtagtgaggaggcttttttggaggcctagggacgtacccaattcgccctatagtgagtcgtatt
acgcgcgctcactggccgtcgttttacaacgtcgtgactgggaaaaccctggcgttacccaacttaatcgccttgcagcacatccccctttcg
ccagctggcgtaatagcgaagaggcccgcaccgatcgcccttcccaacagttgcgcagcctgaatggcgaatgggacgcgccctgtag
cggcgcattaagcgcggcgggtgtggtggttacgcgcagcgtgaccgctacacttgccagcgccctagcgcccgctcctttcgctttcttc
ccttcctttctcgccacgttcgccggctttccccgtcaagctctaaatcgggggctccctttagggttccgatttagtgctttacggcacctcga
ccccaaaaaacttgattagggtgatggttcacgtagtgggccatcgccctgatagacggtttttcgccctttgacgttggagtccacgttcttta
atagtggactcttgttccaaactggaacaacactcaaccctatctcggtctattcttttgatttataagggattttgccgatttcggcctattggtta
aaaaatgagctgatttaacaaaaatttaacgcgaattttaacaaaatattaacgcttacaatttaggtggcacttttcggggaaatgtgcgcgga
acccctatttgtttatttttctaaatacattcaaatatgtatccgctcatgagacaataaccctgataaatgcttcaataatattgaaaaaggaaga
gtatgagtattcaacatttccgtgtcgcccttattcccttttttgcggcattttgccttcctgtttttgctcacccagaaacgctggtgaaagtaaaa
gatgctgaagatcagttgggtgcacgagtgggttacatcgaactggatctcaacagcggtaagatccttgagagttttcgccccgaagaac
gttttccaatgatgagcacttttaaagttctgctatgtggcgcggtattatcccgtattgacgccgggcaagagcaactcggtcgccgcataca
ctattctcagaatgacttggttgagtactcaccagtcacagaaaagcatcttacggatggcatgacagtaagagaattatgcagtgctgccat
aaccatgagtgataacactgcggccaacttacttctgacaacgatcggaggaccgaaggagctaaccgcttttttgcacaacatgggggat
catgtaactcgccttgatcgttgggaaccggagctgaatgaagccataccaaacgacgagcgtgacaccacgatgcctgtagcaatggca
acaacgttgcgcaaactattaactggcgaactacttactctagcttcccggcaacaattaatagactggatggaggcggataaagttgcagg
accacttctgcgctcggcccttccggctggctggtttattgctgataaatctggagccggtgagcgtgggtctcgcggtatcattgcagcact
ggggccagatggtaagccctcccgtatcgtagttatctacacgacggggagtcaggcaactatggatgaacgaaatagacagatcgctga
gataggtgcctcactgattaagcattggtaactgtcagaccaagtttactcatatatactttagattgatttaaaacttcatttttaatttaaaaggat
ctaggtgaagatcctttttgataatctcatgaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgctaccagcggtggtttgtttgccggatcaaga
gctaccaactctttttccgaaggtaactggcttcagcagagcgcagataccaaatactgttcttctagtgtagccgtagttaggccaccacttc
aagaactctgtagcaccgcctacatacctcgctctgctaatcctgttaccagtggctgctgccagtggcgataagtcgtgtcttaccgggttg
gactcaagacgatagttaccggataaggcgcagcggtcgggctgaacggggggttcgtgcacacagcccagcttggagcgaacgacct
acaccgaactgagatacctacagcgtgagctatgagaaagcgccacgcttcccgaagggagaaaggcggacaggtatccggtaagcgg
cagggtcggaacaggagagcgcacgagggagcttccagggggaaacgcctggtatctttatagtcctgtcgggtttcgccacctctgactt
gagcgtcgatttttgtgatgctcgtcaggggggcggagcctatggaaaaacgccagcaacgcggcctttttacggttcctggccttttgctg
gccttttgctcacatgttctttcctgcgttatcccctgattctgtggataaccgtattaccgcctttgagtgagctgataccgctcgccgcagccg
aacgaccgagcgcagcgagtcagtgagcgaggaagcggaagagcgcccaatacgcaaaccgcctctccccgcgcgttggccgattca
ttaatgcagctggcacgacaggtttcccgactggaaagcgggcagtgagcgcaacgcaattaatgtgagttagctcactcattaggcaccc
caggctttacactttatgcttccggctcgtatgttgtgtggaattgtgagcggataacaatttcacacaggaaacagctatgaccatgattacgc
caagcgcgcaattaaccctcactaaagggaacaaaagctggagctgcaagcttaatgtagtcttatgcaatactcttgtagtcttgcaacatg
gtaacgatgagttagcaacatgccttacaaggagagaaaaagcaccgtgcatgccgattggtggaagtaaggtggtacgatcgtgccttat
taggaaggcaacagacgggtctgacatggattggacgaaccactgaattgccgcattgcagagatattgtatttaagtgcctagctcgatac
ataaac
Wild Type SpCas9(D10A): Protein sequence:
SEQ ID NO: 11
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAA
KNLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVAQILDSRMNTK
YDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYN
KHRDKPIREQAENIIHLFTLTNLGAPAAFKYFDTTIDRKRYTSTKEVLDATLIHQSITGLY
ETRIDLSQLGGD,
SpCas9(D10A)-NG: Protein sequence:
SEQ ID NO: 12
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAA
KNLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVAQILDSRMNTK
YDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIRPKRNSDK
LIARKKDWDPKKYGGFVSPTVAYSVLVVAKVEKGKSKKLKSVKELLGITIMERSSFEK
NPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYN
KHRDKPIREQAENIIHLFTLTNLGAPRAFKYFDTTIDRKVYRSTKEVLDATLIHQSITGLY
ETRIDLSQLGGD,
xCas9(3.7)-D10A: Protein sequence:
SEQ ID NO: 13
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDTKLQLSKDTYDDDLDNLLAQIGDQYADLFLAA
KNLSDAILLSDILRVNTEITKAPLSASMIKLYDEHHQDLTLLKALVRQQLPEKYKEIFFD
QSKNGYAGYIDGGASQEEFYKFIKPILEKMDGTEELLVKLNREDLLRKQRTFDNGIIPHQ
IHLGELHAILRRQEDFYPFLKDNREKIEKILTFRIPYYVGPLARGNSRFAWMTRKSEETIT
PWNFEKVVDKGASAQSFIERMTNFDKNLPNEKVLPKHSLLYEYFTVYNELTKVKYVTE
GMRKPAFLSGDQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFIQLIHDDSLTFKEDIQKA
QVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPIDFL
EAKGYKEVKKDLIIKLPKYSLFELENGRKRMLASAGVLQKGNELALPSKYVNFLYLAS
HYEKLKGSPEDNEQKQLFVEQHKHYLDEIIEQISEFSKRVILADANLDKVLSAYNKHRD
KPIREQAENIIHLFTLTNLGAPAAFKYFDTIIDRKRYTSTKEVLDATLIHQSITGLYETRID
LSQLGGD,
SpCas9(D10A)-NGX: Protein sequence:
SEQ ID NO: 14
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKIRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDTKLQLSKDTYDDDLDNLLAQIGDQYADLFLAA
KNLSDAILLSDILRVNTEITKAPLSASMIKLYDEHHQDLTLLKALVRQQLPEKYKEIFFD
QSKNGYAGYIDGGASQEEFYKFIKPILEKMDGTEELLVKLNREDLLRKQRTFDNGIIPHQ
IHLGELHAILRRQEDFYPFLKDNREKIEKILTFRIPYYVGPLARGNSRFAWMTRKSEETIT
PWNFEKVVDKGASAQSFIERMTNFDKNLPNEKVLPKHSLLYEYFTVYNELTKVKYVTE
GMRKPAFLSGDQKKAIVDLLFKTNRKVTVKQLKEDYFKKIECFDSVEISGVEDRFNASL
GTYHDLLKIIKDKDFLDNEENEDILEDIVLTLTLFEDREMIEERLKYAHLFDDKVMKQL
KRRRYTGWGRLSRKLINGIRDKQSGKTILDFLKSDGFANRNFIQLIHDDSLTFKEDIQKA
QVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESIRPKRNSDKLIARK
KDWDPKKYGGFVSPTVAYSVLVVAKVEKGKSKKLKSVKELLGITIMERSSFEKNPIDFL
EAKGYKEVKKDLIIKLPKYSLFELENGRKRMLASARFLQKGNELALPSKYVNFLYLASH
YEKLKGSPEDNEQKQLFVEQHKHYLDEIIEQISEFSKRVILADANLDKVLSAYNKHRDK
PIREQAENIIHLFTLTNLGAPRAFKYFDTTIDRKVYRSTKEVLDATLIHQSITGLYETRIDL
SQLGGD,
SpCas9(D10A)-NGA: Protein sequence:
SEQ ID NO: 15
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAA
KNLSDAILLSDILRVNTEITKAPLSASMIKRYDEHHQDLTLLKALVRQQLPEKYKEIEFD
QSKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVI
EGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVAQILDSRMNTK
YDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIRPKRNSDK
LIARKKDWDPKKYGGFVSPTVAYSVLVVAKVEKGKSKKLKSVKELLGITIMERSSFEK
NPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYN
KHRDKPIREQAENIIHLFTLTNLGAPRAFKYFDTTIDRKQYRSTKEVLDATLIHQSITGLY
ETRIDLSQLGGD,
SpCas9(D10A)-NGC: Protein sequence:
SEQ ID NO: 16
MDKKYSIGLAIGTNSVGWAVITDEYKVPSKKFKVLGNTDRHSIKKNLIGALLFDS
GETAEATRLKRIARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAA
KNLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVD
QELDINRLSDYDVDHTVPQSFLKDDSIDNKVLTRSDKNRGKSDNVPSEEVVKKMKNYW
RQLLNAKIITQRKFDNLTKAERGGLSELDKAGFIKRQLVETRQITKHVAQILDSRMNTK
YDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESIRPKRNSDK
LIARKKDWDPKKYGGFVSPTVAYSVLVVAKVEKGKSKKLKSVKELLGITIMERSSFEK
NPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYN
KHRDKPIREQAENIIHLFTLTNLGAPRAFKYFDTTIDRKEYRSTKEVLDATLIHQSITGLY
ETRIDLSQLGGD,
SpCas9(D10A)-NGX-NGA: Protein sequence:
SEQ ID NO: 17
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDTKLQLSKDTYDDDLDNLLAQIGDQYADLFLAA
KNLSDAILLSDILRVNTEITKAPLSASMIKLYDEHHQDLTLLKALVRQQLPEKYKEIFFD
QSKNGYAGYIDGGASQEEFYKFIKPILEKMDGTEELLVKLNREDLLRKQRTFDNGIIPHQ
IHLGELHAILRRQEDFYPFLKDNREKIEKILTFRIPYYVGPLARGNSRFAWMTRKSEETIT
PWNFEKVVDKGASAQSFIERMTNFDKNLPNEKVLPKHSLLYEYFTVYNELTKVKYVTE
GMRKPAFLSGDQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFIQLIHDDSLTFKEDIQKA
QVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESIRPKRNSDKLIARK
KDWDPKKYGGFVSPTVAYSVLVVAKVEKGKSKKLKSVKELLGITIMERSSFEKNPIDFL
EAKGYKEVKKDLIIKLPKYSLFELENGRKRMLASARFLQKGNELALPSKYVNFLYLASH
YEKLKGSPEDNEQKQLFVEQHKHYLDEIIEQISEFSKRVILADANLDKVLSAYNKHRDK
PIREQAENIIHLFTLTNLGAPRAFKYFDTTIDRKQYRSTKEVLDATLIHQSITGLYETRIDL
SQLGGD,
SpCas9(D10A)-NGX-NGC: Protein sequence:
SEQ ID NO: 18
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDTKLQLSKDTYDDDLDNLLAQIGDQYADLFLAA
KNLSDAILLSDILRVNTEITKAPLSASMIKLYDEHHQDLTLLKALVRQQLPEKYKEIFFD
QSKNGYAGYIDGGASQEEFYKFIKPILEKMDGTEELLVKLNREDLLRKQRTFDNGIIPHQ
IHLGELHAILRRQEDFYPFLKDNREKIEKILTFRIPYYVGPLARGNSRFAWMTRKSEETIT
PWNFEKVVDKGASAQSFIERMTNFDKNLPNEKVLPKHSLLYEYFTVYNELTKVKYVTE
GMRKPAFLSGDQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFIQLIHDDSLTFKEDIQKA
QVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIE
TNGETGEIVWDKGRDFATVRKVLSMPQVNIVKKTEVQTGGFSKESIRPKRNSDKLIARK
KDWDPKKYGGFVSPTVAYSVLVV‘AKVEKGKSKKLKSVKELLGITIMERSSFEKNPIDFL
EAKGYKEVKKDLIIKLPKYSLFELENGRKRMLASARFLQKGNELALPSKYVNFLYLASH
YEKLKGSPEDNEQKQLFVEQHKHYLDEIEQISEFSKRVILADANLDKVLSAYNKHRDK
PIREQAENIIHLFTLTNLGAPRAFKYFDTTIDRKEYRSTKEVLDATLIHQSITGLYETRIDL
SQLGGD,
SpCas9(D10A)-NG-loop: Protein sequence:
SEQ ID: 19
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAA
KNLSDAILLSDILRVNTEITKAPLSASMIKRYDEHHQDLTLLKALVRQQLPEKYKEIFFD
QSKNGYAGYVGADKKLRKRSSKLATEEEFYKFIKPILEKMDGTEELLVKLNREDLLRK
QRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFANRNFMQLI
HDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFF
KTEITLANGEIRKRPLIETNGETGEIVWDKGRDFATVRKVLSMPQVNIVKKTEVQTGGFS
KESIRPKRNSDKLIARKKDWDPKKYGGFVSPTVAYSVLVVAKVEKGKSKKLKSVKELL
GITIMERSSFEKNPIDFLEAKGYKEVKKDLIIKLPKYSLFELENGRKRMLASARFLQKGN
ELALPSKYVNFLYLASHYEKLKGSPEDNEQKQLFVEQHKHYLDEHIEQISEFSKRVILAD
ANLDKVLSAYNKHRDKPIREQAENIIHLFTLTNLGAPRAFKYFDTTIDRKVYRSTKEVLD
ATLIHQSITGLYETRIDLSQLGGD,
SpCas9(D10A)-NGX-loop: Protein sequence:
SEQ ID NO: 20
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKK
NGLFGNLIALSLGLTPNFKSNFDLAEDTKLQLSKDTYDDDLDNLLAQIGDQYADLFLAA
KNLSDAILLSDILRVNTEITKAPLSASMIKLYDEHHQDLTLLKALVRQQLPEKYKEIFFD
QSKNGYAGYVGADKKLRKRSSKLATEEEFYKFIKPILEKMDGTEELLVKLNREDLLRK
QRTFDNGIIPHQIHLGELHAILRRQEDFYPFLKDNREKIEKILTFRIPYYVGPLARGNSRFA
WMTRKSEETITPWNFEKVVDKGASAQSFIERMTNFDKNLPNEKVLPKHSLLYEYFTVY
NELTKVKYVTEGMRKPAFLSGDQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFIQLIHD
DSLTFKEDIQKAQVSGQGDSLHEHIANLAGSPAIKKGILQTVKVVDELVKVMGRHKPEN
IVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESI
RPKRNSDKLIARKKDWDPKKYGGFVSPTVAYSVLVVAKVEKGKSKKLKSVKELLGITI
MERSSFEKNPIDFLEAKGYKEVKKDLIIKLPKYSLFELENGRKRMLASARFLQKGNELA
LPSKYVNFLYLASHYEKLKGSPEDNEQKQLFVEQHKHYLDEIIEQISEFSKRVILADANL
DKVLSAYNKHRDKPIREQAENIIHLFTLTNLGAPRAFKYFDTTIDRKVYRSTKEVLDATL
IHQSITGLYETRIDLSQLGGD,
ScCas9(D10A): Protein sequence:
SEQ ID NO: 21
MEKKYSIGLAIGTNSVGWAVITDDYKVPSKKFKVLGNTNRKSIKKNLMGALLFD
SGETAEATRLKRTARRRYTRRKNRIRYLQEIFANEMAKLDDSFFQRLEESFLVEEDKKN
ERHPIFGNLADEVAYHRNYPTIYHLRKKLADSPEKADLRLIYLALAHIIKFRGHFLIEGKL
NAENSDVAKLFYQLIQTYNQLFEESPLDEIEVDAKGILSARLSKSKRLEKLIAVFPNEKK
NGLFGNIIALALGLTPNFKSNFDLTEDAKLQLSKDTYDDDLDELLGQIGDQYADLFSAA
KNLSDAILLSDILRSNSEVTKAPLSASMVKRYDEHHQDLALLKTLVRQQFPEKYAEIFK
DDTKNGYAGYVGIGIKHRKRTTKLATQEEFYKFIKPILEKMDGAEELLAKLNRDDLLRK
QRTFDNGSIPHQIHLKELHAILRRQEEFYPFLKENREKIEKILTFRIPYYVGPLARGNSRFA
WLTRKSEEAITPWNFEEVVDKGASAQSFIERMTNFDEQLPNKKVLPKHSLLYEYFTVYN
ELTKVKYVTERMRKPEFLSGEQKKAIVDLLFKTNRKVTVKQLKEDYFKKIECFDSVEIIG
VEDRFNASLGTYHDLLKIIKDKDFLDNEENEDILEDIVLTLTLFEDREMIEERLKTYAHLF
DDKVMKQLKRRHYTGWGRLSRKMINGIRDKQSGKTILDFLKSDGFSNRNFMQLIHDDS
LTFKEEIEKAQVSGQGDSLHEQIADLAGSPAIKKGILQTVKIVDELVKVMGHKPENIVIE
MARENQTTTKGLQQSRERKKRIEEGIKELESQILKENPVENTQLQNEKLYLYYLQNGRD
MYVDQELDINRLSDYDVDHIVPQSFIKDDSIDNKVLTRSVENRGKSDNVPSEEVVKKM
KNYWRQLLNAKLITQRKFDNLTKAERGGLSEADKAGFIKRQLVETRQITKHVARILDSR
MNTKRDKNDKPIREVKVITLKSKLVSDFRKDFQLYKVRDINNYHHAHDAYLNAVVGT
ALIKKYPKLESEFVYGDYKVYDVRKMIAKSEQEIGKATAKRFFYSNIMNFFKTEVKLAN
GEIRKRPLIETNGETGEVVWNKEKDFATVRKVLAMPQVNIVKKTEVQTGGFSKESILSK
RESAKLIPRKKGWDTRKYGGFGSPTVAYSILVVAKVEKGKAKKLKSVKVLVGITIMEK
GSYEKDPIGFLEAKGYKDIKKELIFKLPKYSLFELENGRRRMLASATELQKANELVLPQH
LVRLLYYTQNISATTGSNNLGYIEQHREEFKEIFEKIIDFSEKYILKNKVNSNLKSSFDEQF
AVSDSILLSNSFVSLLKYTSFGASGGFTFLDLDVKQGRLRYQTVTEVLDATLIYQSITGL
YETRTDLSQLGGD,
TadA-TadA*: Protein sequence:
SEQ ID NO: 22
SEVEFSHEYWMRHALTLAKRAWDEREVPVGAVLVHNNRVIGEGWNRPIGRHD
PTAHAEIMALRQGGLVMQNYRLIDATLYVTLEPCVMCAGAMIHSRIGRVVFGARDAKT
GAAGSLMDVLHHPGMNHRVEITEGILADECAALLSDFFRMRRQEIKAQKKAQSSTDSG
GSSGGSSGSETPGTSESATPESSGGSSGGSSEVEFSHEYWMRHALTLAKRARDEREVPV
GAVLVLNNRVIGEGWNRAIGLHDPTAHAEIMALRQGGLVMQNYRLIDATLYVTFEPCV
MCAGAMIHSRIGRVVFGVRNAKTGAAGSLMDVLHYPGMNHRVEITEGILADECAALL
CYFFRMPRQVFNAQKKAQSSTD,
TadA*(A56G_V82G): Protein sequence:
SEQ ID: 23
SEVEFSHEYWMRHALTLAKRARDEREVPVGAVLVLNNRVIGEGWNRAIGLHDP
TGHAEIMALRQGGLVMQNYRLIDATLYGTFEPCVMCAGAMIHSRIGRVVFGVRNAKT
GAAGSLMDVLHYPGMNHRVEITEGILADECAALLCYFFRMPRQVFNAQKKAQSSTD,
Gp41-1-N: Protein sequence:
SEQ ID NO: 24
CLDLKTQVQTPQGMKEISNIQVGDLVLSNTGYNEVLNVEPKSKKKSYKITLEDG
KEIICSEEHLFPTQTGEMNISGGLKEGMCLYVKE,
Gp41-1-C: Protein sequence:
SEQ ID NO: 25
MMLKKILKIEELDERELIDIEVSGNHLFYANDILTHNS,
Cfa-N: Protein sequence:
SEQ ID NO: 26
CLSYDTEILTVEYGFLPIGKIVEERIECTVYTVDKNGFVYTQPIAQWHNRGEQEV
FEYCLEDGSIIRATKDHKFMTTDGQMLPIDEIFERGLDLKQVDGLPN,
Cfa-C: Protein sequence:
SEQ ID NO: 27
MVKIISRKSLGTQNVYDIGVEKDHNFLLKNGLVASN,
meCMV:
SEQ ID NO: 28
tcgagCGCGTGATGAGAGCAGCCACTACGGGTCTAGGCTGCCCATGTAAGGAG
GCAAGGCCTGGGGACACCCGAGATGCCTGGTTATAATTAACCCAGACATGTGGCTG
CCCCCCCCCCCCCAACACCTGCTGCCTGCTAAAAATAACCCTGTCCCTGGTGGccCtgc
atgcccACTCACGGGGATTTCCAAGTCTCCACCCCATTGACGTCAATGGGAGTTTGTTT
TGGCACCAAAATCAACGGGACTTTCCAAAATGTCGTAACAACTCCGCCCCATTGAC
GCAAATGGGCGGTAGGCGTGTACGGTGGGAGGTCTATATAAGCAGAGCTGGTTTAG
TGAACCGTCAGATC,
SEMHP1:
SEQ ID NO: 29
CCCTTCAGATTAAAAATAACTGAGGTAAGGGCCTGGGTAGGGGAGGTGGTG
TGAGACGCTCCTGTCTCTCCTCTATCTGCCCATCGGCCCTTTGGGGAGGAGGAATGT
GCCCAAGGACTAAAAAAAGGCCATGGAGCCAGAGGGGCGAGGGCAACAGACCTTT
CATGGGCAAACCTTGGGGCCCTGCTGactgtaGATGAGAGCAGCCACTACGGGTCTAG
GCTGCCCATGTAAGGAGGCAAGGCCTGGGGACACCCGAGATGCCTGGTTATAATTA
ACCCAGACATGTGGCTGCCCCCCCCCCCCCAACACCTGCTGCCTGCTAAAAATAACC
CTGTCCCTGGTGGccctgcatgCCCTCCCTGGGGACAGCCCCTCCTGGCTAGTCACACCC
TGTAGGCTCCTCTATATAACCCAGGGGCACAGGGGCTGCCCtcattctACCACCACCTCC
ACAGCACAGACAGACACTCAGGAGCCAGC,
Wild Type SpCas9(D10A): Nucleotide sequence:
SEQ ID NO: 30
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATGCCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGAGATACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGA
GTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGG
CAGCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGGAAGTGGTG
GACAAGGGCGCTTCCGCCCAGAGCTTCATCGAGCGGATGACCAACTTCGATAAGAA
CCTGCCCAACGAGAAGGTGCTGCCCAAGCACAGCCTGCTGTACGAGTACTTCACCG
TGTATAACGAGCTGACCAAAGTGAAATACGTGACCGAGGGAATGAGAAAGCCCGC
CTTCCTGAGCGGCGAGCAGAAAAAGGCCATCGTGGACCTGCTGTTCAAGACCAACC
GGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGATG
ATCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTAAAG
AGGACATCCAGAAAGCCCAGGTGTCCGGCCAGGGCGATAGCCTGCACGAGCACATT
GCCAATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTGATC
GAAATGGCCAGAGAGAACCAGACCACCCAGAAGGGACAGAAGAACAGCCGCGAG
AGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGA
CTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTC
CGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAAT
GACAAGCTGATCCGGGAAGTGAAAGTGATCACCCTGAAGTCCAAGCTGGTGTCCGA
TTTCCGGAAGGATTTCCAGTTTTACAAAGTGCGCGAGATCAACAACTACCACCACG
CCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGAT
GATCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGG
GCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCTGCCCAAGA
GGAACAGCGATAAGCTGATCGCCAGAAAGAAGGACTGGGACCCTAAGAAGTACGG
CGGCTTCGACAGCCCCACCGTGGCCTATTCTGTGCTGGTGGTGGCCAAAGTGGAAA
AGGGCAAGTCCAAGAAACTGAAGAGTGTGAAAGAGCTGCTGGGGATCACCATCAT
GGAAAGAAGCAGCTTCGAGAAGAATCCCATCGACTTTCTGGAAGCCAAGGGCTACA
AAGAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAACG
AACTGGCCCTGCCCTCCAAATATGTGAACTTCCTGTACCTGGCCAGCCACTATGAGA
AGCTGAAGGGCTCCCCCGAGGATAATGAGCAGAAACAGCTGTTTGTGGAACAGCAC
AAGCACTACCTGGACGAGATCATCGAGCAGATCAGCGAGTTCTCCAAGAGAGTGAT
CCTGGCCGACGCTAATCTGGACAAAGTGCTGTCCGCCTACAACAAGCACCGGGATA
AGCCCATCAGAGAGCAGGCCGAGAATATCATCCACCTGTTTACCCTGACCAATCTG
GGAGCCCCTGCCGCCTTCAAGTACTTTGACACCACCATCGACCGGAAGAGGTACAC
CAGCACCAAAGAGGTGCTGGACGCCACCCTGATCCACCAGAGCATCACCGGCCTGT
ACGAGACACGGATCGACCTGTCTCAGCTGGGAGGTGAC,
SpCas9(D10A)-NG: Nucleotide sequence:
SEQ ID NO: 31
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGICCTICCTGGIGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATGCCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGAGATACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGA
GTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGG
CAGCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGGAAGTGGTG
GACAAGGGCGCTTCCGCCCAGAGCTTCATCGAGCGGATGACCAACTTCGATAAGAA
CCTGCCCAACGAGAAGGTGCTGCCCAAGCACAGCCTGCTGTACGAGTACTTCACCG
TGTATAACGAGCTGACCAAAGTGAAATACGTGACCGAGGGAATGAGAAAGCCCGC
CTTCCTGAGCGGCGAGCAGAAAAAGGCCATCGTGGACCTGCTGTTCAAGACCAACC
GGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGATG
ATCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTAAAG
AGGACATCCAGAAAGCCCAGGTGTCCGGCCAGGGCGATAGCCTGCACGAGCACATT
GCCAATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTGATC
GAAATGGCCAGAGAGAACCAGACCACCCAGAAGGGACAGAAGAACAGCCGCGAG
AGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGA
CTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTC
CGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAAT
GACAAGCTGATCCGGGAAGTGAAAGTGATCACCCTGAAGTCCAAGCTGGTGTCCGA
TTTCCGGAAGGATTTCCAGTTTTACAAAGTGCGCGAGATCAACAACTACCACCACG
CCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGAT
GATCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGG
GCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCgGCCCAAGAG
GAACAGCGATAAGCTGATCGCCAGAAAGAAGGACTGGGACCCTAAGAAGTACGGC
GGCTTCGtCAGCCCCACCGTGGCCTATTCTGTGCTGGTGGTGGCCAAAGTGGAAAAG
GGCAAGTCCAAGAAACTGAAGAGTGTGAAAGAGCTGCTGGGGATCACCATCATGG
AAAGAAGCAGCTTCGAGAAGAATCCCATCGACTTTCTGGAAGCCAAGGGCTACAAA
GAAGTGAAAAAGGACCTGATCATCAAGCTGCCTAAGTACTCCCTGTTCGAGCTGGA
AAACGGCCGGAAGAGAATGCTGGCCTCTGCCCGCttCTGCAGAAGGGAAACGAACT
GGCCCTGCCCTCCAAATATGTGAACTTCCTGTACCTGGCCAGCCACTATGAGAAGCT
GAAGGGCTCCCCCGAGGATAATGAGCAGAAACAGCTGTTTGTGGAACAGCACAAG
CACTACCTGGACGAGATCATCGAGCAGATCAGCGAGTTCTCCAAGAGAGTGATCCT
GGCCGACGCTAATCTGGACAAAGTGCTGTCCGCCTACAACAAGCACCGGGATAAGC
CCATCAGAGAGCAGGCCGAGAATATCATCCACCTGTTTACCCTGACCAATCTGGGA
GCCCCTcggGCCTTCAAGTACTTTGACACCACCATCGACCGGAAGgtGTACcggAGCAC
CAAAGAGGTGCTGGACGCCACCCTGATCCACCAGAGCATCACCGGCCTGTACGAGA
CACGGATCGACCTGTCTCAGCTGGGAGGTGAC,
xCas9(3.7)-D10A, Nucleotide sequence:
SEQ ID NO: 32
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATACCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGCTGTACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGA
GTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGG
CATCATCCCCCACCAGATCCACCTGGGAGAGCTGCACGCCATTCTGCGGCGGCAGG
AAGATTTnACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGAAGGTGGTG
GACAAGGGCGCTTCCGCCCAGAGCTTCATCGAGCGGATGACCAACTTCGATAAGAA
CCTGCCCAACGAGAAGGTGCTGCCCAAGCACAGCCTGCTGTACGAGTACTTCACCG
TGTATAACGAGCTGACCAAAGTGAAATACGTGACCGAGGGAATGAGAAAGCCCGC
CTTCCTGAGCGGCGACCAGAAAAAGGCCATCGTGGACCTGCTGTTCAAGACCAACC
GGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAACG
AGGACATTCTGGAAGATATCGTGCTGACCCTGACACTGTTiTGAGGACAGAGAGATG
ATCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGG
CTTCGCCAACAGAAACTTCATCCAGCTGATCCACGACGACAGCCTGACCTTTAAAG
AGGACATCCAGAAAGCCCAGGTGTCCGGCCAGGGCGATAGCCTGCACGAGCACATT
GCCAATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTGATC
GAAATGGCCAGAGAGAACCAGACCACCCAGAAGGGACAGAAGAACAGCCGCGAG
AGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGA
CTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTC
CGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAAr
GACAAGCTGATCCGGGAAGTGAAAGTGATCACCCTGAAGTCCAAGCTGGTGTCCGA
TTTCCGGAAGGATTTCCAGTTTTACAAAGTGCGCGAGATCAACAACTACCACCACG
CCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGAT
GATCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGG
GCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCTGCCCAAGA
GGAACAGCGATAAGCTGATCGCCAGAAAGAAGGACTGGGACCCTAAGAAGTACGG
CGGCTTCGACAGCCCCACCGTGGCCTATTCTGTGCTGGTGGTGGCCAAAGTGGAAA
AGGGCAAGTCCAAGAAACTGAAGAGTGTGAAAGAGCTGCTGGGGATCACCATCAT
GGAAAGAAGCAGCTTCGAGAAGAATCCCATCGACTTTCTGGAAGCCAAGGGCTACA
AAGAAGTGAAAAAGGACCTGATCATCAAGCTGCCTAAGTACTCCCTGTTCGAGCTG
GAAAACGGCCGGAAGAGAATGCTGGCCTCTGCCGGCGTGCTGCAGAAGGGAAACG
AACTGGCCCTGCCCTCCAAATATGTGAACTTCCTGTACCTGGCCAGCCACTATGAGA
AGCTGAAGGGCTCCCCCGAGGATAATGAGCAGAAACAGCTGTTTGTGGAACAGCAC
AAGCACTACCTGGACGAGATCATCGAGCAGATCAGCGAGTTCTCCAAGAGAGTGAT
CCTGGCCGACGCTAATCTGGACAAAGTGCTGTCCGCCTACAACAAGCACCGGGATA
AGCCCATCAGAGAGCAGGCCGAGAATATCATCCACCTGTTTACCCTGACCAATCTG
GGAGCCCCTGCCGCCTTCAAGTACTTTGACACCACCATCGACCGGAAGAGGTACAC
CAGCACCAAAGAGGTGCTGGACGCCACCCTGATCCACCAGAGCATCACCGGCCTGT
ACGAGACACGGATCGACCTGTCTCAGCTGGGAGGCGAC,
SpCas9(D10A)-NGX: Nucleotide sequence:
SEQ ID NO: 33
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATACCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGCTGTACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGA
GTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGG
CATCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGAAGGTGGTG
GACAAGGGCGCTTCCGCCCAGAGCTTCATCGAGCGGATGACCAACTTCGATAAGAA
CCTGCCCAACGAGAAGGTGCTGCCCAAGCACAGCCTGCTGTACGAGTACTTCACCG
TGTATAACGAGCTGACCAAAGTGAAATACGTGACCGAGGGAATGAGAAAGCCCGC
CTTCCTGAGCGGCGACCAGAAAAAGGCCATCGTGGACCTGCTGTTCAAGACCAACC
GGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGATG
ATCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGG
CTTCGCCAACAGAAACTTCATCCAGCTGATCCACGACGACAGCCTGACCTTTAAAG
AGGACATCCAGAAAGCCCAGGTGTCCGGCCAGGGCGATAGCCTGCACGAGCACATT
GCCAATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTGATC
GAAATGGCCAGAGAGAACCAGACCACCCAGAAGGGACAGAAGAACAGCCGCGAG
AGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGA
CTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTC
CGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAAT
GACAAGCTGATCCGGGAAGTGAAAGTGATCACCCTGAAGTCCAAGCTGGTGTCCGA
TTTCCGGAAGGATTTCCAGTTTTACAAAGTGCGCGAGATCAACAACTACCACCACG
CCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGAT
GATCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGG
GCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCgGCCCAAGAG
GAACAGCGATAAGCTGATCGCCAGAAAGAAGGACTGGGACCCTAAGAAGTACGGC
GGCTTCGtCAGCCCCACCGTGGCCTATTCTGTGCTGGTGGTGGCCAAAGTGGAAAAG
GGCAAGTCCAAGAAACTGAAGAGTGTGAAAGAGCTGCTGGGGATCACCATCATGG
AAAGAAGCAGCTTCGAGAAGAATCCCATCGACTTTCTGGAAGCCAAGGGCTACAAA
GAAGTGAAAAAGGACCTGATCATCAAGCTGCCTAAGTACTCCCTGTTCGAGCTGGA
AAACGGCCGGAAGAGAATGCTGGCCTCTGCCcGCtttCTGCAGAAGGGAAACGAACT
GGCCCTGCCCTCCAAATATGTGAACTTCCTGTACCTGGCCAGCCACTATGAGAAGCT
GAAGGGCTCCCCCGAGGATAATGAGCAGAAACAGCTGTTTGTGGAACAGCACAAG
CACTACCTGGACGAGATCATCGAGCAGATCAGCGAGTTCTCCAAGAGAGTGATCCT
GGCCGACGCTAATCTGGACAAAGTGCTGTCCGCCTACAACAAGCACCGGGATAAGC
CCATCAGAGAGCAGGCCGAGAATATCATCCACCTGTTTACCCTGACCAATCTGGGA
GCCCCTcggGCCTTCAAGTACTTTGACACCACCATCGACCGGAAGgtGTACcggAGCAC
CAAAGAGGTGCTGGACGCCACCCTGATCCACCAGAGCATCACCGGCCTGTACGAGA
CACGGATCGACCTGTCTCAGCTGGGAGGTGAC,
SpCas9(D10A)-NGA: Nucleotide sequence:
SEQ ID NO: 34
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATGCCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGAGATACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGA
GTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGG
CAGCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGGAAGTGGTG
GACAAGGGCGCTTCCGCCCAGAGCTTCATCGAGCGGATGACCAACTTCGATAAGAA
CCTGCCCAACGAGAAGGTGCTGCCCAAGCACAGCCTGCTGTACGAGTACTTCACCG
TGTATAACGAGCTGACCAAAGTGAAATACGTGACCGAGGGAATGAGAAAGCCCGC
CTTCCTGAGCGGCGAGCAGAAAAAGGCCATCGTGGACCTGCTGTTCAAGACCAACC
GGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGATG
ATCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTAAAG
AGGACATCCAGAAAGCCCAGGTGTCCGGCCAGGGCGATAGCCTGCACGAGCACATT
GCCAATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTGATC
GAAATGGCCAGAGAGAACCAGACCACCCAGAAGGGACAGAAGAACAGCCGCGAG
AGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGA
CTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTC
CGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAAT
GACAAGCTGATCCGGGAAGTGAAAGTGATCACCCTGAAGTCCAAGCTGGTGTCCGA
TTTCCGGAAGGATTTCCAGTTTTACAAAGTGCGCGAGATCAACAACTACCACCACG
CCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGA1
GATCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGG
GCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCgGCCCAAGAG
GAACAGCGATAAGCTGATCGCCAGAAAGAAGGACTGGGACCCTAAGAAGTACGGC
GGCTTCGtCAGCCCCACCGTGGCCTATTCTGTGCTGGTGGTGGCCAAAGTGGAAAAG
GGCAAGTCCAAGAAACTGAAGAGTGTGAAAGAGCTGCTGGGGATCACCATCATGG
AAAGAAGCAGCTTCGAGAAGAATCCCATCGACTTTCTGGAAGCCAAGGGCTACAAA
GAAGTGAAAAAGGACCTGATCATCAAGCTGCCTAAGTACTCCCTGTTCGAGCTGGA
AAACGGCCGGAAGAGAATGCTGGCCTCTGCCCGCtCTGCAGAAGGGAAACGAACT
GGCCCTGCCCTCCAAATATGTGAACTTCCTGTACCTGGCCAGCCACTATGAGAAGCT
GAAGGGCTCCCCCGAGGATAATGAGCAGAAACAGCTGTTTGTGGAACAGCACAAG
CACTACCTGGACGAGATCATCGAGCAGATCAGCGAGTTCTCCAAGAGAGTGATCCT
GGCCGACGCTAATCTGGACAAAGTGCTGTCCGCCTACAACAAGCACCGGGATAAGC
CCATCAGAGAGCAGGCCGAGAATATCATCCACCTGTTTACCCTGACCAATCTGGGA
GCCCCTcggGCCTTCAAGTACTTTGACACCACCATCGACCGGAAGcaGTACcggAGCAC
CAAAGAGGTGCTGGACGCCACCCTGATCCACCAGAGCATCACCGGCCTGTACGAGA
CACGGATCGACCTGTCTCAGCTGGGAGGTGAC,
SpCas9(D10A)-NGC: Nucleotide sequence:
SEQ ID NO: 35
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATGCCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGAGATACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGA
GTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGG
CAGCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCCGACC
TTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGGAAGTGGTG
GACAAGGGCGCTTCCGCCCAGAGCTTCATCGAGCGGATGACCAACTTCGATAAGAA
CCTGCCCAACGAGAAGGTGCTGCCCAAGCACAGCCTGCTGTACGAGTACTTCACCG
TGTATAACGAGCTGACCAAAGTGAAATACGTGACCGAGGGAATGAGAAAGCCCGC
CTTCCTGAGCGGCGAGCAGAAAAAGGCCATCGTGGACCTGCTGTTCAAGACCAACC
GGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGATG
ATCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTAAAG
AGGACATCCAGAAAGCCCAGGTGTCCGGCCAGGGCGATAGCCTGCACGAGCACATT
GCCAATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTGATC
GAAATGGCCAGAGAGAACCAGACCACCCAGAAGGGACAGAAGAACAGCCGCGAG
AGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGA
CTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTC
CGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAAT
GACAAGCTGATCCGGGAAGTGAAAGTGATCACCCTGAAGTCCAAGCTGGTGTCCGA
TTTCCGGAAGGATTTCCAGTTTTACAAAGTGCGCGAGATCAACAACTACCACCACG
CCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGAT
GATCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGG
GCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCgGCCCAAGAG
GAACAGCGATAAGCTGATCGCCAGAAAGAAGGACTGGGACCCTAAGAAGTACGGC
GGCTTCGtCAGCCCCACCGTGGCCTATTCTGTGCTGGTGGTGGCCAAAGTGGAAAAG
GGCAAGTCCAAGAAACTGAAGAGTGTGAAAGAGCTGCTGGGGATCACCATCATGG
AAAGAAGCAGCTTCGAGAAGAATCCCATCGACTTTCTGGAAGCCAAGGGCTACAAA
GAAGTGAAAAAGGACCTGATCATCAAGCTGCCTAAGTACTCCCTGTTCGAGCTGGA
AAACGGCCGGAAGAGAATGCTGGCCTCTGCCcGCtttCTGCAGAAGGGAAACGAACT
GGCCCTGCCCTCCAAATATGTGAACTTCCTGTACCTGGCCAGCCACTATGAGAAGCT
GAAGGGCTCCCCCGAGGATAATGAGCAGAAACAGCTGTTTGTGGAACAGCACAAG
CACTACCTGGACGAGATCATCGAGCAGATCAGCGAGTTCTCCAAGAGAGTGATCCT
GGCCGACGCTAATCTGGACAAAGTGCTGTCCGCCTACAACAAGCACCGGGATAAGC
CCATCAGAGAGCAGGCCGAGAATATCATCCACCTGTTTACCCTGACCAATCTGGGA
GCCCCTcggGCCTTCAAGTACTTTGACACCACCATCGACCGGAAGgaGTACcggAGCAC
CAAAGAGGTGCTGGACGCCACCCTGATCCACCAGAGCATCACCGGCCTGTACGAGA
CACGGATCGACCTGTCTCAGCTGGGAGGTGAC,
SpCas9(D10A)-NGX-NGA: Nucleotide sequence:
SEQ ID NO: 36
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATACCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGCTGTACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGA
GTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGG
CATCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGAAGGTGGTG
GACAAGGGCGCTTCCGCCCAGAGCTTCATCGAGCGGATGACCAACTTCGATAAGAA
CCTGCCCAACGAGAAGGTGCTGCCCAAGCACAGCCTGCTGTACGAGTACTTCACCG
TGTATAACGAGCTGACCAAAGTGAAATACGTGACCGAGGGAATGAGAAAGCCCGC
CTTCCTGAGCGGCGACCAGAAAAAGGCCATCGTGGACCTGCTGTTCAAGACCAACC
GGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGATG
ATCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGG
CTTCGCCAACAGAAACTTCATCCAGCTGATCCACGACGACAGCCTGACCTTTAAAG
AGGACATCCAGAAAGCCCAGGTGTCCGGCCAGGGCGATAGCCTGCACGAGCACATT
GCCAATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTGATC
GAAATGGCCAGAGAGAACCAGACCACCCAGAAGGGACAGAAGAACAGCCGCGAG
AGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGA
CTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTC
CGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAAT
GACAAGCTGATCCGGGAAGTGAAAGTGATCACCCTGAAGTCCAAGCTGGTGTCCGA
TTTCCGGAAGGATTTCCAGTTTTACAAAGTGCGCGAGATCAACAACTACCACCACG
CCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGAT
GATCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGG
GCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCgGCCCAAGAG
GAACAGCGATAAGCTGATCGCCAGAAAGAAGGACTGGGACCCTAAGAAGTACGGC
GGCTTCGTCAGCCCCACCGTGGCCTATTCTGTGCTGGTGGTGGCCAAAGTGGAAAAG
GGCAAGTCCAAGAAACTGAAGAGTGTGAAAGAGCTGCTGGGGATCACCATCATGG
AAAGAAGCAGCTTCGAGAAGAATCCCATCGACTTTCTGGAAGCCAAGGGCTACAAA
GAAGTGAAAAAGGACCTGATCATCAAGCTGCCTAAGTACTCCCTGTTCGAGCTGGA
AAACGGCCGGAAGAGAATGCTGGCCTCTGCCcGCtttCTGCAGAAGGGAAACGAACT
GGCCCTGCCCTCCAAATATGTGAACTTCCTGTACCTGGCCAGCCACTATGAGAAGCT
GAAGGGCTCCCCCGAGGATAATGAGCAGAAACAGCTGTTTGTGGAACAGCACAAG
CACTACCTGGACGAGATCATCGAGCAGATCAGCGAGTTCTCCAAGAGAGTGATCCT
GGCCGACGCTAATCTGGACAAAGTGCTGTCCGCCTACAACAAGCACCGGGATAAGC
CCATCAGAGAGCAGGCCGAGAATATCATCCACCTGTTTACCCTGACCAATCTGGGA
GCCCCTcggGCCTTCAAGTACTTTGACACCACCATCGACCGGAAGcaGTACcggAGCAC
CAAAGAGGTGCTGGACGCCACCCTGATCCACCAGAGCATCACCGGCCTGTACGAGA
CACGGATCGACCTGTCTCAGCTGGGAGGTGAC,
SpCas9(D10A)-NGX-NGC, Nucleotide sequence:
SEQ ID NO: 37
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATACCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGCTGTACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGA
GTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGG
CATCATCCCCCACCAGATCCACCTGGGAGAGCTGCACGCCATTCTGCGGCGGCAGG
AAGATTTTTACCCATTCCTGAAGGACAACCGGGAAAAGATCGAGAAGATCCTGACC
TTCCGCATCCCCTACTACGTGGGCCCTCTGGCCAGGGGAAACAGCAGATTCGCCTG
GATGACCAGAAAGAGCGAGGAAACCATCACCCCCTGGAACTTCGAGAAGGTGGTG
GACAAGGGCGCTTCCGCCCAGAGCTTCATCGAGCGGATGACCAACTTCGATAAGAA
CCTGCCCAACGAGAAGGTGCTGCCCAAGCACAGCCTGCTGTACGAGTACTTCACCG
TGTATAACGAGCTGACCAAAGTGAAATACGTGACCGAGGGAATGAGAAAGCCCGC
CTTCCTGAGCGGCGACCAGAAAAAGGCCATCGTGGACCTGCTGTTCAAGACCAACC
GGAAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAACG
AGGACATTCTGGAAGATATCGTGCTGACCCTGACACTGTTTGAGGACAGAGAGATG
ATCGAGGAACGGCTGAAAACCTATGCCCACCTGTTCGACGACAAAGTGATGAAGCA
GCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGG
CTTCGCCAACAGAAACTTCATCCAGCTGATCCACGACGACAGCCTGACCTTTAAAG
AGGACATCCAGAAAGCCCAGGTGTCCGGCCAGGGCGATAGCCTGCACGAGCACATT
GCCAATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTGATC
GAAATGGCCAGAGAGAACCAGACCACCCAGAAGGGACAGAAGAACAGCCGCGAG
AGAATGAAGCGGATCGAAGAGGGCATCAAAGAGCTGGGCAGCCAGATCCTGAAAG
AACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCCGA
CTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTC
CGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAG
CGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAAT
GACAAGCTGATCCGGGAAGTGAAAGTGATCACCCTGAAGTCCAAGCTGGTGTCCGA
TTTCCGGAAGGATTTCCAGTTTTACAAAGTGCGCGAGATCAACAACTACCACCACG
CCCACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAAGAT
GATCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCGG
AAGCGGCCTCTGATCGAGACAAACGGCGAAACCGGGGAGATCGTGTGGGATAAGG
GCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCgGCCCAAGAG
GAACAGCGATAAGCTGATCGCCAGAAAGAAGGACTGGGACCCTAAGAAGTACGGC
GGCTTCGtCAGCCCCACCGTGGCCTATTCTGTGCTGGTGGTGGCCAAAGTGGAAAAG
GGCAAGTCCAAGAAACTGAAGAGTGTGAAAGAGCTGCTGGGGATCACCATCATGG
AAAGAAGCAGCTTCGAGAAGAATCCCATCGACTTTCTGGAAGCCAAGGGCTACAAA
GAAGTGAAAAAGGACCTGATCATCAAGCTGCCTAAGTACTCCCTGTTCGAGCTGGA
AAACGGCCGGAAGAGAATGCTGGCCTCTGCCcGCtttCTGCAGAAGGGAAACGAACT
GGCCCTGCCCTCCAAATATGTGAACTTCCTGTACCTGGCCAGCCACTATGAGAAGCT
GAAGGGCTCCCCCGAGGATAATGAGCAGAAACAGCTGTTTGTGGAACAGCACAAG
CACTACCTGGACGAGATCATCGAGCAGATCAGCGAGTTCTCCAAGAGAGTGATCCT
GGCCGACGCTAATCTGGACAAAGTGCTGTCCGCCTACAACAAGCACCGGGATAAGC
CCATCAGAGAGCAGGCCGAGAATATCATCCACCTGTTTACCCTGACCAATCTGGGA
GCCCCTcggGCCTTCAAGTACTTTGACACCACCATCGACCGGAAGgaGTACcggAGCAC
CAAAGAGGTGCTGGACGCCACCCTGATCCACCAGAGCATCACCGGCCTGTACGAGA
CACGGATCGACCTGTCTCAGCTGGGAGGTGAC,
SpCas9(D10A)-NG-loop: Nucleotide sequence:
SEQ ID: 38
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGrACCCCACCATCrACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATGCCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGAGATACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACgtgggcgccgacaagaagctgcgcaagcgcagctctaaactg
gccacagagGAAGAGTTCTACAAGTTCATCAAGCCCATCCTGGAAAAGATGGACGGCAC
CGAGGAACTGCTCGTGAAGCTGAACAGAGAGGACCTGCTGCGGAAGCAGCGGACC
TTCGACAACGGCAGCATCCCCCACCAGATCCACCTGGGAGAGCTGCACGCCATTCT
GCGGCGGCAGGAAGATTTTTACCCATTCCTGAAGGACAACCGGGAAAAGATCGAGA
AGATCCTGACCTTCCGCATCCCCTACTACGTGGGCCCTCTGGCCAGGGGAAACAGC
AGATTCGCCTGGATGACCAGAAAGAGCGAGGAAACCATCACCCCCTGGAACTTCGA
GGAAGTGGTGGACAAGGGCGCTTCCGCCCAGAGCTTCATCGAGCGGATGACCAACT
TCGATAAGAACCTGCCCAACGAGAAGGTGCTGCCCAAGCACAGCCTGCTGTACGAG
TACTTCACCGTGTATAACGAGCTGACCAAAGTGAAATACGTGACCGAGGGAATGAG
AAAGCCCGCCTTCCTGAGCGGCGAGCAGAAAAAGGCCATCGTGGACCTGCTGTTCA
AGACCAACCGGAAAGTGACCGTGAAGCAGCTGAAAGAGGACTACTTCAAGAAAAT
CGAGTGCTTCGACTCCGTGGAAATCTCCGGCGTGGAAGATCGGTTCAACGCCTCCCT
GGGCACATACCACGATCTGCTGAAAATTATCAAGGACAAGGACTTCCTGGACAATG
AGGAAAACGAGGACATTCTGGAAGATATCGTGCTGACCCTGACACTGTTTGAGGAC
AGAGAGATGATCGAGGAACGGCTGAAAACCTATGCCCACCTGTTCGACGACAAAGT
GATGAAGCAGCTGAAGCGGCGGAGATACACCGGCTGGGGCAGGCTGAGCCGGAAG
CTGATCAACGGCATCCGGGACAAGCAGTCCGGCAAGACAATCCTGGATTTCCTGAA
GTCCGACGGCTTCGCCAACAGAAACTTCATGCAGCTGATCCACGACGACAGCCTGA
CCTTTAAAGAGGACATCCAGAAAGCCCAGGTGTCCGGCCAGGGCGATAGCCTGCAC
GAGCACATTGCCAATCTGGCCGGCAGCCCCGCCATTAAGAAGGGCATCCTGCAGAC
AGTGAAGGTGGTGGACGAGCTCGTGAAAGTGATGGGCCGGCACAAGCCCGAGAAC
ATCGTGATCGAAATGGCCAGAGAGAACCAGACCACCCAGAAGGGACAGAAGAACA
GCCGCGAGAGAATGAAGCGGATCGAAGAGGGCATCAAAGAGCTGGGCAGCCAGAr
CCTGAAAGAACACCCCGTGGAAAACACCCAGCTGCAGAACGAGAAGCTGTACCTGT
ACTACCTGCAGAATGGGCGGGATATGTACGTGGACCAGGAACTGGACATCAACCGG
CTGTCCGACTACGATGTGGACCATATCGTGCCTCAGAGCTTTCTGAAGGACGACTCC
ATCGACAACAAGGTGCTGACCAGAAGCGACAAGAACCGGGGCAAGAGCGACAACG
TGCCCTCCGAAGAGGTCGTGAAGAAGATGAAGAACTACTGGCGGCAGCTGCTGAAC
GCCAAGCTGATTACCCAGAGAAAGTTCGACAATCTGACCAAGGCCGAGAGAGGCG
GCCTGAGCGAACTGGATAAGGCCGGCTTCATCAAGAGACAGCTGGTGGAAACCCGG
CAGATCACAAAGCACGTGGCACAGATCCTGGACTCCCGGATGAACACTAAGTACGA
CGAGAATGACAAGCTGATCCGGGAAGTGAAAGTGATCACCCTGAAGTCCAAGCTGG
TGTCCGATTTCCGGAAGGATTTCCAGTTTTACAAAGTGCGCGAGATCAACAACTACC
ACCACGCCCACGACGCCTACCTGAACGCCGTCGTGGGAACCGCCCTGATCAAAAAG
TACCCTAAGCTGGAAAGCGAGTTCGTGTACGGCGACTACAAGGTGTACGACGTGCG
GAAGATGATCGCCAAGAGCGAGCAGGAAATCGGCAAGGCTACCGCCAAGTACTTCT
TCTACAGCAACATCATGAACTTTTTCAAGACCGAGATTACCCTGGCCAACGGCGAG
ATCCGGAAGCGGCCTCTGATCGAGACAAACGGCGAAACCGGGGAGATCGTGTGGG
ATAAGGGCCGGGATTTTGCCACCGTGCGGAAAGTGCTGAGCATGCCCCAAGTGAAT
ATCGTGAAAAAGACCGAGGTGCAGACAGGCGGCTTCAGCAAAGAGTCTATCCgGCC
CAAGAGGAACAGCGATAAGCTGATCGCCAGAAAGAAGGACTGGGACCCTAAGAAG
TACGGCGGCTTCGtCAGCCCCACCGTGGCCTATTCTGTGCTGGTGGTGGCCAAAGTG
GAAAAGGGCAAGTCCAAGAAACTGAAGAGTGTGAAAGAGCTGCTGGGGATCACCA
TCATGGAAAGAAGCAGCTTCGAGAAGAATCCCATCGACTTTCTGGAAGCCAAGGGC
TACAAAGAAGTGAAAAAGGACCTGATCATCAAGCTGCCTAAGTACTCCCTGTTCGA
GCTGGAAAACGGCCGGAAGAGAATGCTGGCCTCTGCCcGCtttCTGCAGAAGGGAAA
CGAACTGGCCCTGCCCTCCAAATATGTGAACTTCCTGTACCTGGCCAGCCACTATGA
GAAGCTGAAGGGCTCCCCCGAGGATAATGAGCAGAAACAGCTGTTTGTGGAACAGC
ACAAGCACTACCTGGACGAGATCATCGAGCAGATCAGCGAGTTCTCCAAGAGAGTG
ATCCTGGCCGACGCTAATCTGGACAAAGTGCTGTCCGCCTACAACAAGCACCGGGA
TAAGCCCATCAGAGAGCAGGCCGAGAATATCATCCACCTGTTTACCCTGACCAATC
TGGGAGCCCCTcggGCCTTCAAGTACTTTGACACCACCATCGACCGGAAGgtGTACcgg
AGCACCAAAGAGGTGCTGGACGCCACCCTGATCCACCAGAGCATCACCGGCCTGTA
CGAGACACGGATCGACCTGTCTCAGCTGGGAGGTGAC,
SpCas9(D10A)-NGX-loop: Nucleotide sequence:
SEQ ID NO: 39
ATGGACAAGAAGTACAGCATCGGCCTGGCCATCGGCACCAACTCTGTGGGCT
GGGCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCAA
GGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCAC
GAGAAGTACCCCACCATCTACCACCTGAGAAAGAAACTGGTGGACAGCACCGACA
AGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTT
CATCCAGCTGGTGCAGACCTACAACCAGCTGTTCGAGGAAAACCCCATCAACGCCA
GCGGCGTGGACGCCAAGGCCATCCTGTCTGCCAGACTGAGCAAGAGCAGACGGCTG
GAAAATCTGATCGCCCAGCTGCCCGGCGAGAAGAAGAATGGCCTGTTCGGAAACCT
GATTGCCCTGAGCCTGGGCCTGACCCCCAACTTCAAGAGCAACTTCGACCTGGCCG
AGGATACCAAACTGCAGCTGAGCAAGGACACCTACGACGACGACCTGGACAACCT
GCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCC
CCCCTGAGCGCCTCTATGATCAAGCTGTACGACGAGCACCACCAGGACCTGACCCT
GCTGAAAGCTCTCGTGCGGCAGCAGCTGCCTGAGAAGTACAAAGAGATTTTCTTCG
ACCAGAGCAAGAACGGCTACGCCGGCTACgtgggcgccgacaagaagctgcgcaagcgcagctctaaactg
gccacagagGAAGAGTTCTACAAGTTCATCAAGCCCATCCTGGAAAAGATGGACGGCAC
CGAGGAACTGCTCGTGAAGCTGAACAGAGAGGACCTGCTGCGGAAGCAGCGGACC
TTCGACAACGGCATCATCCCCCACCAGATCCACCTGGGAGAGCTGCACGCCATTCT
GCGGCGGCAGGAAGATTTTTACCCATTCCTGAAGGACAACCGGGAAAAGATCGAGA
AGATCCTGACCTTCCGCATCCCCTACTACGTGGGCCCTCTGGCCAGGGGAAACAGC
AGATTCGCCTGGATGACCAGAAAGAGCGAGGAAACCATCACCCCCTGGAACTTCGA
GAAGGTGGTGGACAAGGGCGCTTCCGCCCAGAGCTTCATCGAGCGGATGACCAACT
TCGATAAGAACCTGCCCAACGAGAAGGTGCTGCCCAAGCACAGCCTGCTGTACGAG
TACTTCACCGTGTATAACGAGCTGACCAAAGTGAAATACGTGACCGAGGGAATGAG
AAAGCCCGCCTTCCTGAGCGGCGACCAGAAAAAGGCCATCGTGGACCTGCTGTTCA
AGACCAACCGGAAAGTGACCGTGAAGCAGCTGAAAGAGGACTACTTCAAGAAAAr
CGAGTGCTTCGACTCCGTGGAAATCTCCGGCGTGGAAGATCGGTTCAACGCCTCCCT
GGGCACATACCACGATCTGCTGAAAATTATCAAGGACAAGGACTTCCTGGACAATG
AGGAAAACGAGGACATTCTGGAAGATATCGTGCTGACCCTGACACTGTTTGAGGAC
AGAGAGATGATCGAGGAACGGCTGAAAACCTATGCCCACCTGTTCGACGACAAAGT
GATGAAGCAGCTGAAGCGGCGGAGATACACCGGCTGGGGCAGGCTGAGCCGGAAG
CTGATCAACGGCATCCGGGACAAGCAGTCCGGCAAGACAATCCTGGATTTCCTGAA
GTCCGACGGCTTCGCCAACAGAAACTTCATCCAGCTGATCCACGACGACAGCCTGA
CCTTTAAAGAGGACATCCAGAAAGCCCAGGTGTCCGGCCAGGGCGATAGCCTGCAC
GAGCACATTGCCAATCTGGCCGGCAGCCCCGCCATTAAGAAGGGCATCCTGCAGAC
AGTGAAGGTGGTGGACGAGCTCGTGAAAGTGATGGGCCGGCACAAGCCCGAGAAC
ATCGTGATCGAAATGGCCAGAGAGAACCAGACCACCCAGAAGGGACAGAAGAACA
GCCGCGAGAGAATGAAGCGGATCGAAGAGGGCATCAAAGAGCTGGGCAGCCAGAT
CCTGAAAGAACACCCCGTGGAAAACACCCAGCTGCAGAACGAGAAGCTGTACCTGT
ACTACCTGCAGAATGGGCGGGATATGTACGTGGACCAGGAACTGGACATCAACCGG
CTGTCCGACTACGATGTGGACCATATCGTGCCTCAGAGCTTTCTGAAGGACGACTCC
ATCGACAACAAGGTGCTGACCAGAAGCGACAAGAACCGGGGCAAGAGCGACAACG
TGCCCTCCGAAGAGGTCGTGAAGAAGATGAAGAACTACTGGCGGCAGCTGCTGAAC
GCCAAGCTGATTACCCAGAGAAAGTTCGACAATCTGACCAAGGCCGAGAGAGGCG
GCCTGAGCGAACTGGATAAGGCCGGCTTCATCAAGAGACAGCTGGTGGAAACCCGG
CAGATCACAAAGCACGTGGCACAGATCCTGGACTCCCGGATGAACACTAAGTACGA
CGAGAATGACAAGCTGATCCGGGAAGTGAAAGTGATCACCCTGAAGTCCAAGCTGG
TGTCCGATTTCCGGAAGGATTTCCAGTTTTACAAAGTGCGCGAGATCAACAACTACC
ACCACGCCCACGACGCCTACCTGAACGCCGTCGTGGGAACCGCCCTGATCAAAAAG
TACCCTAAGCTGGAAAGCGAGTTCGTGTACGGCGACTACAAGGTGTACGACGTGCG
GAAGATGATCGCCAAGAGCGAGCAGGAAATCGGCAAGGCTACCGCCAAGTACTTCT
TCTACAGCAACATCATGAACTTTTTCAAGACCGAGATTACCCTGGCCAACGGCGAG
ATCCGGAAGCGGCCTCTGATCGAGACAAACGGCGAAACCGGGGAGATCGTGTGGG
ATAAGGGCCGGGATTTTGCCACCGTGCGGAAAGTGCTGAGCATGCCCCAAGTGAAT
ATCGTGAAAAAGACCGAGGTGCAGACAGGCGGCTTCAGCAAAGAGTCTATCCgGCC
CAAGAGGAACAGCGATAAGCTGATCGCCAGAAAGAAGGACTGGGACCCTAAGAAG
TACGGCGGCTTCGtCAGCCCCACCGTGGCCTATTCTGTGCTGGTGGTGGCCAAAGTG
GAAAAGGGCAAGTCCAAGAAACTGAAGAGTGTGAAAGAGCTGCTGGGGATCACCA
TCATGGAAAGAAGCAGCTTCGAGAAGAATCCCATCGACTTTCTGGAAGCCAAGGGC
TACAAAGAAGTGAAAAAGGACCTGATCATCAAGCTGCCTAAGTACTCCCTGTTCGA
GCTGGAAAACGGCCGGAAGAGAATGCTGGCCTCTGCCcGCtttCTGCAGAAGGGAAA
CGAACTGGCCCTGCCCTCCAAATATGTGAACTTCCTGTACCTGGCCAGCCACTATGA
GAAGCTGAAGGGCTCCCCCGAGGATAATGAGCAGAAACAGCTGTTTGTGGAACAGC
ACAAGCACTACCTGGACGAGATCATCGAGCAGATCAGCGAGTTCTCCAAGAGAGTG
ATCCTGGCCGACGCTAATCTGGACAAAGTGCTGTCCGCCTACAACAAGCACCGGGA
TAAGCCCATCAGAGAGCAGGCCGAGAATATCATCCACCTGTTTACCCTGACCAATC
TGGGAGCCCCTcggGCCTTCAAGTACTTTGACACCACCATCGACCGGAAGgtGTACcgg
AGCACCAAAGAGGTGCTGGACGCCACCCTGATCCACCAGAGCATCACCGGCCTGTA
CGAGACACGGATCGACCTGTCTCAGCTGGGAGGTGAC,
ScCas9(D10A): Nucleotide sequence:
SEQ ID NO: 40
ATGGAGAAGAAGTACAGCATCGGCCTGGCCATCGGCACCAACAGCGTGGGC
TGGGCCGTGATCACCGACGACTACAAGGTGCCCAGCAAGAAGTTCAAGGTGCTGGG
CAACACCAACCGCAAGAGCATCAAGAAGAACCTGATGGGCGCCCTGCTGTTCGACA
GCGGCGAGACCGCCGAGGCCACCCGCCTGAAGCGCACCGCCCGCCGCCGCTACACC
CGCCGCAAGAACCGCATCCGCTACCTGCAGGAGATATTCGCCAACGAGATGGCCAA
GCTGGACGACAGCTTCTTCCAGCGCCTGGAGGAGAGCTTCCTGGTGGAGGAGGACA
AGAAGAACGAGCGCCACCCCATCTTCGGCAACCTGGCCGACGAGGTGGCCTACCAC
CGCAACTACCCCACCATCTACCACCTGCGCAAGAAGCTGGCCGACAGCCCCGAGAA
GGCCGACCTGCGCCTGATCTACCTGGCCCTGGCCCACATCATCAAGTTCCGCGGCCA
CTTCCTGATCGAGGGCAAGCTGAACGCCGAGAACAGCGACGTGGCCAAGCTGTTCT
ACCAGCTGATCCAGACCTACAACCAGCTGTTCGAGGAGAGCCCCCTGGACGAGATC
GAGGTGGACGCCAAGGGCATCCTGAGCGCCCGCCTGAGCAAGAGCAAGCGCCTGG
AGAAGCTGATCGCCGTGTTCCCCAACGAGAAGAAGAACGGCCTGTTCGGCAACATC
ATCGCCCTGGCCCTGGGCCTGACCCCCAACTTCAAGAGCAACTTCGACCTGACCGA
GGACGCCAAGCTGCAGCTGAGCAAGGACACCTACGACGACGACCTGGACGAGCTG
CTGGGCCAGATCGGCGACCAGTACGCCGACCTGTTCAGCGCCGCCAAGAACCTGAG
CGACGCCATCCTGCTGAGCGACATCCTGCGCAGCAACAGCGAGGTGACCAAGGCCC
CCCTGAGCGCCAGCATGGTGAAGCGCTACGACGAGCACCACCAGGACCTGGCCCTG
CTGAAGACCCTGGTGCGCCAGCAGTTCCCCGAGAAGTACGCCGAGATATTCAAGGA
CGACACCAAGAACGGCTACGCCGGCTACGTGGGCATCGGCATCAAGCACCGCAAGC
GCACCACCAAGCTGGCCACCCAGGAGGAGTTCTACAAGTTCATCAAGCCCATCCTG
GAGAAGATGGACGGCGCCGAGGAGCTGCTGGCCAAGCTGAACCGCGACGACCTGC
TGCGCAAGCAGCGCACCTTCGACAACGGCAGCATCCCCCACCAGATCCACCTGAAG
GAGCTGCACGCCATCCTGCGCCGCCAGGAGGAGTTCTACCCCTTCCTGAAGGAGAA
CCGCGAGAAGATCGAGAAGATCCTGACCTTCCGCATCCCCTACTACGTGGGCCCCC
TGGCCCGCGGCAACAGCCGCTTCGCCTGGCTGACCCGCAAGAGCGAGGAGGCCATC
ACCCCCTGGAACTTCGAGGAGGTGGTGGACAAGGGCGCCAGCGCCCAGAGCTTCAT
CGAGCGCATGACCAACTTCGACGAGCAGCTGCCCAACAAGAAGGTGCTGCCCAAGC
ACAGCCTGCTGTACGAGTACTTCACCGTGTACAACGAGCTGACCAAGGTGAAGTAC
GTGACCGAGCGCATGCGCAAGCCCGAGTTCCTGAGCGGCGAGCAGAAGAAGGCCA
TCGTGGACCTGCTGTTCAAGACCAACCGCAAGGTGACCGTGAAGCAGCTGAAGGAG
GACTACTTCAAGAAGATCGAGTGCTTCGACAGCGTGGAGATCATCGGCGTGGAGGA
CCGCTTCAACGCCAGCCTGGGCACCTACCACGACCTGCTGAAGATCATCAAGGACA
AGGACTTCCTGGACAACGAGGAGAACGAGGACATCCTGGAGGACATCGTGCTGACC
CTGACCCTGTTCGAGGACCGCGAGATGATCGAGGAGCGCCTGAAGACCTACGCCCA
CCTGTTCGACGACAAGGTGATGAAGCAGCTGAAGCGCCGCCACTACACCGGCTGGG
GCCGCCTGAGCCGCAAGATGATCAACGGCATCCGCGACAAGCAGAGCGGCAAGAC
CATCCTGGACTTCCTGAAGAGCGACGGCTTCAGCAACCGCAACTTCATGCAGCTGA
TCCACGACGACAGCCTGACCTTCAAGGAGGAGATCGAGAAGGCCCAGGTGAGCGG
CCAGGGCGACAGCCTGCACGAGCAGATCGCCGACCTGGCCGGCAGCCCCGCCATCA
AGAAGGGCATCCTGCAGACCGTGAAGATCGTGGACGAGCTGGTGAAGGTGATGGG
CCACAAGCCCGAGAACATCGTGATCGAGATGGCCCGCGAGAACCAGACCACCACC
AAGGGCCTGCAGCAGAGCCGCGAGCGCAAGAAGCGCATCGAGGAGGGCATCAAGG
AGCTGGAGAGCCAGATCCTGAAGGAGAACCCCGTGGAGAACACCCAGCTGCAGAA
CGAGAAGCTGTACCTGTACTACCTGCAGAACGGCCGCGACATGTACGTGGACCAGG
AGCTGGACATCAACCGCCTGAGCGACTACGACGTGGACCACATCGTGCCCCAGAGC
TTCATCAAGGACGACAGCATCGACAACAAGGTGCTGACCCGCAGCGTGGAGAACCG
CGGCAAGAGCGACAACGTGCCCAGCGAGGAGGTGGTGAAGAAGATGAAGAACTAC
TGGCGCCAGCTGCTGAACGCCAAGCTGATCACCCAGCGCAAGTTCGACAACCTGAC
CAAGGCCGAGCGCGGCGGCCTGAGCGAGGCCGACAAGGCCGGCTTCATCAAGCGC
CAGCTGGTGGAGACCCGCCAGATCACCAAGCACGTGGCCCGCATCCTGGACAGCCG
CATGAACACCAAGCGCGACAAGAACGACAAGCCCATCCGCGAGGTGAAGGTGATC
ACCCTGAAGAGCAAGCTGGTGAGCGACTTCCGCAAGGACTTCCAGCTGTACAAGGT
GCGCGACATCAACAACTACCACCACGCCCACGACGCCTACCTGAACGCCGTGGTGG
GCACCGCCCTGATCAAGAAGTACCCCAAGCTGGAGAGCGAGTTCGTGTACGGCGAC
TACAAGGTGTACGACGTGCGCAAGATGATCGCCAAGAGCGAGCAGGAGATCGGCA
AGGCCACCGCCAAGCGCTTCTTCTACAGCAACATCATGAACTTCTTCAAGACCGAG
GTGAAGCTGGCCAACGGCGAGATCCGCAAGCGCCCCCTGATCGAGACCAACGGCG
AGACCGGCGAGGTGGTGTGGAACAAGGAGAAGGACTTCGCCACCGTGCGCAAGGT
GCTGGCCATGCCCCAGGTGAACATCGTGAAGAAGACCGAGGTGCAGACCGGCGGCT
TCAGCAAGGAGAGCATCCTGAGCAAGCGCGAGAGCGCCAAGCTGATCCCCCGCAA
GAAGGGCTGGGACACCCGCAAGTACGGCGGCTTCGGCAGCCCCACCGTGGCCTACA
GCATCCTGGTGGTGGCCAAGGTGGAGAAGGGCAAGGCCAAGAAGCTGAAGAGCGT
GAAGGTGCTGGTGGGCATCACCATCATGGAGAAGGGCAGCTACGAGAAGGACCCC
ATCGGCTTCCTGGAGGCCAAGGGCTACAAGGACATCAAGAAGGAGCTGATCTTCAA
GCTGCCCAAGTACAGCCTGTTCGAGCTGGAGAACGGCCGCCGCCGCATGCTGGCCA
GCGCCACCGAGCTGCAGAAGGCCAACGAGCTGGTGCTGCCCCAGCACCTGGTGCGC
CTGCTGTACTACACCCAGAACATCAGCGCCACCACCGGCAGCAACAACCTGGGCTA
CATCGAGCAGCACCGCGAGGAGTTCAAGGAGATATTCGAGAAGATCATCGACTTCA
GCGAGAAGTACATCCTGAAGAACAAGGTGAACAGCAACCTGAAGAGCAGCTTCGA
CGAGCAGTTCGCCGTGAGCGACAGCATCCTGCTGAGCAACAGCTTCGTGAGCCTGC
TGAAGTACACCAGCTTCGGCGCCAGCGGCGGCTTCACCTTCCTGGACCTGGACGTG
AAGCAGGGCCGCCTGCGCTACCAGACCGTGACCGAGGTGCTGGACGCCACCCTGAT
CTACCAGAGCATCACCGGCCTGTACGAGACCCGCACCGACCTGAGCCAGCTGGGCG
GCGAC,
TadA-TadA* Nucleotide sequence:
SEQ ID NO: 41
TCTGAAGTCGAGTTTAGCCACGAGTATTGGATGAGGCACGCACTGACCCTGG
CAAAGCGAGCATGGGATGAAAGAGAAGTCCCCGTGGGCGCCGTGCTGGTGCACAA
CAATAGAGTGATCGGAGAGGGATGGAACAGGCCAATCGGCCGCCACGACCCTACC
GCACACGCAGAGATCATGGCACTGAGGCAGGGAGGCCTGGTCATGCAGAATTACCG
CCTGATCGATGCCACCCTGTATGTGACACTGGAGCCATGCGTGATGTGCGCAGGAG
CAATGATCCACAGCAGGATCGGAAGAGTGGTGTTCGGAGCACGGGACGCCAAGAC
CGGCGCAGCAGGCTCCCTGATGGATGTGCTGCACCACCCCGGCATGAACCACCGGG
TGGAGATCACAGAGGGAATCCTGGCAGACGAGTGCGCCGCCCTGCTGAGCGATTTC
TTTAGAATGCGGAGACAGGAGATCAAGGCCCAGAAGAAGGCACAGAGCTCCACCG
ACTCTGGAGGATCTAGCGGAGGATCCTCTGGAAGCGAGACACCAGGCACAAGCGA
GTCCGCCACACCAGAGAGCTCCGGCGGCTCCTCCGGAGGATCCTCTGAGGTGGAGT
TTTCCCACGAGTACTGGATGAGACATGCCCTGACCCTGGCCAAGAGGGCACGCGAT
GAGAGGGAGGTGCCTGTGGGAGCCGTGCTGGTGCTGAACAATAGAGTGATCGGCG
AGGGCTGGAACAGAGCCATCGGCCTGCACGACCCAACAGCCCATGCCGAAATTATG
GCCCTGAGACAGGGCGGCCTGGTCATGCAGAACTACAGACTGATTGACGCCACCCT
GTACGTGACATTCGAGCCTTGCGTGATGTGCGCCGGCGCCATGATCCACTCTAGGAT
CGGCCGCGTGGTGTTTGGCGTGAGGAACGCAAAAACCGGCGCCGCAGGCTCCCTGA
TGGACGTGCTGCACTACCCCGGCATGAATCACCGCGTCGAAATTACCGAGGGAATC
CTGGCAGATGAATGTGCCGCCCTGCTGTGCTATTTCTTTCGGATGCCTAGACAGGTG
TTCAATGCTCAGAAGAAGGCCCAGAGCTCCACCGAC,
TadA*(A56G_V82G) Nucleotide sequence:
SEQ ID: 42
TCTGAGGTGGAGTTTTCCCACGAGTACTGGATGAGACATGCCCTGACCCTGG
CCAAGAGGGCACGCGATGAGAGGGAGGTGCCTGTGGGAGCCGTGCTGGTGCTGAA
CAATAGAGTGATCGGCGAGGGCTGGAACAGAGCCATCGGCCTGCACGACCCAACA
GgCCATGCCGAAATTATGGCCCTGAGgCAGGGCGGCCTGGTCATGCAGAACTACAG
ACTGATTGACGCCACCCTGTACGgGACATTCGAGCCTTGCGTGATGTGCGCCGGCGC
CATGATCCACTCTAGGATCGGCCGCGTGGTGTTTGGCGTGAGGAACGCAAAAACCG
GCGCCGCAGGCTCCCTGATGGACGTGCTGCACTACCCCGGCATGAATCACCGCGTC
GAAATTACCGAGGGAATCCTGGCAGATGAATGTGCCGCCCTGCTGTGCTATTTCTTT
CGGATGCCTAGACAGGTGTTCAATGCTCAGAAGAAGGCCCAGAGCTCCACCGAC,
Gp41-1-N: Nucleotide sequence:
SEQ ID NO: 43
TGTTTGGATCTGAAAACGCAAGTTCAAACGCCACAGGGTATGAAAGAAATAT
CCAATATACAGGTCGGCGATCTCGTCTTGTCTAACACTGGCTATAACGAGGTGCTGA
ATGTATTTCCAAAAAGCAAGAAAAAAAGTTACAAGATAACTCTGGAAGATGGAAA
AGAAATTATCTGTTCTGAGGAGCATCTGTTTCCGACCCAAACAGGGGAGATGAATA
TCAGTGGCGGTCTCAAAGAGGGTATGTGTTTGTATGTCAAGGaataa,
Gp41-1-C: Nucleotide sequence:
SEQ ID NO: 44
ATGATGCTCAAGAAGATCCTCAAGATTGAAGAGTTGGACGAGCGCGAGCTT
ATAGACATAGAAGTCAGTGGTAATCACCTTTTCTACGCAAATGACATTTTGACTCAC
AACTCC,
Cfa-N: Nucleotide sequence:
SEQ ID NO: 45
TGTCTCAGTTATGAcACCGAAATCCTGACAGTCGAGTATGGAtTtCTGCCGATC
GGCAAGATTGTGGAGgAGAGAATTGAATGTACGGTCTATAcgGTCGACAAgAATGGTt
tCgTCTACACCCAACCAaTTGCTCAATGGCATaATCGAGGGGAGCAGGAGGTGTTTGA
GTATTGCCTGGAGGACGGGTCAaTCATTAGAGCTACAAAGGACCATAAGTTTATGAC
AacCGATGGTCAAATGCTGCCGATAGATGAAATATTCGAAAGGGgACTGGATCTTAa
GCaAGTCGATggCCTTCCAaac,
Cfa-C, Nucleotide sequence:
SEQ ID NO: 46
ATGgTcAAgATTatCAgcCGCAAATcCTTGGGGAcACAGAATGTATATGACATCG
GCGTGGAAaaGGATCACAATTTTctgCTGAAGAATGGTcTTgTTGCTtccAAt,
Tad A,
SEQ ID NO: 47
SEVEFSHEYWMRHALTLAKRARDEREVPVGAVLVLNNRVIGEGWNRAIGLHDP
TAHAEIMALRQGGLVMQNYRLIDATLYVTFEPCVMCAGAMIHSRIGRVVFGVRNAKT
GAAGSLMDVLHYPGMNHRVEITEGILADECAALLCYFFRMPRQVFNAQKKAQSSTD,
SEQ ID NO: 48
TGAGTATCATCGTGTGAAAGCTgAGGGGACGAGGCAGGCCTATAA
SEQ ID NO: 49
AAAAACATCAACTTCAGCCATCCATTTCTTCAGGGTTTGTATGTG
SEQ ID NO: 50
TATATCATAATGAAAACGCCGCCATTTCTCAACAGATCTGTCAAA
SEQ ID NO: 51
CTGTGTGAAATGGCTGCAAATCgATGGTTGAGCTCTGAGATTTGG
SEQ ID NO: 52
GTTCTGCTTTTGCTACTACTCACGTTTCCATGTTGTCCCCCTCTA
SEQ ID NO: 53
ATTTTATGGCCTTTTGCAACTCgACCAGAAAAAAAGCAGCTTTGG
SEQ ID NO: 54
TGAGGAGATCGCCCACGGGCTGCCAGGATCCCTTGATCACCTCAG
SEQ ID NO: 55
GGCTGCTCTGTCAGAAATATTCgTACAGTCTCAAGAGTACTCATG
SEQ ID NO: 56
GTGTAGGCATAGCTCTTGAATCgAGGCTTAGGGGAAGAAGTTCTC
SEQ ID NO: 57
CCTGTTCTTCAGTAAGACGTTGCCATTTGAGAAGGATGTCTTGTA
SEQ ID NO: 58
GCCATTTTAGGCTTTTTACTTACTTGTCTGTAGCTCTTTCTCTCT
SEQ ID NO: 59
CTAGTTTCTCACACATGACACACCTGTTCTTCAGTAAGACGTTGC
SEQ ID NO: 60
GTGAAGTTGATTACATTAACCTgTGGATAATTACGAGTTGATTGT
SEQ ID NO: 61
ttttgtatATCTGAGTTAAACTgCTCCAATTCCTTCAAAGGAATG
SEQ ID NO: 62
TCTGCAATATAAGCTGCCAACTgCTTGTCAATGAATGTGAGGGAC
SEQ ID NO: 63
GGACTGGGGTTCCAGTCTCATCCAGTCTAGGAAGAGGGCCGCTTC
SEQ ID NO: 64
AGTCGTTGTGTGGCTGACTGCTgGCAAACCACACTATTCCAGTCA
SEQ ID NO: 65
ATTTGtgtctttctgagaaactgttcaGCTTCTGTTAGCCACTGA
SEQ ID NO: 66
AAGAACCCAGCGGTCTTCTGTCCATCTACAGATGTTTGCCCATCG
SEQ ID NO: 67
AGACTTTTTCCGAAGTTCACTCCACTTGAAGTTCATGTTATCCAA
SEQ ID NO: 68
TACCTGTTGGCACATGTGATCCCACTGAGTGTTAAGTTCTTTGAG
SEQ ID NO: 69
CAAAGGGCCTTCTGCAGTCTTCgGAGTTTCATGGCAGTCCTATAA
SEQ ID NO: 70
ATGGTTAATGTCTAACCTTTATCCACTGGAGATTTGTCTGCTTGA
SEQ ID NO: 71
CAAAATAATCTGACCTTAAGTTgTTCTTCCAAAGCAGCAGTTGCG
SEQ ID NO: 72
CATCTACAGATGTTTGCCCATCgATCTCCCAATACCTGGAGAAGA
SEQ ID NO: 73
AAGGTGTTCTTGTACTTCATCCCACTGATTCTGAATTCTTTCAac
SEQ ID NO: 74
GAAGGTGTTCTTGTACTTCATCCCACTGATTCTGAATTCTTTCAa
SEQ ID NO: 75
GCTATGCTTTGAATTTTTAATCgTTCAATTTGAGGTTGAAGATCT
SEQ ID NO: 76
TCTAGGAGGCGCCTCCCATCCTgTAGGTCACTGAAGAGGTTCTCA
SEQ ID NO: 77
GTGTAATTACCATTCACCATCTgTTCCACCAGGGCCTGAGCTGAT
SEQ ID NO: 78
TGAGCATGCTTTACCAGGATCTgTTCCCTTGTGGTCACCGTAGTT
SEQ ID NO: 79
CTACTGTATAGGGACCCTCCTTCCATGACTCAAGCTTGGCTCTGG
SEQ ID NO: 80
CAGCTTCTTCCTTAGCTTCCAGCCATTGTGTTGAATCCTTTAACA
SEQ ID NO: 81
AGCTGCCCAAGGTCTTTTATTTgAGCTTCAATTTCTCCTTGTTTC
SEQ ID NO: 82
TGCCAGTAACAACTCACAATTTgTGCAAAGTTGAGTCTTCGAAAC
SEQ ID NO: 83
AGAGTAACAGTCTGAGTAGGAGctaaaatattttgggtttttgca
SEQ ID NO: 84
CTTCAGCAAAAAAAGTACTCACgCAGAATCTACTGGCCAGAAGTT
SEQ ID NO: 85
ACATCTACATTTGTCTGCCACTgGCGGAGGTCTTTGGCCAACTGC
SEQ ID NO: 86
CTGAGATAGTATAGGCCACTTTgTTGCTCTTGCAGAGAACTTTGT
SEQ ID NO: 87
TAGCTGTCCTTTACACACTTTACCTGTTGAGAATAGTGCATTTGA
SEQ ID NO: 88
GCCTGGGCTTCCTGAGGCATTTgAGCTGCGTCCACCTTGTCTGCA
SEQ ID NO: 89
TGCACTGGCAGGTAGCCCATTCgGGGATGCTTCGCAAAATACCTT
SEQ ID NO: 90
TTATAGTTCCACATTCAATTACcTCTGGGCTCCTGGTAGAGTTTC
SEQ ID NO: 91
CGTCAGGCTGGCGTCAAACTTAcCGGAGTGCAATATTCCACCATG
SEQ ID NO: 92
taTCCAAAAGTGTGTCAGCCTgAATGATCCACTTTGTGATGTGG
SEQ ID NO: 93
TGTAGCCACACCAGAAGTTCCTgCAGAGAAAGGTGCAGACGCTTC
SEQ ID NO: 94
TGTAAGGATTTTTCAGTCTCCTgGGCAGACTGGATGCTCTGTTCA
SEQ ID NO: 95
AGAATGGGATCCAGTATACTTAcAGGCTCCAATAGTGGTCAGTCC
SEQ ID NO: 96
TTCAGAGGCGCAATTTCTCCTCgAAGTGCCTGTGTGCAATAGTCA
SEQ ID NO: 97
GTTTCTTCCAAAGCAGCCTCTCgCTCACTCACCCTGCAAAGGACC
SEQ ID NO: 98
CTGAACTTCTCAGCTTTTTCTCgCTCTATGGCCTGCAGCATGAGA
SEQ ID NO: 99
AGATTTAACCACTCTTCTGCTCgGGAGGTGACAGCTATCCAGTTA
SEQ ID NO: 100
tgttttatctttatttcctctCgCTTTCTCTCATCTGTGATTCTT
SEQ ID NO: 101
CCAGCTGGGAGGAGAGCTTCTTcCAGCGTCCCTCAATTTCTTCAA
SEQ ID NO: 102
ACACAGCTTCTGAGCGAGTAATcCAGCTGTGAAGTTCAGTTATAT
SEQ ID NO: 103
AAGTAAACGGTTTACCGCCTTCcACTCAGAGCTCAGATCTTCTAA
SEQ ID NO: 104
aaatagaaaaattagatgacttgccaaaggtcacaaaGGTAACTG
SEQ ID NO: 105
TTGACTTTCTCGAGGTGATCTTgGAGAGAGTCAATGAGGAGATCG
SEQ ID NO: 106
TCTAAAATCATCTTACTTTCTTgTAGACGCTGCTCAAAATTGGCT
SEQ ID NO: 107
GAATTGACCCTGACTTGTTCTTgTTCTAGATCTTCTTGAAGCACc
SEQ ID NO: 108
TGGATGGCTTCAATGCTCACTTgTTGAGGCAAAACTTGGAAGAGT
SEQ ID NO: 109
AACAGTCCTCTACTTCTTCCCAcCAAAGCATTTTGAAAAGTGTAT
SEQ ID NO: 110
AGGCCTCCTTTCTGGCATAGACcttccacaaaacaaacaaacaaa
SEQ ID NO: 111
TTTGGTTTCTGACTGCTGGACCcATGTCCTGATGGCACTCATGGT
SEQ ID NO: 112
ATCTTACTTTCTTGTAGACGCTgCTCAAAATTGGCTGGTTTCTGG
SEQ ID NO: 113
AGATTTTTCACTTATCTTCATAcCTCTTCATGTAGTTCCCTCCAA
SEQ ID NO: 114
CTGTTCAGTTGTTCTGAGGCTTgTTTGATGCTATCTGCATTAACA
SEQ ID NO: 115
CAGCATTAATATACACGACTTAcATCTGTACTTGTCTTCCAAATG
SEQ ID NO: 116
TCATGACTTGTCAAATCAGATTgGATTTTCTGTTGGGAGGATAGC
SEQ ID NO: 117
ATCTGCTCCAATTGTTGTAGCTgATTATAGAAAGCGATGATGTTG
SEQ ID NO: 118
CAAATTTGCTCTCAATTTCCCGcCAGCGCTTGCTGAGCTGGATCT
SEQ ID NO: 119
CATTCAAAGCCAGGCCATCAGAcCAGCTGGTGGTGAAGTTGATTA
SEQ ID NO: 120
ttcatcTCTTCAACTGCTTTCTgTAATTCATCTGGAGTTTTATAT
SEQ ID NO: 121
ATTGAAAGCTAGAAAGTACATAcGGCCAGTTTTTGAAGACTTGAT
SEQ ID NO: 122
TTCAAATACTGGCCAATACTTAcAGCAAAGGGCCTTCTGCAGTCT
SEQ ID NO: 123
GTTGTCTGTGTTAGTGATGGCTgAGTGGTGGTGACAGCCTGTGAA
SEQ ID NO: 124
TCATCAGCCTGCCTCTTGTACTgATACCACTGATGAGAAATTTCT
SEQ ID NO: 125
TACTGTATAGGGACCCTCCTTCcATGACTCAAGCTTGGCTCTGGC
SEQ ID NO: 126
ATGTTGAATGCATGTTCCAGTCgTTGTGTGGCTGACTGCTGGCAA
SEQ ID NO: 127
TGCCATTTGAGAAGGATGTCTTgTAAAAGAACCCAGCGGTCTTCT
SEQ ID NO: 128
GAGACTTTTTCCGAAGTTCACTcCACTTGAAGTTCATGTTATCCA
SEQ ID NO: 129
TTAGCAACTGGCAGAATTCGATcCACCGGCTGTTCAGTTGTTCTG
SEQ ID NO: 130
TTGCCACATCTACATTTGTCTGcCACTGGCGGAGGTCTTTGGCCA
SEQ ID NO: 131
TCCCATTCAGCCTAGTGCAGAGcCACTGGTAGTTGGTGGTTAGAG
SEQ ID NO: 132
GACTTACTGGAAAGAAAGTGCTgAGATGCTGGACCAAAGTCCCTG
SEQ ID NO: 133
TTTAATCGTTCAATTTGAGGTTgAAGATCTGATAGCCGGTTGACT
SEQ ID NO: 134
AAAGAGATTGTCTATACCTGTTgGCACATGTGATCCCACTGAGTG
SEQ ID NO: 135
AAGTTTTTGGACTAAATTATCCcAACACCGGGCAAAGTTATCCAG
SEQ ID NO: 136
AGCTCAGCATCCCGGGGACTCTgGGGAGAGGTGGGCATCATTTCA
SEQ ID NO: 137
TTGTCCCCCTCTAAGACAGTCTgCACTGGCAGGTAGCCCATTCGG
SEQ ID NO: 138
TTCGCAAAATACCTTTTGGTTCgAAATTTGTTTTTTAGTACCTTG
SEQ ID NO: 139
TGCAACTCGACCAGAAAAAAAGcAGCTTTGGCAGATGTCATAATT
SEQ ID NO: 140
TTGCAGATGTTACATTTGGCCTgATGCTTGGCAGTTTCTGCAGCA
SEQ ID NO: 141
AAATAAAAACATGCCATACGTAcGTATCATAAACATTCAGCAGCC
SEQ ID NO: 142
TACTTACAGCAAAGGGCCTTCTgCAGTCTTCGGAGTTTCATGGCA
SEQ ID NO: 143
TTGACCTCCTCAGCCTGCTTTCgTAGAAGCCGAGTGACATTCTGG
SEQ ID NO: 144
ATTCAATTACCTCTGGGCTCCTgGTAGAGTTTCTCTAGTCCTTCC
SEQ ID NO: 145
AATGCCTGACTTACTTGCCATTgTTTCATCAGCTCTTTTACTCCC
SEQ ID NO: 146
TGTACTTCATCCCACTGATTCTgAATTCTTTCAactagaataaaa
SEQ ID NO: 147
TGCTTCATTACCTTCACTGGCTgAGTGGCTGGTTTTTCCTTGTAC
SEQ ID NO: 148
TTTAATTGTTTGAGAATTCCCTgGCGCAGGGGCAACTCTTCCACC
SEQ ID NO: 149
atatgtgttaCCTACCCTTGTCgGTCCTTGTacattttgttaact
SEQ ID NO: 150
TCAAGCTGGGAGAGAGCTTCCTgTAGCTTCACCCTTTCCACAGGC
SEQ ID NO: 151
ATGTCAATCCGACCTGAGCTTTgTTGTAGACTATCTTTTATATTc
SEQ ID NO: 152
GTTGTAGACTATCTTTTATATTctgtaatataaaaattttaaaac
SEQ ID NO: 153
TCCCGCCAGCGCTTGCTGAGCTgGATCTGAGTTGGCTCCACTGCC
SEQ ID NO: 154
TTATGTTTTGTCTGTAACAGCTgctgttttatctttatttcctct
SEQ ID NO: 155
TGTTTTGTCTGTAACAGCTgctgttttatctttatttcctctCGC
SEQ ID NO: 156
TTTCTCTCATCTGTGATTCTTTgTTGTAAGTTGTCTCCTCTTTGC
SEQ ID NO: 157
aCCTTAAGCACGTCTTCTTTTTgCtggggtttctttttctctgat
SEQ ID NO: 158
TTAAGCACGTCTTCTTTTTGCtggggtttctttttctctgattca
SEQ ID NO: 159
ATACTCTTCAGGTGCACCTTCTgTTTCTCAATCTCTTTTTGAGTA
SEQ ID NO: 160
AGCTGTGACTGTACTACTTCCTgTTCCACACTCTTTGTTTCCAAT
SEQ ID NO: 161
TTGGCTGGTTTCTGGAATAATCgAAACTTCATGGAGACATCTTGT
SEQ ID NO: 162
TGCATCTCTGATAGATCTTTCTgGAGGCTTACAGTTTTCTCCAAA
SEQ ID NO: 163
ACAGTGAAAGAGATTGTCTATAcCTGTTGGCACATGTGATCCCAC
SEQ ID NO: 164
AAGGCATCATATAAAAATCTTAcTCTGCACTGTTTCAGCTGCTTT
SEQ ID NO: 165
CTTACTCTGCACTGTTTCAGCTgCTTTTTTAGAATTTCTGAATCC
SEQ ID NO: 166
TCTTGAATTACCTGAATTTTTCgGAGTTTATTCATTTGCTCCTCT
SEQ ID NO: 167
TGTTGCTCTTGCAGAGAACTTTgTAAAgcctaaaaaacaattttt
SEQ ID NO: 168
ATTGGTGGCAAAGTGTCAAAAAcTTtatcaaaagggaaaaaagaa
SEQ ID NO: 169
TAGGCTTTTTACTTACTTGTCTgTAGCTCTTTCTCTCTGGCCTGC
SEQ ID NO: 170
GCCTGCACATCAGAAAAGACTTgCTTAAAATGATTTGTAAAGGCC
SEQ ID NO: 171
ATGGAAGGAGAAGAGATTCTTAcCTTACAAATTTTTAACTGACTT
SEQ ID NO: 172
GGTGGTGGGTTGGATTTTCAACcAGTTTTCAGCAGTAGTTGTCAT
SEQ ID NO: 173
TCGATCCACCGGCTGTTCAGTTgTTCTGAGGCTTGTTTGATGCTA
SEQ ID NO: 174
TGAGCTGATCTGCTGGCATCTTgCAGTTTTCTGAACTTCTCAGCT
SEQ ID NO: 175
ATAAAAGCTTAAGATGCTCTCAcCTTTTCCTAATTTCAGAATCCA
SEQ ID NO: 176
ATTTCAGAATCCACAGTAATCTgCCTCTTCTTttggggaggtggt
SEQ ID NO: 177
TGATAATTGGTATCACTAACCTgTGCTGTACTCTTTTCAAGTTTT
SEQ ID NO: 178
TCCAGCCATGCTTCCGTCTTCTgGGTCACTGACTTATTCTTCAGT
SEQ ID NO: 179
GAAGGATGTCTTGTAAAAGAACcCAGCGGTCTTCTGTCCATCTAC
SEQ ID NO: 180
TGTTCTTGTTCTAGATCTTCTTgAAGCACctgaaagataaaatgt
SEQ ID NO: 181
CCTACCTTATGTTGTTGTACTTgGCGTTTTAGGTCTTCAAGATCA
SEQ ID NO: 182
tctttcttctgtttttgttagCcAGTCATTCAACTCTTTCAGTTT
SEQ ID NO: 183
attaaaaacaaataaggacTTAcTTGCTTTGTTTTTCCATGCTAG
SEQ ID NO: 184
caaataaggacTTACTTGCTTTgTTTTTCCATGCTAGCTACCCTG
SEQ ID NO: 185
TTTAGGAGATTCATCTGCTCTtgtacttcagtttcttcatcttct
SEQ ID NO: 186
ACATCATTAGAAATCTCTCCTTgTGCTTGCAATGTGTCCTCAGCA
SEQ ID NO: 187
TGGTAGTCCAGAAATTTACCAAcCTTCAGGATCGAGTAGTTTCTC
SEQ ID NO: 188
tatttttcattacatttttgaCcTACATGtggaaataaattttca
SEQ ID NO: 189
CCATTCATCAGGATTCTTACCTgCCAGTGGAGGATTATATTCCAA
SEQ ID NO: 190
tttcttaaaaataagtcaCATAcCAGTTTTTGCCCTGTCAGGCCT
SEQ ID NO: 191
aataagtcaCATACCAGTTTTTgCCCTGTCAGGCCTTCGAGGAGG
SEQ ID NO: 192
GTAAAGTAACAAACCATTCTTAcCTTAGAAAATTGTGCATTTACC
SEQ ID NO: 193
TTTACTAAGCAAAATAATCTGAcCTTAAGTTGTTCTTCCAAAGCA
SEQ ID NO: 194
ACGGATCCTCCCTGTTCGTCCCcTATTATGAAGAATCAAAGCAGA
SEQ ID NO: 195
TTCTCAACAGATCTGTCAAATCgCCTGCAGGTAAAAGCATATGGA
SEQ ID NO: 196
CTGTCAAATCCATCATGTACCCcTGACAAAGAAGGAAGTTAACAA
SEQ ID NO: 197
TCTCAATATGCtgcttcccaaactgaaattaaaaaaaatacacto
SEQ ID NO: 198
CTTAATTCATCATCTTTCAGCTgTAGCCACACCAGAAGTTCCTGC
SEQ ID NO: 199
GTCAAGACATTCATTTCCTTTCgCATCTTACGGGACAATTTCAAG
SEQ ID NO: 200
TGTGTCCTCAGCAGAAAGAAGCcACGATAATACTTCTTCTAAAGC
SEQ ID NO: 201
TAGAAAGCGATGATGTTGTTCTgATACTCCAGCCAGTTAAGTCTC
SEQ ID NO: 202
CTCTCTAAGGAAATCAAGATCTgGGCAGGACTACGAGGCTGGCTC
SEQ ID NO: 203
TCAAAAGTTTCCATGTGTTTCTgGTATTCCTTAATTGTACAGAGA
SEQ ID NO: 204
ACTGTTTCCATTACAGTTGTCTgTGTTAGTGATGGCTGAGTGGTG
SEQ ID NO: 205
TTTAGTACCTTGGCAAAGTCTCgAACATCTTCTCCTGATGTAGTC
SEQ ID NO: 206
ATTTGTGCAAAGTTGAGTCTTCgAAACTGAGCAAATTTGCTCTCA
SEQ ID NO: 207
TGGGGACGCCTCTGTTCCAAATcCTGCATTGTTGCCTGTAAGAAC
SEQ ID NO: 208
GTCTCCTATGAACTCGAGAAGCcGCAAAaccaaggaagagaaaga
SEQ ID NO: 209
GAGAGTTTGGTTTCTGACTGCTgGACCCATGTCCTGATGGCACTC
SEQ ID NO: 210
TGCGTATTTGCCACCAGAAATAcATACCACACAATGATTTAGCTG
SEQ ID NO: 211
TTTGGGTTATCCTCTGAATGTCgCATCAAATTTTCAAGTGACTGA
SEQ ID NO: 212
AGGACACGGATCCTCCCTGTTCgTCCCCTATTATGAAGAATCAAA
SEQ ID NO: 213
TGCTTGTTAAAAAACTTACTTCgATCCGTAATGATTGTTCTAGCC
SEQ ID NO: 214
TTTTGCTCCACATCTTTTCCTAcCTAATGTTGAGAGACTTTTTCC
SEQ ID NO: 215
CCTGCCAGTGGAGGATTATATTcCAAATCAAACCAAGAGTCAGTT
SEQ ID NO: 216
GGATAATTACGAGTTGATTGTCgGACCCAGCTCAGGAGAATCTTT
SEQ ID NO: 217
TTTAGACTGGGCTGAATTGTCTgAATATCACTGACTAAAagctaa
SEQ ID NO: 218
GTACTACTTACATTATTGTTCTgCAAAACCCGCAGTGCCTTGTTG
SEQ ID NO: 219
TTCATTTGCTCCTCTAGCTTTTgACAATGCTCAACCAGCTGGGAG
SEQ ID NO: 220
TCAATCTGAGACAGGACTCTTTgGGCAGCCTCCTTCCCCTGAtta
SEQ ID NO: 221
TTCagctcctctttcttcttctgcaaTTCCCGATCAATTTCCTAT
SEQ ID NO: 222
AAAGCTaAGGGGACGAGGCAGGC
SEQ ID NO: 223
GAAAGCTaAGGGGACGAGGCAGG
SEQ ID NO: 224
AAATaGATGGCTGAAGTTGATGT
SEQ ID NO: 225
GAAATaGATGGCTGAAGTTGATG
SEQ ID NO: 226
AAGAAATaGATGGCTGAAGTTGA
SEQ ID NO: 227
CTGAAGAAATaGATGGCTGAAGTTGAT
SEQ ID NO: 228
AAATaGCGGCGTTTTCATTATGA
SEQ ID NO: 229
GAGAAATaGCGGCGTTTTCATTATGAT
SEQ ID NO: 230
AAATCaATGGTTGAGCTCTGAGA
SEQ ID NO: 231
GCAAATCaATGGTTGAGCTCTGA
SEQ ID NO: 232
TGCAAATCaATGGTTGAGCTCTGAGAT
SEQ ID NO: 233
AACaTGAGTAGTAGCAAAAGCAG
SEQ ID NO: 234
GAAACaTGAGTAGTAGCAAAAGC
SEQ ID NO: 235
GGAAACaTGAGTAGTAGCAAAAG
SEQ ID NO: 236
TGGAAACaTGAGTAGTAGCAAAA
SEQ ID NO: 237
AACTCaACCAGAAAAAAAGCAGC
SEQ ID NO: 238
CAACTCaACCAGAAAAAAAGCAG
SEQ ID NO: 239
AAGGGATCCTGaCAGCCCGTGGGCGAT
SEQ ID NO: 240
AATATTCaTACAGTCTCAAGAGT
SEQ ID NO: 241
AATCaAGGCTTAGGGGAAGAAGT
SEQ ID NO: 242
GAATCaAGGCTTAGGGGAAGAAG
SEQ ID NO: 243
TGAATCaAGGCTTAGGGGAAGAA
SEQ ID NO: 244
TTGAATCaAGGCTTAGGGGAAGA
SEQ ID NO: 245
CTTGAATCaAGGCTTAGGGGAAGAAGT
SEQ ID NO: 246
AATGaCAACGTCTTACTGAAGAA
SEQ ID NO: 247
AAATGaCAACGTCTTACTGAAGA
SEQ ID NO: 248
CAAATGaCAACGTCTTACTGAAG
SEQ ID NO: 249
TCAAATGaCAACGTCTTACTGAA
SEQ ID NO: 250
ACAAaTAAGTAAAAAGCCTAAAA
SEQ ID NO: 251
ACAGaTGTGTCATGTGTGAGAAA
SEQ ID NO: 252
AACAGaTGTGTCATGTGTGAGAA
SEQ ID NO: 253
GAACAGaTGTGTCATGTGTGAGA
SEQ ID NO: 254
AGAACAGaTGTGTCATGTGTGAG
SEQ ID NO: 255
ACCTaTGGATAATTACGAGTTGA
SEQ ID NO: 256
AACCTaTGGATAATTACGAGTTG
SEQ ID NO: 257
TTAACCTaTGGATAATTACGAGT
SEQ ID NO: 258
TTAACCTaTGGATAATTACGAGTTGAT
SEQ ID NO: 259
ACTaCTCCAATTCCTTCAAAGGA
SEQ ID NO: 260
AACTaCTCCAATTCCTTCAAAGG
SEQ ID NO: 261
AAACTaCTCCAATTCCTTCAAAGGAAT
SEQ ID NO: 262
ACTaCTTGTCAATGAATGTGAGG
SEQ ID NO: 263
AACTaCTTGTCAATGAATGTGAG
SEQ ID NO: 264
CAACTaCTTGTCAATGAATGTGA
SEQ ID NO: 265
CCAACTaCTTGTCAATGAATGTG
SEQ ID NO: 266
GCCAACTaCTTGTCAATGAATGT
SEQ ID NO: 267
ACTaGATGAGACTGGAACCCCAG
SEQ ID NO: 268
TAGACTaGATGAGACTGGAACCCCAGT
SEQ ID NO: 269
ACTGCTaGCAAACCACACTATTCCAGT
SEQ ID NO: 270
agaaactattcaGCTTCTGTTAG
SEQ ID NO: 271
AGATaGACAGAAGACCGCTGGGT
SEQ ID NO: 272
TAGATaGACAGAAGACCGCTGGG
SEQ ID NO: 273
GTAGATaGACAGAAGACCGCTGG
SEQ ID NO: 274
TGTAGATaGACAGAAGACCGCTG
SEQ ID NO: 275
CTGTAGATaGACAGAAGACCGCTGGGT
SEQ ID NO: 276
AGTaGAGTGAACTTCGGAAAAAG
SEQ ID NO: 277
AAGTaGAGTGAACTTCGGAAAAA
SEQ ID NO: 278
CAAGTaGAGTGAACTTCGGAAAA
SEQ ID NO: 279
TCAAGTaGAGTGAACTTCGGAAA
SEQ ID NO: 280
TCAAGTaGAGTGAACTTCGGAAAAAGT
SEQ ID NO: 281
AGTaGGATCACATGTGCCAACAG
SEQ ID NO: 282
CTCAGTaGGATCACATGTGCCAA
SEQ ID NO: 283
TCAGTaGGATCACATGTGCCAACAGGT
SEQ ID NO: 284
AGTCTTCaGAGTTTCATGGCAGT
SEQ ID NO: 285
CTGCAGTCTTCaGAGTTTCATGGCAGT
SEQ ID NO: 286
AGTGaATAAAGGTTAGACATTAA
SEQ ID NO: 287
AGTTaTTCTTCCAAAGCAGCAGT
SEQ ID NO: 288
AAGTTaTTCTTCCAAAGCAGCAG
SEQ ID NO: 289
TTAAGTTaTTCTTCCAAAGCAGC
SEQ ID NO: 290
CTTAAGTTaTTCTTCCAAAGCAGCAGT
SEQ ID NO: 291
ATCaATCTCCCAATACCTGGAGA
SEQ ID NO: 292
CATCaATCTCCCAATACCTGGAG
SEQ ID NO: 293
CCATCaATCTCCCAATACCTGGA
SEQ ID NO: 294
CCCATCaATCTCCCAATACCTGG
SEQ ID NO: 295
GCCCATCaATCTCCCAATACCTG
SEQ ID NO: 296
ATCAGTaGGATGAAGTACAAGAA
SEQ ID NO: 297
AATCAGTaGGATGAAGTACAAGA
SEQ ID NO: 298
ATCAGTGaGATGAAGTACAAGAA
SEQ ID NO: 299
ATCaTTCAATTTGAGGTTGAAGA
SEQ ID NO: 300
AATCaTTCAATTTGAGGTTGAAG
SEQ ID NO: 301
TAATCaTTCAATTTGAGGTTGAA
SEQ ID NO: 302
TTAATCaTTCAATTTGAGGTTGA
SEQ ID NO: 303
TTTAATCaTTCAATTTGAGGTTG
SEQ ID NO: 304
ATCCTaTAGGTCACTGAAGAGGT
SEQ ID NO: 305
CATCCTaTAGGTCACTGAAGAGG
SEQ ID NO: 306
CCATCCTaTAGGTCACTGAAGAG
SEQ ID NO: 307
TCCCATCCTaTAGGTCACTGAAGAGGT
SEQ ID NO: 308
ATCTaTTCCACCAGGGCCTGAGC
SEQ ID NO: 309
ACCATCTaTTCCACCAGGGCCTG
SEQ ID NO: 310
ATCTaTTCCACCAGGGCCTGAGCTGAT
SEQ ID NO: 311
ATCTaTTCCCTTGTGGTCACCGT
SEQ ID NO: 312
GATCTaTTCCCTTGTGGTCACCG
SEQ ID NO: 313
GATCTaTTCCCTTGTGGTCACCGTAGT
SEQ ID NO: 314
ATGaAAGGAGGGTCCCTATACAG
SEQ ID NO: 315
GTCATGaAAGGAGGGTCCCTATACAGT
SEQ ID NO: 316
ATGaCTGGAAGCTAAGGAAGAAG
SEQ ID NO: 317
AATGaCTGGAAGCTAAGGAAGAA
SEQ ID NO: 318
CAATGaCTGGAAGCTAAGGAAGA
SEQ ID NO: 319
ACAATGaCTGGAAGCTAAGGAAG
SEQ ID NO: 320
CACAATGaCTGGAAGCTAAGGAA
SEQ ID NO: 321
ATTTaAGCTTCAATTTCTCCTTG
SEQ ID NO: 322
ATTTaTGCAAAGTTGAGTCTTCG
SEQ ID NO: 323
attttaaCTCCTACTCAGACTGT
SEQ ID NO: 324
tattttaaCTCCTACTCAGACTG
SEQ ID NO: 325
CACaCAGAATCTACTGGCCAGAA
SEQ ID NO: 326
CTCACaCAGAATCTACTGGCCAG
SEQ ID NO: 327
CACTaGCGGAGGTCTTTGGCCAA
SEQ ID NO: 328
CACTTTaTTGCTCTTGCAGAGAA
SEQ ID NO: 329
CCACTTTaTTGCTCTTGCAGAGA
SEQ ID NO: 330
CAGaTAAAGTGTGTAAAGGACAG
SEQ ID NO: 331
CAACAGaTAAAGTGTGTAAAGGA
SEQ ID NO: 332
TCAACAGaTAAAGTGTGTAAAGG
SEQ ID NO: 333
CATTTaAGCTGCGTCCACCTTGT
SEQ ID NO: 334
GCATTTaAGCTGCGTCCACCTTG
SEQ ID NO: 335
CCATTCaGGGATGCTTCGCAAAA
SEQ ID NO: 336
CCCATTCaGGGATGCTTCGCAAA
SEQ ID NO: 337
CCCAGAaGTAATTGAATGTGGAA
SEQ ID NO: 338
GCCCAGAaGTAATTGAATGTGGA
SEQ ID NO: 339
CCGaTAAGTTTGACGCCAGCCTG
SEQ ID NO: 340
ACTCCGaTAAGTTTGACGCCAGC
SEQ ID NO: 341
CACTCCGaTAAGTTTGACGCCAG
SEQ ID NO: 342
CCTaAATGATCCACTTTGTGATG
SEQ ID NO: 343
CCTaCAGAGAAAGGTGCAGACGC
SEQ ID NO: 344
TCCTaCAGAGAAAGGTGCAGACG
SEQ ID NO: 345
GTTCCTaCAGAGAAAGGTGCAGA
SEQ ID NO: 346
AGTTCCTaCAGAGAAAGGTGCAG
SEQ ID NO: 347
CCTaGGCAGACTGGATGCTCTGT
SEQ ID NO: 348
TCCTaGGCAGACTGGATGCTCTG
SEQ ID NO: 349
CTATTGGAGCCTaTAAGTATACTGGAT
SEQ ID NO: 350
CTCaAAGTGCCTGTGTGCAATAG
SEQ ID NO: 351
CTCCTCaAAGTGCCTGTGTGCAA
SEQ ID NO: 352
CTCCTCaAAGTGCCTGTGTGCAATAGT
SEQ ID NO: 353
TTTCTCCTCaAAGTGCCTGTGTGCAAT
SEQ ID NO: 354
CTCaCTCACTCACCCTGCAAAGG
SEQ ID NO: 355
TCTCaCTCACTCACCCTGCAAAG
SEQ ID NO: 356
CTCTCaCTCACTCACCCTGCAAA
SEQ ID NO: 357
CCTCTCaCTCACTCACCCTGCAA
SEQ ID NO: 358
CTCaCTCTATGGCCTGCAGCATG
SEQ ID NO: 359
TTTCTCaCTCTATGGCCTGCAGC
SEQ ID NO: 360
TTTTCTCaCTCTATGGCCTGCAG
SEQ ID NO: 361
CTCaGGAGGTGACAGCTATCCAG
SEQ ID NO: 362
CTGCTCaGGAGGTGACAGCTATCCAGT
SEQ ID NO: 363
ctctCaCTTTCTCTCATCTGTGA
SEQ ID NO: 364
cctctCaCTTTCTCTCATCTGTG
SEQ ID NO: 365
tcctctCaCTTTCTCTCATCTGT
SEQ ID NO: 366
ttcctCtCaCTTTCTCTCATCTGTGAT
SEQ ID NO: 367
CTGaAAGAAGCTCTCCTCCCAGC
SEQ ID NO: 368
GCTGaAAGAAGCTCTCCTCCCAG
SEQ ID NO: 369
CTGaATTACTCGCTCAGAAGCTG
SEQ ID NO: 370
AGCTGaATTACTCGCTCAGAAGC
SEQ ID NO: 371
CAGCTGaATTACTCGCTCAGAAG
SEQ ID NO: 372
ACAGCTGaATTACTCGCTCAGAA
SEQ ID NO: 373
CTGAGTaGAAGGCGGTAAACCGT
SEQ ID NO: 374
TCTGAGTaGAAGGCGGTAAACCG
SEQ ID NO: 375
cttaccaaaggtcacaaaGGTAA
SEQ ID NO: 376
gacttaccaaaggtcacaaaGGT
SEQ ID NO: 377
tgacttaccaaaggtcacaaaGG
SEQ ID NO: 378
atgacttaccaaaggtcacaaaG
SEQ ID NO: 379
agatgacttaccaaaggtcacaaaGGT
SEQ ID NO: 380
CTTaGAGAGAGTCAATGAGGAGA
SEQ ID NO: 381
TCTTaGAGAGAGTCAATGAGGAG
SEQ ID NO: 382
ATCTTaGAGAGAGTCAATGAGGA
SEQ ID NO: 383
GATCTTaGAGAGAGTCAATGAGG
SEQ ID NO: 384
TGATCTTaGAGAGAGTCAATGAG
SEQ ID NO: 385
GATCTTaGAGAGAGTCAATGAGGAGAT
SEQ ID NO: 386
CTTaTAGACGCTGCTCAAAATTG
SEQ ID NO: 387
TTTCTTaTAGACGCTGCTCAAAA
SEQ ID NO: 388
CTTTCTTaTAGACGCTGCTCAAA
SEQ ID NO: 389
TACTTTCTTaTAGACGCTGCTCAAAAT
SEQ ID NO: 390
CTTaTTCTAGATCTTCTTGAAGC
SEQ ID NO: 391
CTTaTTGAGGCAAAACTTGGAAG
SEQ ID NO: 392
TCACTTaTTGAGGCAAAACTTGG
SEQ ID NO: 393
CTCACTTaTTGAGGCAAAACTTG
SEQ ID NO: 394
ACTTaTTGAGGCAAAACTTGGAAGAGT
SEQ ID NO: 395
CTTTGaTGGGAAGAAGTAGAGGA
SEQ ID NO: 396
GCTTTGaTGGGAAGAAGTAGAGG
SEQ ID NO: 397
TGCTTTGaTGGGAAGAAGTAGAG
SEQ ID NO: 398
gaaaGTCTATGCCAGAAAGGAGG
SEQ ID NO: 399
ggaaaGTCTATGCCAGAAAGGAG
SEQ ID NO: 400
tggaaaGTCTATGCCAGAAAGGA
SEQ ID NO: 401
gtggaaaGTCTATGCCAGAAAGG
SEQ ID NO: 402
tgtggaaaGTCTATGCCAGAAAG
SEQ ID NO: 403
GACATaGGTCCAGCAGTCAGAAA
SEQ ID NO: 404
GGACATaGGTCCAGCAGTCAGAA
SEQ ID NO: 405
AGGACATaGGTCCAGCAGTCAGA
SEQ ID NO: 406
GACGCTaCTCAAAATTGGCTGGT
SEQ ID NO: 407
AGACGCTaCTCAAAATTGGCTGG
SEQ ID NO: 408
TGTAGACGCTaCTCAAAATTGGCTGGT
SEQ ID NO: 409
GAGaTATGAAGATAAGTGAAAAA
SEQ ID NO: 410
AGAGaTATGAAGATAAGTGAAAA
SEQ ID NO: 411
AAGAGaTATGAAGATAAGTGAAA
SEQ ID NO: 412
GAAGAGaTATGAAGATAAGTGAA
SEQ ID NO: 413
TGAAGAGaTATGAAGATAAGTGA
SEQ ID NO: 414
GAAGAGaTATGAAGATAAGTGAAAAAT
SEQ ID NO: 415
GAGGCTTaTTTGATGCTATCTGC
SEQ ID NO: 416
GATaTAAGTCGTGTATATTAATG
SEQ ID NO: 417
CAGATaTAAGTCGTGTATATTAA
SEQ ID NO: 418
GTACAGATaTAAGTCGTGTATATTAAT
SEQ ID NO: 419
GATTaGATTTTCTGTTGGGAGGA
SEQ ID NO: 420
AGATTaGATTTTCTGTTGGGAGG
SEQ ID NO: 421
CAGATTaGATTTTCTGTTGGGAG
SEQ ID NO: 422
TCAGATTaGATTTTCTGTTGGGA
SEQ ID NO: 423
TCAGATTaGATTTTCTGTTGGGAGGAT
SEQ ID NO: 424
GCTaATTATAGAAAGCGATGATG
SEQ ID NO: 425
TAGCTaATTATAGAAAGCCGATGA
SEQ ID NO: 426
GTAGCTaATTATAGAAAGCGATG
SEQ ID NO: 427
TTGTAGCTaATTATAGAAAGCGATGAT
SEQ ID NO: 428
GCTGaCGGGAAATTGAGAGCAAA
SEQ ID NO: 429
CGCTGaCGGGAAATTGAGAGCAA
SEQ ID NO: 430
AGCGCTGaCGGGAAATTGAGAGC
SEQ ID NO: 431
AGCGCTGaCGGGAAATTGAGAGCAAAT
SEQ ID NO: 432
GCTGaTCTGATGGCCTGGCTTTG
SEQ ID NO: 433
AGCTGaTCTGATGGCCTGGCTTTGAAT
SEQ ID NO: 434
GCTTTCTaTAATTCATCTGGAGT
SEQ ID NO: 435
AACTGCTTTCTaTAATTCATCTGGAGT
SEQ ID NO: 436
GGCCaTATGTACTTTCTAGCTTTCAAT
SEQ ID NO: 437
GGCCCTTTGCTaTAAGTATTGGCCAGT
SEQ ID NO: 438
GGCTaAGTGGTGGTGACAGCCTG
SEQ ID NO: 439
GATGGCTaAGTGGTGGTGACAGC
SEQ ID NO: 440
GTACTaATACCACTGATGAGAAA
SEQ ID NO: 441
TGTACTaATACCACTGATGAGAA
SEQ ID NO: 442
TTGTACTaATACCACTGATGAGA
SEQ ID NO: 443
CTTGTACTaATACCACTGATGAGAAAT
SEQ ID NO: 444
GTCATaGAAGGAGGGTCCCTATACAGT
SEQ ID NO: 445
GTCaTTGTGTGGCTGACTGCTGG
SEQ ID NO: 446
AGTCaTTGTGTGGCTGACTGCTG
SEQ ID NO: 447
CCAGTCaTTGTGTGGCTGACTGC
SEQ ID NO: 448
TCCAGTCaTTGTGTGGCTGACTG
SEQ ID NO: 449
GTCTTaTAAAAGAACCCAGCGGT
SEQ ID NO: 450
TGTCTTaTAAAAGAACCCAGCGG
SEQ ID NO: 451
ATGTCTTaTAAAAGAACCCAGCG
SEQ ID NO: 452
GGATGTCTTaTAAAAGAACCCAGCGGT
SEQ ID NO: 453
GTGaAGTGAACTTCGGAAAAAGT
SEQ ID NO: 454
AGTGaAGTGAACTTCGGAAAAAG
SEQ ID NO: 455
AAGTGaAGTGAACTTCGGAAAAA
SEQ ID NO: 456
CAAGTGaAGTGAACTTCGGAAAA
SEQ ID NO: 457
TCAAGTGaAGTGAACTTCGGAAA
SEQ ID NO: 458
TCAAGTGaAGTGAACTTCGGAAAAAGT
SEQ ID NO: 459
GTGaATCGAATTCTGCCAGTTGC
SEQ ID NO: 460
GGTGaATCGAATTCTGCCAGTTG
SEQ ID NO: 461
GCCGGTGaATCGAATTCTGCCAG
SEQ ID NO: 462
GTGaCAGACAAATGTAGATGTGG
SEQ ID NO: 463
AGTGaCAGACAAATGTAGATGTG
SEQ ID NO: 464
CAGTGaCAGACAAATGTAGATGT
SEQ ID NO: 465
CCAGTGaCAGACAAATGTAGATG
SEQ ID NO: 466
CTCCGCCAGTGaCAGACAAATGTAGAT
SEQ ID NO: 467
GTGaCTCTGCACTAGGCTGAATG
SEQ ID NO: 468
CAGTGaCTCTGCACTAGGCTGAA
SEQ ID NO: 469
CCAGTGaCTCTGCACTAGGCTGA
SEQ ID NO: 470
ACCAGTGaCTCTGCACTAGGCTG
SEQ ID NO: 471
TACCAGTGaCTCTGCACTAGGCTGAAT
SEQ ID NO: 472
GTGCTaAGATGCTGGACCAAAGT
SEQ ID NO: 473
AGTGCTaAGATGCTGGACCAAAG
SEQ ID NO: 474
AAGTGCTaAGATGCTGGACCAAA
SEQ ID NO: 475
GAAAGTGCTaAGATGCTGGACCAAAGT
SEQ ID NO: 476
GTTaAAGATCTGATAGCCGGTTG
SEQ ID NO: 477
GAGGTTaAAGATCTGATAGCCGG
SEQ ID NO: 478
TGAGGTTaAAGATCTGATAGCCG
SEQ ID NO: 479
TTTGAGGTTaAAGATCTGATAGCCGGT
SEQ ID NO: 480
GTTaGCACATGTGATCCCACTGA
SEQ ID NO: 481
TGTTaGCACATGTGATCCCACTG
SEQ ID NO: 482
CTGTTaGCACATGTGATCCCACTGAGT
SEQ ID NO: 483
GTTaGGATAATTTAGTCCAAAAA
SEQ ID NO: 484
TGTTaGGATAATTTAGTCCAAAA
SEQ ID NO: 485
GTGTTaGGATAATTTAGTCCAAA
SEQ ID NO: 486
GGTGTTaGGATAATTTAGTCCAA
SEQ ID NO: 487
TAAGaTCAGATTATTTTGCTTAG
SEQ ID NO: 488
TAATAaGGGACGAACAGGGAGGA
SEQ ID NO: 489
ATAATAaGGGACGAACAGGGAGG
SEQ ID NO: 490
CATAATAaGGGACGAACAGGGAG
SEQ ID NO: 491
TCATAATAAGGGACGAACAGGGAGGAT
SEQ ID NO: 492
TCAAATCaCCTGCAGGTAAAAGC
SEQ ID NO: 493
TCAaGGGTACATGATGGATTTGA
SEQ ID NO: 494
GTCAaGGGTACATGATGGATTTG
SEQ ID NO: 495
CTTCTTTGTCAaGGGTACATGATGGAT
SEQ ID NO: 496
tcaatttgggaagcaGCATATTG
SEQ ID NO: 497
TCAGCTaTAGCCACACCAGAAGT
SEQ ID NO: 498
TTCAGCTaTAGCCACACCAGAAG
SEQ ID NO: 499
TCTTTCAGCTaTAGCCACACCAGAAGT
SEQ ID NO: 500
TCCTTTCaCATCTTACGGGACAA
SEQ ID NO: 501
ATTTCCTTTCaCATCTTACGGGACAAT
SEQ ID NO: 502
TCGTaGCTTCTTTCTGCTGAGGA
SEQ ID NO: 503
ATCGTaGCTTCTTTCTGCTGAGG
SEQ ID NO: 504
TATCGTaGCTTCTTTCTGCTGAG
SEQ ID NO: 505
TTATCGTaGCTTCTTTCTGCTGA
SEQ ID NO: 506
TCTaATACTCCAGCCAGTTAAGT
SEQ ID NO: 507
TTCTaATACTCCAGCCAGTTAAG
SEQ ID NO: 508
GTTCTaATACTCCAGCCAGTTAA
SEQ ID NO: 509
TTGTTCTaATACTCCAGCCAGTTAAGT
SEQ ID NO: 510
TGATGTTGTTCTaATACTCCAGCCAGT
SEQ ID NO: 511
TCTaGGCAGGACTACGAGGCTGG
SEQ ID NO: 512
ATCTaGGCAGGACTACGAGGCTG
SEQ ID NO: 513
AGATCTaGGCAGGACTACGAGGC
SEQ ID NO: 514
AAGATCTaGGCAGGACTACGAGG
SEQ ID NO: 515
TCTaGTATTCCTTAATTGTACAG
SEQ ID NO: 516
TGTTTCTaGTATTCCTTAATTGT
SEQ ID NO: 517
TCTaTGTTAGTGATGGCTGAGTG
SEQ ID NO: 518
GTCTaTGTTAGTGATGGCTGAGT
SEQ ID NO: 519
TGTCTaTGTTAGTGATGGCTGAG
SEQ ID NO: 520
TTGTCTaTGTTAGTGATGGCTGA
SEQ ID NO: 521
GTTGTCTaTGTTAGTGATGGCTG
SEQ ID NO: 522
TGTCTaTGTTAGTGATGGCTGAGTGGT
SEQ ID NO: 523
AGTTGTCTaTGTTAGTGATGGCTGAGT
SEQ ID NO: 524
TCTCaAACATCTTCTCCTGATGT
SEQ ID NO: 525
GTCTCaAACATCTTCTCCTGATG
SEQ ID NO: 526
AAGTCTCaAACATCTTCTCCTGA
SEQ ID NO: 527
GTCTCaAACATCTTCTCCTGATGTAGT
SEQ ID NO: 528
TCTTCaAAACTGAGCAAATTTGC
SEQ ID NO: 529
GTCTTCaAAACTGAGCAAATTTG
SEQ ID NO: 530
TGCAGaATTTGGAACAGAGGCGT
SEQ ID NO: 531
ATGCAGaATTTGGAACAGAGGCG
SEQ ID NO: 532
AATGCAGaATTTGGAACAGAGGC
SEQ ID NO: 533
TGCaGCTTCTCGAGTTCATAGGA
SEQ ID NO: 534
TTGCaGCTTCTCGAGTTCATAGG
SEQ ID NO: 535
TGCTaGACCCATGTCCTGATGGC
SEQ ID NO: 536
CTGCCTaGACCCATGTCCTGATGG
SEQ ID NO: 537
ACTGCTaGACCCATGTCCTGATG
SEQ ID NO: 538
TTCTGACTGCTaGACCCATGTCCTGAT
SEQ ID NO: 539
TGGTATaTATTTCTGGTGGCAAA
SEQ ID NO: 540
GTGGTATaTATTTCTGGTGGCAA
SEQ ID NO: 541
GTGTGGTATaTATTTCTGGTGGCAAAT
SEQ ID NO: 542
TGTCaCATCAAATTTTCAAGTGA
SEQ ID NO: 543
ATGTCaCATCAAATTTTCAAGTG
SEQ ID NO: 544
TGTTCaTCCCCTATTATGAAGAA
SEQ ID NO: 545
CTGTTCaTCCCCTATTATGAAGA
SEQ ID NO: 546
CCTGTTCaTCCCCTATTATGAAG
SEQ ID NO: 547
CCCTGTTCaTCCCCTATTATGAAGAAT
SEQ ID NO: 548
TTACTTCaATCCGTAATGATTGT
SEQ ID NO: 549
TTAGaTAGGAAAAGATGTGGAGC
SEQ ID NO: 550
ATTAGaTAGGAAAAGATGTGGAG
SEQ ID NO: 551
CATTAGaTAGGAAAAGATGTGGA
SEQ ID NO: 552
ACATTAGaTAGGAAAAGATGTGG
SEQ ID NO: 553
TTGaAATATAATCCTCCACTGGC
SEQ ID NO: 554
TTTGaAATATAATCCTCCACTGG
SEQ ID NO: 555
ATTTGaAATATAATCCTCCACTG
SEQ ID NO: 556
TTGTCaGACCCAGCTCAGGAGAA
SEQ ID NO: 557
ATTGTCaGACCCAGCTCAGGAGA
SEQ ID NO: 558
GATTGTCaGACCCAGCTCAGGAG
SEQ ID NO: 559
TGATTGTCaGACCCAGCTCAGGAGAAT
SEQ ID NO: 560
TTGTCTaAATATCACTGACTAAA
SEQ ID NO: 561
ATTGTCTaAATATCACTGACTAA
SEQ ID NO: 562
TTGTTCTaCAAAACCCGCAGTGC
SEQ ID NO: 563
TTTaACAATGCTCAACCAGCTGG
SEQ ID NO: 564
TTTTaACAATGCTCAACCAGCTG
SEQ ID NO: 565
GCTTTTaACAATGCTCAACCAGC
SEQ ID NO: 566
AGCTTTTaACAATGCTCAACCAG
SEQ ID NO: 567
TTTaGGCAGCCTCCTTCCCCTGA
SEQ ID NO: 568
CTTTaGGCAGCCTCCTTCCCCTG
SEQ ID NO: 569
tttcttcttctacaaTTCCCGATCAAT
SEQ ID NO: 570
GTTATCTCCTGTTCTGCAGC
SEQ ID NO: 571
GTTTATGTCACCAGAGTAAC
SEQ ID NO: 572
GAGGTAATAGAGCCAAGCCCT
SEQ ID NO: 573
GCAAGAATTCCACTTTTCACTTCCT
SEQ ID NO: 574
CTGTCATCTCCAAACTAGAAATGC
SEQ ID NO: 575
GCAGCCTCTTGCTCACTTACTC
SEQ ID NO: 576
GATGACAGGCAGGGGCACCG
SEQ ID NO: 577
TTCCAGTGGTTCAATGGTCA
SEQ ID NO: 578
CTTTCAACCCGAACGGAGAC
SEQ ID NO: 579
GAGCGAGCAGCGTCTTCGAG
SEQ ID NO: 580
GCAGACGGCAGTCACTAGGG
SEQ ID NO: 581
GGGAAGCTGGGTGAATGGAG
SEQ ID NO: 582
AGCTGTTTGGGAGGTCAGAA
SEQ ID NO: 583
AGGGAGCAGGAAAGTGAGGT
SEQ ID NO: 584
GTCGCAGGACAGCTTTTCCT
SEQ ID NO: 585
TGTAGCTACGCCTGTGATGG
SEQ ID NO: 586
TGCCCTGAGATCTTTTCCTC
SEQ ID NO: 587
GATCCAGGTGCTGCAGAAGG
SEQ ID NO: 588
CTCTTGCCTCCACTGGTTGT
SEQ ID NO: 589
TCGGTAGGATGCCCTACATC
SEQ ID NO: 590
ATCCTACAGCATGGTGGCTG
SEQ ID NO: 591
AGTGGTCTCCGGAAACCTCCGCGCCCCGCAAC
SEQ ID NO: 592
TCCTTGAAGAAGATGGTGCG
SEQ ID NO: 593
ACACTCTTTCCCTACACGACGCTCTTCCGATCTGAACTCATTACTGCTGCCCA
GA
SEQ ID NO: 594
GTGACTGGAGTTCAGACGTGTGCTCTTCCGATCGACCTGTTCGGCTTCTTCCT
TA
SEQ ID NO: 595
ACACTCTTTCCCTACACGACGCTCTTCCGATCTAAATTTCCACTGTCTTCTCTT
GAGT
SEQ ID NO: 596
GTGACTGGAGTTCAGACGTGTGCTCTTCCGATCGCTTGCCTCTGACCTGTCCT
AT
SEQ ID NO: 597
ACACTCTTTCCCTACACGACGCTCTTCCGATCTGTGACTAGGGGCAAAGCAA
GAT
SEQ ID NO: 598
GTGACTGGAGTTCAGACGTGTGCTCTTCCGATCCTTCCAAACTTTCTGCCCAT
TC
SEQ ID NO: 599
ACACTCTTTCCCTACACGACGCTCTTCCGATCTAACACAGCGTGCTCTTTCCT
TAG
SEQ ID NO: 600
GTGACTGGAGTTCAGACGTGTGCTCTTCCGATCGTTCAGAAGAACATCCCGT
TGAC
SEQ ID NO: 601
AATGATACGGCGACCACCGAGATCTACACTCTTTCCCTACACGAC
SEQ ID NO: 602
CAAGCAGAAGACGGCATACGAGATCTTGTAGTGACTGGAGTTCAGACGT
SEQ ID NO: 603
CAAGCAGAAGACGGCATACGAGATCAGATCGTGACTGGAGTTCAGACGT
SEQ ID NO: 604
CAAGCAGAAGACGGCATACGAGATCCGTCCGTGACTGGAGTTCAGACGT
SEQ ID NO: 605
CAAGCAGAAGACGGCATACGAGATATGTCAGTGACTGGAGTTCAGACGT
SEQ ID NO: 606
CAAGCAGAAGACGGCATACGAGATGTCCGCGTGACTGGAGTTCAGACGT
SEQ ID NO: 607
CAAGCAGAAGACGGCATACGAGATTTAGGCGTGACTGGAGTTCAGACGT
SEQ ID NO: 608
CAAGCAGAAGACGGCATACGAGATCGATGTGTGACTGGAGTTCAGACGT
SEQ ID NO: 609
CAAGCAGAAGACGGCATACGAGATTGACCAGTGACTGGAGTTCAGACGT
SEQ ID NO: 610
CAAGCAGAAGACGGCATACGAGATAGTCAAGTGACTGGAGTTCAGACGT
SEQ ID NO: 611
CAAGCAGAAGACGGCATACGAGATAGTTCCGTGACTGGAGTTCAGACGT
SEQ ID NO: 612
CAAGCAGAAGACGGCATACGAGATGATCAGGTGACTGGAGTTCAGACGT
SEQ ID NO: 613
CAAGCAGAAGACGGCATACGAGATACAGTGGTGACTGGAGTTCAGACGT
SEQ ID NO: 614
CAAGCAGAAGACGGCATACGAGATTATACTGTGACTGGAGTTCAGACGT
SEQ ID NO: 615
CAAGCAGAAGACGGCATACGAGATCAACAAGTGACTGGAGTTCAGACGT
SEQ ID NO: 616
CAAGCAGAAGACGGCATACGAGATGTTGTTGTGACTGGAGTTCAGACGT
SEQ ID NO: 617
CAAGCAGAAGACGGCATACGAGATTCGGTTGTGACTGGAGTTCAGACGT
SEQ ID NO: 618
CAAGCAGAAGACGGCATACGAGATAGTATTGTGACTGGAGTTCAGACGT
SEQ ID NO: 619
CAAGCAGAAGACGGCATACGAGATTCTTGTGTGACTGGAGTTCAGACGT
SEQ ID NO: 620
GAACAGCTGCAGAACAGGAGATAACAG
SEQ ID NO: 621
GTTaTCTCCTGTTCTGCAGCTGT
SEQ ID NO: 622
ATGACAGGCAGGGGCACCGCGG
SEQ ID NO: 623
GAGCGAGCAGCGTCTTCGAGAGT
SEQ ID NO: 624
GCAGACGGCAGTCACTAGGGGGC
SEQ ID NO: 625
GTCGCAGGACAGCTTTTCCTAGA
SEQ ID NO: 626
GGGAAGCTGGGTGAATGGAGCGA
SEQ ID NO: 627
GATCCAGGTGCIGCAGAAGGGAT
SEQ ID NO: 628
GTTATCTCCTGCTCTGCAGCAGA
SEQ ID NO: 629
GATATCTCCTGTTCTGCAGGAGA
SEQ ID NO: 630
GGATTTCCAAGTCTCCACCC
SEQ ID NO: 631
TCCCACCGTACACGCCTAC
SEQ ID NO: 632
GGTTTCAGACAAAATCAAAAAGAAGGAAGGTGCTCACATTCCTTAAATTAAG
GA
SEQ ID NO: 633
GGTTTTAGACAAAATCAAAAAGAAGGAAGGTGCTCACATTCCTTAAATTAAG
GA
SEQ ID NO: 634
GTGCTCACATTCCTTAAATTAAGG
SEQ ID NO: 635
GGCTCACATTCCTTAAATTAAGGA

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A fusion protein comprising a Cas9 nickase and at least one nucleotide deaminase,
wherein the Cas9 nickase comprises amino acid substitutions L1111R, D1135V, G1218R, A1322R, and T1337R with at least one additional amino acid substitution A262T, R324L, S409I, E480K, E543D, E1219V or M694I when compared to SEQ ID NO: 11.

2. (canceled)

3. (canceled)

4. The fusion protein of claim 1, wherein the Cas9 nickase comprises the amino acid sequence selected from the group consisting of SEQ ID NOs: 12-20.

5. The fusion protein of claim 1, wherein the Cas9 nickase when in conjunction with a bound guide RNA (gRNA) specifically binds to a target nucleic acid sequence.

6. The fusion protein of claim 1, wherein the Cas9 nickase recognizes an NGX protospacer adjacent motif (PAM) sequence wherein NGX comprises NGA, NGC, NGT, or NGG.

7. The fusion protein of claim 1, wherein the nucleotide deaminase is a cytidine deaminase or an adenine deaminase.

8. The fusion protein of claim 7, wherein the adenine deaminase is a dimeric adenine deaminase or a monomeric adenine deaminase domain.

9. The fusion protein of claim 8, wherein the monomeric adenine deaminase comprises amino acid substitutions A56G and V82G when compared to SEQ ID NO: 47.

10. The fusion protein of claim 7, wherein the adenine deaminase comprises the amino acid sequence set forth in SEQ ID NO: 22 or 23.

11. A system for base editing comprising:
a first nucleotide sequence encoding a N-terminal portion of a Cas9 nickase fused at its C-terminus to an intein-N; and
a second nucleotide sequence encoding an intein-C fused to the N-terminus of a C-terminal portion of the Cas9 nickase;
wherein the first nucleotide sequence further comprises a nucleotide sequence encoding a nucleotide deaminase fused to the N-terminus of the N-terminal portion of the Cas9 nickase,
wherein the Cas9 nickase comprises amino acid substitutions L1111R, D1135V, G1218R, A1322R, and T1337R with at least one additional amino acid substitution A262T, R324L, S409I, E480K, E543D, E1219V or M694I when compared to SEQ ID NO: 11.

12. (canceled)

13. (canceled)

14. The system of claim 11, wherein the Cas9 nickase comprises the amino acid sequence selected from the group consisting of SEQ ID NOs: 12-20.

15. The system of claim 11, wherein the Cas9 nickase recognizes an NGX protospacer adjacent motif (PAM) sequence, wherein NGX comprises NGA, NGC, NGT, or NGG.

16. The system of claim 11, wherein the N-terminal portion of the Cas9 nickase comprises the amino acid positions 2-573 of the amino acid sequence selected from the group consisting of SEQ ID NOs: 12-20 and the C-terminal portion of the Cas9 nickase comprises the amino acid positions 574-1368 of the amino acid sequence selected from the group consisting of SEQ ID NOs: 12-20.

17.-23. (canceled)

24. A composition comprising:
a first recombinant adeno-associated virus (AAV) particle comprising a first nucleotide sequence encoding a N-terminal portion of a Cas9 nickase fused at its C-terminus to an intein-N; and
a second recombinant AAV particle comprising a second nucleotide sequence encoding an intein-C fused to the N-terminus of a C-terminal portion of the Cas9 nickase;
wherein first nucleotide sequence further comprises a nucleotide sequence encoding a nucleotide deaminase fused to the N-terminus of the N-terminal portion of the Cas9 nickase,
wherein the Cas9 nickase comprises amino acid substitutions L1111R, D1135V, G1218R, A1322R, and T1337R with at least one additional amino acid substitution A262T, R324L, S409I, E480K, E543D, E1219V or M694I when compared to SEQ ID NO: 11.

25.-36. (canceled)

37. A method of treating a genetic disease in a subject, comprising administering a therapeutically effective amount of the composition of claim 24.

38. The method of claim 37, wherein the genetic disease is a muscular dystrophy or spinal muscular atrophy.

39. The method of claim 38, wherein the muscular dystrophy is Duchenne muscular dystrophy.

40. The method of claim 38, wherein the muscular dystrophy is dysferlinopathy.

41. (canceled)

42. (canceled)

43. The fusion protein of claim 1, further comprising the amino acid substitution R1335Q when compared to SEQ ID NO: 11.

44. The fusion protein of claim 1, further comprising the amino acid substitution R1335E when compared to SEQ ID NO: 11.
